(19) United States
(12) Patent Application Publication
(10) Pub. No.: US 2023/0061083 A1
(43) Pub. Date: Mar. 2, 2023

(54) KRAS G12C INHIBITOR COMPOUND AND USE THEREOF

(71) Applicant: Evopoint Biosciences Co., Ltd., Jiangsu (CN)

(72) Inventors: Yonghan HU, Jiangsu (CN); Xin LI, Jiangsu (CN); Jinfeng ZHAO, Jiangsu (CN); Yuchuan WU, Beijing (CN); Xiao LIU, Beijing (CN); Xi CHEN, Beijing (CN)

(73) Assignee: Evopoint Biosciences Co., Ltd., Jiangsu (CN)

(21) Appl. No.: 17/779,684

(22) PCT Filed: Nov. 27, 2020
(86) PCT No.: PCT/CN2020/132158

§ 371 Date: May 25, 2022

(30) Foreign Application Priority Data
Nov. 29, 2019 (CN) 201911201945.9
Mar. 6, 2020 (CN) 202010152172.6
Jun. 19, 2020 (CN) 202010564547.X

(51) Int. Cl. C07D 471/04

(52) U.S. Cl. CPC C07D 471/04

(57) ABSTRACT

A compound with the structure of formula I or a pharmaceutically acceptable salt, ester, isomer, solvate, prodrug, or isotope label thereof:

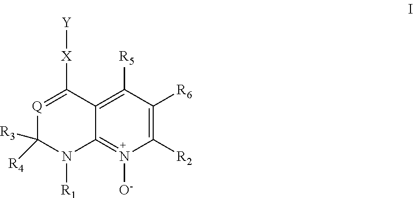

the KRAS G12C inhibitor compound has a good inhibitory effect on KRAS mutations, and can be used for the prevention and/or treatment of KRAS G12C-mediated diseases.

### TECHNICAL FIELD

The present invention relates to a novel KRAS G12C inhibitor compound and use of the inhibitor compound for preventing or treating a KRAS G12C-mediated disease.

### BACKGROUND

The Kirsten rat sarcoma virus gene homolog (KRAS) mutation was first described in the NSCLC gene in 1984. KRAS protein is a membrane-binding protein located on the inside of the cell membrane and, at the same time, on the EGFR signaling path, and is very important for the development and progression of tumors. KRAS protein normally has no activity of binding to GDP. When signals are transmitted to KRAS protein by extracellular growth differentiation factors, the protein's activity of binding to GTP is enhanced, leading to binding of the protein to GTP and thus to activation of the protein and opening of the signal system. Intracellular proteins are necessary for signaling in the processes such as growth, proliferation and angiogenesis of tumor cells. The KRAS gene is a determinant of signaling proteins. KRAS mutant genes code for abnormal proteins, which stimulate and facilitate the growth and spreading of malignant tumor cells, and are not affected by upstream EGFR signals. KRAS mutations promote proliferation, transformation and apoptosis resistance of cells by activating a variety of downstream signal transduction pathways in the cells such as the RAS-RAF-MEK-MAPK and P13K-AKT-mTOR pathways, thereby causing tumor development and progression.

According to the COSMIC statistics, about 30% of all human tumors, including 90% of pancreatic cancer cases, 45% of colon cancer cases and 35% of non-small cell lung cancer cases, involve point mutations in the KRAS gene. 80% of KRAS mutations occur at codon 12, resulting in single amino acid substitutions, i.e., substitutions of alanine (A), cysteine (C), aspartic acid (D), serine (S), arginine (R) and valine (V) for glycine (G), with the substitution of cysteine (C) for glycine (G) the most common. The KRAS G12C mutant protein is expressed in a large proportion of patients with lung cancer, especially those with non-small cell lung cancer (14%), and also in patients with colorectal cancer (4%) and patients with pancreatic cancer (2%).

The KRAS G12C mutation is getting more and more attention from experts and scholars as its high expression in tumor patients will also lead to resistance to other targeted drugs. However, the research and development of inhibitor drugs directly targeted at the KRAS G12C mutation is challenging for the biochemical complexity. The KRAS G12C target has been nothing short of synonymous with “undruggable” target in oncology, and has been regarded as “Qomolangma” in pharmaceutics. There has been no solution to the druggability problem over the last 30 years.

The research and development of new drugs is a rapidly developing area. The discovery of drug candidates is accelerated by the progress of technology. For these drug candidates, pharmacodynamic evaluations are necessary. Besides, the drug metabolism and kinetic properties are also very important indexes for new drug screening. An ideal drug needs to have a long duration of drug action and good bioavailability. A large number of drug candidates are eliminated each year because of poor pharmacokinetic parameters and metabolic characteristics. Therefore, the metabolic characteristics and pharmacokinetic parameters are important evaluation indexes for determining whether the candidate drug can be a patent medicine, and good pharmacokinetic parameters and metabolic characteristics are essential for lead compounds with development prospects. Therefore, KRAS G12C inhibitors with good pharmacokinetic characteristics provided would likely be more effective in vivo for pharmacodynamic effects.

### SUMMARY

### Problems to be Solved by the Present Invention

In order to solve the technical problems described above, the present invention is intended to provide a novel KRAS G12C inhibitor and use of the inhibitor for treating a KRAS G12C-mediated disease such as cancer.

### Solutions to the Problems

In order to solve the technical problems described above, the present invention provides the following technical solutions:

In one aspect, the present invention provides a compound having a structure of formula I or a pharmaceutically acceptable salt, ester, isomer, solvate, prodrug or isotopically labeled compound thereof:

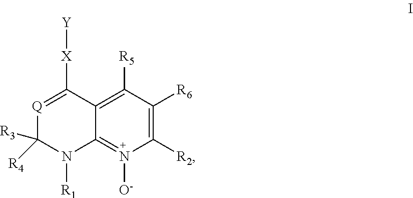

wherein,

R₁ is selected from C₆₋₁₀ aryl and 5- to 10-membered heteroaryl unsubstituted or substituted with R₇;

R₂ is selected from C₆₋₁₀ aryl and 5- to 10-membered heteroaryl unsubstituted or substituted with R₈;

R₃ and R₄ are each independently selected from hydrogen, deuterium, and C₁₋₆ alkyl, or R₃ and R₄ are joined to form 3- to 7-membered cycloalkyl or 3- to 7-membered heterocycloalkyl unsubstituted or optionally substituted with 1-3 substituents selected from deuterium, halogen, hydroxy and C₁₋₆ alkyl, or R₃ and R₄ form ═O, ═S, ═N—CN or ═CH₂;

R₅ and R₆ are each independently selected from hydrogen, deuterium and halogen;

each R₇ and R₈ are independently selected from hydrogen, deuterium, cyano, halogen, hydroxy, amino, C₁₋₆ alkyl, —NHC₁₋₆ alkyl, —N(C₁₋₆ alkyl)₂, C₃₋₆ cycloalkyl, C₁₋₆ alkoxy, C₂₋₆ alkenyl, C₂₋₆ alkynyl and —COOC₁₋₆ alkyl, wherein the amino, alkyl, cycloalkyl, alkenyl, and alkynyl are unsubstituted or substituted with 1-3 substituents selected from halogen, hydroxy, amino, acetyl and deuterium atom;

X is a 4- to 9-membered heterocyclyl unsubstituted or substituted with R₉, wherein X is a divalent group, as can be seen from the structure of formula I; each R9 is independently selected from hydrogen, deuterium, cyano, halogen, hydroxy, amino, C₁₋₆ alkyl and C₁₋₆ alkoxy, wherein the amino and alkyl are unsubstituted or substituted with 1-3 substituents selected from halogen, cyano, hydroxy, amino and deuterium atom;

Y is

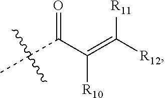

wherein R₁₀, R₁₁ and R₁₂ are each independently selected from hydrogen, deuterium, halogen, cyano, C₁₋₆ alkyl, C₃₋₆ cycloalkyl, 3- to 7-membered heterocyclyl, C₂₋₆ alkenyl, C₂₋₆ alkynyl, acetyl, propionyl, butyryl, and —COOC₁₋₆ alkyl, wherein the alkyl, cycloalkyl, alkenyl, alkynyl, acetyl, propionyl and butyryl are unsubstituted or substituted with 1-3 substituents selected from deuterium, halogen, cyano, hydroxy, amino, C₁₋₆ alkyl, —NHC₁₋₆ alkyl, —N(C₁₋₆ alkyl)₂ and 3- to 7-membered heterocyclyl; or R₁₀ and R₁₂ are joined to each other to form a triple bond;

Q is N or C-Q′, wherein Q′ is selected from hydrogen, deuterium, cyano, halogen and C₁₋₆ alkyl.

### Effects of the Invention

The novel KRAS G12C inhibitor compound provided by the present invention has a good inhibitory effect on KRAS mutation, and can be used for preventing and/or treating a KRAS G12C-mediated disease.

### DETAILED DESCRIPTION

In a first major aspect, the present invention provides a compound having a structure of formula I or a pharmaceutically acceptable salt, ester, isomer, solvate, prodrug or isotopically labeled compound thereof:

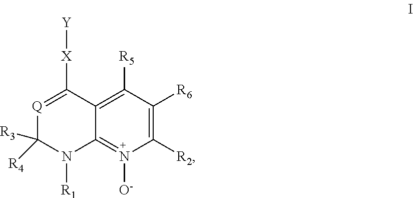

wherein,

R₁ is selected from C₆₋₁₀ aryl and 5- to 10-membered heteroaryl unsubstituted or substituted with R₇;

R₂ is selected from C₆₋₁₀ aryl and 5- to 10-membered heteroaryl unsubstituted or substituted with R₈;

R₃ and R₄ are each independently selected from hydrogen, deuterium, and C₁₋₆ alkyl, or R₃ and R₄ are joined to form 3- to 7-membered cycloalkyl or 3- to 7-membered heterocycloalkyl unsubstituted or optionally substituted with 1-3 substituents selected from deuterium, halogen, hydroxy and C₁₋₆ alkyl, or R₃ and R₄ form ═O, ═S, ═N—CN or ═CH₂;

R₅ and R₆ are each independently selected from hydrogen, deuterium and halogen;

each R₇ and R₈ are independently selected from hydrogen, deuterium, cyano, halogen, hydroxy, amino, C₁₋₆ alkyl, —NHC₁₋₆ alkyl, —N(C₁₋₆ alkyl)₂, C₃₋₆ cycloalkyl, C₁₋₆ alkoxy, C₂₋₆ alkenyl, C₂₋₆ alkynyl and —COOC₁₋₆ alkyl, wherein the amino, alkyl, cycloalkyl, alkenyl, and alkynyl are unsubstituted or substituted with 1-3 substituents selected from halogen, hydroxy, amino, acetyl and deuterium atom;

X is a 4- to 9-membered heterocyclic ring unsubstituted or substituted with R₉, wherein X is a divalent group, as can be seen from the structure of formula I; each R₉ is independently selected from hydrogen, deuterium, cyano, halogen, hydroxy, amino, C₁₋₆ alkyl and C₁₋₆ alkoxy, wherein the amino and alkyl are unsubstituted or substituted with 1-3 substituents selected from halogen, cyano, hydroxy, amino and deuterium atom;

Y is

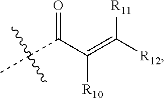

wherein R₁₀, R₁₁ and R₁₂ are each independently selected from hydrogen, deuterium, halogen, cyano, C₁₋₆ alkyl, C₃₋₆ cycloalkyl, 3- to 7-membered heterocyclyl, C₂₋₆ alkenyl, C₂₋₆ alkynyl, acetyl, propionyl, butyryl, and —COOC₁₋₆ alkyl, wherein the alkyl, cycloalkyl, alkenyl, alkynyl, acetyl, propionyl and butyryl are unsubstituted or substituted with 1-3 substituents selected from deuterium, halogen, cyano, hydroxy, amino, C₁₋₆ alkyl, —NHC₁₋₆ alkyl, —N(C₁₋₆ alkyl)₂ and 3- to 7-membered heterocyclyl; or R₁₀ and R₁₂ are joined to each other to form a triple bond;

Q is N or C-Q′, wherein Q′ is selected from hydrogen, deuterium, cyano, halogen and C₁₋₆ alkyl.

For the sake of clear description of the present invention, the terms referred to will now be defined as follows.

In the present invention, the term “C₁₋₆ alkyl”, alone or in combination, refers to a saturated linear or branched alkyl group containing 1-6 carbon atoms, including, for example, methyl, ethyl, propyl, isopropyl, butyl, sec-butyl, isobutyl, tert-butyl, n-pentyl, 2-pentyl, 3-pentyl, 2-methyl-2-butyl, 3-methyl-2-butyl, 3-methyl-1-butyl, 2-methyl-1-butyl, n-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl. In some specific embodiments, “C₁₋₆ alkyl” is preferably a saturated linear or branched alkyl group containing 1-4 carbon atoms. Preferably, “C₁₋₆ alkyl” is any one of methyl, ethyl, n-propyl, isopropyl and tert-butyl. Similarly, the term “C₁₋₃ alkyl”, alone or in combination, refers to a saturated linear or branched alkyl group containing 1-3 carbon atoms, including methyl, ethyl, propyl, isopropyl, etc.

The term “3- to 7-membered cycloalkyl”, alone or in combination, refers to a cycloalkyl group having 3 to 7 carbon atoms, including, for example, cyclopropyl, cyclobutyl, cyclopentyl, cyclohexyl and cycloheptyl, etc. Particular “C₃₋₇ cycloalkyl” is cyclopropyl, cyclobutyl, cyclopentyl, cyclohexyl, etc. In some specific embodiments, the “3- to 7-membered cycloalkyl” is preferably a cycloalkyl group having 3-6 carbon atoms.

The term “amino”, alone or in combination, refers to a primary amino group (—NH₂), a secondary amino group (—NH—) or a tertiary amino group

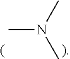

The term “C₁₋₆ alkoxy”, alone or in combination, refers to a group C₁₋₆ alkyl-O—, wherein the “C₁₋₆ alkyl” is as defined above; for example, it includes, but is not limited to, methoxy (—OCH₃), ethoxy (—OCH₂CH₃), n-propoxy (—OCH₂CH₂CH₃), isopropoxy (—OCH(CH₃)₂), n-butoxy (—OCH₂CH₂CH₂CH₃), sec-butoxy (—OCH(CH₃)CH₂CH₃), isobutoxy (—OCH₂CH(CH₃)₂), tert-butoxy (—OC(CH₃)₃), n-pentoxy (—OCH₂CH₂CH₂CH₂CH₃), neopentoxy (—OCH₂C(CH₃)₃), etc.

The term “halogen”, alone or in combination, refers to fluorine, chlorine, bromine or iodine. In some specific embodiments, the “halogen” is preferably fluorine, chlorine or bromine.

The term “heterocycloalkyl”, also referred to as “heterocyclyl”, refers to a saturated or partially unsaturated (containing 1 or 2 double bonds) non-aromatic cyclic group consisting of carbon atoms and heteroatoms such as nitrogen, oxygen or sulfur, which may be monocyclic, bicyclic bridged or spiro group. In the present invention, preferably, the heterocycloalkyl contains 2-11 carbon atoms and 1, 2, 3 or 4 heteroatoms; the nitrogen, carbon or sulfur atoms in the heterocycloalkyl may be optionally oxidized. The hydrogen atoms in the “heterocycloalkyl” are independently optionally substituted with one or more substituents described in the present invention. The “heterocycloalkyl” may be linked to a parent molecule through any ring atom in the ring.

The term “4- to 9-membered heterocyclyl” refers to a monocyclic, fused, bridged or spiro ring containing 4-9 carbon atoms and heteroatoms or heteroatom groups selected from N, O, S(O)ₘ (where m is an integer from 0 to 2) and containing no double bond or containing 1 or 2 double bonds, e.g., azetidinyl, oxetanyl, pyrrolidinyl, tetrahydrofuryl, tetrahydrothienyl, piperidinyl, morpholinyl, piperazinyl, thiomorpholinyl, tetrahydropyranyl, 1,1-dioxothiomorpholinyl, bicyclo [4.1.0]heptyl, etc. containing no double bond or containing 1 or 2 double bonds. In some specific embodiments, the “4- to 9-membered heterocyclyl” is preferably a monocyclic, fused, bridged or spiro ring having 6-7 carbon atoms and heteroatoms or heteroatom groups and containing no double bond or containing 1 or 2 double bonds.

The term “aryl” refers to any stable 6- to 10-membered monocyclic or bicyclic aromatic group, including, for example, phenyl, naphthyl, tetrahydronaphthyl, 2,3-dihydroindenyl or biphenyl. The hydrogen atoms in the “aryl” are independently optionally substituted with one or more substituents described in the present invention.

The term “heteroaryl” refers to an aromatic cyclic group formed by replacement of a carbon atom in the ring with at least one heteroatom or heteroatom group selected from N, O, S(O)ₘ (where m is an integer from 0 to 2). The heteroaromatic group may be a 5- to 7-membered monocyclic or 7-12 bicyclic group. In the present invention, the heteroaryl preferably contains 1, 2, 3 or 4 heteroatoms; examples thereof include thienyl, pyridinyl, pyrimidinyl, pyrazinyl, pyridazinyl, pyridine N-oxide group (i.e.,

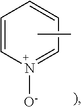

pyridonyl, pyrazinonyl, pyrimidonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, benzothienyl, indolyl, benzimidazolyl, benzothiazolyl, benzofuranyl, quinolyl, isoquinolyl, quinazolinyl, etc. The hydrogen atoms in the “heteroaryl” are independently optionally substituted with one or more substituents described in the present invention.

The term “5- to 10-membered heteroaryl” refers to a heteroaromatic ring having 5-10 carbon atoms and heteroatoms or heteroatom groups, wherein the heteroaromatic ring is as defined above. Similarly, the term “5- to 6-membered heteroaryl” refers to a heteroaromatic ring having 5-6 carbon atoms and heteroatoms or heteroatom groups, wherein the heteroaromatic ring is as defined above.

The term “C₆₋₁₀ aryl” refers to an aryl group having 6-10 carbon atoms, wherein the aryl is as defined above.

The term “cyano”, alone or in combination, refers to the group —CN.

The term “hydroxy”, alone or in combination, refers to the group —OH.

The term “isomer” encompasses all isomeric forms including enantiomers, diastereoisomers, tautomers and geometric isomers (including cis-trans isomers). Thus, individual stereochemical isomers or mixtures of enantiomers, diastereoisomers, tautomers or geometric isomers (or cis-trans isomers) of the compounds designed in the present invention all fall within the scope of the present invention.

The term “pharmaceutically acceptable salt” means that the compounds of the present invention are present in the form of their pharmaceutically acceptable salts, including acid addition salts and base addition salts. Pharmaceutically acceptable salts are described in pharmaceutical salts described by S. M. Berge in J. Pharmaceutical Sciences (Vol. 66: pp. 1-19, 1977). In the present invention, pharmaceutically acceptable non-toxic acid addition salts refer to salts formed by reaction of the compounds of the present invention with organic or inorganic acids including, but not limited to, hydrochloric acid, sulfuric acid, hydrobromic acid, hydroiodic acid, phosphoric acid, nitric acid, perchloric acid, acetic acid, oxalic acid, maleic acid, fumaric acid, tartaric acid, benzenesulfonic acid, methanesulfonic acid, salicylic acid, succinic acid, citric acid, lactic acid, propionic acid, benzoic acid, p-toluenesulfonic acid, malic acid, etc. Pharmaceutically acceptable non-toxic base addition salts refer to salts formed by reaction of the compounds of the present invention with organic or inorganic bases, including but not limited to alkali metal salts, such as lithium, sodium or potassium salts; alkaline earth metal salts, such as calcium or magnesium salts; and organic base salts, such as ammonium salts or N⁺(C₁₋₆ alkyl)₄ salts formed by reaction with N group-containing organic bases, preferably lithium hydroxide, sodium hydroxide, potassium hydroxide, sodium carbonate, sodium bicarbonate, potassium carbonate, potassium bicarbonate, magnesium carbonate, calcium carbonate, aqueous ammonia, triethylamine, tetrabutylammonium hydroxide, etc.

The term “solvate” refers to a compound formed by association of the compound of the present invention with one or more solvent molecules. Solvents for forming solvates include, but are not limited to, water, methanol, ethanol, isopropanol, ethyl acetate, tetrahydrofuran, N,N-dimethylformamide, dimethylsulfoxide, etc. When the solvent is water, the compound formed by association of the compound of the present invention is a hydrate. That is, the term “hydrate” refers to a compound formed by association of the compound of the present invention with water. The “pharmaceutically acceptable salt” can be synthesized using a general chemical method.

The term “ester” is used to refer to organic esters, including monoesters, diesters, triesters and, more generally, polyesters.

The term “prodrug” refers to a chemical derivative of the compound of the present invention, which is converted in vivo into a compound of formula I, II or III by chemical reaction.

The term “isotopically labeled compound” refers to a compound in which one or more atoms in the compound are replaced by atoms having an atomic mass or mass number different from the atomic mass or mass number usually found in nature; examples of isotopes include isotopes of hydrogen, carbon, nitrogen, etc. In some specific embodiments, the isotopically labeled compound includes isotopic derivatives obtained by substitution of 1-6 deuterium atoms (D) for the hydrogen atoms in formula I and isotopic derivatives obtained by substitution of 1-3 carbon-14 atoms (14C) for the carbon atoms in formula (I).

The word “comprise” and variations thereof such as “comprises” or “comprising” should be understood in an open, non-exclusive sense, i.e., “comprising but not limited to”.

The terms referred to in the present invention are defined above. Those skilled in the art may also interpret the above terms with reference to the prior art. Further description is made below based on the contents of the present invention and the definitions of the terms.

In one preferred embodiment, the above Q is N.

In one preferred embodiment, the above R₁ is selected from C₆₋₁₀ aryl and 5- to 10-membered heteroaryl unsubstituted or substituted with R₇, wherein the substitution with R₇ occurs at an ortho position of the atom in the C₆₋₁₀ aryl and 5- to 10-membered heteroaryl connected to the N atom at position 4 in the ring

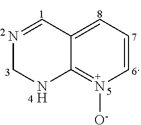

In one preferred embodiment, the above R₁ is selected from C₆₋₁₀ aryl and 5- to 6-membered heteroaryl unsubstituted or substituted with R₇, wherein the 5- to 6-membered heteroaryl contains 1-3 heteroatoms or heteroatom groups selected from N, O, S(O)ₘ, wherein m is an integer from 0 to 2.

In one preferred embodiment, the above R₁ is selected from C₆₋₁₀ aryl and 5- to 10-membered heteroaryl unsubstituted or substituted with R₇, wherein the C₆₋₁₀ aryl is selected from phenyl, naphthyl, tetrahydronaphthyl and 2,3-dihydroindenyl; the 5- to 10-membered heteroaryl is selected from thienyl, pyridinyl, pyridine N-oxide group, pyrimidinyl, pyrazinyl, pyridazinyl, pyridonyl, pyrazinonyl, pyrimidinonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuranyl, quinolinyl, isoquinolinyl and quinazolinyl.

In one more preferred embodiment, the C₆₋₁₀ aryl is phenyl; the 5- to 10-membered heteroaryl is selected from pyridinyl and pyrimidinyl.

In one preferred embodiment, each R₇ is independently selected from hydrogen, deuterium, cyano, halogen, hydroxy, amino, C₁₋₆ alkyl, C₃₋₆ cycloalkyl and C₁₋₆ haloalkyl. Preferably, each R₇ is independently selected from hydrogen, deuterium, methyl, CH₂F, CHF₂, CF₃, ethyl, propyl, isopropyl, butyl, sec-butyl, isobutyl, tert-butyl, cyclopropyl, cyclobutyl, cyclopentyl and cyclohexyl.

In one preferred embodiment, the above R₂ is selected from C₆₋₁₀ aryl and 5- to 6-membered heteroaryl unsubstituted or substituted with R₈, wherein the 5- to 6-membered heteroaryl contains 1-3 heteroatoms or heteroatom groups selected from N, O, S(O)ᵣ, wherein r is 0, 1 or 2.

In one preferred embodiment, the above R₂ is selected from C₆₋₁₀ aryl and 5- to 10-membered heteroaryl unsubstituted or substituted with R₈, wherein the C₆₋₁₀ aryl is selected from phenyl, naphthyl, tetrahydronaphthyl and 2,3-dihydroindenyl; the 5- to 10-membered heteroaryl is selected from thienyl, pyridinyl, pyridine N-oxide group, pyrimidinyl, pyrazinyl, pyridazinyl, pyridonyl, pyrazinonyl, pyrimidinonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, benzothienyl, indolyl, benzimidazolyl, benzothiazolyl, benzofuranyl, quinolinyl, isoquinolinyl and quinazolinyl. Preferably, R₂ is selected from phenyl, imidazolyl, pyrrolyl, pyridine N-oxide group, pyridinyl, pyridonyl, naphthyl, quinolinyl, isoquinolinyl and quinazolinyl unsubstituted or substituted with R₈.

In one preferred embodiment, each R₈ is independently selected from hydrogen, deuterium, cyano, halogen, hydroxy, amino, C₁₋₆ alkyl, —NHC₁₋₆ alkyl, —N(C₁₋₆ alkyl)₂ and C₁₋₆ alkoxy, wherein the amino and alkyl are unsubstituted or substituted with 1-3 halogens, hydroxy groups, amino groups, acetyl groups or deuterium atoms. Preferably, each R₈ is independently selected from hydrogen, deuterium, fluorine, chlorine, hydroxy and amino.

In one preferred embodiment, R₃ and R₄ are independently selected from hydrogen, deuterium and C₁₋₆ alkyl, or R₃ and R₄ are joined to form cyclopropyl, or R₃ and R₄ form ═O, —S or ═N—CN; preferably, R₃ and R₄ form ═O.

In one preferred embodiment, R₅ and R₆ are each independently selected from hydrogen, deuterium, fluorine, and chlorine.

In one preferred embodiment, X is a 4- to 9-membered heterocyclyl unsubstituted or substituted with 1-3 R₉, wherein the atom in the 4- to 9-membered heterocyclyl connected to Y is N.

In one preferred embodiment, X is a 4- to 9-membered heterocyclyl unsubstituted or substituted with 1-3 R₉, wherein the 4- to 9-membered heterocyclyl includes a monocyclic ring, a fused ring, a bridged ring and a spiro ring.

In one more preferred embodiment, X is a 6- to 7-membered heterocyclic ring unsubstituted or substituted with 1-3 R₉, wherein the 6- to 7-membered heterocyclic ring contains no double bond or contains 1 or 2 double bonds; preferably, X is a 6- to 7-membered heterocyclic ring unsubstituted or substituted with 1-2 R₉, wherein the 6- to 7-membered heterocyclic ring is selected from

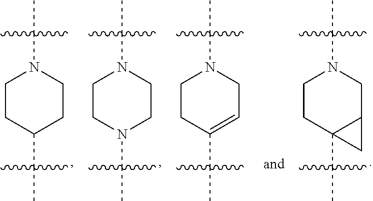

In one preferred embodiment, Q is N or C-Q′, wherein Q′ is selected from hydrogen, deuterium and cyano.

In one preferred embodiment, each R₉ is independently selected from hydrogen, deuterium, methyl, ethyl, —CH₂OH, —CH₂CN and —CH₂F.

In one preferred embodiment, X is the following groups:

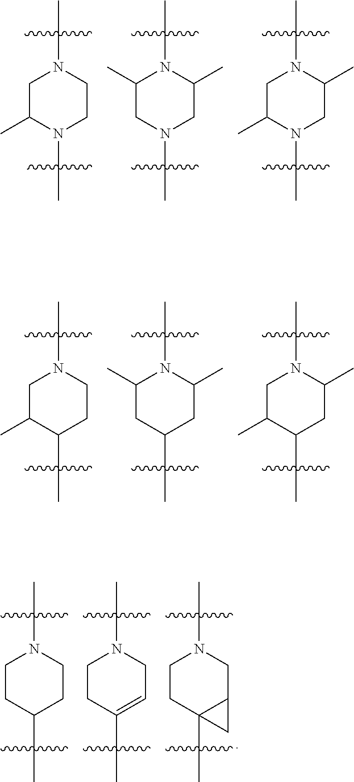

In one preferred embodiment, Y is

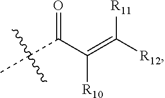

wherein R₁₀ is selected from hydrogen, deuterium and fluorine, R₁₁ is selected from hydrogen or deuterium, and R₁₂ is selected from hydrogen, deuterium, acetyl, dimethylaminomethyl, piperidinyl and aminocyclopropyl; preferably, Y is selected from

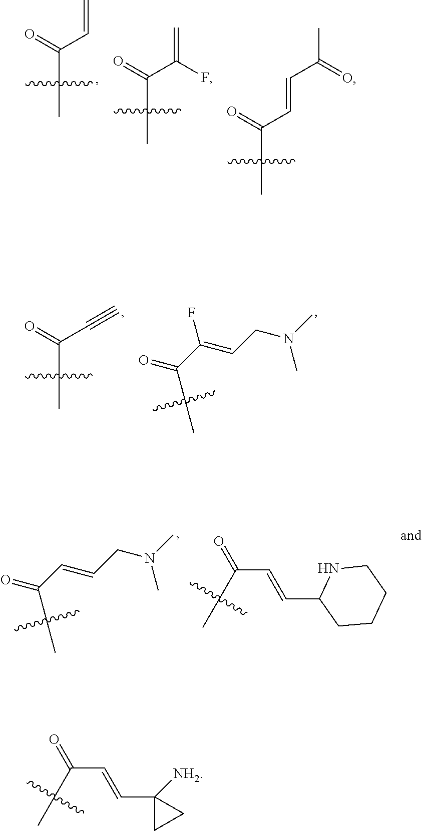

In one preferred embodiment, R₁ is selected from C₆₋₁₀ aryl and 5- to 10-membered heteroaryl unsubstituted or substituted with 1-3 R₇;

R₂ is selected from C₆₋₁₀ aryl and 5- to 10-membered heteroaryl unsubstituted or substituted with 1-3 R₈;

R₃ and R₄ are each independently selected from hydrogen, deuterium and C₁₋₆ alkyl, or R₃ and R₄ are joined to form cyclopropyl, or R₃ and R₄ form ═O;

R₅ and R₆ are each independently selected from hydrogen, deuterium and halogen;

each R₇ is independently selected from hydrogen, deuterium, cyano, halogen, hydroxy, amino, C₁₋₆ alkyl and C₃₋₆ cycloalkyl;

each R₈ is independently selected from hydrogen, deuterium, cyano, halogen, hydroxy, amino, C₁₋₆ alkyl, —NHC₁₋₆ alkyl, —N(C₁₋₆ alkyl)₂ and C₁₋₆ alkoxy, wherein the amino and alkyl are unsubstituted or substituted with 1-3 halogens, hydroxy groups, amino groups, acetyl groups and deuterium atoms;

X is a 6- to 7-membered heterocyclic ring unsubstituted or substituted with 1-3 R₉, wherein the atom in the 6- to 7-membered heterocyclic ring connected to Y is N, and each R₉ is independently selected from hydrogen, deuterium, methyl, ethyl, —CH₂OH, —CH₂CN and —CH₂F;

Y is

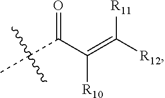

wherein R₁₀ is selected from hydrogen, deuterium and fluorine, R₁₁ is selected from hydrogen and deuterium, and R₁₂ is selected from acetyl, dimethylaminomethyl, piperidinyl and aminocyclopropyl;

Q is N or C-Q′, wherein Q′ is selected from hydrogen, deuterium and cyano.

In one preferred embodiment, R₁ is selected from C₆₋₁₀ aryl and 5- to 10-membered heteroaryl unsubstituted or substituted with 1-3 R₇, wherein the C₆₋₁₀ aryl is selected from phenyl, naphthyl, tetrahydronaphthyl and 2,3-dihydroindenyl; the 5- to 10-membered heteroaryl is selected from thienyl, pyridinyl, pyridine N-oxide group, pyrimidinyl, pyrazinyl, pyridazinyl, pyridonyl, pyrazinonyl, pyrimidinonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuranyl, quinolinyl, isoquinolinyl and quinazolinyl;

R₂ is selected from C₆₋₁₀ aryl and 5- to 10-membered heteroaryl unsubstituted or substituted with 1-3 R₈, wherein the C₆₋₁₀ aryl is selected from phenyl, naphthyl, tetrahydronaphthyl and 2,3-dihydroindenyl; the 5- to 10-membered heteroaryl is selected from thienyl, pyridinyl, pyridine N-oxide group, pyrimidinyl, pyrazinyl, pyridazinyl, pyridonyl, pyrazinonyl, pyrimidinonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuranyl, quinolinyl, isoquinolinyl and quinazolinyl;

R₃ and R₄ form ═O;

R₅ and R₆ are each independently selected from hydrogen, deuterium, chlorine and fluorine;

each R₇ is independently selected from hydrogen, deuterium, methyl, ethyl, propyl, isopropyl, butyl, sec-butyl, isobutyl, tert-butyl, cyclopropyl, cyclobutyl, cyclopentyl and cyclohexyl;

each R₈ is independently selected from hydrogen, deuterium, fluorine, chlorine, hydroxy and amino;

X is a 6- to 7-membered heterocyclic ring unsubstituted or substituted with 1-2 R₉, wherein the 6- to 7-membered heterocyclic ring is selected from

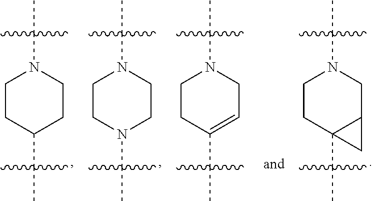

and each R₉ is independently selected from hydrogen, deuterium, methyl, ethyl, —CH₂CN, —CH₂OH and —CH₂F;

Y is

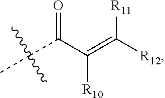

wherein R₁₀ is selected from hydrogen, deuterium and fluorine, R₁₁ is selected from hydrogen and deuterium, and R₁₂ is selected from hydrogen, deuterium, acetyl, dimethylaminomethyl, piperidinyl and aminocyclopropyl;

Q is N or C-Q′, wherein Q′ is selected from hydrogen, deuterium and cyano.

In one preferred embodiment, the compound of formula I has a structure of formula I-A, I-B, I-C, I-D, I-E or I-F:

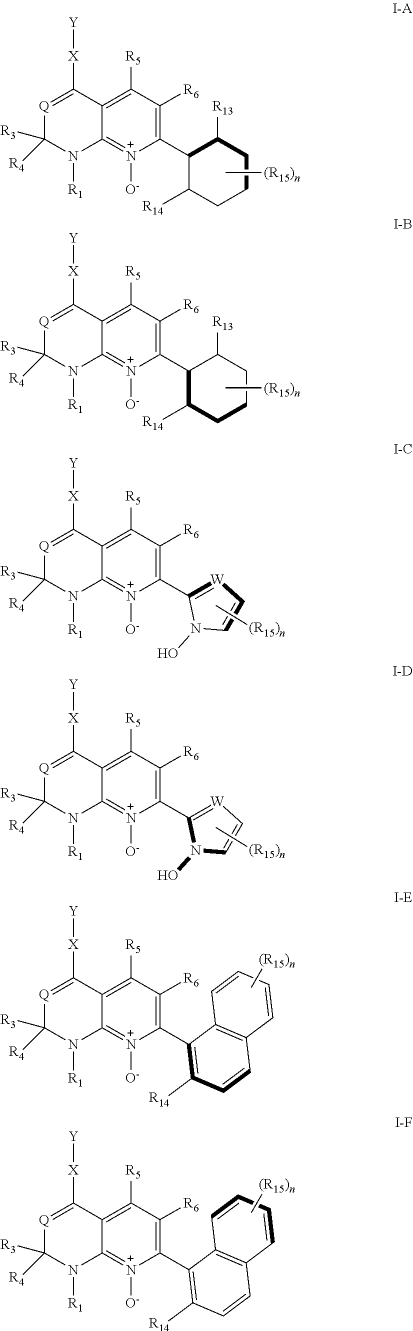

wherein R₁₃ and each R₁₅ are each independently selected from hydrogen, deuterium, cyano, halogen, hydroxy, amino, C₁₋₆ alkyl, —NHC₁₋₆ alkyl, —N(C₁₋₆ alkyl)₂ and C₁₋₆ alkoxy, wherein the amino and alkyl are unsubstituted or substituted with 1-3 substituents selected from halogen, hydroxy, amino, acetyl and deuterium atom; n is an integer from 0 to 3; R₁₄ is selected from hydrogen, deuterium, fluorine, hydroxy and amino; W is selected from N, CH, CCH₃, CC₂H₅ and CCH(CH₃)₂. Preferably, the compound of formula I has a structure of formula I-A or I-B, wherein n is 0; one of R₁₃ and R₁₄ is hydrogen, and the other is hydroxy or F, or R₁₃ and R₁₄ are both hydroxy or F, or one of R₁₃ and R₁₄ is hydroxy and the other is F; preferably, R₁₃ and R₁₄ are both hydroxy or F, or one of R₁₃ and R₁₄ is hydroxy, and the other is F; more preferably, one of R₁₃ and R₁₄ is hydroxy, and the other is F.

The present invention further provides a compound or a pharmaceutically acceptable salt, ester, hydrate, solvate, stereoisomer, tautomer, cis-trans isomer, isotopically labeled compound or prodrug thereof, wherein the compound is any one of:

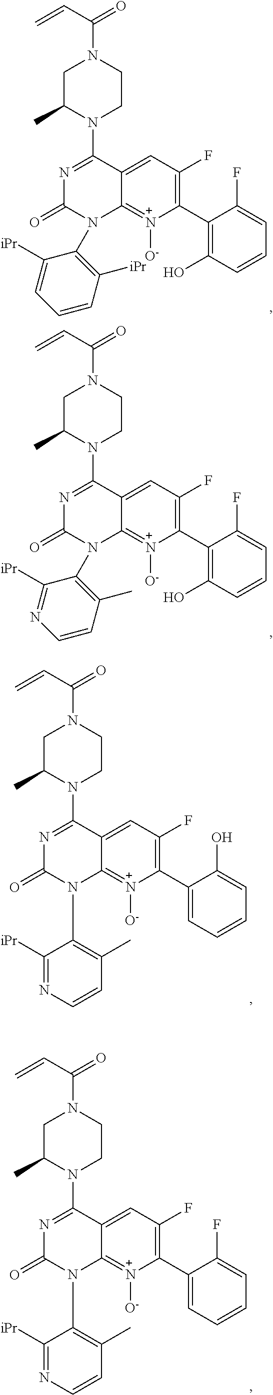

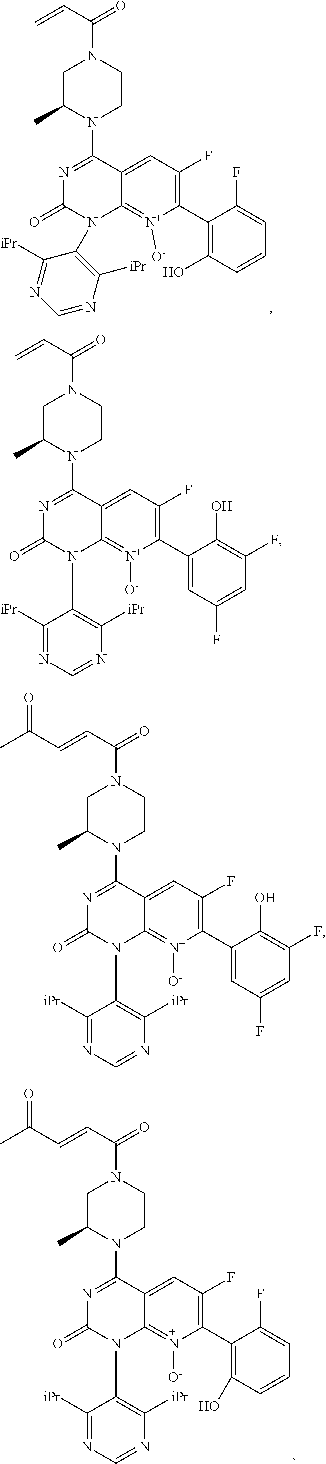

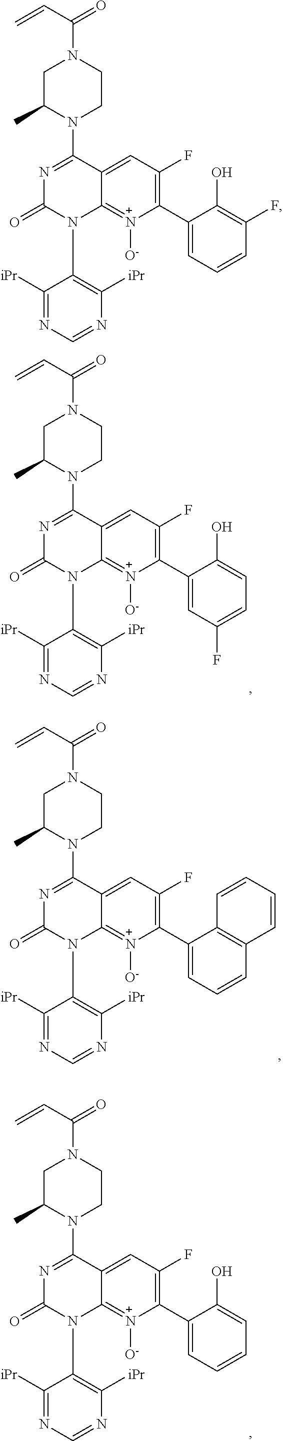

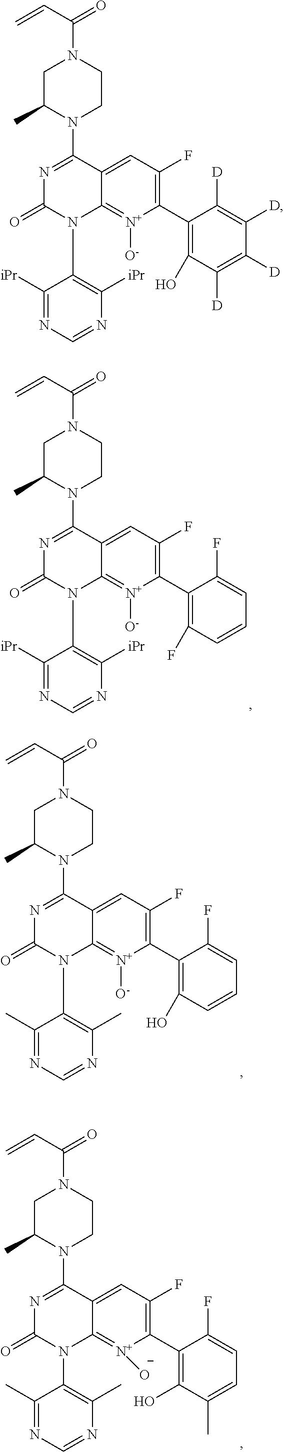

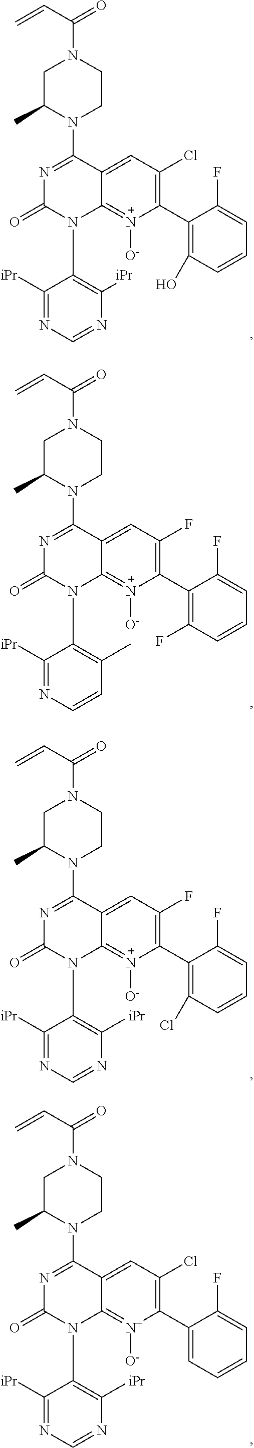

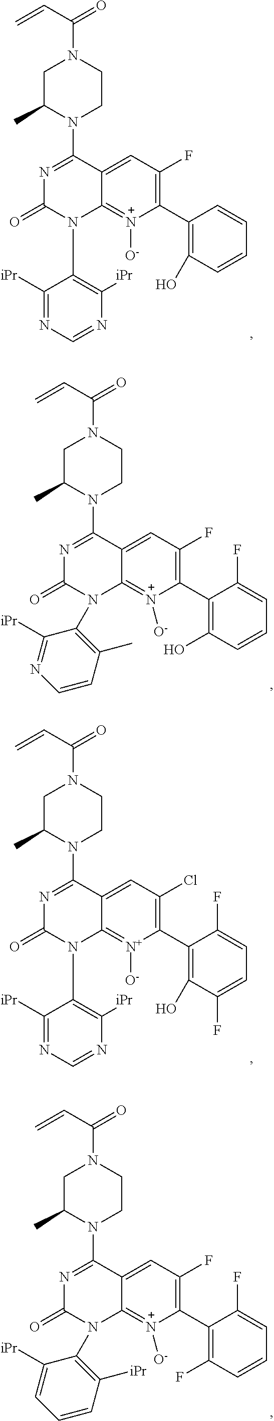

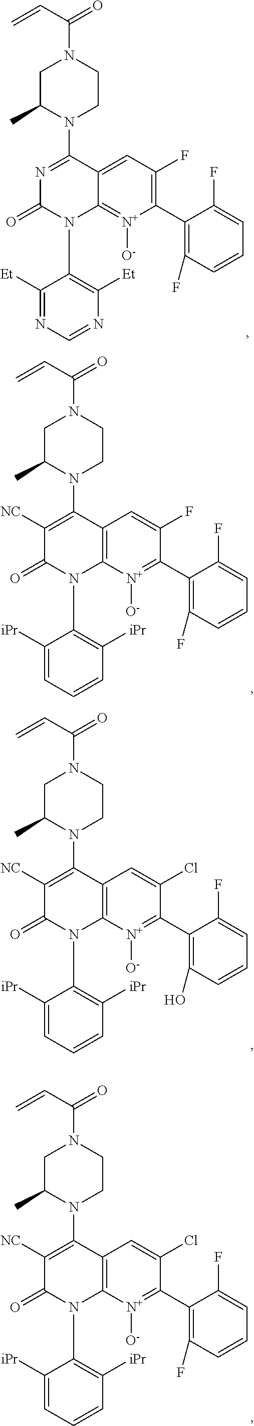

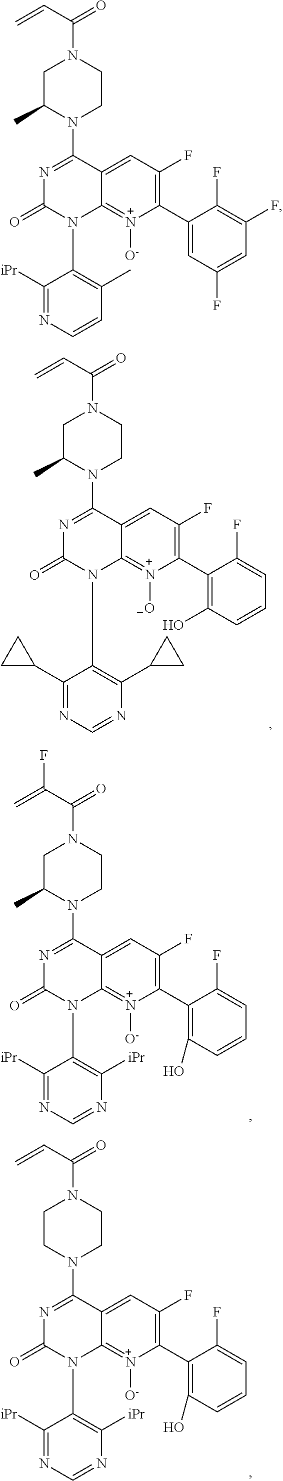

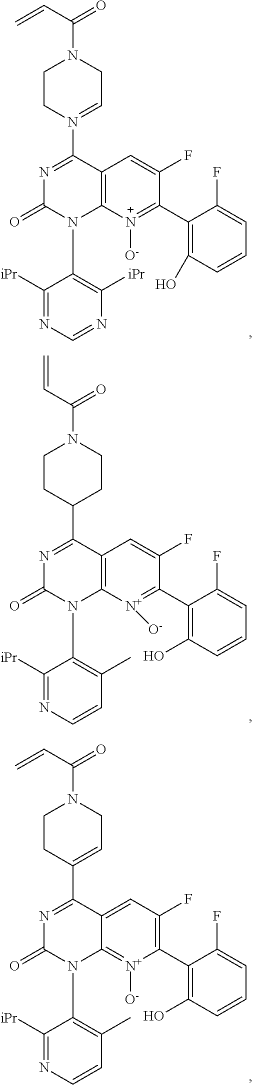

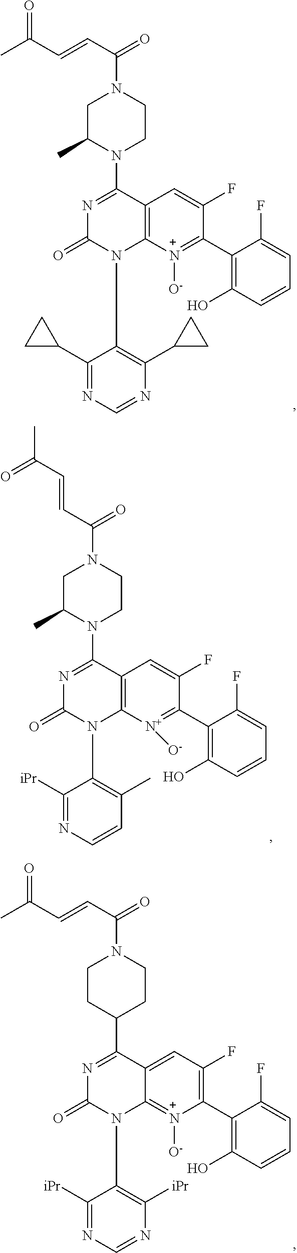 [SMILES: CC(=O)/C=C/C(=O)N1CCN(c2nc(=O)n(-c3c(C4CC4)ncnc3C3CC3)c3c2cc(F)c(-c2c(O)cccc2F)[n+]3[O-])[C@@H](C)C1 ; CC(=O)/C=C/C(=O)N1CCN(c2nc(=O)n(-c3c(C)ccnc3C(C)C)c3c2cc(F)c(-c2c(O)cccc2F)[n+]3[O-])[C@@H](C)C1 ; CC(=O)/C=C/C(=O)N1CCC(c2nc(=O)n(-c3c(C(C)C)ncnc3C(C)C)c3c2cc(F)c(-c2c(O)cccc2F)[n+]3[O-])CC1]

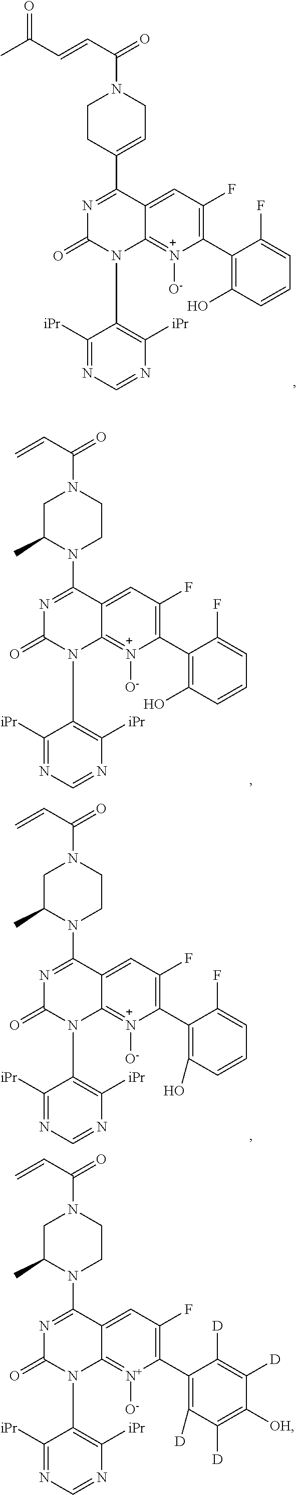

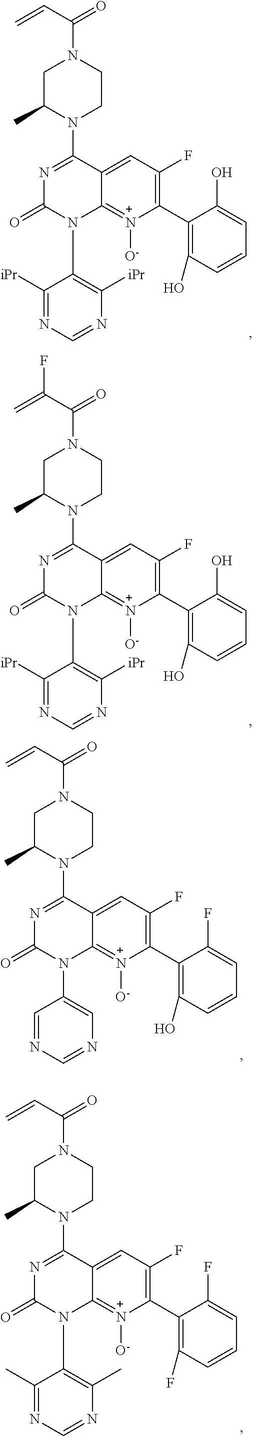

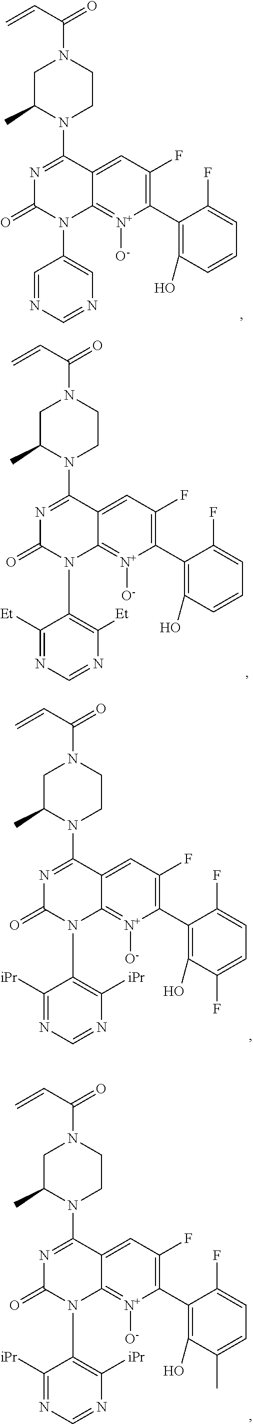

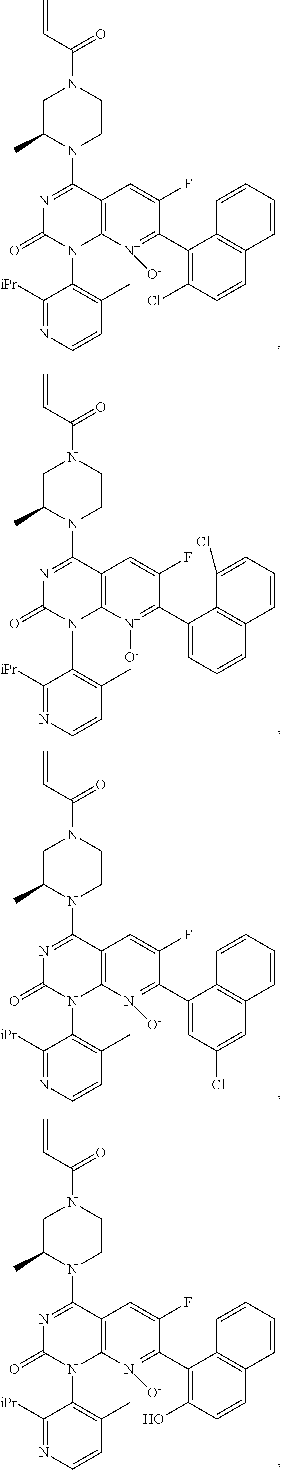

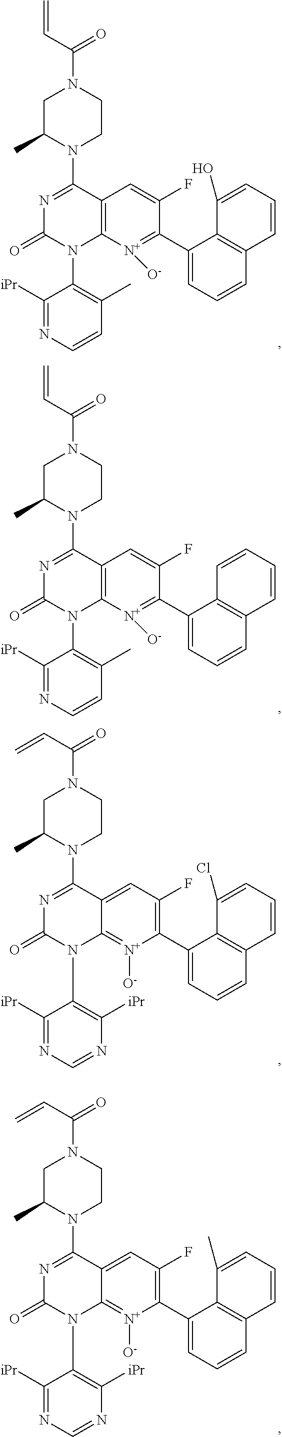

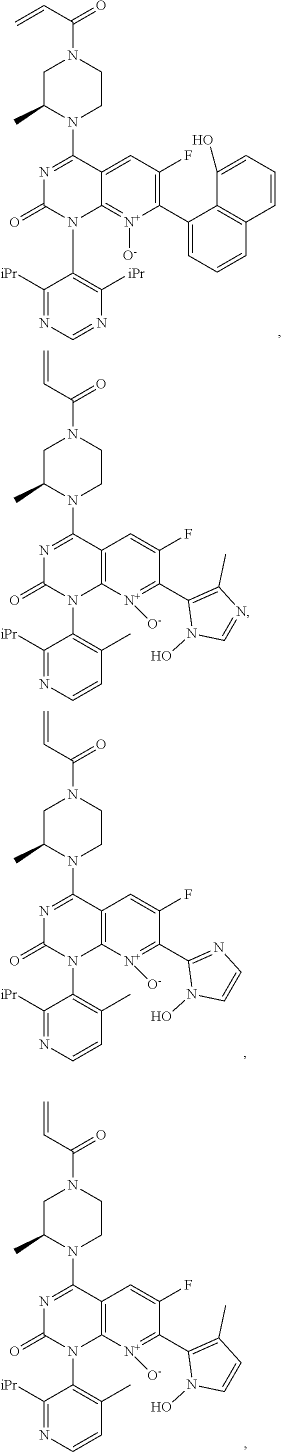

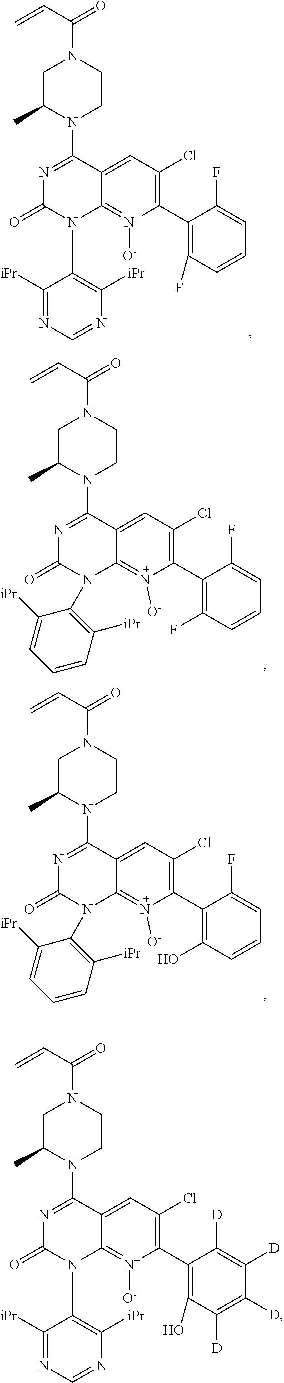

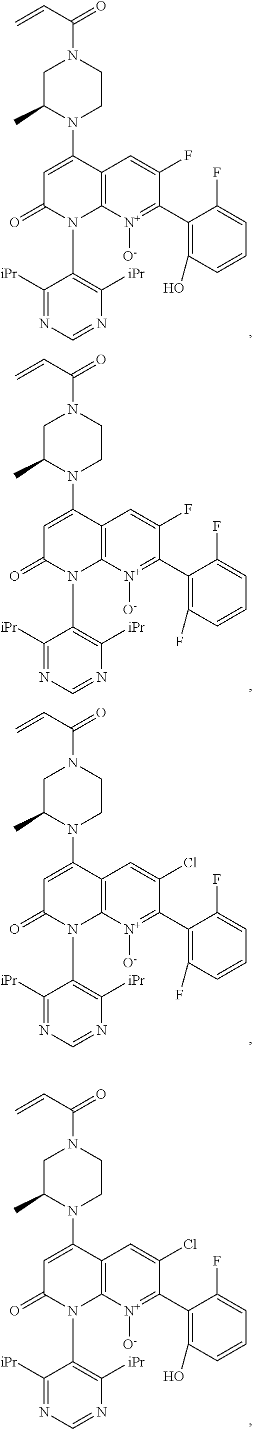

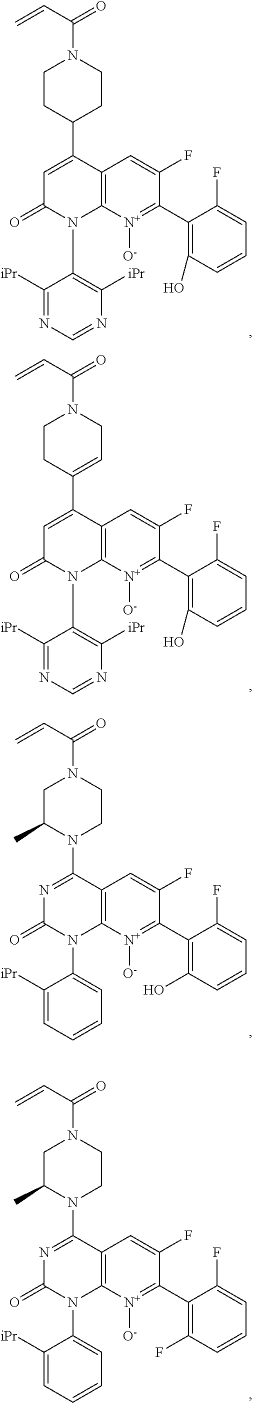

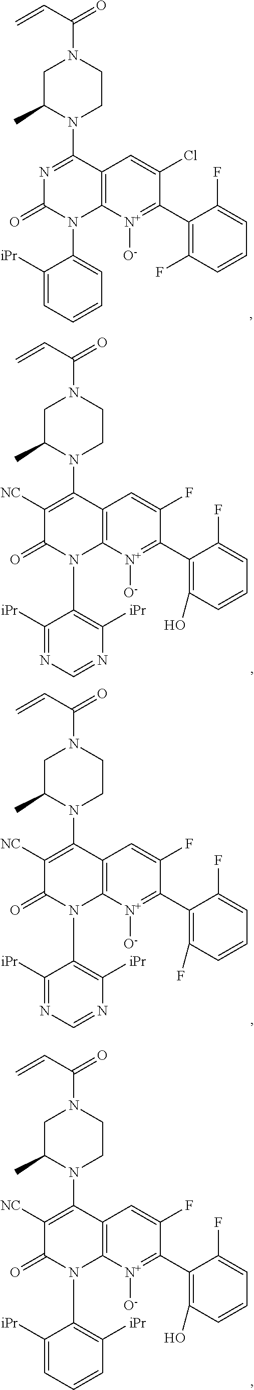

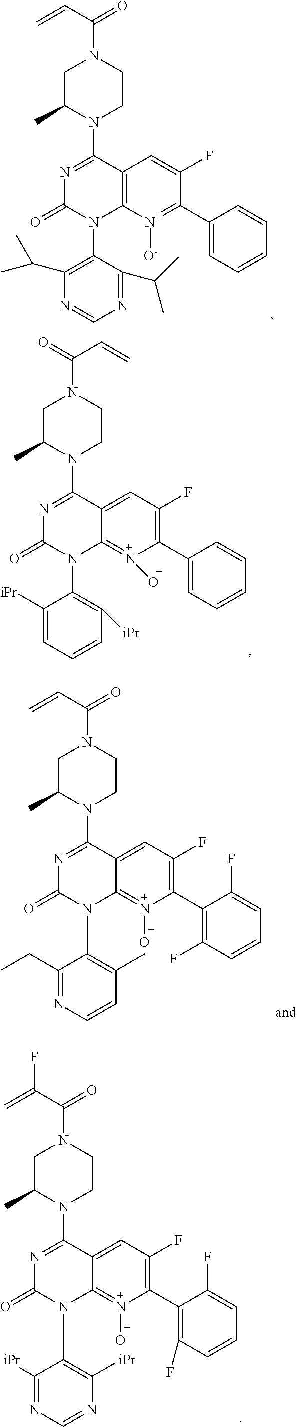

The present invention further provides a pharmaceutical composition comprising the compound or the pharmaceutically acceptable salt, ester, isomer, solvate, hydrate, prodrug or isotopiccally labeled compound thereof according to any one of the embodiments above. In some embodiments, the pharmaceutical composition comprises one or more compounds of the present application or pharmaceutically acceptable salts, esters, isomers, solvates, hydrates, prodrugs or isotopically labeled compounds thereof, and a pharmaceutically acceptable excipient. The pharmaceutically acceptable excipient refers to those excipients that do not cause significant irritation to an organism and will not impair the biological activity and properties of the active compound. Conventional excipients in the art may be used.

The present invention further provides use of the compound or the pharmaceutically acceptable salt, ester, hydrate, solvate, stereoisomer, tautomer, cis-trans isomer, isotopiccally labeled compound or prodrug thereof or the pharmaceutical composition in preparing a medicament for preventing and/or treating a KRAS G12C-mediated disease. Preferably, the disease includes lung cancer, pancreatic cancer, pancreatic ductal carcinoma, colon cancer, rectal cancer, appendiceal cancer, esophageal squamous carcinoma, head and neck squamous carcinoma, breast cancer and other solid tumors.

The present application further provides a method for preventing and/or treating a KRAS G12C-mediated disease, the method comprising administering to a mammal, preferably a human, in need of such treatment and/or prevention a therapeutically effective amount of a compound of formula I or a pharmaceutically acceptable salt, ester, hydrate, solvate, isomer, isotopically labeled compound or prodrug thereof, or a pharmaceutical composition thereof.

The present invention further provides an exemplary method for preparing the above compounds, the method comprising the following steps:

(1) reacting a compound 1 with oxalyl chloride and R₁—NH₂ to give a compound 2;

(2) reacting the compound 2 under the action of a base to give a compound 3;

(3) reacting the compound 3 under the action of POCl₃ to give a compound 4;

(4) subjecting the compound 4 to a coupling reaction to give a compound 5;

(5) reacting the compound 5 with R₂—B(OH)₂ to give a compound 6;

(6) reacting the compound 6 under the action of a protonic acid to give a compound 7; and

(7) subjecting the compound 7 to an acylation reaction to give a compound 8;

The reaction scheme is as follows:

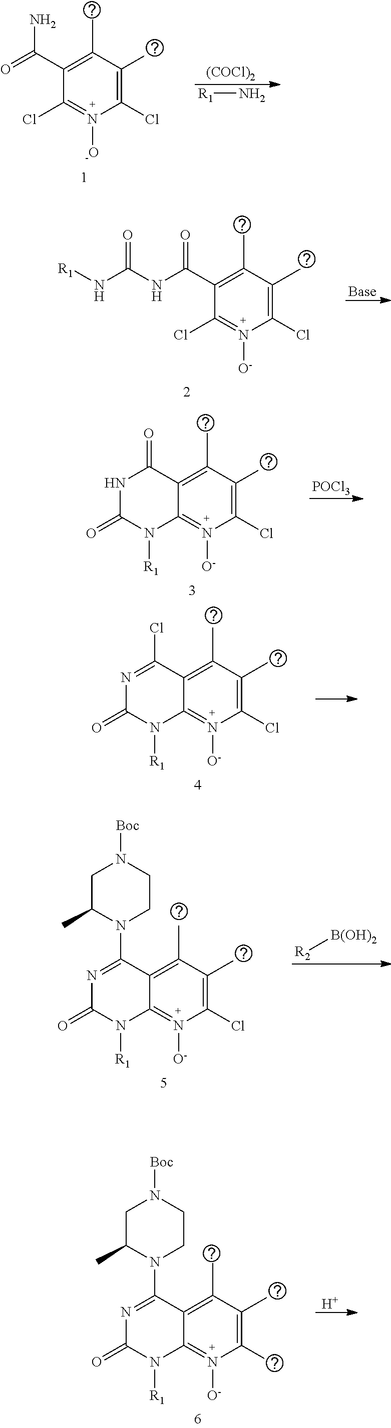

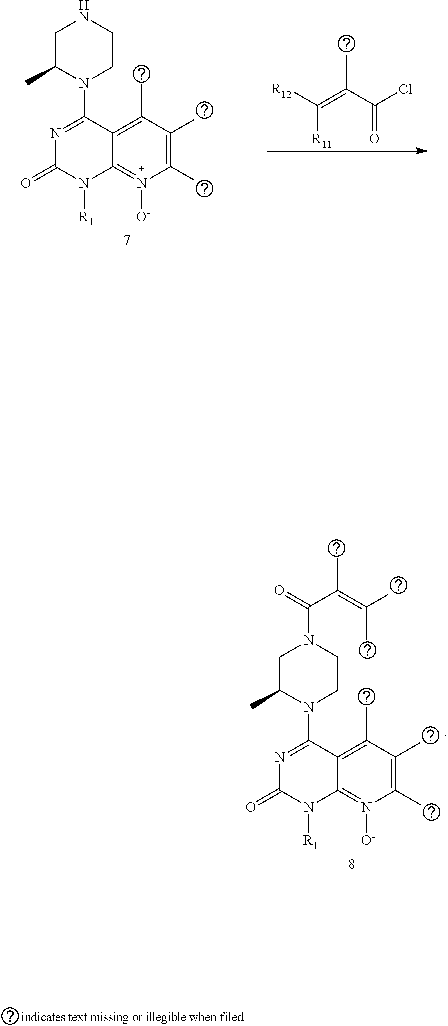

In one preferred embodiment, the base in step (2) is a common inorganic base in the art; preferably, the inorganic base is selected from one or more of KHDMS sodium carbonate, sodium bicarbonate, potassium carbonate, potassium bicarbonate, cesium carbonate, sodium hydroxide, potassium hydroxide and lithium hydroxide. The protonic acid in step (6) is a common protonic acid in the art, e.g., hydrochloric acid, sulfuric acid, nitric acid, phosphoric acid, formic acid, acetic acid, trifluoroacetic acid or hydrobromic acid.

The following examples can be used to further describe the present invention, but should not be construed as limiting the scope of the present invention.

### Example 1. SZ-014088A & SZ-014088B

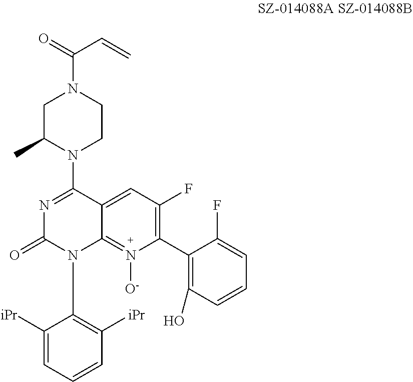

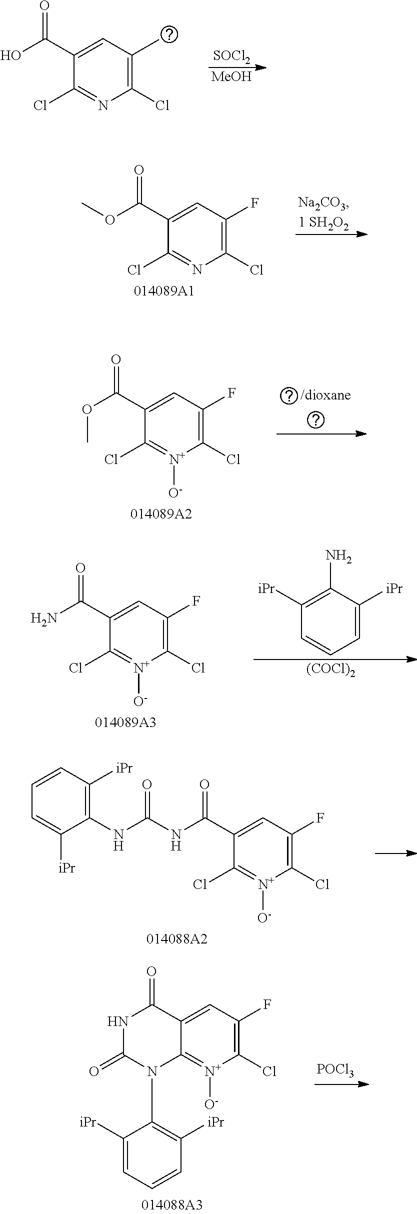

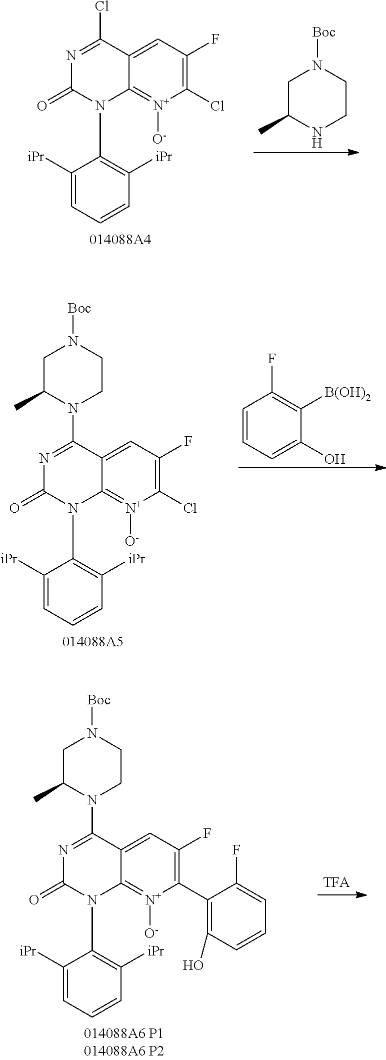 [SMILES: CC(C)c1cccc(C(C)C)c1-n1c(=O)nc(Cl)c2cc(F)c(Cl)[n+]([O-])c21 ; C[C@H]1CN(C(=O)OC(C)(C)C)CCN1 ; CC(C)c1cccc(C(C)C)c1-n1c(=O)nc(N2CCN(C(=O)OC(C)(C)C)C[C@@H]2C)c2cc(F)c(Cl)[n+]([O-])c21 ; OB(O)c1c(O)cccc1F ; CC(C)c1cccc(C(C)C)c1-n1c(=O)nc(N2CCN(C(=O)OC(C)(C)C)C[C@@H]2C)c2cc(F)c(-c3c(O)cccc3F)[n+]([O-])c21]

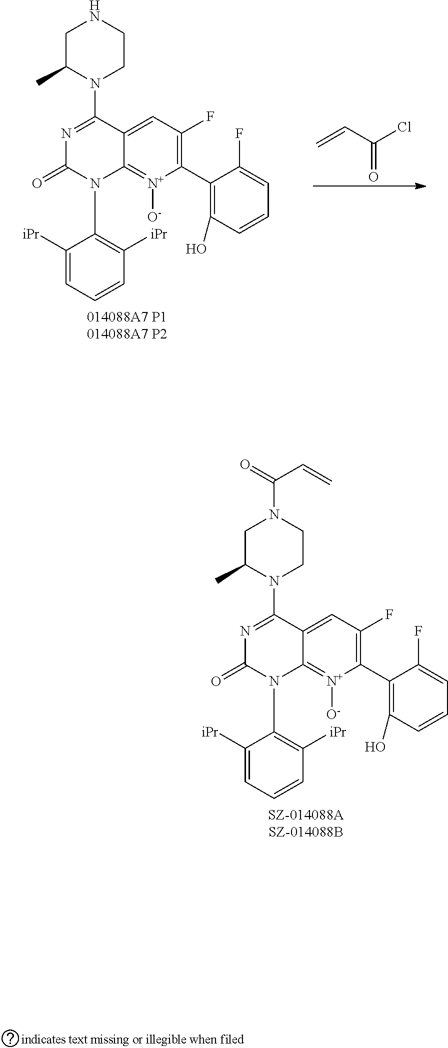

### Step 1: Synthesis of 014089A1

The compound 2,6-dichloro-5-fluoronicotinic acid (90.0 g, 0.43 mol) was dissolved in methanol (500 mL). Thionyl chloride (102.3 g, 0.86 mol) was added dropwise at 0° C., and N,N-dimethylformamide (4.0 g, 0.043 mol) was added. The reaction mixture was stirred at room temperature overnight. The reaction mixture was concentrated. The residue was dissolved in ice water, and the resulting solution was extracted with dichloromethane. The organic phase was dried over anhydrous sodium sulfate, and filtered. The filtrate was concentrated to give crude compound 014089A1 as an oil (65.0 g, 68% yield).

### Step 2: Synthesis of 014089A2

Compound 014089A1 (90 g, 0.4 mol) was dissolved in acetonitrile (900 mL). Sodium percarbonate (63.4 g, 0.4 mol) and trifluoromethanesulfonic anhydride (225.6 g, 0.8 mol) were added at 0° C. The reaction mixture was stirred at room temperature for 16 h. The reaction mixture was concentrated. The residue was dissolved in water, and the resulting solution was extracted with ethyl acetate (600 mL×3). The organic phase was washed successively with saturated aqueous sodium bicarbonate solution (500 mL×2) and saturated brine (500 mL), dried over anhydrous magnesium sulfate, and filtered under vacuum. The filtrate was concentrated. The residue was slurried with diethyl ether (500 mL) to give compound 014089A2 as a white solid (110 g, 100% yield). LCMS (M+H)⁺ m/z calculated 240.0. found 240.0. ¹H NMR (DMSO-d₆, 400 MHz): δ 8.02 (d, J=10.8 Hz, 1H), 3.94 (s, 3H).

### Step 3: Synthesis of 014089A3

Compound 014089A2 (110 g, 0.46 mol) was dissolved in 1,4-dioxane (350 mL). Aqueous ammonia (350 mL) was added at room temperature. The reaction mixture was stirred at room temperature for 2 h, with a large amount of white solid precipitating. The reaction mixture was filtered under vacuum, and the filtrate was concentrated. Then, the residue was slurried with saturated brine (300 mL). The two resulting filter cakes were combined and dried to give compound 014089A3 as a white solid (34 g, 100% yield). LCMS (M+H)⁺ m/z calculated 225.0. found 225.0. ¹H NMR (DMSO-d₆, 400 MHz): δ 8.125 (s, 1H), 8.034 (s, 1H), 7.81 (d, J=7.2 Hz, 1H).

### Step 4: Synthesis of 014088A2

Compound 014089A3 (20.0 g, 0.088 mol) was dissolved in tetrahydrofuran (200 mL). Oxalyl chloride (13.4 g, 0.088 mol) was added at room temperature. The reaction mixture was heated at reflux for 1 h. The reaction mixture was cooled to 0° C. 2,6-diisopropylaniline (15.56 g, 0.088 mol) was added. The reaction mixture was stirred at room temperature for 1 h. The pH of the reaction mixture was adjusted to neutrality with saturated sodium bicarbonate solution, followed by extraction with ethyl acetate (200 mL×3). The organic phases were combined, dried over anhydrous sodium sulfate, and filtered. The filtrate was concentrated under reduced pressure. The residue was purified by silica gel column chromatography (petroleum ether/ethyl acetate=5:1) to give compound 014088A2 as a yellow solid (8.3 g, 44% yield). LCMS (M+H)⁺ m/z calculated 428.2. found 428.2. ¹HNMR (DMSO-d₆, 400 MHz): δ 11.29 (br s, 1H), 9.38 (br s, 1H), 8.11 (d, J=6.8 Hz, 1H), 7.30 (t, J=7.6 Hz, 1H), 7.20 (d, J=7.6 Hz, 2H), 3.14-3.07 (m, 2H), 1.14-1.10 (m, 12H).

### Step 5: Synthesis of 014088A3

Compound 014088A2 (8.3 g, 21.17 mmol) was dissolved in anhydrous tetrahydrofuran (100 mL). KHMDS (1 M, 46.6 mL) was added dropwise under an ice bath. The reaction mixture was stirred at room temperature for 1 h. The reaction mixture was poured into water, and the pH of the resulting mixture was adjusted to neutrality with 2 M aqueous HCl solution, followed by extraction with ethyl acetate (100 mL×3). The organic phases were combined, dried over anhydrous sodium sulfate, and filtered. The filtrate was concentrated under reduced pressure. The residue was purified by silica gel column chromatography (petroleum ether/ethyl acetate=4:1) to give compound 014088A3 as a pale yellow solid (3.5 g, 46% yield). LCMS (M+H)⁺ m/z calculated 392. found 392.2. ¹H NMR (DMSO-d₆, 400 MHz): δ 12.52 (s, 1H), 8.09 (d, J=9.6 Hz, 1H), 7.34-7.25 (m, 1H), 7.21-7.16 (m, 2H), 2.80-2.76 (m, 2H), 1.12-1.09 (m, 6H), 1.01-0.99 (m, 6H).

### Step 6: Synthesis of 014088A4

Compound 014088A3 (1.0 g, 2.55 mmol) was dissolved in anhydrous acetonitrile (30 mL). POCl₃ (1.17 g, 7.65 mmol) and DIPEA (986 mg, 7.65 mmol) were added under an ice bath. The reaction mixture was heated to 60° C. and allowed to react for 1 h. The reaction mixture was cooled to room temperature and concentrated to give compound 014088A4 as a brown oil (1.46 g, 100% yield). The crude product was directly used in the next step.

### Step 7: Synthesis of 014088A5

The crude compound 014088A4 (1.46 g, 2.55 mmol) was dissolved in acetonitrile (30 mL). (S)-4-N-tert-butoxycarbonyl-2-methylpiperazine (1.1 g, 5.1 mmol) and diisopropylethylamine (986 mg, 7.65 mmol) were added. The reaction mixture was stirred at room temperature overnight. The reaction mixture was concentrated, and the residue was purified by silica gel column chromatography (petroleum ether/ethyl acetate=4:1) to give 014088A5 (1.0 g, 68.5% yield). LCMS (M+H)⁺ m/z calculated 574.2. found 574.2. ¹H NMR (DMSO-d₆, 400 MHz): δ 7.91 (d, J=10.4 Hz, 1H), 7.29 (t, J=10.4 Hz, 1H), 7.16-7.12 (m, 2H), 4.78-4.76 (m, 1H), 4.10-3.99 (m, 2H), 3.87-3.82 (m, 1H), 3.63-3.61 (m, 1H), 3.36-3.34 (m, 1H), 3.10-3.0 (m, 1H), 2.61-2.53 (m, 2H), 1.47 (s, 9H), 1.16-1.18 (m, 3H), 1.09-0.91 (m, 12H).

### Step 8: Synthesis of 014088A6 P1 & 014088A6 P2

Compound 014088A5 (270 mg, 0.50 mmol), 2-fluoro-6-hydroxyphenylboronic acid (155 mg, 1.0 mmol), potassium phosphate (212 mg, 1.0 mmol) and Sphos (61.5 mg, 0.15 mmol) were dissolved in 1,4-dioxane (10 mL). The system was purged with nitrogen several times, and then Pd₂(dba)₃ (46 mg, 0.05 mmol) was added. The system was purged with nitrogen several times, and then the reaction mixture was stirred at 80° C. for 3 h, cooled to room temperature, and filtered. The filtrate was diluted with ethyl acetate (100 mL) and washed with saturated brine. The organic phase was dried over anhydrous sodium sulfate and filtered. The filtrate was concentrated under reduced pressure, and the resulting residue was purified by preparative high performance liquid chromatography (column model: xbridge C8 SN. 1271372511401 waters method: waters-7 xbridge C8 5 μm 19×150 mm 50-80%, A: H₂O (0.1 NH₄HCO₃), B: ACN, MS, flowrate 15 m/min 18 min-GT 12 min) to give isomers 014088A6 P1 (first eluted isomer) (RT: 9.90 min (50 mg)) and 014088A6 P2 (second eluted isomer) (RT: 10.45 min (70 mg)) as yellow solids (120 mg in total, 37% yield).

### Compound 014088A6 P1:

LCMS (M+H)⁺ m/z calculated 650.3. found 650.3. ¹H NMR (DMSO-d₆, 400 MHz): δ 10.32 (s, 1H), 7.82 (d, J=10.8 Hz, 1H), 7.36 (q, J=10.8 Hz, 1H), 7.21 (t, J=10.4 Hz, 1H), 7.09-7.06 (m, 2H), 6.80-6.72 (m, 2H), 4.82-4.80 (m, 1H), 4.17-3.99 (m, 2H), 3.89-3.84 (m, 1H), 3.69-3.61 (m, 1H), 3.15-3.06 (m, 2H), 2.65-2.57 (m, 2H), 1.48 (s, 9H), 1.34 (d, J=8.8 Hz, 3H), 1.10-1.01 (m, 12H).

### Compound 014088A6 P2:

LCMS (M+H)⁺ m/z calculated 650.3. found 650.3. ¹H NMR (DMSO-d₆, 400 MHz): δ 7.95 (d, J=11.6 Hz, 1H), 7.36 (q, J=10.8 Hz, 1H), 7.21 (t, J=10.4 Hz, 1H), 7.07-6.86 (m, 4H), 4.82-4.80 (m, 1H), 4.15-3.99 (m, 2H), 3.90-3.83 (m, 1H), 3.71-3.62 (m, 1H), 3.15-3.06 (m, 2H), 2.70-2.53 (m, 2H), 1.48 (s, 9H), 1.34 (d, J=8.8 Hz, 3H), 1.10-1.03 (m, 6H), 0.99-0.83 (m, 6H).

### Step 9: Synthesis of 014088A7 P1 & 014088A7 P2

Compound 014088A6 P1 (100 mg, 0.154 mmol) was dissolved in dichloromethane (3 mL). Trifluoroacetic acid (526 mg, 4.62 mmol) was added under an ice bath. The ice bath was removed, and the reaction mixture was stirred at room temperature (20° C.) for 2 h, and concentrated under reduced pressure and a water bath at 30° C. to give compound 014088A7 P1 as a yellow oil (80 mg, crude product), which was directly used in the next step without purification. LCMS (M+H)⁺ m/z calculated 550.3. found 550.3.

Compound 014088A6 P2 (150 mg, 0.23 mmol) was dissolved in dichloromethane (3 mL). Trifluoroacetic acid (786 mg, 6.90 mmol) was added under an ice bath. The ice bath was removed, and the reaction mixture was stirred at room temperature (20° C.) for 2 h, and concentrated under reduced pressure and a water bath at 30° C. to give compound 014088A7 P2 as a yellow oil (110 mg, crude product), which was directly used in the next step without purification. LCMS (M+H)⁺ m/z calculated 550.3. found 550.3.

### Step 10: Synthesis of SZ-014088A & SZ-014088B

The crude compound 014088A7 P1 (80 mg, 0.145 mmol) was dissolved in dichloromethane (3 mL). Acryloyl chloride (11 mg, 0.138 mmol) was slowly added under an ice bath, followed by diisopropylethylamine (56 mg, 0.435 mmol). After dropwise addition, the reaction phase was stirred at 0° C. for 10 min. The reaction mixture was diluted with dichloromethane (30 mL). Saturated ammonium chloride solution (20 mL) and saturated sodium bicarbonate solution (3 mL) were added. The organic phase was separated, washed twice with water (2×20 mL), dried over anhydrous magnesium sulfate, and filtered. The filtrate was concentrated under reduced pressure, and the residue was purified by preparative high performance liquid chromatography (ammonium bicarbonate) to give compound SZ-014088A as a pale yellow solid (25 mg, 28.7% yield).

Liquid chromatography-mass spectrometry [mobile phase: elution was carried out with a gradient from 95% water (containing 0.1% trifluoroacetic acid) and 5% acetonitrile to 5% water (containing 0.1% trifluoroacetic acid) and 95% acetonitrile at a flow rate of 1.5 mL per minute at a column temperature of 40° C. for 6 min. Column: waters XBridge C18 3.5 μm, 50×4.6 mm. The purity was 97.43%, Rt=4.159 min. LCMS (M+H)⁺ m/z calculated 604.3. found 604.3. ¹H NMR (DMSO-d₆, 400 MHz): δ 10.29 (s, 1H), 7.83 (m, 1H), 7.33 (q, J=8.4 Hz, 1H), 7.18 (t, J=8.0 Hz, 1H), 7.06-7.03 (m, 2H), 6.87-6.80 (m, 1H), 6.76-6.69 (m, 2H), 6.22-6.18 (m, 1H), 5.76 (dd, J₁=10.4 Hz, J₂=2.4 Hz, 1H), 4.82-4.80 (m, 1H), 4.41-4.28 (m, 1H), 4.17-3.99 (m, 2H), 3.65-3.42 (m, 2H), 3.22-3.04 (m, 1H), 2.60-2.51, m, 2H), 1.29, d, J=6.0 Hz, 3H), 1.07-0.89, m, 12H).

The crude compound 014088A7P2 (110 mg, 0.20 mmol) was dissolved in dichloromethane (4 mL). Acryloyl chloride (17 mg, 0.19 mmol) was slowly added under an ice bath, followed by diisopropylethylamine (77 mg, 0.60 mmol). After dropwise addition, the reaction phase was stirred at 0° C. for 10 min. The reaction mixture was diluted with dichloromethane (50 mL). Saturated ammonium chloride solution (20 mL) and saturated sodium bicarbonate solution (3 mL) were added. The organic phase was separated, washed twice with water (2×20 mL), dried over anhydrous magnesium sulfate, and filtered. The filtrate was concentrated under reduced pressure, and the residue was purified by preparative high performance liquid chromatography (ammonium bicarbonate) to give compound SZ-014088B as a pale yellow solid (30 mg, 25.0% yield).

Liquid chromatography-mass spectrometry [mobile phase: elution was carried out with a gradient from 95% water (containing 0.1% trifluoroacetic acid) and 5% acetonitrile to 5% water (containing 0.1% trifluoroacetic acid) and 95% acetonitrile at a flow rate of 1.5 mL per minute at a column temperature of 40° C. for 6 min. Column: waters XBridge C18 3.5 μm, 50×4.6 mm. The purity was 91.22%, Rt=4.285 min. LCMS (M+H)⁺ m/z calculated 604.3. found 604.3. ¹H NMR (DMSO-d₆, 400 MHz): δ 7.97-7.92 (m, 1H), 7.33 (q, J=6.8 Hz, 1H), 7.18 (t, J=8.0 Hz, 1H), 7.04-6.82 (m, 5H), 6.22-6.18 (m, 1H), 5.78-5.74 (m, 1H), 4.82-4.80 (m, 1H), 4.44-4.29 (m, 1H), 4.14-4.01 (m, 2H), 3.68-3.32 (m, 2H), 3.25-2.98 (m, 1H), 2.68-2.55 (m, 2H), 1.27-1.24 (m, 3H), 1.19-1.00 (m, 6H), 0.94-0.75 (m, 6H).

### Example 2. SZ-014089A/B

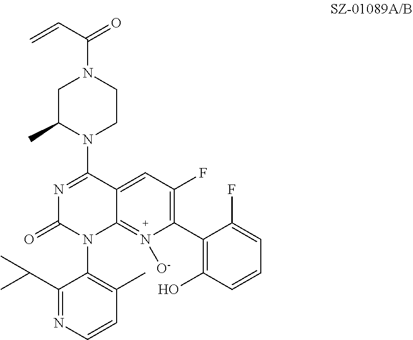

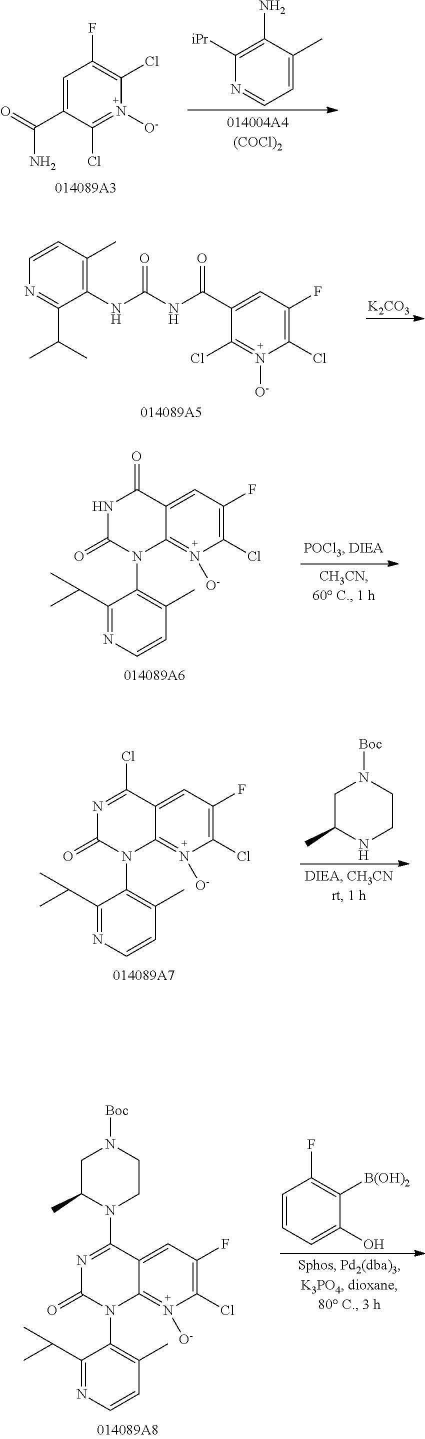

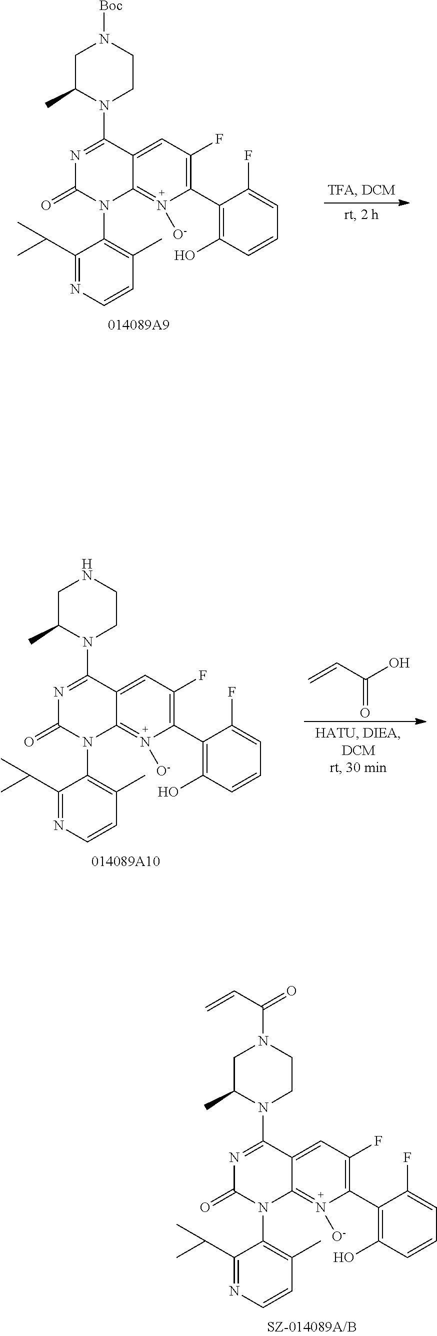

### Step 1: Synthesis of 014089A5

Compound 014089A3 (5.0 g, 0.022 mol) was dissolved in 50 mL of tetrahydrofuran. Oxalyl chloride (3.4 g, 0.026 mol) was added at 0° C. The reaction mixture was stirred at 75° C. for 1 h, and removed from the heat to cool to room temperature. A solution of 014004A4 (3 g, 0.022 mol) in tetrahydrofuran was added dropwise at 0° C. The reaction mixture was stirred at 0° C. for 1 h. The reaction mixture was quenched with saturated aqueous sodium bicarbonate solution (50 mL) and then extracted with ethyl acetate (100 mL×2). The organic phases were combined, dried over anhydrous magnesium sulfate, and filtered under vacuum. The filtrate was concentrated, and the residue was purified by column chromatography (DCM/MeOH=100/1 to 70/1) to give compound 014089A5 as a white solid (3.6 g, 40.4% yield). LCMS (M+H)⁺ m/z calculated 401.0. found 401.0.

### Step 2: Synthesis of 014089A6

Compound 014089A5 (3.6 g, 0.009 mol) was dissolved in 40 mL of N,N-dimethylformamide. Potassium carbonate (2.5 g, 0.018 mol) was added at room temperature. The reaction mixture was stirred at room temperature for 15 h. 400 mL of water was added. The aqueous phase was adjusted to pH 6-7 with 1 N HCl solution and extracted with ethyl acetate (300 mL×2). The organic phases were combined, washed with saturated brine (200 mL×2), dried over anhydrous magnesium sulfate, and filtered under vacuum. The filtrate was concentrated, and the residue was purified by column chromatography (DCM/MeOH=100/1 to 70/1) to give compound 014089A6 as a white solid (2 g, 61.4% yield). LCMS (M+H)⁺ m/z calculated 365.1. found 365.1.

### Step 3: Synthesis of 014089A7

Compound 014089A6 (700 mg, 1.92 mmol) was dissolved in 10 mL of anhydrous acetonitrile. Phosphorus oxychloride (885 mg, 5.77 mmol) and N,N-diisopropylethylamine (744 mg, 5.77 mmol) were added under an ice water bath. After dropwise addition, the reaction mixture was stirred at 60° C. for 1 h, cooled to room temperature and concentrated to dryness by rotary evaporation to give 014089A7 as a brown oily residue (735 mg, crude product), which was used in the next step without purification. LCMS (M+H)⁺ m/z calculated 383.0. found 383.0.

### Step 4: Synthesis of 014089A8

Compound 014089A7 (735 mg, 1.92 mmol) was dissolved in 10 mL of anhydrous acetonitrile. N,N-diisopropylethylamine (744 mg, 5.77 mmol) and (S)-4-N-tert-butoxycarbonyl-2-methylpiperazine (1.44 g, 7.2 mmol) were added under an ice water. The ice bath was removed, and the reaction mixture was stirred at room temperature (20° C.) for 1 h. Then, ethyl acetate (150 mL) was added, followed by washing with saturated brine. The aqueous phase was further extracted with ethyl acetate (100 mL×2), and the organic phases were combined and dried over anhydrous sodium sulfate. The filtrate was concentrated under reduced pressure, and the resulting residue was purified by silica gel column chromatography (petroleum ether/ethyl acetate=1:1) to give compound 014089A8 as a pale yellow solid (650 mg, 62% yield). LCMS (M+H)⁺ m/z calculated 547.2. found 547.4.

### Step 5: Synthesis of 014089A9

Compound 014089A8 (600 mg, 1.10 mmol), 2-fluoro-6-hydroxyphenylboronic acid (600 mg, 3.85 mmol), potassium phosphate (450 mg, 2.12 mmol) and 2-dicyclohexylphosphine-2′,6′-dimethoxybiphenyl (150 mg, 0.37 mmol) were dissolved in 1,4-dioxane (30 mL). The system was purged with nitrogen several times, and then tris(dibenzylidene-BASE acetone)dipalladium(0) (90 mg, 0.098 mmol) was added. The system was purged with nitrogen several times, and then the reaction mixture was stirred at 80° C. for 3 h, cooled to room temperature, and filtered. The filtrate was diluted with ethyl acetate (100 mL) and washed with saturated brine. The organic phases were combined and dried over anhydrous sodium sulfate. The filtrate was concentrated under reduced pressure, and the resulting residue was purified by silica gel column chromatography (petroleum ether/ethyl acetate 1:2) to give compound 014089A9 as a yellow solid (350 mg, crude product, containing starting materials, ligand with a molecular weight of 427, two isomer products). LCMS (M+H)⁺ m/z calculated 623.3. found 623.3.

### Step 6: Synthesis of 014089A10

The crude compound 014089A9 (300 mg, 0.48 mmol) was dissolved in dichloromethane (6 mL). Trifluoroacetic acid (1.65 g, 14.47 mmol) was added under an ice bath. The ice bath was removed, and the reaction mixture was stirred at room temperature (20° C.) for 2 h, adjusted to pH 8 with saturated sodium bicarbonate, and extracted with dichloromethane (10 mL×2). The organic phases were combined and dried over anhydrous sodium sulfate. The filtrate was concentrated under reduced pressure to give 014089A10 as a yellow solid (250 mg, crude product), which was used in the next step without purification. LCMS (M+H)⁺ m/z calculated 523.2. found 523.2.

### Step 7: Synthesis of SZ-014089A/B

The crude compound 014089A10 (90 mg, 0.17 mmol) was dissolved in dichloromethane (3.0 mL). 2-(7-Azobenzotriazole)-N,N,N′,N′-tetramethyluronium hexafluorophosphate (48 mg, 0.13 mmol), N,N-diisopropylethylamine (22 mg, 0.17 mmol) and acrylic acid (4 mg, 0.057 mmol) were added. The reaction mixture was stirred at room temperature (25° C.) for 30 min. The reaction mixture was diluted with 20 mL of dichloromethane, washed with water, and concentrated under reduced pressure. The residue was purified by preparative high performance liquid chromatography (ammonium bicarbonate) to give compound SZ-014089A/B as a yellow solid (1.8 mg, 1.7% yield). Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm]. The sum of purity was 88.47% for 4 isomers. LCMS (M+H)⁺ m/z calculated 577.2. found 577.3.

### Example 3. SZ-014010A/B

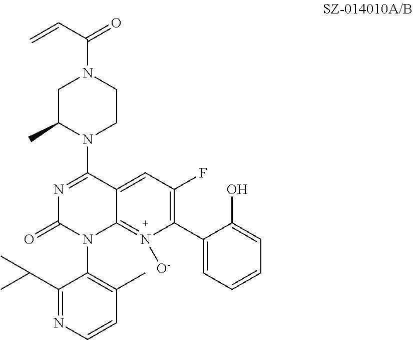

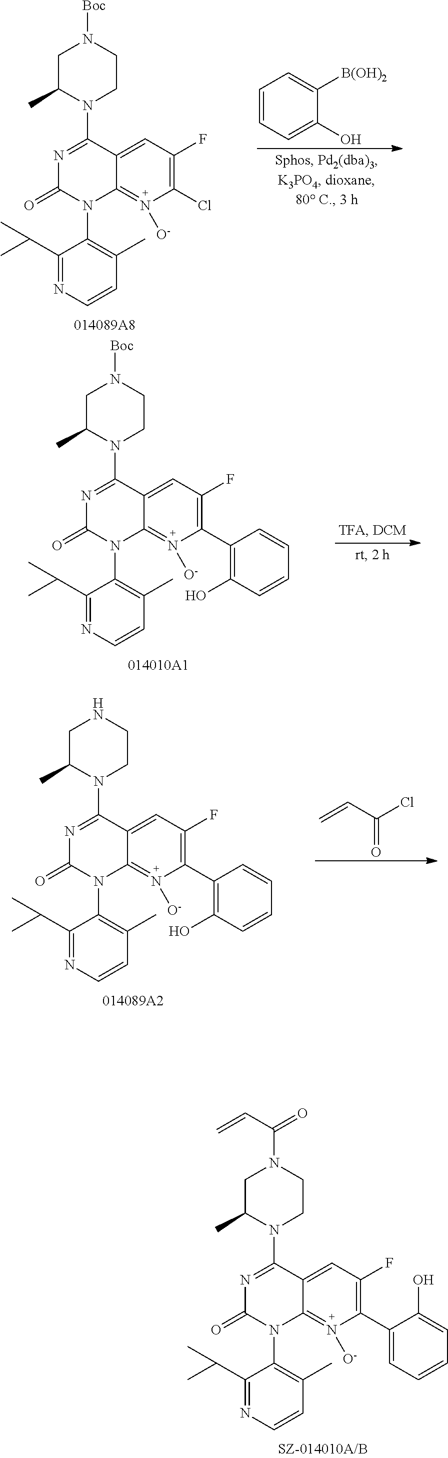

### Step 1: Synthesis of 014010A1

Compound 014089A8 (550 mg, 0.99 mmol), 2-hydroxyphenylboronic acid (352 mg, 2.53 mmol), potassium phosphate (418 mg, 1.98 mmol) and Sphos (121 mg, 0.297 mmol) were dissolved in 1,4-dioxane (20 mL). The system was purged with nitrogen several times, and then Pd₂(dba)₃ (99 mg, 0.099 mmol) was added. The reaction mixture was stirred at 95° C. for 5 h, cooled to room temperature, and filtered. The filtrate was diluted with ethyl acetate (100 mL) and washed with saturated brine. The organic phases were combined and dried over anhydrous sodium sulfate. The filtrate was concentrated under reduced pressure, and the resulting residue was purified by HPLC and lyophilized to give compound 014010A1 as a yellow solid (190 mg, 31.2% yield). LCMS (M+H)⁺ m/z calculated 605.3. found 605.3.

### Step 2: Synthesis of 014010A2

Compound 014010A1 (190 mg, 0.31 mmol) was dissolved in dichloromethane (3 mL). Trifluoroacetic acid (1 mL) was added under an ice bath. The ice bath was removed, and the reaction mixture was stirred at room temperature for 1 h, and concentrated (with toluene to remove trifluoroacetic acid) to give 014010A2 as a yellow oil, which was directly used in the next step without purification. (280 mg, crude product). LCMS (M+H)⁺ m/z calculated 505.3. found 505.3.

### Step 3: Synthesis of SZ-014010A/B

The crude compound 014010A2 (280 mg, 0.55 mmol) was dissolved in dichloromethane (3.0 mL). Acryloyl chloride (36 mg, 0.38 mmol) and N,N-diisopropylethylamine (72 mg, 0.55 mmol) were added. The reaction mixture was stirred at 0° C. for 10 min. The reaction mixture was diluted with 20 mL of dichloromethane, washed with water, and concentrated under reduced pressure. The residue was purified by preparative high performance liquid chromatography (ammonium bicarbonate) to give SZ-014010A/B as a yellow solid (120 mg). Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 15 min. Column: waters XBridge C18 3.5 μm, 150×4.6 mm]. The purity was 97.36%, Rt=9.590 min. LCMS (M+H)⁺ m/z calculated 559.3. found 559.3. ¹H NMR (DMSO-d₆, 400 MHz): δ 9.60 (br, 1H), 8.252 (dd, J1=4.8. Hz, J2=2.0 Hz, 1H), 7.90-7.87 (m, 1H), 7.35-7.31 (td, 1H), 7.19 (br, 1H), 7.03 (t, J=4.6, 1H), 6.93-6.81 (m, 3H), 6.21 (d, J=16.4, 1H), 5.76 (dd, J1=10.4. Hz, J2=2.4 Hz, 1H), 4.87 (br, 1H), 4.42-4.01 (m, 3H), 3.71-3.40 (m, 2H), 3.25-3.02 (m, 1H), 2.79-2.67 (m, 1H), 2.07-2.01 (m, 3H), 1.33-1.29 (m, 3H), 1.03-1.01 (m, 6H).

### Example 4. SZ-014011A/B

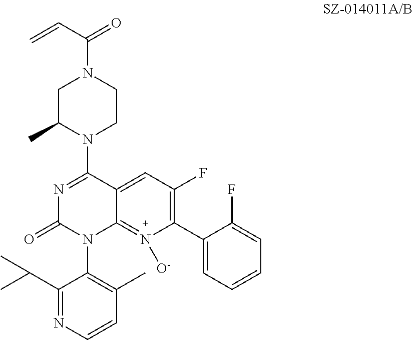

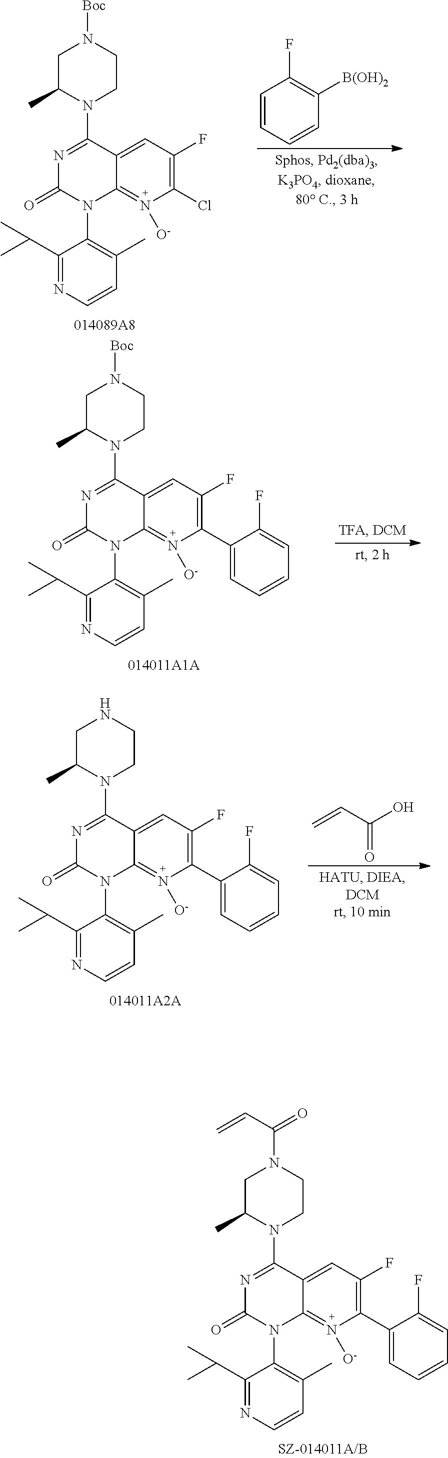

### Step 1: Synthesis of 014011A1A

Compound 014089A8 (500 mg, 0.91 mmol), 2-fluorophenylboronic acid (384 mg, 2.74 mmol), potassium phosphate (386 mg, 1.82 mmol) and Sphos (119 mg, 0.29 mmol) were dissolved in 1,4-dioxane (30 mL). The system was purged with nitrogen several times, and then Pd₂(dba)₃ (83 mg, 0.091 mmol) was added. The system was purged with nitrogen several times, and then the reaction mixture was stirred at 80° C. for 3 h, cooled to room temperature, and filtered. The filtrate was diluted with ethyl acetate (100 mL) and washed with saturated brine (100 mL). The organic phase was dried over anhydrous sodium sulfate and filtered. The filtrate was concentrated under reduced pressure, and the resulting residue was purified by preparative high performance liquid chromatography (ammonium bicarbonate) to give compound 014010A1A as a yellow solid (182 mg, 33% yield). Preparation conditions: liquid chromatography-mass spectrometry [mobile phase: elution was carried out with a gradient from 65% water (containing 0.1% ammonium bicarbonate) and 35% acetonitrile to 25% water (containing 0.1% ammonium bicarbonate) and 75% acetonitrile at a flow rate of 15 mL per minute at a column temperature of 40° C. for 18 min. Column: waters XBridge C8, 5 μm, 19×150 mm] mass spectrometry monitoring. LCMS (M+H⁺) m/z calculated 607.3. found 607.3.

### Step 2: Synthesis of 014011A2A

Compound 014011A1A (182 mg, 0.3 mmol) was dissolved in dichloromethane (3 mL). Trifluoroacetic acid (1 mL) was added under an ice bath. The ice bath was removed, and the reaction mixture was stirred at room temperature for 2 h, adjusted to pH 8 with saturated sodium bicarbonate, and extracted with dichloromethane (20 mL×3). The organic phases were combined and dried over anhydrous sodium sulfate. The filtrate was concentrated under reduced pressure to give 014011A2A as a yellow solid, which was used in the next step without purification. (146 mg, crude product). LCMS (M+H)⁺ m/z 507.3.

### Step 3: Synthesis of SZ-014011A/B

The crude compound 01411A2A (146 mg, 0.29 mmol) was dissolved in dichloromethane (10.0 mL). HATU (164 mg, 0.43 mmol), N,N-diisopropylethylamine (112 mg, 0.87 mmol) and acrylic acid (21 mg, 0.29 mmol) were added. The reaction mixture was stirred at room temperature for 10 min. The reaction mixture was diluted with 20 mL of dichloromethane, washed with water, and concentrated under reduced pressure. The residue was purified by preparative high performance liquid chromatography (ammonium bicarbonate) to give compound SZ-014011A/B as a yellow solid (30 mg, 18.6% yield). Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm]. The purity was 94.77%, Rt=3.223 min. LCMS (M+H)⁺ m/z 561.3. ¹H NMR (DMSO-d₆, 400 MHz): δ 8.25 (dd, J1=4.6. Hz, J2=2.0 Hz, 1H), 7.91 (br, 1H), 7.61-7.56 (m, 1H), 7.50-7.41 (m, 1H), 7.39-7.32 (m, 2H), 7.03 (t, J=4.6, 1H), 6.88-6.84 (m, 1H), 6.21 (d, J=16.4, 1H), 5.76 (dd, J1=10.4. Hz, J2=2.4 Hz, 1H), 4.88 (br, 1H), 4.30-4.01 (m, 3H), 3.71-3.40 (m, 2H), 3.25-3.04 (m, 1H), 2.81-2.67 (m, 1H), 2.07-1.97 (m, 3H), 1.33-1.30 (m, 3H), 1.09-1.01 (m, 6H).

### Example 5. SZ-014079A & SZ-014079B

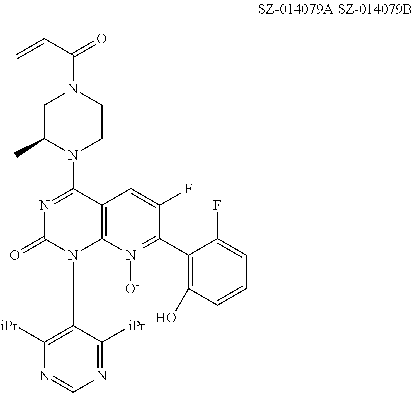 [SMILES: C=CC(=O)N1CCN(c2nc(=O)n(-c3c(C(C)C)ncnc3C(C)C)c3c2cc(F)c(-c2c(O)cccc2F)[n+]3[O-])[C@@H](C)C1]

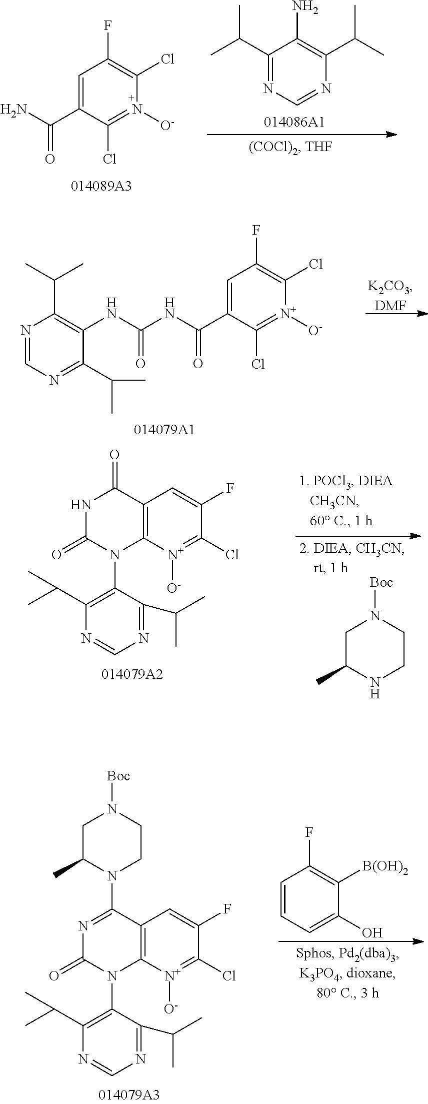

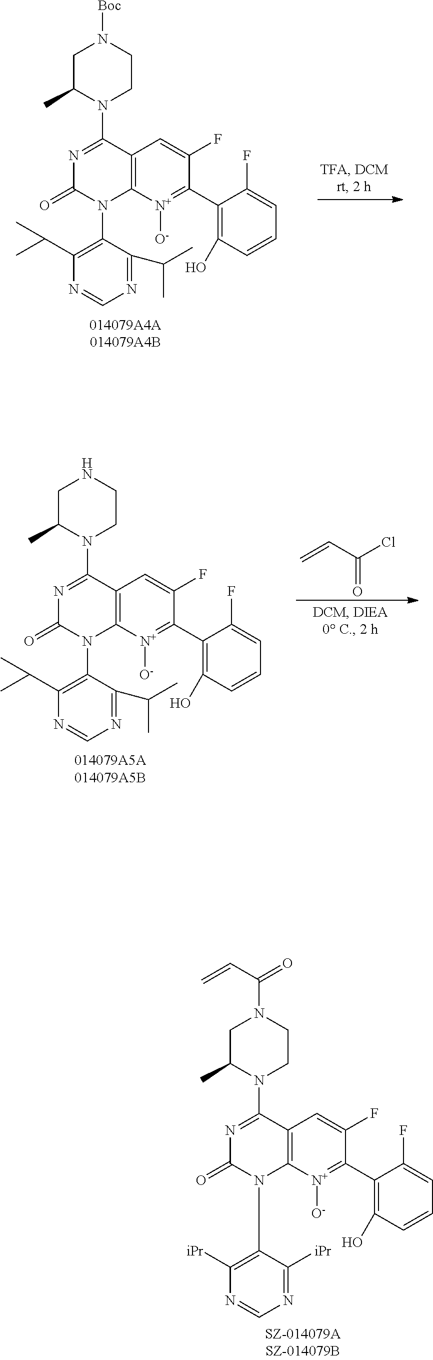

### Step 1: Synthesis of 014079A1

Compound 014089A3 (1.9 g, 0.0083 mol) was dissolved in tetrahydrofuran (30 mL). Oxalyl chloride (1.3 g, 0.0096 mol) was added at 0° C. The reaction mixture was stirred at 75° C. for 1 h, and removed from the heat to cool to room temperature. A solution of compound 014086A1 (1.5 g, 0.0083 mol) in tetrahydrofuran (30 mL) was added dropwise at 0° C. The reaction mixture was stirred at 0° C. for 1 h. The reaction mixture was quenched with saturated aqueous sodium bicarbonate solution (50 mL) and then extracted with ethyl acetate (100 mL×2). The organic phases were combined, dried over anhydrous magnesium sulfate, and filtered. The filtrate was concentrated to give crude compound 014079A1 as a yellow oil (3.7 g, crude product). LCMS (M+H)⁺ m/z calculated 430.0. found 430.0.

### Step 2: Synthesis of 014079A2

Compound 014079A1 (3.7 g, 0.0086 mol) was dissolved in N,N-dimethylformamide (30 mL). Potassium carbonate (2.4 g, 0.017 mol) was added at room temperature. The reaction mixture was stirred at room temperature for 15 h. Water (300 mL) was added. The resulting mixture was adjusted to pH 6-7 with 1 N HCl solution and extracted with ethyl acetate (300 mL×2). The organic phase was washed with saturated brine (200 mL×2), dried over anhydrous magnesium sulfate, and filtered under vacuum. The filtrate was concentrated, and the residue was purified by column chromatography (dichloromethane: methanol=100:1 to 80:1) to give compound 014079A2 as a yellow solid (1.35 g, 40% yield). LCMS (M+H)⁺ m/z calculated 394.0. found 394.0. ¹HNMR (DMSO-d₆, 400 MHz): δ 12.7 (s, 1H), 9.02 (s, 1H), 8.16-8.14 (d, J=6.8 Hz, 1H), 3.07-3.04 (m, 2H), 1.10-1.08 (d, J=6.4 Hz, 6H), 1.03-1.01 (d, J=6.4 Hz, 6H).

### Step 3: Synthesis of 014079A3

Compound 014079A2 (830 mg, 2.11 mmol) was dissolved in anhydrous acetonitrile (10 mL). Phosphorus oxychloride (971 mg, 6.34 mmol) and N,N-diisopropylethylamine (818 mg, 6.34 mmol) were added under an ice bath. After dropwise addition, the reaction mixture was stirred at 60° C. for 1 h, cooled to room temperature and concentrated to give a brown oily compound (868 mg, crude product), which was used in the next step without purification. The above brown oily compound (868 mg, 2.11 mmol) was dissolved in anhydrous acetonitrile (10 mL). N,N-diisopropylethylamine (818 mg, 6.34 mmol) and (S)-4-N-tert-butoxycarbonyl-2-methylpiperazine (379 mg, 2.53 mmol) were added. The reaction mixture was stirred at room temperature for 1 h. Ethyl acetate (150 mL) was then added, followed by washing with saturated brine (100 mL). The aqueous phase was extracted with ethyl acetate (100 mL×2). The organic phases were combined, dried over anhydrous sodium sulfate, and filtered. The residue was purified by silica gel column chromatography (petroleum ether/ethyl acetate=1:1) to give compound 014079A3 as a yellow solid (470 mg, 39% yield). LCMS (M+H)⁺ m/z calculated 576.4. found 576.4.

### Step 4: Synthesis of 014079A4A & 014079A4B

Compound 014079A3 (370 mg, 0.64 mmol) and 2-fluoro-6-hydroxyphenylboronic acid (349 mg, 2.25 mmol) were dissolved in 1,4-dioxane (30 mL). Potassium phosphate (273 mg, 1.29 mmol) and Sphos (79 mg, 0.19 mmol) were added. The system was purged with nitrogen several times, and then Pd₂(dba)₃ (90 mg, 0.098 mmol) was added. The system was purged with nitrogen three times, and then the reaction mixture was stirred at 80° C. for 3 h, cooled to room temperature, and filtered. The filtrate was diluted with ethyl acetate (100 mL) and washed with saturated brine (100 mL). The organic phase was dried over anhydrous sodium sulfate and filtered. The filtrate was concentrated under reduced pressure, and the resulting residue was purified by preparative high performance liquid chromatography (ammonium bicarbonate) to give compounds 014079A4A (first eluted isomer) (60 mg, 14% yield) and 014079A4B (second eluted isomer) (62 mg, 15% yield) as yellow solids.

Preparation conditions: liquid chromatography-mass spectrometry [mobile phase: elution was carried out with a gradient from 65% water (containing 0.1% ammonium bicarbonate) and 35% acetonitrile to 20% water (containing 0.1% ammonium bicarbonate) and 80% acetonitrile at a flow rate of 15 mL per minute at a column temperature of 40° C. for 11 min. Column: waters XBridge C18, 5 μm, 19×150 mm] 214 nm.

Compound 014079A4A: LCMS (M+H)⁺ m/z calculated 652.3. found 652.3. Rt=9.4 min. ¹HNMR (DMSO-d₆, 400 MHz): δ 10.38-10.37 (m, 1H), 8.88 (s, 1H), 7.87 (d, J=8.4 Hz, 1H), 7.38-7.32 (m, 1H), 6.78-6.71 (m, 2H), 4.85-4.84 (m, 1H), 4.18-4.17 (m, 1H), 3.99-3.97 (m, 1H), 3.87-3.82 (m, 1H), 3.73-3.61 (m, 2H), 3.20-3.05 (m, 1H), 2.87-2.73 (m, 2H), 1.45 (s, 9H), 1.34 (d, J=6.8 Hz, 3H), 1.08-1.04 (m, 6H), 1.02 (d, J=6.8 Hz, 6H).

Compound 014079A4B: LCMS (M+H)⁺ m/z calculated 652.3. found 652.3. Rt=10.6 min. ¹HNMR (DMSO-d₆, 400 MHz): δ 8.87 (s, 1H), 8.02 (d, J=8.4 Hz, 1H), 7.37-7.31 (m, 1H), 6.99-6.87 (m, 3H), 4.85-4.84 (m, 1H), 4.17-4.14 (m, 1H), 4.01-3.99 (m, 1H), 3.89-3.61 (m, 3H), 3.12-3.01 (m, 1H), 2.92-2.79 (m, 2H), 1.45 (s, 9H), 1.32 (d, J=6.8 Hz, 3H), 1.07-1.02 (m, 6H), 0.97-0.94 (m, 6H).

### Step 5: Synthesis of 014079A5A & 014079A5B

Compound 014079A4A (32 mg, 0.049 mmol) was dissolved in dichloromethane (2 mL). Trifluoroacetic acid (200 mg, 1.75 mmol) was added under an ice bath. The ice bath was removed, and the reaction mixture was stirred at room temperature (20° C.) for 2 h, and concentrated to give compound 014079A5A as a yellow solid, which was directly used in the next step without purification. (27 mg, crude product). LCMS (M+H)⁺ m/z calculated 552.3. found m/z 552.3.

Compound 014079A4B (34 mg, 0.052 mmol) was dissolved in dichloromethane (2 mL). Trifluoroacetic acid (200 mg, 1.75 mmol) was added under an ice bath. The ice bath was removed, and the reaction mixture was stirred at room temperature (20° C.) for 2 h, and concentrated under reduced pressure to give compound 014079A5B as a yellow solid, which was directly used in the next step without purification. (29 mg, crude product). LCMS (M+H)⁺ m/z calculated 552.3. found m/z 552.4.

### Step 6: Synthesis of SZ-014079A & SZ-014079B

The crude compound 014079A5A (27 mg, 0.049 mmol) was dissolved in dichloromethane (6.0 mL). Acryloyl chloride (12.5 mg, 0.14 mmol) and N,N-diisopropylethylamine (6 mg, 0.047 mmol) were added under an ice bath. The reaction mixture was stirred under the ice bath for 2 h. The reaction mixture was washed with saturated sodium bicarbonate (20 mL). The organic phase was separated and concentrated to dryness by rotary evaporation, and the residue was purified by preparative high performance liquid chromatography (ammonium bicarbonate) to give compound SZ-014079A as a yellow solid (10 mg, 33% yield).

Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm]. The purity was 89.13%, Rt=3.366 min. LCMS (M+H)⁺ m/z calculated 606.3. found 606.3. ¹H NMR (DMSO-d₆, 400 MHz): δ 10.39-10.37 (m, 1H), 8.88 (s, 1H), 7.92-7.90 (m, 1H), 7.38-7.32 (m, 1H), 6.90-6.71 (m, 3H), 6.21 (d, J=16.4 Hz, 1H), 5.76 (dd, J=10.4, 2.4 Hz, 1H), 4.89-4.86 (m, 1H), 4.43-4.01 (m, 3H), 3.75-3.41 (m, 2H), 3.24-3.04 (m, 1H), 2.86-2.74 (m, 2H), 1.32 (d, J=6.4 Hz, 3H), 1.08-1.02 (m, 12H).

The crude compound 014079A5B (94 mg, 0.17 mmol) was dissolved in dichloromethane (4.0 mL). Acryloyl chloride (60 mg, 0.11 mmol) and N,N-diisopropylethylamine (122 mg, 0.95 mmol) were added under an ice bath. The reaction mixture was stirred under the ice bath for 2 h. The reaction mixture was washed with saturated sodium bicarbonate (20 mL). The organic phase was separated and concentrated to dryness by rotary evaporation, and the residue was purified by preparative high performance liquid chromatography (ammonium bicarbonate) to give compound SZ-014079B as a yellow solid (50 mg, 49% yield).

Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm]. The purity was 97.05%, Rt=3.771 min.

LCMS (M+H)⁺ m/z calculated 606.3. found 606.3. ¹H NMR (DMSO-d₆, 400 MHz): δ 8.87 (s, 1H), 8.06 (d, J=8.0 Hz, 1H), 7.37-7.41 (m, 1H), 6.99-6.85 (m, 4H), 6.21 (d, J=16.4 Hz, 1H), 5.77 (dd, J=10.4, 2.0 Hz, 1H), 4.89 (m, 1H), 4.24-4.03 (m, 3H), 3.77 (m, 1H), 3.64-3.39 (m, 1H), 3.24-3.01 (m, 1H), 2.93-2.80 (m, 2H), 1.30 (d, J=6.8 Hz, 3H), 1.07-1.02 (m, 6H), 0.98-0.94 (m, 6H).

### Example 6. SZ-014016A & SZ-014016B

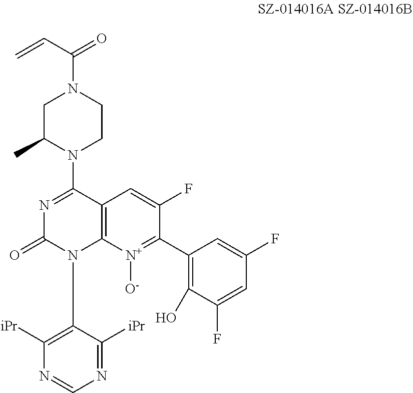 [SMILES: C=CC(=O)N1CCN(c2nc(=O)n(-c3c(C(C)C)ncnc3C(C)C)c3c2cc(F)c(-c2cc(F)cc(F)c2O)[n+]3[O-])[C@@H](C)C1]

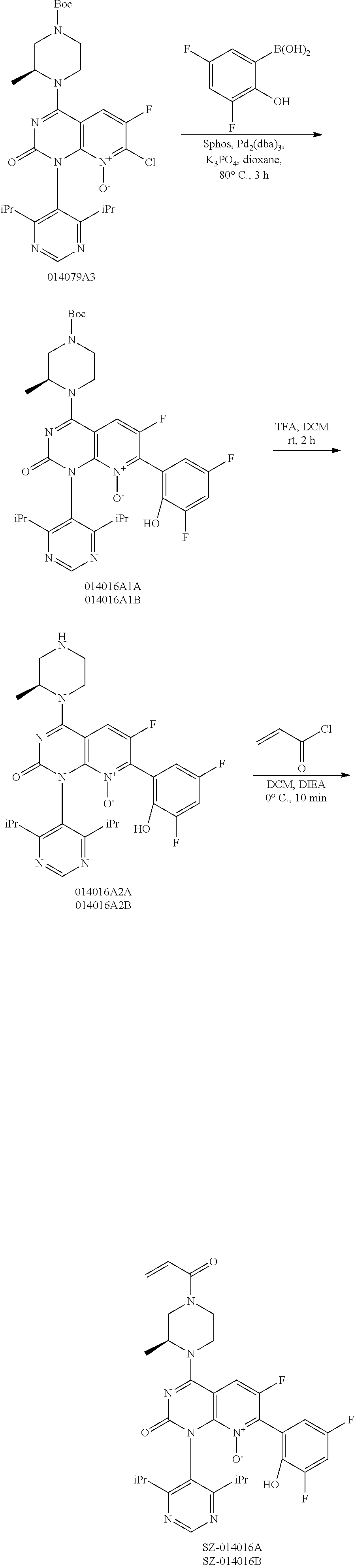

### Step 1: Synthesis of 014016A1A & 014016A1B

Compound 014079A3 (1.2 g, 2.08 mmol) and 3,5-difluoro-2-hydroxyphenylboronic acid (900 mg, 5.18 mmol) were dissolved in 1,4-dioxane (15 mL). Potassium phosphate (880 mg, 4.15 mmol) and 2-dicyclohexylphosphine-2′,6′-dimethoxy-1,1′-biphenyl (260 mg, 0.63 mmol) were added. The system was purged with nitrogen several times, and then tris(dibenzylidene-BASE acetone)dipalladium (180 mg, 0.19 mmol) was added. The system was purged with nitrogen three times, and then the reaction mixture was stirred at 95° C. for 5 h, cooled to room temperature, and filtered. The filtrate was diluted with ethyl acetate (100 mL) and washed with saturated brine (100 mL). The organic phase was dried over anhydrous sodium sulfate and filtered. The filtrate was concentrated under reduced pressure, and the resulting residue was purified by preparative high performance liquid chromatography (ammonium bicarbonate) to give compounds 014016A1A (first eluted isomer) (320 mg, 23% yield) and 014016A1B (second eluted isomer) (190 mg, 14% yield) as yellow solids.

Preparation conditions: liquid chromatography-mass spectrometry [mobile phase: elution was carried out with a gradient from 65% water (containing 0.1% ammonium bicarbonate) and 35% acetonitrile to 20% water (containing 0.1% ammonium bicarbonate) and 80% acetonitrile at a flow rate of 15 mL per minute at a column temperature of 40° C. for 11 min. Column: waters XBridge C18, 5 μm, 19×150 mm] 214 nm. Compound 014016A1A: LCMS (M+H)⁺ m/z calculated 670.3. found 670.3.

¹HNMR (DMSO-d₆, 400 MHz): δ 10.02 (s, 1H), 8.90 (s, 1H), 7.95 (br s, 1H), 7.47-7.41 (m, 1H), 6.93 (d, J=8.4 Hz, 1H), 4.85 (br s, 1H), 4.16-4.15 (m, 1H), 4.02-3.99 (m, 1H), 3.87-3.84 (m, 1H), 3.73-3.61 (m, 2H), 3.13-3.09 (m, 1H), 2.84-2.73 (m, 2H), 1.45 (m, 9H), 1.33 (d, J=6.4 Hz, 3H), 1.07-1.03 (m, 12H). Compound 014016A1B: LCMS (M+H)⁺ m/z calculated 670.3. found 670.3. ¹HNMR (DMSO-d₆, 400 MHz): δ 8.88 (s, 1H), 8.04 (d, J=8.8 Hz, 1H), 7.45-7.39 (m, 1H), 7.18-7.01 (m, 1H), 6.98-6.93 (m, 1H), 4.83 (br s, 1H), 4.16-4.12 (m, 1H), 4.01-3.99 (m, 1H), 3.86-3.83 (m, 1H), 3.72-3.65 (m, 1H), 3.56-3.39 (m, 1H), 3.10-3.09 (m, 1H), 2.85-2.75 (m, 2H), 1.45 (s, 9H), 1.31 (d, J=6.4 Hz, 3H), 1.06-1.02 (m, 6H), 0.95-0.91 (m, 6H).

### Step 2: Synthesis of 014016A2A & 014016A2B

Compound 014016A1A (320 mg, 0.48 mmol) was dissolved in dichloromethane (3 mL). Trifluoroacetic acid (1 mL) was added under an ice bath. The ice bath was removed, and the reaction mixture was stirred at room temperature (20° C.) for 2 h. Half of the reaction mixture was concentrated under reduced pressure to give 014016A2A as a yellow oil (120 mg, crude product), which was directly used in the next step without purification. LCMS (M+H)⁺ m/z calculated 570.3. found 570.3.

Compound 014016A1B (190 mg, 0.28 mmol) was dissolved in dichloromethane (2 mL). Trifluoroacetic acid (0.7 mL) was added under an ice bath. The ice bath was removed, and the reaction mixture was stirred at room temperature (20° C.) for 2 h. Half of the reaction mixture was concentrated under reduced pressure to give compound 014016A2B as a yellow oil (70 mg, crude product), which was directly used in the next step without purification. LCMS (M+H)⁺ m/z calculated 570.3. found 570.3.

### Step 3: Synthesis of SZ-014016A & SZ-014016B

The crude compound 014016A2A (120 mg, 0.21 mmol) was dissolved in dichloromethane (3.0 mL). Acryloyl chloride (19 mg, 0.21 mmol) and N,N-diisopropylethylamine (40.5 mg, 0.32 mmol) were added under an ice bath. The reaction mixture was stirred under the ice bath for 10 min. The reaction mixture was washed with saturated sodium bicarbonate (20 mL). The organic phase was separated and concentrated to dryness by rotary evaporation, and the residue was purified by preparative high performance liquid chromatography (ammonium bicarbonate) to give compound SZ-014016A as a yellow solid (56 mg, 42% yield).

Liquid chromatography-mass spectrometry [mobile phase: elution was carried out with a gradient from 60% water (containing 0.02% ammonium acetate) and 40% acetonitrile to 40% water (containing 0.02% ammonium acetate) and 60% acetonitrile at a flow rate of 1.0 mL per minute at a column temperature of 40° C. for 15 min. Column: waters XBridge C18 3.5 μm, 150×4.6 mm]. The purity was 92.13%, Rt=7.863 min. LCMS (M+H)⁺ m/z calculated 624.2. found 624.2. ¹H NMR (DMSO-d₆, 400 MHz): δ 10.02 (br s, 1H), 8.90 (s, 1H), 7.99 (br s, 1H), 7.47-7.41 (m, 1H), 6.95-6.92 (m, 1H), 6.88-6.81 (m, 1H), 6.23-6.19 (m, 1H), 5.77 (dd, J=10.4, 2.4 Hz, 1H), 4.89 (br s, 1H), 4.44-4.02 (m, 3H), 3.76-3.61 (m, 1H), 3.43-3.31 (m, 1H), 3.24-3.07 (m, 1H), 2.89-2.83 (m, 2H), 1.31 (d, J=6.4 Hz, 3H), 1.07-1.04 (m, 12H).

The crude compound 014016A2B (70 mg, 0.12 mmol) was dissolved in dichloromethane (3.0 mL). Acryloyl chloride (11 mg, 0.12 mmol) and N,N-diisopropylethylamine (23 mg, 0.18 mmol) were added under an ice bath. The reaction mixture was stirred under the ice bath for 10 min. The reaction mixture was washed with saturated sodium bicarbonate (20 mL). The organic phase was separated and concentrated to dryness by rotary evaporation, and the residue was purified by preparative high performance liquid chromatography (ammonium bicarbonate) to give compound SZ-014016B as a yellow solid (35 mg, 45% yield).

Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm]. The purity was 92.12%, Rt=3.889 min. LCMS (M+H)⁺ m/z calculated 624.2. found 624.2. ¹H NMR (DMSO-d₆, 400 MHz): δ 8.88 (s, 1H), 8.09-8.07 (br s, 1H), 7.45-7.39 (m, 1H), 7.16-7.10 (m, 1H), 6.98-6.93 (m, 1H), 6.88-6.85 (m, 1H), 6.23-6.19 (m, 1H), 5.77 (dd, J=10.4, 2.4 Hz, 1H), 4.87 (br s, 1H), 4.44-4.41 (m, 1H), 4.32-4.02 (m, 2H), 3.75 (br s, 1H), 3.64-3.39 (m, 1H), 3.26-3.01 (m, 1H), 2.88-2.76 (m, 2H), 1.30 (d, J=6.8 Hz, 3H), 1.06-1.02 (m, 6H), 0.96-0.92 (m, 6H).

### Example 7. SZ-014028A & SZ-014028B

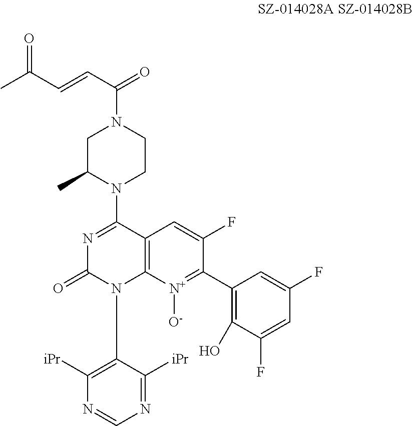 [SMILES: CC(=O)/C=C/C(=O)N1CCN(c2nc(=O)n(-c3c(C(C)C)ncnc3C(C)C)c3c2cc(F)c(-c2cc(F)cc(F)c2O)[n+]3[O-])[C@@H](C)C1]

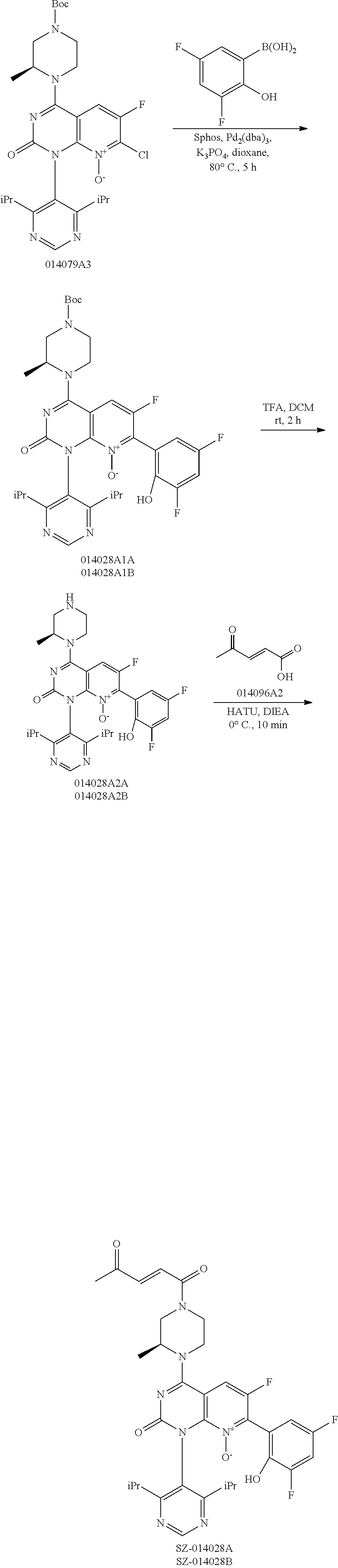

### Step 1: Synthesis of 014028A1A & 014028A1B

Compound 014079A3 (1.2 g, 2.08 mmol) and 3,5-difluoro-2-hydroxyphenylboronic acid (900 mg, 5.18 mmol) were dissolved in 1,4-dioxane (15 mL). Potassium phosphate (880 mg, 4.15 mmol) and 2-dicyclohexylphosphine-2′,6′-dimethoxy-1,1′-biphenyl (260 mg, 0.63 mmol) were added. The system was purged with nitrogen several times, and then tris(dibenzylidene-BASE acetone)dipalladium (180 mg, 0.19 mmol) was added. The system was purged with nitrogen three times, and then the reaction mixture was stirred at 95° C. for 5 h, cooled to room temperature, and filtered. The filtrate was diluted with ethyl acetate (100 mL) and washed with saturated brine (100 mL). The organic phase was dried over anhydrous sodium sulfate and filtered. The filtrate was concentrated under reduced pressure, and the resulting residue was purified by preparative high performance liquid chromatography (ammonium bicarbonate) to give compounds 014028A1A (first eluted isomer) (320 mg, 23% yield) and 014028A1B (second eluted isomer) (190 mg, 14% yield) as yellow solids.

Preparation conditions: liquid chromatography-mass spectrometry [mobile phase: elution was carried out with a gradient from 65% water (containing 0.1% ammonium bicarbonate) and 35% acetonitrile to 20% water (containing 0.1% ammonium bicarbonate) and 80% acetonitrile at a flow rate of 15 mL per minute at a column temperature of 40° C. for 11 min. Column: waters XBridge C18, 5 μm, 19×150 mm] 214 nm.

Compound 014028A1A: LCMS (M+H)⁺ m/z calculated 670.3. found 670.3. ¹HNMR (DMSO-d₆, 400 MHz): δ 10.02 (s, 1H), 8.90 (s, 1H), 7.95 (br s, 1H), 7.47-7.41 (m, 1H), 6.93 (d, J=8.4 Hz, 1H), 4.85 (br s, 1H), 4.16-4.15 (m, 1H), 4.02-3.99 (m, 1H), 3.87-3.84 (m, 1H), 3.73-3.61 (m, 2H), 3.13-3.09 (m, 1H), 2.84-2.73 (m, 2H), 1.45 (s, 9H), 1.33 (d, J=6.4 Hz, 3H), 1.07-1.03 (m, 12H). Compound 014028A1B: LCMS (M+H)⁺ m/z calculated 670.3. found 670.3. ¹HNMR (DMSO-d₆, 400 MHz): δ 8.88 (s, 1H), 8.04 (d, J=8.8 Hz, 1H), 7.45-7.39 (m, 1H), 7.18-7.01 (m, 1H), 6.98-6.93 (m, 1H), 4.83 (br s, 1H), 4.16-4.12 (m, 1H), 4.01-3.99 (m, 1H), 3.86-3.83 (m, 1H), 3.72-3.65 (m, 1H), 3.56-3.39 (m, 1H), 3.10-3.09 (m, 1H), 2.85-2.75 (m, 2H), 1.45 (s, 9H), 1.31 (d, J=6.4 Hz, 3H), 1.06-1.02 (m, 6H), 0.95-0.91 (m, 6H).

### Step 2: Synthesis of 014028A2A & 014028A2B

Compound 014028A1A (320 mg, 0.48 mmol) was dissolved in dichloromethane (3 mL). Trifluoroacetic acid (1 mL) was added under an ice bath. The ice bath was removed, and the reaction mixture was stirred at room temperature (20° C.) for 2 h. Half of the reaction mixture was transferred out, and 5 mL of dichloromethane was added thereto, followed by slow addition of 10 mL of saturated sodium bicarbonate solution. The organic phase was separated, washed twice with brine (10 mL×2), dried over magnesium sulfate, and filtered. The resulting filtrate was concentrated to give 014028A2A as a yellow solid (120 mg, crude product), which was directly used in the next step without purification. LCMS (M+H)⁺ m/z calculated 570.3. found 570.3.

Compound 014028A1B (190 mg, 0.28 mmol) was dissolved in dichloromethane (2 mL). Trifluoroacetic acid (0.7 mL) was added under an ice bath. The ice bath was removed, and the reaction mixture was stirred at room temperature (20° C.) for 2 h. Half of the reaction mixture was transferred out, and 5 mL of dichloromethane was added thereto, followed by slow addition of 10 mL of saturated sodium bicarbonate solution. The organic phase was separated, washed twice with brine (10 mL×2), dried over magnesium sulfate, and filtered. The resulting filtrate was concentrated to give 014028A2B as a yellow solid (70 mg, crude product), which was directly used in the next step without purification. LCMS (M+H)⁺ m/z calculated 570.3. found 570.3.

### Step 3: Synthesis of SZ-014028A & SZ-014028B

The crude compound 014028A2A (120 mg, 0.21 mmol) was dissolved in dichloromethane (6.0 mL). 014096A2 (48 mg, 0.32 mmol), 2-(7-benzotriazole oxide)-N,N,N′,N′-tetramethyluronium hexafluorophosphate (160 mg, 0.42 mmol) and N,N-diisopropylethylamine (82 mg, 0.63 mmol) were added under an ice bath. The reaction mixture was stirred under the ice bath for 10 min. The reaction mixture was washed with saturated sodium bicarbonate (30 mL). The organic phase was separated and concentrated to dryness by rotary evaporation, and the residue was purified by preparative high performance liquid chromatography (ammonium bicarbonate) to give compound SZ-014028A as a yellow solid (13 mg, 9% yield).

Liquid chromatography-mass spectrometry [mobile phase: elution was carried out with a gradient from 80% water (containing 0.1% trifluoroacetic acid) and 20% acetonitrile to 30% water (containing 0.1% trifluoroacetic acid) and 70% acetonitrile at a flow rate of 1.0 mL per minute at a column temperature of 40° C. for 6 min. Column: waters XBridge C18 3.5 μm, 4.6×50 mm]. The purity was 88.69%, Rt=3.770 min. LCMS (M+H)⁺ m/z calculated 666.3. found 666.3. ¹H NMR (DMSO-d₆, 400 MHz): δ 10.04 (s, 1H), 8.90 (s, 1H), 7.99 (br s, 1H), 8.00-7.98 (m, 1H), 7.50-7.39 (m, 2H), 6.93 (d, J=8.4 Hz, 1H), 6.81-6.76 (m, 1H), 4.90 (br s, 1H), 4.41-4.31 (m, 1H), 4.27-4.60 (m, 2H), 3.80 (br s, 1H), 3.72-3.45 (m, 1H), 3.22-3.17 (m, 1H), 2.87 (br s, 1H), 2.39 (m, 3H), 1.34-1.32 (m, 3H). 1.07-1.04 (m, 12H).

The crude compound 014028A2B (70 mg, 0.12 mmol) was dissolved in dichloromethane (5.0 mL). 014096A2 (27 mg, 0.18 mmol), 2-(7-benzotriazole oxide)-N,N,N′,N′-tetramethyluronium hexafluorophosphate (90 mg, 0.24 mmol) and N,N-diisopropylethylamine (46 mg, 0.36 mmol) were added under an ice bath. The reaction mixture was stirred under the ice bath for 10 min. The reaction mixture was washed with saturated sodium bicarbonate (20 mL). The organic phase was separated and concentrated to dryness by rotary evaporation, and the residue was purified by preparative high performance liquid chromatography (ammonium bicarbonate) to give compound SZ-014028B as a yellow solid (7 mg, 8% yield).

Liquid chromatography-mass spectrometry [mobile phase: elution was carried out with a gradient from 70% water (containing 0.02% ammonium acetate) and 30% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm]. The purity was 86.92%, Rt=3.300 min.

LCMS (M+H)⁺ m/z calculated 666.3. found 666.2. ¹H NMR (DMSO-d₆, 400 MHz): δ 8.88 (s, 1H), 8.11-8.05 (m, 1H), 7.45-7.39 (m, 2H), 7.14-7.11 (m, 1H), 6.99-6.94 (m, 1H), 6.81-6.75 (m, 1H), 4.89 (br s, 1H), 4.41-4.31 (m, 1H), 4.24-4.05 (m, 2H), 3.83-3.77 (m, 1H), 3.68-3.45 (m, 1H), 3.26-3.14 (m, 1H), 2.85-2.78 (m, 2H), 2.39 (s, 3H), 1.33-1.30 (m, 3H), 1.06-1.02 (m, 6H), 0.96-0.92 (m, 6H).

### Example 8. SZ-014041A & SZ-014041B

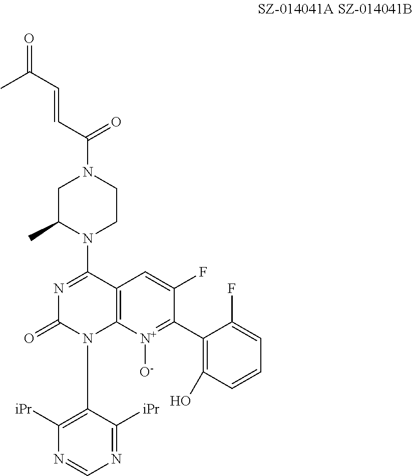 [SMILES: CC(=O)/C=C/C(=O)N1CCN(c2nc(=O)n(-c3c(C(C)C)ncnc3C(C)C)c3c2cc(F)c(-c2c(O)cccc2F)[n+]3[O-])[C@@H](C)C1]

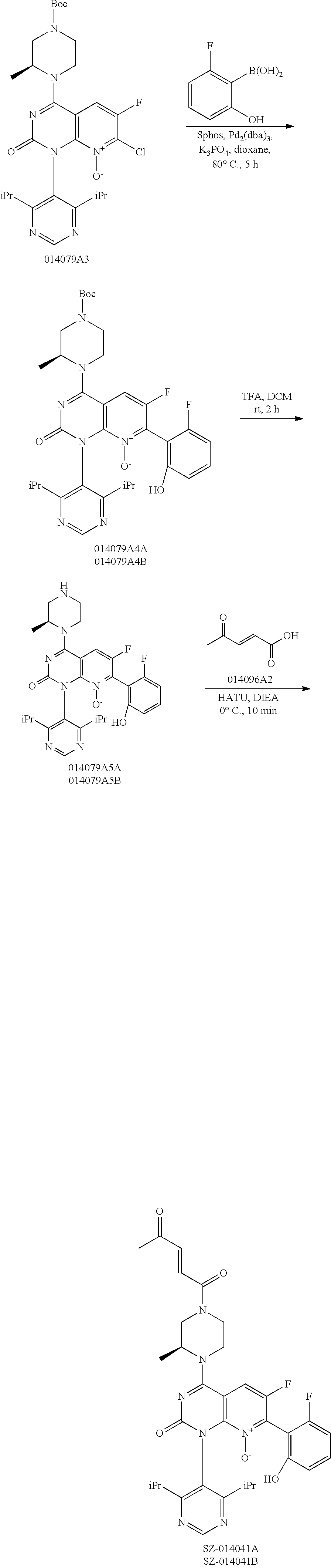

### Step 1: Synthesis of 014079A4A & 014079A4B

Compound 014079A3 (370 mg, 0.64 mmol) and 2-fluoro-6-hydroxyphenylboronic acid (349 mg, 2.25 mmol) were dissolved in 1,4-dioxane (30 mL). Potassium phosphate (273 mg, 1.29 mmol) and 2-dicyclohexylphosphine-2′,6′-dimethoxy-1,1′-biphenyl (79 mg, 0.19 mmol) were added. The system was purged with nitrogen several times, and then tris(dibenzylidene-BASE acetone)dipalladium (90 mg, 0.098 mmol) was added. The system was purged with nitrogen three times, and then the reaction mixture was stirred at 80° C. for 3 h, cooled to room temperature, and filtered. The filtrate was diluted with ethyl acetate (100 mL) and washed with saturated brine (100 mL). The organic phase was dried over anhydrous sodium sulfate and filtered. The filtrate was concentrated under reduced pressure, and the resulting residue was purified by preparative high performance liquid chromatography (ammonium bicarbonate) to give compounds 014079A4A (first eluted isomer) (85 mg, 20% yield) and 014079A4B (second eluted isomer) (125 mg, 29% yield) as yellow solids. Preparation conditions: liquid chromatography-mass spectrometry [mobile phase: elution was carried out with a gradient from 65% water (containing 0.1% ammonium bicarbonate) and 35% acetonitrile to 20% water (containing 0.1% ammonium bicarbonate) and 80% acetonitrile at a flow rate of 15 mL per minute at a column temperature of 40° C. for 11 min. Column: waters XBridge C18, 5 μm, 19×150 mm] 214 nm.

Compound 014079A4A: LCMS (M+H)⁺ m/z calculated 652.3. found 652.3. Rt=9.4 min. ¹HNMR (DMSO-d₆, 400 MHz): δ 10.38-10.37 (m, 1H), 8.88 (s, 1H), 7.87 (d, J=8.4 Hz, 1H), 7.38-7.32 (m, 1H), 6.78-6.71 (m, 2H), 4.85-4.84 (m, 1H), 4.18-4.17 (m, 1H), 3.99-3.97 (m, 1H), 3.87-3.82 (m, 1H), 3.73-3.61 (m, 2H), 3.20-3.05 (m, 1H), 2.87-2.73 (m, 2H), 1.45 (s, 9H), 1.34 (d, J=6.8 Hz, 3H), 1.08-1.04 (m, 6H), 1.02 (d, J=6.8 Hz, 6H).

Compound 014079A4B: LCMS (M+H)⁺ m/z calculated 652.3. found 652.3. Rt=10.6 min. ¹HNMR (DMSO-d₆, 400 MHz): δ 8.87 (s, 1H), 8.02 (d, J=8.4 Hz, 1H), 7.37-7.31 (m, 1H), 6.99-6.87 (m, 3H), 4.85-4.84 (m, 1H), 4.17-4.14 (m, 1H), 4.01-3.99 (m, 1H), 3.89-3.61 (m, 3H), 3.12-3.01 (m, 1H), 2.92-2.79 (m, 2H), 1.45 (s, 9H), 1.32 (d, J=6.8 Hz, 3H), 1.07-1.02 (m, 6H), 0.97-0.94 (m, 6H).

### Step 2: Synthesis of 014079A5A

Compound 014079A4A (85 mg, 0.136 mmol) was dissolved in dichloromethane (2 mL). Trifluoroacetic acid (1 mL) was added under an ice bath. The ice bath was removed, and the reaction mixture was stirred at room temperature (20° C.) for 2 h. The reaction phase was concentrated under reduced pressure and a water bath at 30° C. to give a yellow oil, and 5 mL of dichloromethane was added. 10 mL of saturated aqueous sodium bicarbonate solution was slowly added. The organic phase was separated, washed twice with brine (10 mL×2), dried over magnesium sulfate, and filtered. The filtrate was concentrated under reduced pressure to give 014079A5A as a yellow solid (65 mg, crude product), which was used in the next step without purification. LCMS (M+H)⁺ m/z calculated 552.2. found 552.2.

### Step 3: Synthesis of SZ-014041A

Compounds 014079A5A (65 mg, 0.117 mmol) and 014096A2 (16.0 mg, 0.140 mmol) and 2-(7-benzotriazole oxide)-N,N,N′,N′-tetramethyluronium hexafluorophosphate (53.4 mg, 0.140 mmol) were dissolved in dichloromethane (2.0 mL), to which N,N-diisopropylethylamine (30.3 mg, 0.234 mmol) were added. The reaction phase was stirred at room temperature (25° C.) for 2 h. The reaction mixture was concentrated to remove N,N-dimethylformamide. The residue was purified by preparative high performance liquid chromatography to give compound SZ-014041A as a white solid (10.5 mg, 14% yield).

Liquid chromatography-mass spectrometry [mobile phase: elution was carried out with a gradient from 60% water (containing 0.02% ammonium acetate) and 40% acetonitrile to 40% water (containing 0.02% ammonium acetate) and 60% acetonitrile at a flow rate of 1.0 mL per minute at a column temperature of 40° C. for 15 min. Column: waters XBridge C18 3.5 μm, 50×4.6 mm]. The purity was 91.14%, Rt=6.324 min.

LCMS (M+H)⁺ m/z calculated 648.3. found 648.3. ¹HNMR (DMSO-d₆, 400 MHz): δ 10.39 (br s, 1H), 8.88 (s, 1H), 7.93-7.87 (m, 1H), 7.49-7.42 (m, 1H), 7.39-7.32 (m, 1H), 6.81-6.71 (m, 3H), 4.92-4.90 (m, 1H), 4.40-4.04 (m, 3H), 3.81-3.45 (m, 2H), 3.28-3.16 (m, 1H), 2.87-2.75 (m, 2H), 2.38 (s, 3H), 1.34-1.23 (m, 3H), 1.08-1.02 (m, 12H).

Compound SZ-014041B was synthesized as a yellow solid (9.5 mg, 11% yield) from compound 014079A5B by referring to the synthesis of SZ-014041A.

Liquid chromatography-mass spectrometry [mobile phase: elution was carried out with a gradient from 20% water (containing 0.02% ammonium acetate) and 80% acetonitrile to 70% water (containing 0.02% ammonium acetate) and 30% acetonitrile at a flow rate of 1.5 mL per minute at a column temperature of 40° C. for 6.5 min. Column: waters XBridge C18 3.5 μm, 50×4.6 mm]. The purity was 93.36%, Rt=3.806 min. LCMS (M+H)⁺ m/z calculated 648.3. found 648.2. ¹HNMR (DMSO-d₆, 400 MHz): δ8.87 (s, 1H), 8.09-8.03 (m, 1H), 7.50-7.31 (m, 2H), 6.98-6.88 (m, 2H), 6.81-6.75 (m, 1H), 4.91-4.88 (m, 1H), 4.41-4.06 (m, 3H), 3.84-3.45 (m, 2H), 3.26-3.14 (m, 1H), 2.93-2.80 (m, 2H), 2.38 (s, 3H), 1.33-1.24 (m, 3H), 1.07-0.94 (m, 12H).

### Example 9. SZ-014043

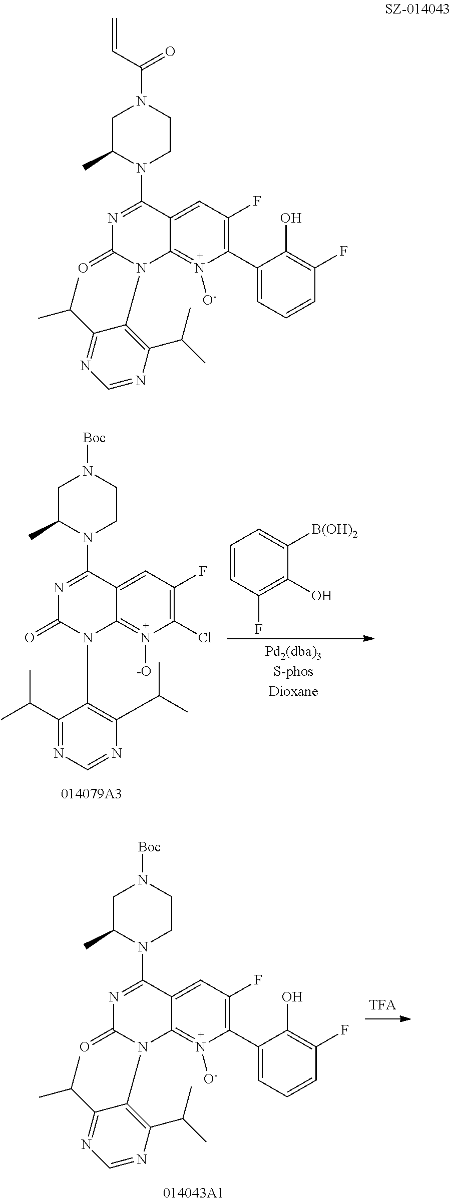

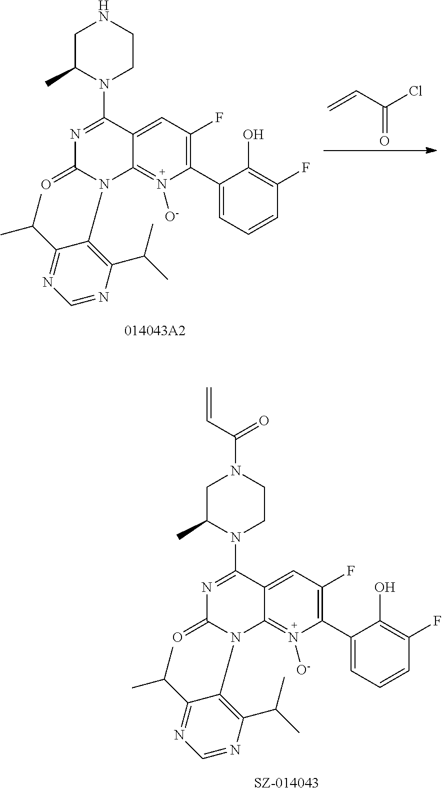

### Step 1: Synthesis of 014043A1

Compound 014079A3 (100 mg, 0.17 mmol), 2-hydroxy-3-fluorophenylboronic acid (100 mg, 0.61 mmol), potassium phosphate (74 mg, 0.35 mmol) and Sphos (43 mg, 0.11 mmol) were dissolved in 1,4-dioxane (2 mL). The system was purged with nitrogen several times, and then Pd₂(dba)₃ (32 mg, 0.04 mmol) was added. The reaction mixture was stirred at 95° C. for 2 h, cooled to room temperature, and filtered. The filtrate was diluted with ethyl acetate (10 mL) and washed with saturated brine. The organic phases were combined and dried over anhydrous sodium sulfate. The filtrate was concentrated under reduced pressure, and the resulting residue was purified by medium-pressure liquid chromatography to give compound 014043A1 as a yellow solid (30 mg, 27% yield). LCMS (M+H)⁺ m/z calculated 652.3. found 652.3.

### Step 2: Synthesis of 014043A2

Compound 014043A5 (30 mg, 0.05 mmol) was dissolved in dichloromethane (1 mL). Trifluoroacetic acid (0.2 mL) was added under an ice bath. The ice bath was removed, and the reaction mixture was stirred at room temperature for 1 h, and concentrated to give 014043A2 as a yellow oil (30 mg, crude product), which was directly used in the next step without purification. LCMS (M+H)⁺ m/z calculated 552.3. found 552.3.

### Step 3: Synthesis of SZ-014043

The crude compound 014043A2 (30 mg, 0.05 mmol) was dissolved in dichloromethane (1.0 mL). A solution of acryloyl chloride (4.5 mg, 0.05 mmol) in dichloromethane and N,N-diisopropylethylamine (26 mg, 0.2 mmol) were added at 0° C. The reaction mixture was stirred at 0° C. for 10 min. The reaction mixture was diluted with 20 mL of dichloromethane, washed with water, and concentrated under reduced pressure. The residue was purified by preparative high performance liquid chromatography (ammonium bicarbonate) to give SZ-014043 as a yellow solid (7 mg). LCMS (M+H)⁺ m/z calculated 606.3. found 606.3. ¹H NMR (DMSO-d₆, 400 MHz): δ 10.06-10.04 (m, 1H), 8.93 (s, 1H), 8.01 (s, 1H), 7.36-7.34 (m, 1H), 7.04-6.88 (m, 3H), 6.21 (d, J=16.4 Hz, 1H), 5.76 (dd, J=10.4, 2.4 Hz, 1H), 4.89-4.86 (m, 1H), 4.43-4.01 (m, 3H), 3.75-3.41 (m, 2H), 3.24-3.04 (m, 1H), 2.86-2.74 (m, 2H), 1.32 (d, J=6.4 Hz, 3H), 1.08-1.02 (m, 12H).

### Example 10. SZ-014044

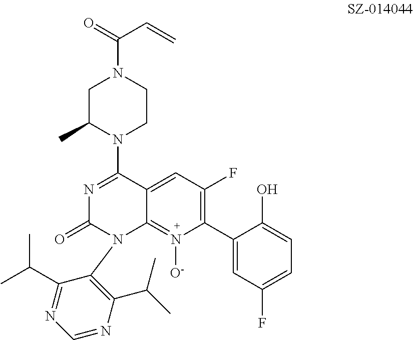

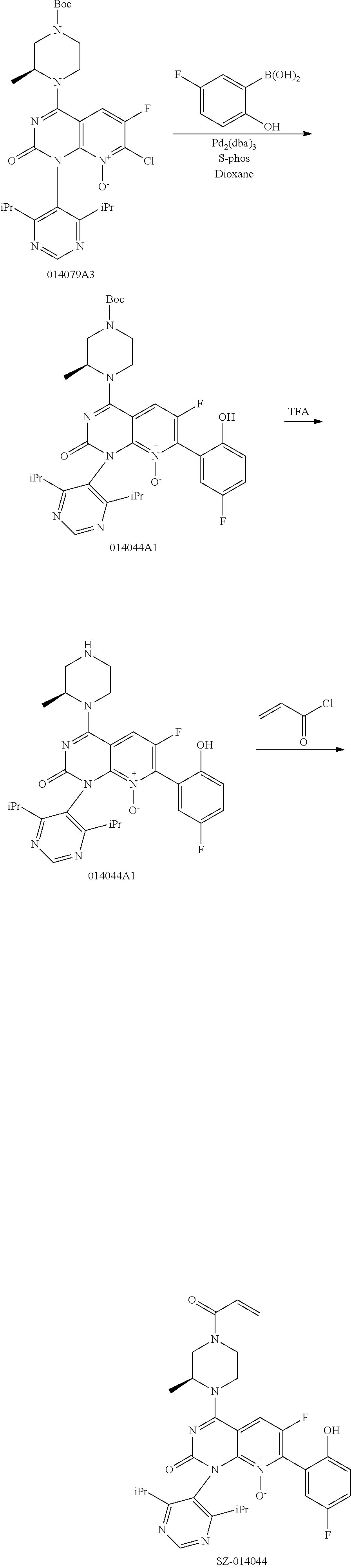

### Step 1: Synthesis of 014044A1

Compound 014079A3 (130 mg, 0.23 mmol), 2-hydroxy-5-fluorophenylboronic acid (130 mg, 0.79 mmol), potassium phosphate (96 mg, 0.45 mmol) and Sphos (56 mg, 0.13 mmol) were dissolved in 1,4-dioxane (2 mL). The system was purged with nitrogen several times, and then Pd₂(dba)₃ (42 mg, 0.04 mmol) was added. The reaction mixture was stirred at 95° C. for 2 h, cooled to room temperature, and filtered. The filtrate was diluted with ethyl acetate (10 mL) and washed with saturated brine. The organic phases were combined and dried over anhydrous sodium sulfate. The filtrate was concentrated under reduced pressure, and the resulting residue was purified by medium-pressure liquid chromatography to give compound 014043A1 as a yellow solid (30 mg, 20% yield). LCMS (M+H)⁺ m/z calculated 652.3. found 652.3.

### Step 2: Synthesis of 014044A2

Compound 014044A1 (30 mg, 0.05 mmol) was dissolved in dichloromethane (1 mL). Trifluoroacetic acid (0.2 mL) was added under an ice bath. The ice bath was removed, and the reaction mixture was stirred at room temperature for 1 h, and concentrated to give 014044A2 as a yellow oil (30 mg, crude product), which was directly used in the next step without purification. LCMS (M+H)⁺ m/z calculated 552.3. found 552.3.

### Step 3: Synthesis of SZ-014044

The crude compound 014044A2 (30 mg, 0.05 mmol) was dissolved in dichloromethane (1.0 mL). A solution of acryloyl chloride (4.5 mg, 0.05 mmol) in dichloromethane and N,N-diisopropylethylamine (26 mg, 0.2 mmol) were added at 0° C. The reaction mixture was stirred at 0° C. for 10 min. The reaction mixture was diluted with 20 mL of dichloromethane, washed with water, and concentrated under reduced pressure. The residue was purified by preparative high performance liquid chromatography (ammonium bicarbonate) to give SZ-014044 as a yellow solid (7 mg). LCMS (M+H)⁺ m/z calculated 606.3. found 606.3. ¹H NMR (DMSO-d₆, 400 MHz): δ 9.89 (s, 1H), 8.90 (s, 1H), 7.94 (s, 1H), 7.21-7.17 (m, 1H), 7.02-6.82 (m, 3H), 6.21 (d, J=16.4 Hz, 1H), 5.76 (dd, J=10.4, 2.4 Hz, 1H), 4.89-4.86 (m, 1H), 4.43-4.01 (m, 3H), 3.75-3.41 (m, 2H), 3.24-3.04 (m, 1H), 2.86-2.74 (m, 2H), 1.32 (d, J=6.4 Hz, 3H), 1.08-1.02 (m, 12H).

### Example 11. SZ-014013AB

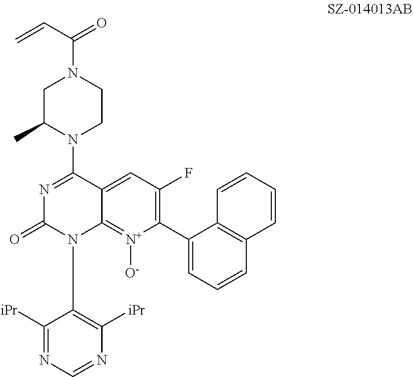 [SMILES: C=CC(=O)N1CCN(c2nc(=O)n(-c3c(C(C)C)ncnc3C(C)C)c3c2cc(F)c(-c2cccc4ccccc24)[n+]3[O-])[C@@H](C)C1]

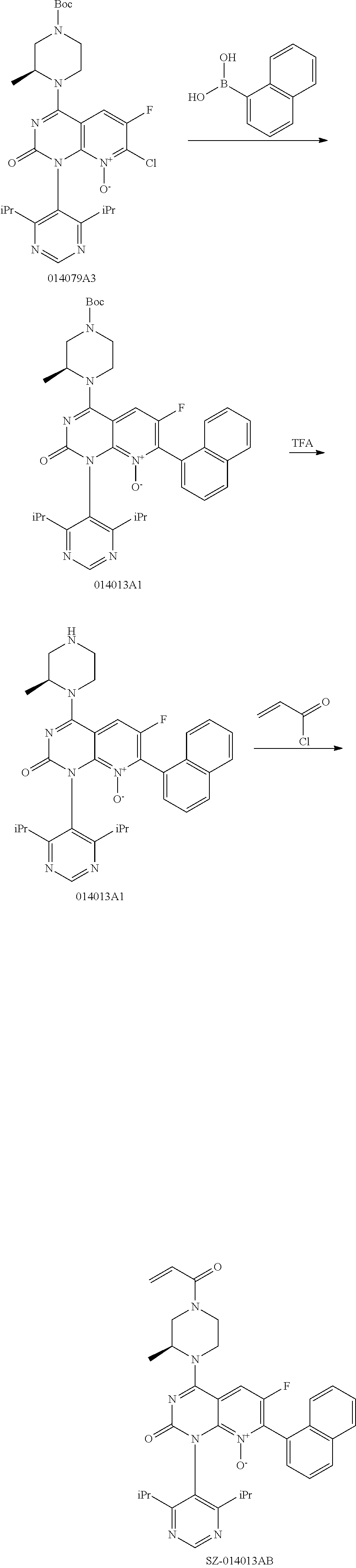

### Step 1: Synthesis of 014013A1

Compound 014079A3 (200 mg, 0.35 mmol), 1-naphthylboronic acid (120 mg, 0.70 mmol), potassium phosphate (148 mg, 0.70 mmol) and 2-dicyclohexylphosphine-2′,6′-dimethoxybiphenyl (42.8 mg, 0.105 mmol) were dissolved in 1,4-dioxane (10 mL). The system was purged with nitrogen several times, and then tris(dibenzylideneacetone)dipalladium (64 mg, 0.07 mmol) was added. The system was purged with nitrogen several times, and then the reaction mixture was stirred at 90° C. for 15 h, cooled to room temperature, and filtered. The filtrate was diluted with ethyl acetate (30 mL) and washed with saturated brine. The organic phases were combined and dried over anhydrous sodium sulfate. The filtrate was concentrated under reduced pressure, and the resulting residue was purified by column chromatography (PE:EA=2:1 to 1:1), followed by concentration to give crude compound 014013A1 as a yellow solid (200 mg, 86.0% yield). LCMS (M+H)⁺ m/z calculated 668.3. found 668.3.

### Step 2: Synthesis of 01407713A2

The crude compound 014013A1 (200 mg, 0.30 mmol) was dissolved in dichloromethane (2 mL). Trifluoroacetic acid (1.37 g, 12.0 mmol) was added under an ice bath. The ice bath was removed, and the reaction mixture was stirred at room temperature for 2 h, and concentrated to remove trifluoroacetic acid. The resulting yellow oil 014013A2 (170 mg, 100% yield) was directly used in the next step without purification. LCMS (M+H)⁺ m/z calculated 568.3. found 568.3.

### Step 3: Synthesis of SZ-014013AB

The crude compound 014013A2 (170 mg, 0.30 mmol) was dissolved in dichloromethane (2.0 mL). Acryloyl chloride (24.3 mg, 0.27 mmol) and N,N-diisopropylethylamine (116 mg, 0.90 mmol) were added. The reaction mixture was stirred at 0° C. for 5 min. The reaction mixture was diluted with dichloromethane (50 mL), quenched with saturated ammonium chloride solution (20 mL), washed with water (20 mL×2), and concentrated under reduced pressure. The residue was purified by preparative high performance liquid chromatography (ammonium bicarbonate) to give SZ-014013AB as a yellow solid (10.0 mg, 28.4% yield).

Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 4.6×50 mm]. The purity was 95.71%, Rt=4.016 min. LCMS (M+H)⁺ m/z calculated 622.2. found 622.2. ¹H NMR (DMSO-d₆, 400 MHz): δ 8.80 (s, 1H), 8.09 (d, J=8.4 Hz, 1H), 8.05-8.00 (m, 2H), 7.63 (t, J=8.0 Hz, 1H), 7.57-7.53 (m, 1H), 7.49-7.39 (m, 3H), 6.94-6.84 (m, 1H), 6.22 (d, J=16.0 Hz, 1H), 5.76 (dd, J₁=10.0 Hz, J₂=2.0 Hz, 1H), 4.98-4.90 (br s, 1H), 4.47-4.31 (m, 2H), 4.22-4.06 (m, 1H), 3.82-3.44 (m, 2H), 3.29-2.98 (m, 2H), 2.84-2.74 (m, 1H), 1.35 (d, J=6.4 Hz, 3H), 1.00-0.89 (m, 12H).

### Example 12. SZ-014031A

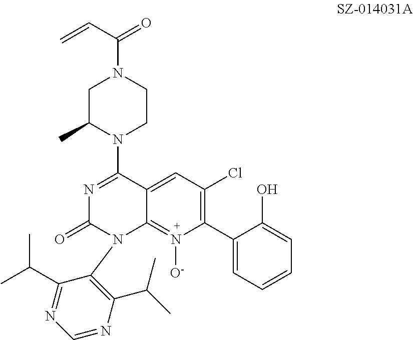

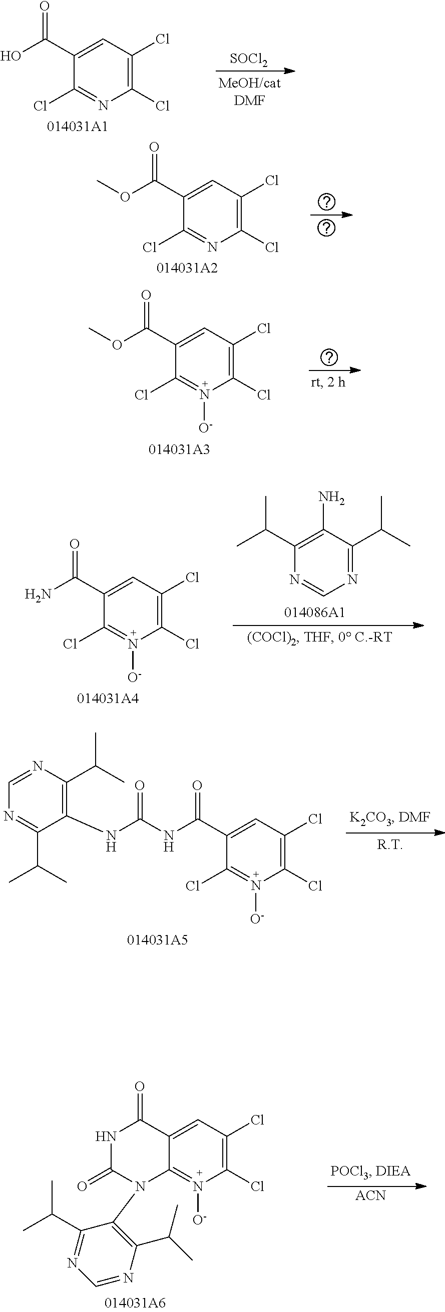

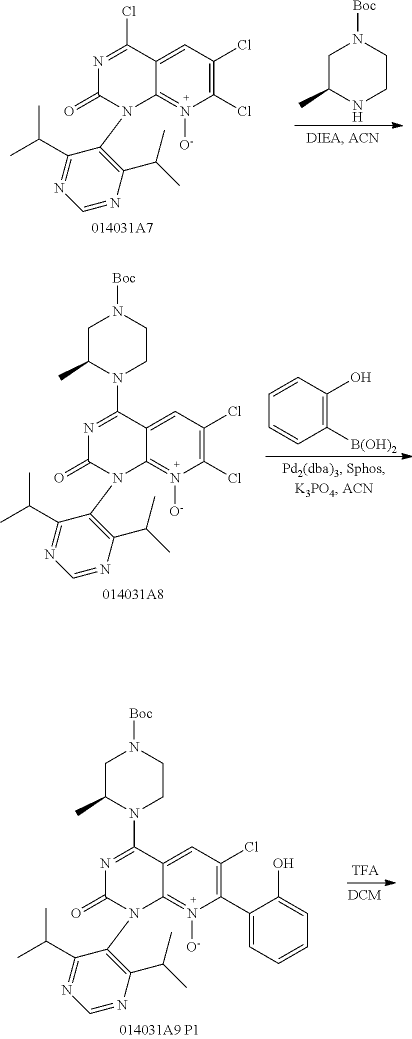

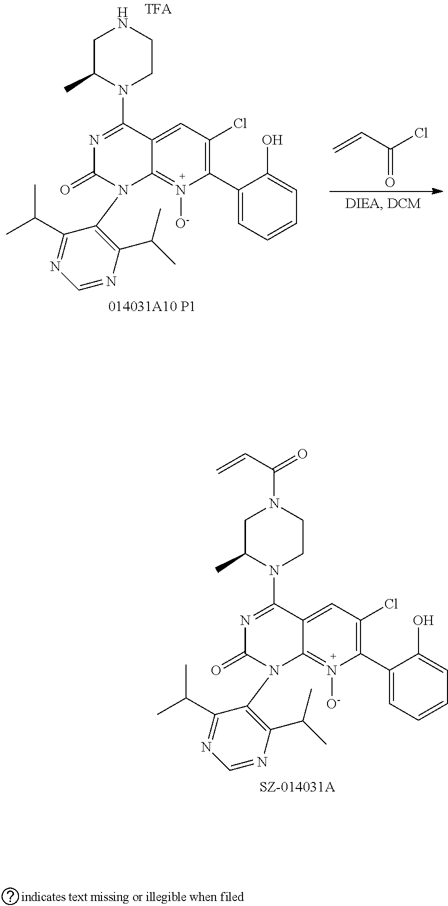

### Step 1: Synthesis of 014031A2

Compound 2,5,6-trichloronicotinic acid 014031A1 (10.0 g, 44.25 mmol) was dissolved in methanol (80 mL), to which two drops of N,N-dimethylformamide were added. Thionyl chloride (15.8 g, 132.75 mmol) was added dropwise at 0° C. After addition, the reaction mixture was heated to 70° C. and stirred for 2 h. The reaction mixture was cooled and then concentrated. Water (40 mL) was added to the residue, followed by extraction with dichloromethane (30 mL×3). The organic phases were combined, dried over anhydrous sodium sulfate, and filtered. The filtrate was concentrated to give crude compound 014031A2 as a white solid (10.5 g, 99% yield). LCMS (M+H)⁺ m/z calculated 239.9. found 240.0. ¹H NMR (DMSO-d₆, 400 MHz): δ 8.57 (s, 1H), 3.90 (s, 3H).

### Step 2: Synthesis of 014031A3

Compound 014031A2 (10.5 g, 43.75 mmol) was dissolved in acetonitrile (180 mL). Sodium percarbonate (6.9 g, 43.75 mmol) and trifluoromethanesulfonic anhydride (24.7 g, 87.50 mmol) were added at 0° C. The reaction mixture was stirred at room temperature overnight. The reaction mixture was concentrated. The residue was dissolved in water (50 mL), and the resulting solution was extracted with dichloromethane (30 mL×3). The organic phases were combined and concentrated, and the residue was purified by silica gel column chromatography (ethyl acetate/petroleum ether=24%-33%) to separate compound 014031A3 as a white solid (3.9 g, 34% yield). LCMS (M+H)⁺ m/z calculated 255.9. found 255.7. ¹H NMR (DMSO-d₆, 400 MHz): δ 8.04 (s, 1H), 3.91 (s, 3H).

### Step 3: Synthesis of 014031A4

Compound 014031A3 (4.9 g, 19.14 mmol) was dissolved in 1,4-dioxane (20 mL). Aqueous ammonia (20 mL) was added at room temperature. The reaction mixture was stirred at room temperature for 2 h, with a large amount of white solid precipitating. The reaction mixture was filtered, and the filter cake was collected and dried to give compound 014031A4 as a white solid (2.37 g, 51% yield). LCMS (M+H)⁺ m/z calculated 240.9. found 240.9. ¹H NMR (DMSO-d₆, 400 MHz): δ 8.12 (s, 1H), 8.02 (s, 1H), 7.85 (s, 1H).

### Step 4: Synthesis of 014031A5

Compound 014031A4 (2.37 g, 9.83 mol) was dissolved in tetrahydrofuran (40 mL). Oxalyl chloride (2.5 g, 19.66 mmol) was added at room temperature. The reaction mixture was heated at reflux for 1 h until it became clear and transparent. The reaction mixture was cooled to room temperature and concentrated. The residue was dissolved in tetrahydrofuran (30 mL). 4,6-Diisopropylpyrimidin-5-amine 014086A1 (2.6 g, 14.75 mmol) was added at 0° C. The reaction mixture was stirred at 0° C. for 1 h, and the reaction was completed. The reaction mixture was filtered, and the filter cake was rinsed with n-hexane, collected and dried to give compound 014031A5 as a yellow solid (4.4 g, 100% yield). LCMS (M+H)⁺ m/z calculated 446.0. found 445.8. ¹HNMR (DMSO-d₆, 400 MHz): δ 11.45 (s, 1H), 9.69 (s, 1H), 8.99 (s, 1H), 8.17 (s, 1H), 3.29-3.17 (m, 2H), 1.18-1.16 (m, 12H).

### Step 5: Synthesis of 014031A6

Compound 014031A5 (4.4 g, 9.88 mmol) was dissolved in N,N-dimethylformamide (40 mL). Potassium carbonate (2.7 g, 19.77 mmol) was added at room temperature. The reaction mixture was stirred at room temperature overnight. Water (50 mL) was added to the reaction mixture, which was then adjusted to pH 6 with 1 M aqueous HCl solution and extracted with ethyl acetate (40 mL×3). The organic phases were combined, washed with saturated brine, and concentrated under reduced pressure. The residue was purified by silica gel column chromatography (ethyl acetate/petroleum ether=40%-50%) to give compound 014031A6 as a yellow solid (4.1 g, 100% yield). LCMS (M+H)⁺ m/z calculated 410.1. found 409.8. ¹H NMR (DMSO-d₆, 400 MHz): δ 12.67 (s, 1H), 9.00 (s, 1H), 8.19 (s, 1H), 3.05-3.02 (m, 2H), 1.08 (d, J=6.8 Hz, 6H), 1.01 (d, J=6.4 Hz, 6H).

### Step 6: Synthesis of 014031A7

Compound 014031A6 (4.1 g, 10.00 mmol) was dissolved in anhydrous acetonitrile (80 mL). Phosphorus oxychloride (4.6 g, 30.00 mmol) and N,N′-diisopropylethylamine (3.9 g, 30.00 mmol) were added under an ice bath. The reaction mixture was heated to 60° C. and allowed to react for 1 h. The reaction mixture was cooled to room temperature and concentrated to give compound 014031A7 as a brown oil. The crude product was directly used in the next step.

### Step 7: Synthesis of 014031A8

The crude compound 014031A7 was dissolved in acetonitrile (80 mL). (S)-4-N-tert-butoxycarbonyl-2-methylpiperazine (3.0 g, 15.00 mmol) and diisopropylethylamine (3.9 g, 30.00 mmol) were added. The reaction mixture was stirred at 0° C. for 1 h. The reaction mixture was concentrated, and the residue was purified by silica gel column chromatography (ethyl acetate/petroleum ether=65%-80%) to give 014031A8 (2.3 g, 39% yield). LCMS (M+H)⁺ m/z calculated 592.2. found 591.9. ¹H NMR (DMSO-d₆, 400 MHz): δ 8.95 (s, 1H), 8.02 (s, 1H), 4.86-4.80 (m, 1H), 4.13-4.09 (m, 1H), 4.05-3.97 (m, 1H), 3.84-3.81 (m, 1H), 3.77-3.68 (m, 1H), 3.27-3.00 (m, 2H), 2.83-2.75 (m, 2H), 1.44 (s, 9H), 1.31 (d, J=6.4 Hz, 3H), 1.07-0.91 (m, 12H).

### Step 8: Synthesis of 014031A9 P1

Compound 014031A8 (200 mg, 0.34 mmol), 6-hydroxyphenylboronic acid (117 mg, 0.84 mmol), potassium phosphate (216 mg, 1.02 mmol) and 2-dicyclohexylphosphine-2′,6′-dimethoxybiphenyl (42 mg, 0.102 mmol) were dissolved in acetonitrile (20 mL). The system was purged with nitrogen several times, and then tris(dibenzylideneacetone)dipalladium (31 mg, 0.034 mmol) was added. The system was purged with nitrogen several times, and then the reaction mixture was stirred at 80° C. for 5 h. The reaction mixture was cooled to room temperature and concentrated to remove the solvent. Water (15 mL) and ethyl acetate (15 mL) were added, followed by extraction with ethyl acetate (10 mL×3). The organic phases were combined and concentrated under reduced pressure. The residue was purified by preparative high performance liquid chromatography to give compound 014031A9 P1 as a yellow solid (35 mg, 16% yield). LCMS (M+H)⁺ m/z calculated 650.3. found 650.1. ¹H NMR (DMSO-d₆, 400 MHz): δ 9.72 (d, J=5.6 Hz, 1H), 8.86 (s, 1H), 7.93 (d, J=6.0 Hz, 1H), 7.28-7.22 (m, 1H), 6.99-6.84 (m, 3H), 4.90-4.80 (m, 1H), 4.22-4.15 (m, 1H), 4.05-3.95 (m, 1H), 3.87-3.70 (m, 2H), 3.20-3.06 (m, 2H), 2.89-2.72 (m, 2H), 1.45 (s, 9H), 1.36-1.33 (m, 3H), 1.09-1.01 (m, 12H).

### Step 9: Synthesis of 014031A10 P1

Compound 014031A9 P1 (32 mg, 0.05 mmol) was dissolved in dichloromethane (10 mL). Trifluoroacetic acid (2 mL) was added under an ice bath. The ice bath was removed, and the reaction mixture was stirred at room temperature (20° C.) for 45 min. The reaction mixture was concentrated under reduced pressure to give compound 014031A10 P1 as a yellow oil (crude product, 53 mg, 100% yield), which was directly used in the next step without purification. LCMS (M+H)⁺ m/z calculated 550.2. found 550.0.

### Step 10: Synthesis of SZ-014031A

The crude compound 014031A10 P1 (crude product, 53 mg, 0.05 mmol) was dissolved in dichloromethane (3 mL). Acryloyl chloride (4.5 mg, 0.05 mmol) was slowly added under an ice bath, followed by diisopropylethylamine (32 mg, 0.25 mmol). After dropwise addition, the reaction phase was stirred at 0° C. for 15 min. Saturated ammonium chloride solution (8 mL) was added to the reaction mixture, followed by extraction with dichloromethane (5 mL×3). The organic phases were combined, washed twice with water (10 mL×2), dried over anhydrous sodium sulfate, and filtered. The filtrate was concentrated under reduced pressure, and the residue was purified by preparative high performance liquid chromatography (ammonium bicarbonate) to give compound SZ-014031A as a pale yellow solid (10 mg, 33% yield).

Liquid chromatography-mass spectrometry [mobile phase: elution was carried out with a gradient from 70% water (containing 0.02% ammonium acetate) and 30% acetonitrile to 40% water (containing 0.02% ammonium acetate) and 60% acetonitrile at a flow rate of 1.5 mL per minute at a column temperature of 40° C. for 6 min. Column: waters XBridge C18 3.5 μm, 50×4.6 mm. The purity was 88.46%, Rt=2.755 min. LCMS (M+H)⁺ m/z calculated 604.2. found 604.2. ¹H NMR (DMSO-d₆, 400 MHz): δ9.72 (d, J=6.8 Hz, 1H), 8.86 (s, 1H), 7.96 (s, 1H), 7.28-7.24 (m, 1H), 6.98 (d, J=7.6 Hz, 1H), 6.90-6.81 (m, 3H), 6.20 (d, J=16.0 Hz, 1H), 5.76 (dd, J₁=10.4 Hz, J₂=2.4 Hz, 1H), 4.90-4.85 (m, 1H), 4.41-4.00 (m, 3H), 3.78-3.39 (m, 2H), 3.25-3.02 (m, 1H), 2.89-2.72 (m, 2H), 1.34-131 (m, 3H), 1.08-1.01 (m, 12H).

### Example 13. SZ-014051AB

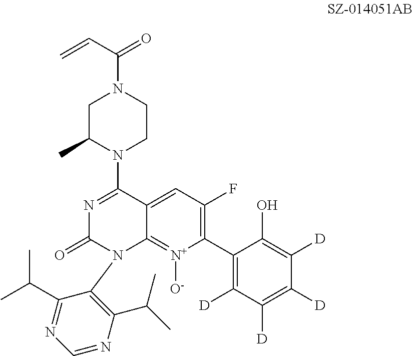 [SMILES: [2H]c1c([2H])c([2H])c(-c2c(F)cc3c(N4CCN(C(=O)C=C)C[C@@H]4C)nc(=O)n(-c4c(C(C)C)ncnc4C(C)C)c3[n+]2[O-])c(O)c1[2H]]

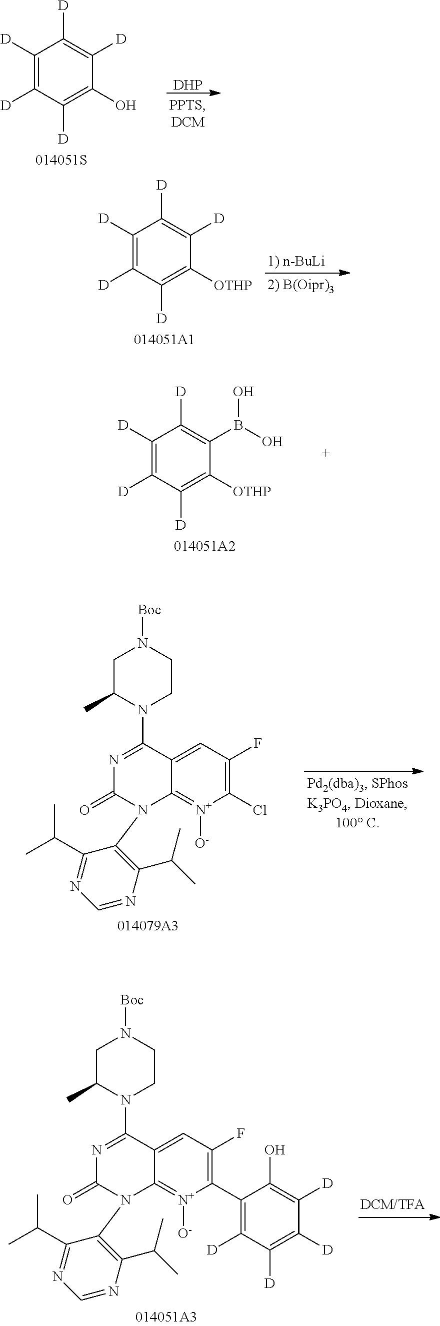

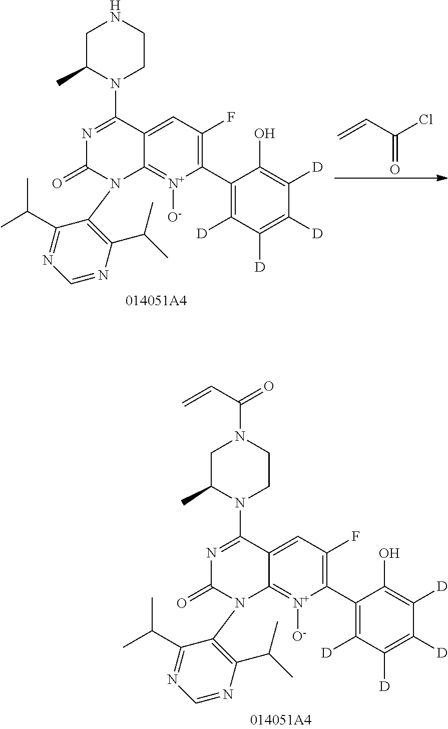

### Step 1: Synthesis of 014051A1

Compound 014051S (2.5 g, 25.2 mmol) was dissolved in dichloromethane (50 mL). 3,4-Dihydropyran (6.4 g, 75.6 mmol) and pyridinium p-toluenesulfonate (633 mg, 2.52 mmol) were added. The reaction mixture was stirred at room temperature overnight. The reaction mixture was washed with saturated brine. The organic phases were combined, dried over anhydrous sodium sulfate, and concentrated to give crude compound 014051A1 as a colorless oil (5.0 g, 100% yield). No MS response value.

### Step 2: Synthesis of 014051A2

Compound 014051A1 (2.5 g, 13.67 mmol) was dissolved in tetrahydrofuran (25 mL), and the resulting solution was cooled to −78° C. n-Butyllithium (8.2 mL, 20.50 mmol) was slowly added dropwise. The reaction mixture was stirred for 15 min. The reaction mixture was allowed to naturally warm up to 25° C. and maintained for 1 h. The reaction mixture was cooled to −78° C. Triisopropyl borate (6.4 g, 34.17 mmol) was added. The reaction mixture was allowed to naturally warm up to room temperature and stirred for 2 h. The reaction mixture was quenched by addition of water and extracted with ethyl acetate. The organic phase was dried over anhydrous sodium sulfate. The concentrate was purified by preparative high performance liquid chromatography to give compound 014051A2 as an off-white solid (400 mg, 40% yield). No MS response value.

### Step 3: Synthesis of 014051A3

Compound 014051A2 (394 mg, 2.78 mmol), 014079A3 (800 mg, 1.39 mmol), potassium phosphate (884 mg, 1.39 mmol) and 2-dicyclohexylphosphine-2′,6′-dimethoxybiphenyl (228 mg, 0.28 mmol) were dissolved in 1,4-dioxane (25 mL). The system was purged with nitrogen several times, and then tris(dibenzylideneacetone)dipalladium (254 mg, 0.28 mmol) was added. The system was purged with nitrogen several times, and then the reaction mixture was stirred at 100° C. overnight, cooled to room temperature, and filtered. The filtrate was diluted with ethyl acetate (30 mL) and washed with saturated brine. The organic phases were combined and dried over anhydrous sodium sulfate. The filtrate was concentrated under reduced pressure, and the resulting residue was purified by column chromatography (DCM:methanol=20:1), followed by concentration to give a crude product, which was then purified by preparative high performance liquid chromatography to give compound 014051A3 as a yellow solid (120 mg, 13.5% yield). LCMS (M+H)⁺ m/z calculated 638.3. found 638.3. ¹H NMR (DMSO-d₆, 400 MHz): δ9.82 (s, 1H), 8.89 (s, 1H), 7.91-7.88 (d, J=10.8 Hz, 2H), 4.86 (brs, 1H), 4.21-4.17 (m, 1H), 4.03-3.99 (m, 1H), 3.97-3.69 (m, 3H), 3.18-3.07 (m, 1H), 2.83-2.77 (m, 2H), 1.42 (s, 9H), 1.35 (d, J=8.4 Hz, 3H), 1.06-1.04 (m, 12H).

### Step 4: Synthesis of 014051A4

The crude compound 014051A3 (100 mg, 0.156 mmol) was dissolved in dichloromethane (4 mL). Trifluoroacetic acid (2 mL) was added under an ice bath. The ice bath was removed, and the reaction mixture was stirred at room temperature for 2 h, and concentrated (with toluene to remove trifluoroacetic acid) to give 014051A4 as a yellow oil (120 mg, crude product), which was directly used in the next step without purification. LCMS (M+H)⁺ m/z calculated 538.3. found 538.3.

### Step 5: Synthesis of SZ-014051AB

The crude compound 014051A4 (120 mg, 0.156 mmol) was dissolved in dichloromethane (5.0 mL). Acryloyl chloride (14 mg, 0.156 mmol) and N,N-diisopropylethylamine (60.3 mg, 0.47 mmol) were added. The reaction mixture was stirred at 0° C. for 10 min. The reaction mixture was diluted with 20 mL of dichloromethane, washed with water, and concentrated under reduced pressure. The residue was purified by preparative high performance liquid chromatography (ammonium bicarbonate) to give SZ-014051AB as a yellow solid (52 mg, 56% yield). Liquid chromatography-mass spectrometry [mobile phase: elution was carried out with a gradient from 95% water (containing 0.02% ammonium acetate) and 5% acetonitrile to 5% water (containing 0.02% ammonium acetate) and 95% acetonitrile at a flow rate of 1.5 mL per minute at a column temperature of 40° C. for 6 min. Column: waters XBridge C18 5 μm, 4.5×50 mm]. The purity was 95.31%, Rt=3.465 min. LCMS (M+H)⁺ m/z calculated 592.3. found 592.3. ¹H NMR (DMSO-d₆, 400 MHz): δ9.85 (s, 1H), 8.92 (s, 1H), 7.97-7.94 (d, J=9.6 Hz, 1H), 6.96-6.84 (m, 1H), 6.24 (d, J=21.6 Hz, 1H), 5.80 (dd, J₁=14.0 Hz, J₂=2.8 Hz, 1H), 4.92 (br s, 1H), 4.48-4.04 (m, 3H), 3.77-3.74 (m, 1H), 3.77-3.45 (m, 1H), 3.33-3.27 (m, 1H), 2.85-2.77 (m, 2H), 1.35 (d, J=8.8 Hz, 3H), 1.08-0.96 (m, 12H).

### Example 14. SZ-014053

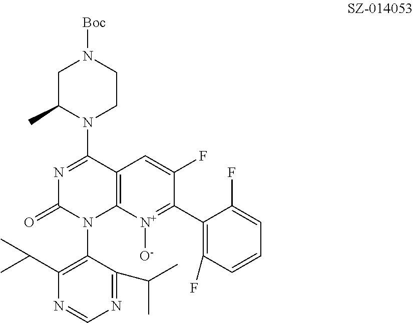 [SMILES: CC(C)c1ncnc(C(C)C)c1-n1c(=O)nc(N2CCN(C(=O)OC(C)(C)C)C[C@@H]2C)c2cc(F)c(-c3c(F)cccc3F)[n+]([O-])c21]

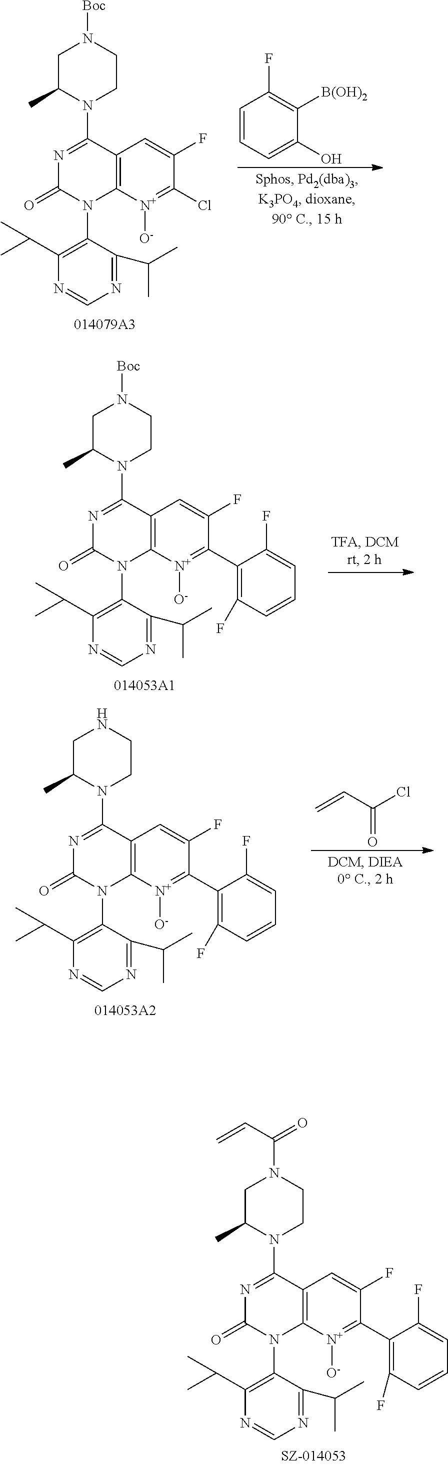

### Step 1: Synthesis of 014053A1

Compound 014079A3 (15 g, 26.04 mmol), 2,6-difluorophenylboronic acid (14.3 g, 65.10 mmol), potassium phosphate (11 g, 52.08 mmol) and 2-dicyclohexylphosphine-2′,6′-dimethoxybiphenyl (Sphos) (6.4 g, 15.62 mmol) were dissolved in 1,4-dioxane (200 mL). Pd₂(dba)₃ (7.2 g, 7.81 mmol) was added. The system was purged with nitrogen several times, and then the reaction mixture was stirred at 120° C. for 15 h, cooled to room temperature, and filtered. The filtrate was diluted with ethyl acetate (300 mL) and washed with saturated brine. The organic phases were combined and dried over anhydrous sodium sulfate. The filtrate was concentrated under reduced pressure, and the resulting residue was purified by column chromatography (PE:EA=2:1 to 1:1), followed by concentration to give compound 014053A1 as a yellow solid (5.7 g, about 55% purity, about 21.8% yield). LCMS (M+H)⁺ m/z calculated 654.3. found 654.3. ¹H NMR (DMSO-d₆, 400 MHz): δ 8.90 (s, 1H), 7.98 (d, J=8.4 Hz, 1H), 7.73-7.66 (m, 1H), 7.31 (t, J=8.8 Hz, 2H), 4.87 (br, 1H), 4.23-4.20 (m, 1H), 3.87-3.84 (m, 2H), 3.73-3.65 (m, 3H), 2.86-2.81 (m, 2H), 1.45 (m, 9H), 1.35 (d, J=6.4 Hz, 3H), 1.08-1.05 (m, 6H), 1.01 (t, J=6.4 Hz, 6H).

### Step 2: Synthesis of 014053A2

The crude compound 014053A1 (5.7 g, 8.71 mmol) was dissolved in dichloromethane (60 mL). Trifluoroacetic acid (20 mL) was added under an ice bath. The ice bath was removed, and the reaction mixture was stirred at room temperature for 2 h, and concentrated (with toluene to remove trifluoroacetic acid) to give 014053A2 as a yellow oil (6.6 g, crude product), which was directly used in the next step without purification. LCMS (M+H)⁺ m/z calculated 554.3. found 554.3.

### Step 3: Synthesis of SZ-014053

The crude compound 014053A2 (6.6 g, 11.9 mmol) was dissolved in dichloromethane (100 mL). Acryloyl chloride (646 mg, 7.1 mmol) and N,N-diisopropylethylamine (1.22 g, 9.5 mmol) were added. The reaction mixture was stirred at 0° C. for 10 min. The reaction mixture was diluted with 100 mL of dichloromethane, washed with water, and concentrated under reduced pressure. The residue was purified by preparative high performance liquid chromatography (ammonium bicarbonate). An enrichment liquid was prepared and concentrated by rotary evaporation to remove acetonitrile. The aqueous phase was extracted with dichloromethane, followed by concentration to give SZ-014053 as a yellow solid (703 mg, 20.5% overall yield over two steps).

Liquid chromatography-mass spectrometry [mobile phase: elution was carried out with a gradient from 95% water (containing 0.02% ammonium acetate) and 5% acetonitrile to 5% water (containing 0.02% ammonium acetate) and 95% acetonitrile at a flow rate of 1.5 mL per minute at a column temperature of 40° C. for 6 min. Column: waters XBridge C18 5 μm, 4.5×50 mm]. The purity was 95.8%, Rt=3.690 min. LCMS (M+H)⁺ m/z calculated 608.3. found 608.3. ¹H NMR (DMSO-d₆, 400 MHz): δ 8.90 (s, 1H), 8.03-8.01 (m, 1H), 7.73-7.66 (m, 1H), 7.31 (t, J=8.8 Hz, 2H), 6.89-6.85 (m, 1H), 6.23-6.18 (m, 1H), 5.77 (dd, J=10.4, 2.4 Hz, 1H), 4.90 (br, 1H), 4.43-4.02 (m, 3H), 3.77-3.74 (m, 1H), 3.63-3.31 (m, 1H), 3.24-3.07 (m, 1H), 2.86-2.81 (m, 2H), 1.32 (d, J=6.4 Hz, 3H), 1.06 (t, J=6 Hz, 6H), 1.02-0.99 (m, 6H).

### Example 15. SZ-014055

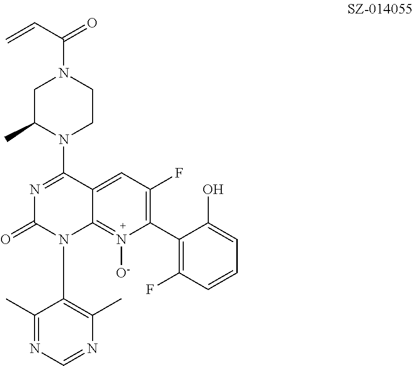

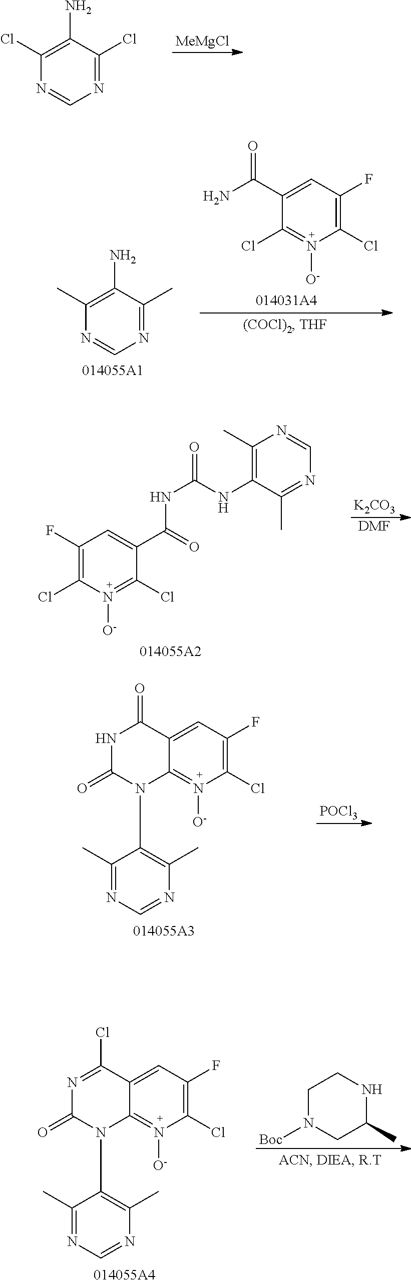

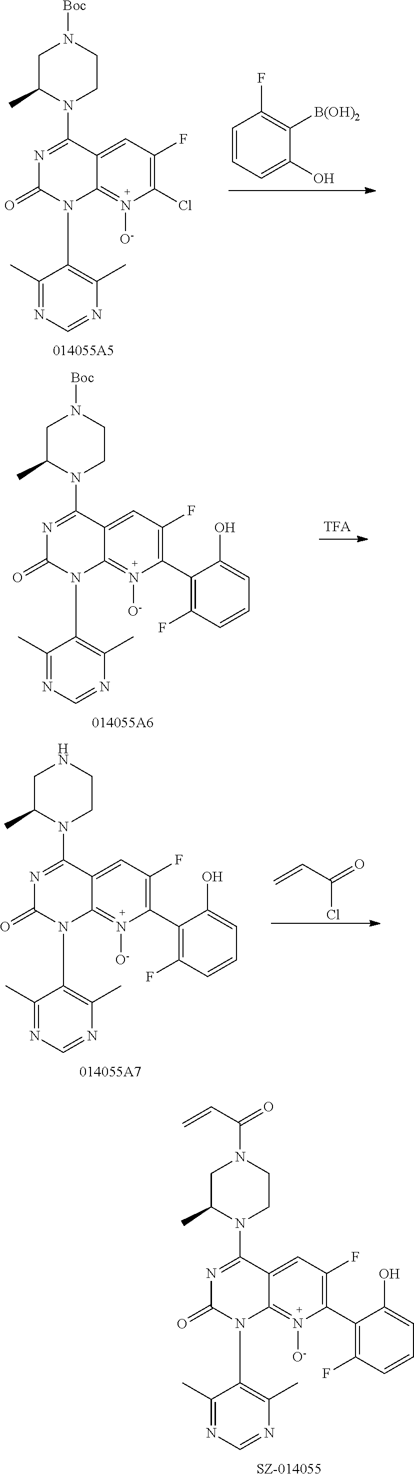

### Step 1: Synthesis of 014055A1

The compound 4,6-dichloro-5-aminopyrimidine (10.0 g, 60.98 mmol) was dissolved in 200 mL of tetrahydrofuran. [1,1′-bis(diphenylphosphino)ferrocene]palladium dichloride (8.9 g, 12.2 mmol) was added, followed by dropwise addition of methylmagnesium chloride (1 N, 183 mL, 366 mmol) at 0° C. The reaction mixture was heated to 70° C. under nitrogen and allowed to react overnight. The reaction mixture was cooled to room temperature, quenched with saturated ammonium chloride, and filtered. The filtrate was concentrated under reduced pressure, and the residue was purified by silica gel column chromatography (dichloromethane:methanol=10:1) to give 014055A1 as a black solid (1.6 g, 21% yield). LCMS (M+H)⁺ m/z calculated 124.1. found 124.2.

### Step 2: Synthesis of 014055A2

Compound 014089A3 (9.3 g, 41.5 mmol) was dissolved in 60 mL of tetrahydrofuran. Oxalyl chloride (6.3 g, 49.8 mmol) was added at room temperature. The reaction mixture was heated at reflux for 1 h. The reaction mixture was cooled to 0° C., and the crude compound 014055A1 (5.1 g) was added. The mixture was stirred at room temperature for 1 h. The pH of the reaction mixture was adjusted to neutrality with saturated sodium bicarbonate solution, followed by extraction with ethyl acetate. The extract was dried over anhydrous sodium sulfate and filtered. The filtrate was concentrated under reduced pressure. The residue was purified by silica gel column chromatography (petroleum ether:ethyl acetate=5:1) to give 014055A2 as a red solid (3.6 g, 23% yield). LCMS (M+H)⁺ m/z calculated 374.0. found 374.1.

### Step 3: Synthesis of 014055A3

Compound 014055A2 (1.8 g, 4.83 mmol) was dissolved in N,N-dimethylformamide (15 mL). Anhydrous potassium carbonate (1.3 g, 9.65 mmol) was added. The reaction mixture was stirred at room temperature overnight. The reaction mixture was poured into water, and the pH of the resulting mixture was adjusted to neutrality with 2 N HCl, followed by extraction with ethyl acetate. The extract was dried over anhydrous sodium sulfate and filtered. The filtrate was concentrated under reduced pressure. The residue was purified by silica gel column chromatography (petroleum ether:ethyl acetate=1:1) to give 014055A3 as a red oil (1.1 g, 69% yield). LCMS (M+H)⁺ m/z calculated 338.0. found 338.1. ¹HNMR (DMSO-d₆, 400 MHz): δ 12.67 (s, 1H), 8.81 (s, 1H), 8.14 (d, J=6.8 Hz, 1H), 2.26 (s, 6H).

### Step 4: Synthesis of 014055A4

Compound 014055A3 (2.2 g, 6.5 mmol) was dissolved in anhydrous acetonitrile (5 mL). Phosphorus oxychloride (3.07 g, 19.6 mmol) and N,N′-diisopropylethylamine (2.53 g, 19.6 mmol) were added under an ice bath. The reaction mixture was heated to 65° C. and allowed to react for 1 h. The reaction mixture was concentrated to dryness by rotary evaporation to give 014055A4 as a red oil (2.3 g, 100% yield). The crude product was directly used in the next step. LCMS (M+H)⁺ m/z calculated 356.0. found 356.1.

### Step 5: Synthesis of 014055A5

The crude compound 014055A4 (2.2 g, 6.5 mmol) was dissolved in acetonitrile (15 mL). (S)-4-N-tert-butoxycarbonyl-2-methylpiperazine (2.6 g, 13.0 mmol) and DIPEA (3 mL) were added. The reaction mixture was stirred at 0° C. for 1 h. The reaction mixture was concentrated to dryness by rotary evaporation, and the residue was purified by silica gel column chromatography (petroleum ether:ethyl acetate=1:1) to give 014055A5 as a red oil (2.8 g, 84% yield). LCMS (M+H)⁺ m/z calculated 520.2. found 520.3.

### Step 6: Synthesis of 014055A6

Compound 014086A5 (519 mg, 1 mmol) and 2-fluoro-6-hydroxyphenylboronic acid (390 mg, 2.5 mmol) were dissolved in acetonitrile (10 mL). Potassium phosphate (636 mg, 3.0 mmol), tris(dibenzylideneacetone)dipalladium (91.6 mg, 0.1 mmol) and 2-dicyclohexylphosphine-2′,6′-dimethoxybiphenyl (123 mg, 0.3 mmol) were added. The reaction mixture was allowed to react at 80° C. under nitrogen for 6 h. The reaction mixture was cooled and then filtered. The filtrate was concentrated, and the residue was purified by silica gel column chromatography (petroleum ether:ethyl acetate=1:2) to give 014055A6 as a red solid (150 mg, 25% yield). LCMS (M+H)⁺ m/z calculated 596.2. found 596.4. ¹HNMR (DMSO-d₆, 400 MHz): δ 10.48 (br s, 1H), 8.70 (s, 1H), 7.87 (q, J=4 Hz, 1H), 7.36 (dd, J₁=15.6 Hz, J₂=8.4 Hz, 1H), 6.78-6.72 (m, 2H), 4.86-4.85 (m, 1H), 4.16 (d, J=13.6 Hz, 1H), 3.96-3.82 (m, 2H), 3.70-3.69 (m, 1H), 3.13-3.06 (m, 2H), 2.19-2.11 (m, 6H), 1.48 (s, 9H), 1.38-1.23 (m, 3H).

### Step 7: Synthesis of 014055A7

Compound 014055A6 (150 mg, 0.25 mmol) was dissolved in dichloromethane (2 mL). Trifluoroacetic acid (2 mL) was added. The reaction mixture was stirred at room temperature for 2 h. The reaction mixture was concentrated to dryness to give 125 mg of trifluoroacetate salt 014055A7 as a red oil, which was directly used in the next step. LCMS (M+H)⁺ m/z calculated 496.2. found 496.3.

### Step 8: Synthesis of SZ-014055

Compound 014055A7 (115 mg, 0.232 mmol) was dissolved in anhydrous dichloromethane (5 mL). Acryloyl chloride (21 mg, 0.232 mmol) was added, followed by N,N-diisopropylethylamine (45 mg, 0.348 mmol). The reaction mixture was stirred at room temperature for 0.5 h. The reaction mixture was extracted with dichloromethane. The organic phase was concentrated to dryness by rotary evaporation, and the residue was purified by preparative high performance liquid chromatography to give SZ-014055 as a yellow solid (3.5 mg, 5.9% yield).

Liquid chromatography-mass spectrometry [mobile phase: elution was carried out with a gradient from 85% water (containing 0.02% ammonium acetate) and 15% acetonitrile to 40% water (containing 0.02% ammonium acetate) and 60% acetonitrile at a flow rate of 1.5 mL per minute at a column temperature of 40° C. for 6 min. Column: waters XBridge C18 3.5 μm, 50×4.6 mm. The purity was 81.87%, Rt=3.523 min. LCMS (M+H)⁺ m/z calculated 550.2. found 550.2. ¹HNMR (DMSO-d₆, 400 MHz): δ 10.47 (br s, 1H), 8.71 (s, 1H), 7.92-7.91 (m, 1H), 7.36 (q, J=8 Hz, 1H), 6.87-6.73 (m, 3H), 6.21 (d, J=15.2 Hz, 1H), 5.76 (dd, J₁=10.4 Hz, J₂=2.0 Hz, 1H), 4.91-4.88 (m, 1H), 4.42-4.16 (m, 2H), 4.03-4.11 (m, 1H), 3.74-3.51 (m, 2H), 3.22-3.09 (m, 1H), 2.32-2.05 (m, 6H), 1.33-1.24 (m, 3H).

### Example 16. SZ-014032A & SZ-014032B

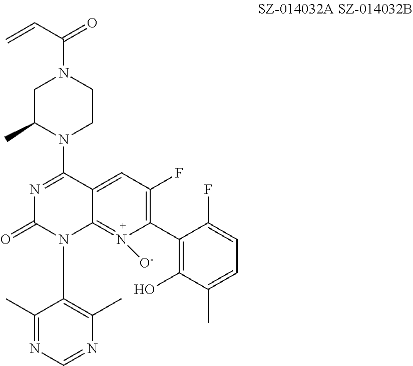

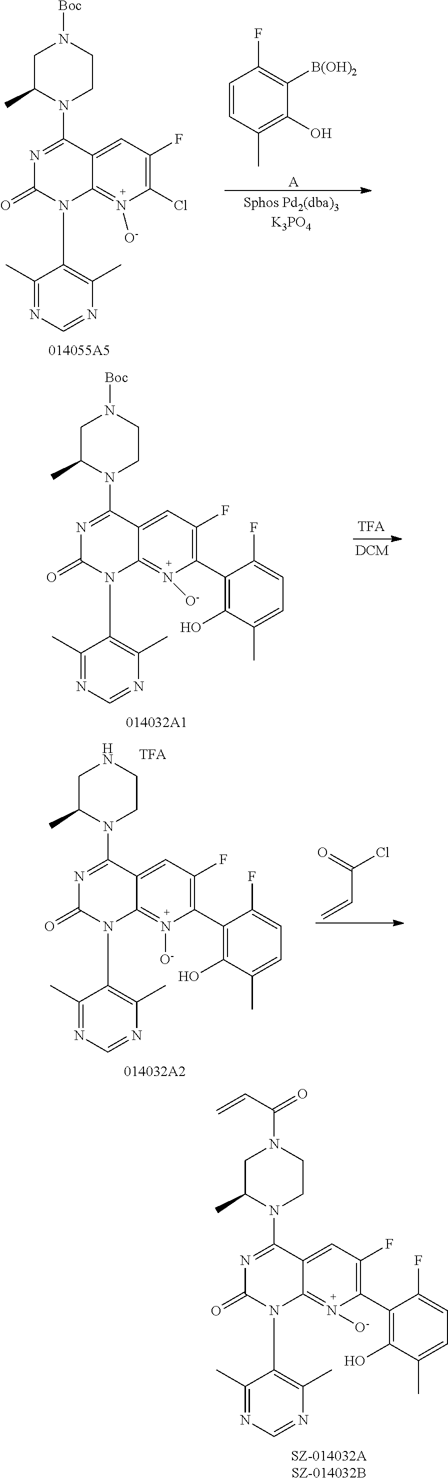

### Step 1: Synthesis of 014032A1

Compound 014055A5 (366 mg, 0.71 mmol), (6-fluoro-2-hydroxy-3-methyl)phenylboronic acid (120 mg, 0.71 mmol), potassium phosphate (449 mg, 2.1 mmol) and 2-dicyclohexylphosphine-2′,6′-dimethoxybiphenyl (86.8 mg, 0.21 mmol) were dissolved in 1,4-dioxane (10 mL). The system was purged with nitrogen several times, and then tris(dibenzylideneacetone)dipalladium (64.7 mg, 0.07 mmol) was added. The system was purged with nitrogen several times, and then the reaction mixture was stirred at 90° C. for 15 h, cooled to room temperature, and filtered. The filtrate was diluted with ethyl acetate (30 mL) and washed with saturated brine (20 mL×2). The organic phases were combined and dried over anhydrous sodium sulfate. The filtrate was concentrated under reduced pressure, and the resulting residue was purified by column chromatography (PE:EA=2:1 to 1:1), followed by concentration to give crude compound 014032A1 as a yellow solid (86 mg, about 30% purity, 6.0% yield). LCMS (M+H)⁺ m/z calculated 610.2. found 610.2.

### Step 2: Synthesis of 014032A2

The crude compound 014032A1 (86 mg, 30% purity, crude product, 0.04 mmol) was dissolved in dichloromethane (2 mL). Trifluoroacetic acid (182 mg, 1.6 mmol) was added under an ice bath. The ice bath was removed, and the reaction mixture was stirred at room temperature for 2 h, and concentrated to remove trifluoroacetic acid. The resulting yellow oil was purified by preparative high performance liquid chromatography (trifluoroacetic acid) to give 014032A2 as a yellow solid (9 mg, 41.8% yield). LCMS (M+H)⁺ m/z calculated 510.2. found 510.2.

### Step 3: Synthesis of SZ-014032AB

The crude compound 014032A2 (9 mg, 0.018 mmol) was dissolved in dichloromethane (1.0 mL). Acryloyl chloride (1.5 mg, 0.017 mmol) and N,N-diisopropylethylamine (7.0 mg, 0.054 mmol) were added. The reaction mixture was stirred at 0° C. for 5 min. The reaction mixture was diluted with 20 mL of dichloromethane, quenched with saturated ammonium chloride solution (10 mL), and washed with water (10 mL×2). The organic phase was concentrated under reduced pressure, and the resulting residue was purified by preparative high performance liquid chromatography (column model: xbridge C8 SN. 1271372511401 waters method: prep-5 xbridge C18 5 μm 19×150 mm 15-50% B, A: H₂O (0.1% NH₄HCO₃), B: ACN, 214, flowrate 15 m/min GT 12 min) to give isomers SZ-014032A (first eluted isomer) (RT: 10.64 min (1.9 mg)) and SZ-014032B (second eluted isomer) (RT: 11.34 min (1.5 mg)) as yellow solids (3.4 mg in total, 35% yield).

SZ-014032A: liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 4.6×50 mm]. The purity was 80.76%, Rt=2.930 min. LCMS (M+H)⁺ m/z calculated 564.2. found 564.3.
SZ-014032B: liquid chromatography-mass spectrometry [mobile phase: elution was carried out with a gradient from 80% water (containing 0.02% ammonium acetate) and 20% acetonitrile to 40% water (containing 0.02% ammonium acetate) and 60% acetonitrile at a flow rate of 1.5 mL per minute at a column temperature of 40° C. for 6 min. Column: waters XBridge C18 3.5 μm, 4.6×50 mm], Rt=3.264 min. LCMS (M+H)⁺ m/z calculated 564.2. found 564.2.

### Example 17. SZ-014066A & SZ-014066B

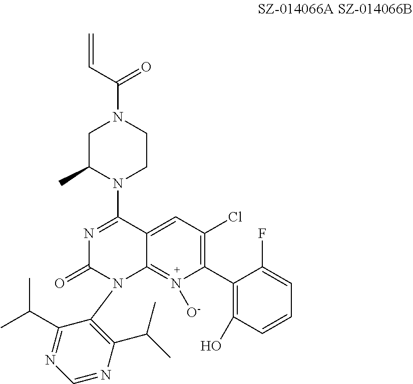

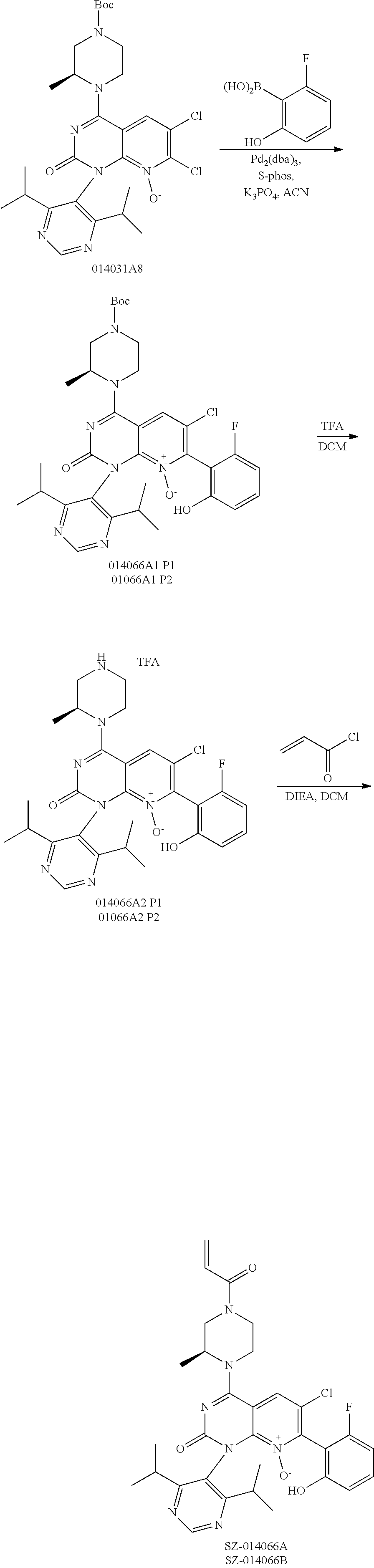

### Step 1: Synthesis of 014066A1 P1 & 014066A1 P2

Compound 014031A8 (500 mg, 0.844 mmol), 2-fluoro-6-hydroxyphenylboronic acid (329 mg, 2.11 mmol), potassium phosphate (534 mg, 2.52 mmol) and 2-dicyclohexylphosphine-2′,6′-dimethoxybiphenyl (104 mg, 0.25 mmol) were dissolved in acetonitrile (40 mL). The system was purged with nitrogen several times, and then tris(dibenzylideneacetone)dipalladium (77 mg, 0.084 mmol) was added. The system was purged with nitrogen several times, and then the reaction mixture was stirred at 80° C. for 5 h, cooled to room temperature, and filtered. The filtrate was diluted with ethyl acetate (100 mL) and washed with saturated brine. The organic phase was dried over anhydrous sodium sulfate and filtered. The filtrate was concentrated under reduced pressure, and the resulting residue was purified by preparative high performance liquid chromatography (column model: xbridge C8 SN. 1271372511401 waters; preparation method: RP-PREP-3×bridge C8 5 μm 19×150 mm 45-70%, A: H₂O (0.1% NH₄HCO₃), B: ACN, 214, flowrate 15 m/min 15 min-GT 10 min) to give isomers 014066A1 P1 (first eluted isomer) (RT: 9.24 min (44 mg)) and 014066A1 P2 (second eluted isomer) (RT: 10.93 min (106 mg)) as yellow solids (150 mg in total, 26% yield).

### Compound 014066A1 P1:

LCMS (M+H)⁺ m/z calculated 668.3. found 668.0. ¹H NMR (DMSO-d₆, 400 MHz): δ 10.30 (s, 1H), 8.86 (s, 1H), 7.96 (s, 1H), 7.30 (q, J=8.0 Hz, 1H), 6.72 (dd, J₁=19.2 Hz, J₂=8.0 Hz, 2H), 4.87-4.86 (m, 1H), 4.22-4.16 (m, 1H), 3.98-3.71 (m, 3H), 3.27-3.07 (m, 2H), 2.84-2.75 (m, 2H), 1.45 (s, 9H), 1.35 (d, J=6.0 Hz, 3H), 1.07-0.99 (m, 12H).

### Compound 014066A1 P2:

LCMS (M+H)⁺ m/z calculated 668.3. found 668.0.

¹H NMR (DMSO-d₆, 400 MHz): 8.86 (s, 1H), 8.06 (s, 1H), 7.43-7.41 (m, 1H), 6.90-6.82 (m, 2H), 4.88-4.84 (m, 1H), 4.19-4.15 (m, 1H), 4.03-3.98 (m, 1H), 3.83-3.75 (m, 2H), 3.22-3.00 (m, 2H), 2.93-2.80 (m, 2H), 1.45 (s, 9H), 1.34-1.31 (m, 3H), 1.08-1.01 (m, 12H).

### Step 2: Synthesis of 014066A2 P1 & 014066A2 P2

Compound 014066A1 P1 (44 mg, 0.066 mmol) was dissolved in dichloromethane (6 mL). Trifluoroacetic acid (1 mL) was added under an ice bath. The ice bath was removed, and the reaction mixture was stirred at room temperature (20° C.) for 0.5 h. The reaction mixture was concentrated under reduced pressure to give compound 014066A2 P1 as a yellow solid (crude product, 48 mg, 100%), which was directly used in the next step without purification. LCMS (M+H)⁺ m/z calculated 567.2. found 568.0.

Compound 014066A1 P2 (106 mg, 0.159 mmol) was dissolved in dichloromethane (10 mL). Trifluoroacetic acid (2 mL) was added under an ice bath. The ice bath was removed, and the reaction mixture was stirred at room temperature (20° C.) for 1 h, and concentrated under reduced pressure to give compound 014066A2 P2 as a brown solid (crude product, 160 mg, 100%), which was directly used in the next step without purification. LCMS (M+H)⁺ m/z calculated 567.2. found 568.0.

### Step 3: Synthesis of SZ-014066A & SZ-014066B

The crude compound 014066A2 P1 (crude product, 48 mg, 0.066 mmol) was dissolved in dichloromethane (5 mL). Acryloyl chloride (6 mg, 0.066 mmol) was slowly added under an ice bath, followed by diisopropylethylamine (43 mg, 0.33 mmol). After dropwise addition, the reaction mixture was stirred at 0° C. for 15 min. Saturated ammonium chloride solution (10 mL) was added, followed by extraction with dichloromethane (15 mL×3). The organic phases were combined, washed with water (10 mL), and concentrated under reduced pressure, and the residue was purified by preparative high performance liquid chromatography (ammonium bicarbonate) to give compound SZ-014066A as a yellow solid (7 mg, 17% yield).

Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm. The purity was 85.90%, Rt=3.404 min.

LCMS (M+H)⁺ m/z calculated 622.2. found 622.1. ¹H NMR (DMSO-d₆, 400 MHz): δ 10.29 (s, 1H), 8.87 (s, 1H), 7.98 (s, 1H), 7.30 (dd, J₁=15.6 Hz, J₂=8.4 Hz, 1H), 6.87-6.68 (m, 3H), 6.20 (d, J₁=16.8, 1H), 5.76 (dd, J₁=10.0 Hz, J₂=2.0 Hz, 1H), 4.98-4.88 (m, 1H), 4.42-3.99 (m, 3H), 3.81-3.35 (m, 2H), 3.28-3.02 (m, 1H), 2.86-2.73 (m, 2H), 1.33 (d, J=6.4 Hz, 3H), 1.07-1.00 (m, 12H).

The crude compound 014066A2P2 (crude product, 160 mg, 0.09 mmol) was dissolved in dichloromethane (5 mL). Acryloyl chloride (8.1 mg, 0.09 mmol) was slowly added under an ice bath, followed by diisopropylethylamine (58 mg, 0.45 mmol). After dropwise addition, the reaction phase was stirred at 0° C. for 15 min. Saturated ammonium chloride solution (10 mL) was added, followed by extraction with dichloromethane (3×10 mL). The organic phases were combined, washed with water (10 mL), and concentrated under reduced pressure, and the residue was purified by preparative high performance liquid chromatography (ammonium bicarbonate) to give compound SZ-014066B as a yellow solid (19 mg, 34% yield), which was further purified by preparative high performance liquid chromatography (ammonium bicarbonate) to give compound SZ-014066B as a white solid (4 mg, 7% yield).

Liquid chromatography-mass spectrometry [mobile phase: elution was carried out with a gradient from 80% water (containing 0.1% trifluoroacetic acid) and 20% acetonitrile to 30% water (containing 0.1% trifluoroacetic acid) and 70% acetonitrile at a flow rate of 1.5 mL per minute at a column temperature of 40° C. for 6 min. Column: waters XBridge C18 3.5 μm, 50×4.6 mm. The purity was 85.65%, Rt=3.908 min.

LCMS (M+H)⁺ m/z calculated 622.2. found 622.3. ¹H NMR (DMSO-d₆, 400 MHz): ¹H NMR (DMSO-d₆, 400 MHz): 8.86 (s, 1H), 8.09 (s, 1H), 7.30 (dd, J₁=15.2 Hz, J₂=8.4 Hz, 1H), 6.97-6.83 (m, 3H), 6.21 (d, J₁=12.4, 1H), 5.76 (dd, J₁=10.0 Hz, J₂=2.0 Hz, 1H), 4.98-4.88 (m, 1H), 4.43-3.99 (m, 3H), 3.84-3.58 (m, 1H), 3.22-3.00 (m, 2H), 2.96-2.82 (m, 2H), 1.31 (d, J=6.4 Hz, 3H), 1.07-1.01 (m, 6H), 0.99-0.92 (m, 6H).

### Example 18. SZ-014077AB

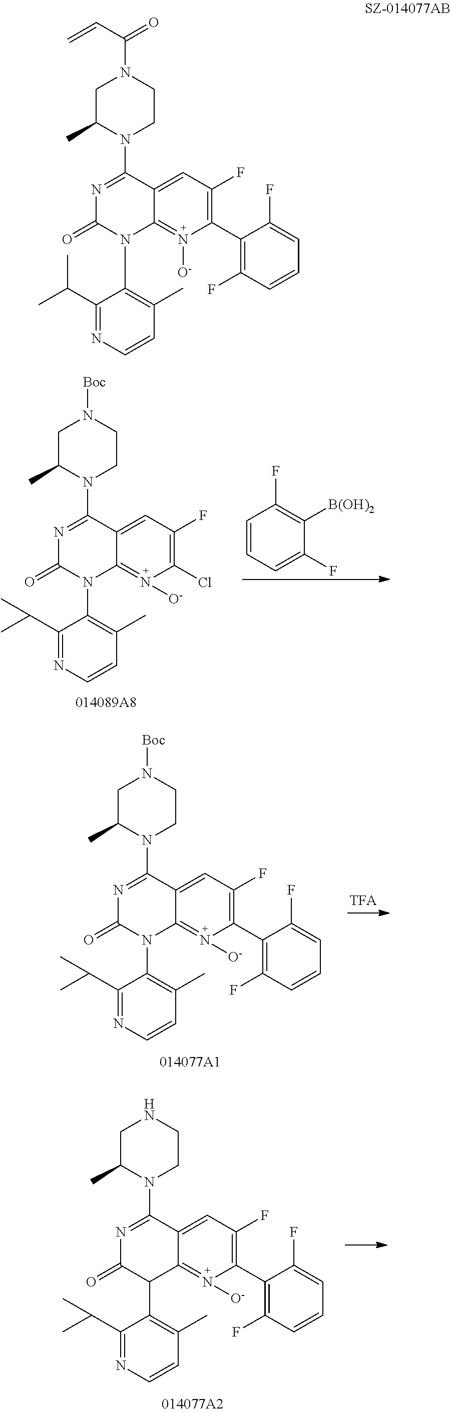

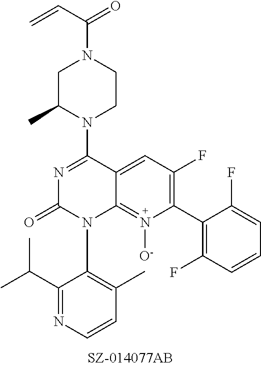

### Step 1: Synthesis of 014077A1

Compound 014089A8 (275 mg, 0.5 mmol), 2,6-difluorophenylboronic acid (160 mg, 1.0 mmol), potassium phosphate (212 mg, 1.0 mmol) and 2-dicyclohexylphosphine-2′,6′-dimethoxybiphenyl (61 mg, 0.15 mmol) were dissolved in 1,4-dioxane (10 mL). The system was purged with nitrogen several times, and then tris(dibenzylideneacetone)dipalladium (92 mg, 0.10 mmol) was added. The system was purged with nitrogen several times, and then the reaction mixture was stirred at 90° C. for 15 h, cooled to room temperature, and filtered. The filtrate was diluted with ethyl acetate (30 mL) and washed with saturated brine. The organic phases were combined and dried over anhydrous sodium sulfate. The filtrate was concentrated under reduced pressure, and the resulting residue was purified by column chromatography (PE:EA=2:1 to 1:1), followed by concentration to give crude compound 014077A1 as a yellow solid (40 mg, 12.8% yield). LCMS (M+H)⁺ m/z calculated 625.3. found 625.3. ¹H NMR (CDCl₃, 400 MHz): δ 8.45 (d, J=6.4 Hz, 1H), 7.55-7.45 (m, 1H), 7.41-7.37 (m, 1H), 7.06-7.00 (m, 3H), 4.93-4.81 (m, 1H), 4.40-4.03 (m, 3H), 3.76-3.67 (m, 1H), 3.35-3.11 (m, 2H), 2.76-2.67 (m, 1H), 2.28 (d, J=4.2 Hz, 3H), 1.56 (s, 9H), 1.53-1.50 (m, 3H), 1.25-1.16 (m, 6H).

### Step 2: Synthesis of 014077A2

The crude compound 014077A1 (40 mg, 0.064 mmol) was dissolved in dichloromethane (2 mL). Trifluoroacetic acid (290 mg, 2.56 mmol) was added under an ice bath. The ice bath was removed, and the reaction mixture was stirred at room temperature for 2 h, and concentrated to remove trifluoroacetic acid. The resulting yellow oil 014077A2 (32 mg, crude product) was directly used in the next step without purification. LCMS (M+H)⁺ m/z calculated 525.2. found 525.2.

### Step 3: Synthesis of SZ-014077AB

The crude compound 014077A2 (32 mg, 0.061 mmol) was dissolved in dichloromethane (2.0 mL). Acryloyl chloride (5.2 mg, 0.058 mmol) and N,N-diisopropylethylamine (23.6 mg, 0.183 mmol) were added. The reaction mixture was stirred at 0° C. for 5 min. The reaction mixture was diluted with dichloromethane (20 mL), quenched with saturated ammonium chloride solution (10 mL), washed with water (10 mL×2), and concentrated under reduced pressure. The residue was purified by preparative high performance liquid chromatography (ammonium bicarbonate) to give SZ-014077AB as a yellow solid (10.0 mg, 28.4% yield). Liquid chromatography-mass spectrometry [mobile phase: elution was carried out with a gradient from 80% water (containing 0.02% ammonium acetate) and 20% acetonitrile to 20% water (containing 0.02% ammonium acetate) and 80% acetonitrile at a flow rate of 1.5 mL per minute at a column temperature of 40° C. for 6 min. Column: waters XBridge C18 3.5 μm, 4.6×50 mm]. The purity was 92.96%, Rt=3.117 min. LCMS (M+H)⁺ m/z calculated 579.2. found 579.2.

¹H NMR (DMSO-d₆, 400 MHz): δ 8.26 (dd, J₁=4.8 Hz, J₂=2.0 Hz, 1H), 8.01-7.91 (m, 1H), 7.72-7.65 (m, 1H), 7.33-7.26 (m, 2H), 7.03 (t, J=4.0 Hz, 1H), 6.87-6.81 (m, 1H), 6.20 (d, J=16.8 Hz, 1H), 5.76 (dd, J₁=10.4 Hz, J₂=2.4 Hz, 1H), 4.95-4.87 (br s, 1H), 4.42-4.01 (m, 3H), 3.72-3.40 (m, 2H), 3.26-2.98 (m, 1H), 2.77-2.67 (m, 1H), 2.01 (d, J=7.6 Hz, 3H), 1.34-1.29 (m, 3H), 1.06-0.83 (m, 6H).

### Example 19. SZ-014082AB

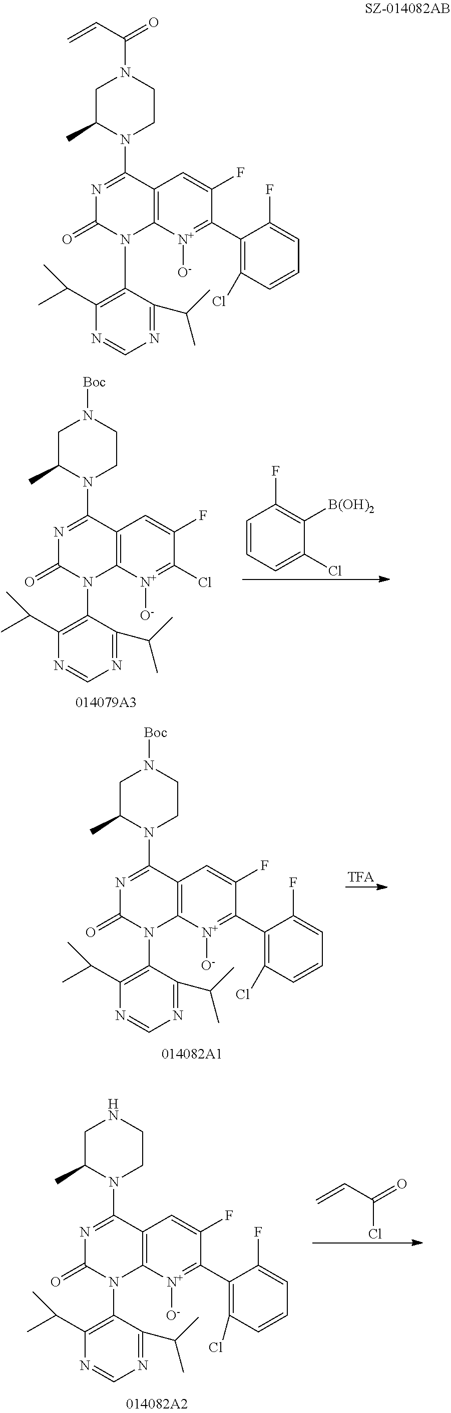

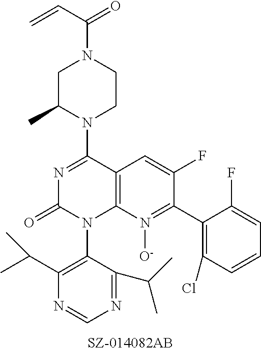

### Step 1: Synthesis of 014082A1

Compound 014079A3 (250 mg, 0.43 mmol), 2-fluoro-6-chlorophenylboronic acid (150 mg, 0.86 mmol), potassium phosphate (182 mg, 0.86 mmol) and 2-dicyclohexylphosphine-2′,6′-dimethoxybiphenyl (53 mg, 0.13 mmol) were dissolved in 1,4-dioxane (6 mL). The system was purged with nitrogen several times, and then tris(dibenzylideneacetone)dipalladium (78 mg, 0.086 mmol) was added. The system was purged with nitrogen several times, and then the reaction mixture was stirred at 90° C. for 5 h, cooled to room temperature, and filtered. The filtrate was diluted with ethyl acetate (30 mL) and washed with saturated brine. The organic phases were combined and dried over anhydrous sodium sulfate. The filtrate was concentrated under reduced pressure, and the resulting residue was purified by column chromatography (PE:EA=2:1 to 1:1), followed by concentration to give compound 014082A1 as a pale yellow solid (100 mg, 34.5% yield). LCMS (M+H)⁺ m/z calculated 670.2. found 670.2. ¹H NMR (CDCl₃, 400 MHz): δ 8.87 (s, 1H), 8.02-8.00 (m, 1H), 7.68-7.62 (m, 2H), 7.56-7.48 (m, 1H), 4.93-4.83 (m, 1H), 4.24-3.85 (m, 2H), 3.80-3.65 (m, 3H), 3.20-3.07 (m, 1H), 2.92-2.78 (m, 2H), 1.49 (s, 9H), 1.38-1.34 (m, 3H), 1.06-0.98 (m, 12H).

### Step 2: Synthesis of 014082A2

The crude compound 014082A1 (100 mg, 0.15 mmol) was dissolved in dichloromethane (3 mL). Trifluoroacetic acid (512 mg, 4.5 mmol) was added under an ice bath. The ice bath was removed, and the reaction mixture was stirred at room temperature for 2 h, and concentrated to remove trifluoroacetic acid. The resulting yellow oil 014082A2 (100 mg, crude product) was directly used in the next step without purification. LCMS (M+H)⁺ m/z calculated 570.2. found 570.2.

### Step 3: Synthesis of SZ-014082AB

The crude compound 014082A2 (100 mg, 0.150 mmol) was dissolved in dichloromethane (8.0 mL). Acryloyl chloride (12.2 mg, 0.135 mmol) was added, followed by N,N-diisopropylethylamine (58.1 mg, 0.45 mmol). The reaction mixture was stirred at 0° C. for 5 min. The reaction mixture was diluted with dichloromethane (20 mL), quenched with saturated ammonium chloride solution (10 mL), washed with water (10 mL×2), and concentrated under reduced pressure. The residue was purified by preparative high performance liquid chromatography (ammonium bicarbonate) to give SZ-014082AB as a yellow solid (10.0 mg, 28.4% yield).

Liquid chromatography-mass spectrometry [mobile phase: elution was carried out with a gradient from 70% water (containing 0.02% ammonium acetate) and 30% acetonitrile to 40% water (containing 0.02% ammonium acetate) and 60% acetonitrile at a flow rate of 1.5 mL per minute at a column temperature of 40° C. for 6 min. Column: waters XBridge C18 3.5 μm, 4.6×50 mm], 83.13% purity, Rt=3.523 min (isomer 10.68%, Rt=4.605 min). LCMS (M+H)⁺ m/z calculated 624.2. found 623.8. 1H NMR (DMSO-d6, 400 MHz): δ 8.89 (s, 1H), 8.05-8.03 (m, 1H), 7.68-7.62 (m, 1H), 7.52 (d, J=8.0 Hz, 1H), 7.42 (t, J=8.4 Hz, 1H), 6.91-6.82 (m, 1H), 6.20 (d, J=17.2 Hz, 1H), 5.76 (dd, J₁=10.0 Hz, J₂=2.0 Hz, 1H), 4.94-4.89 (br s, 1H), 4.44-4.28 (m, 2H), 4.18-4.02 (m, 1H), 3.77-3.59 (m, 1H), 3.51-3.39 (m, 1H), 3.28-3.07 (m, 1H), 2.88-2.81 (m, 2H), 1.30 (d, J=6.4 Hz, 3H), 1.07 (td, J₁=6.4 Hz, J₂=2.0 Hz, 6H), 1.02 (td, J₁=6.4 Hz, J₂=2.0 Hz, 6H).

### Example 20. SZ-014108AB

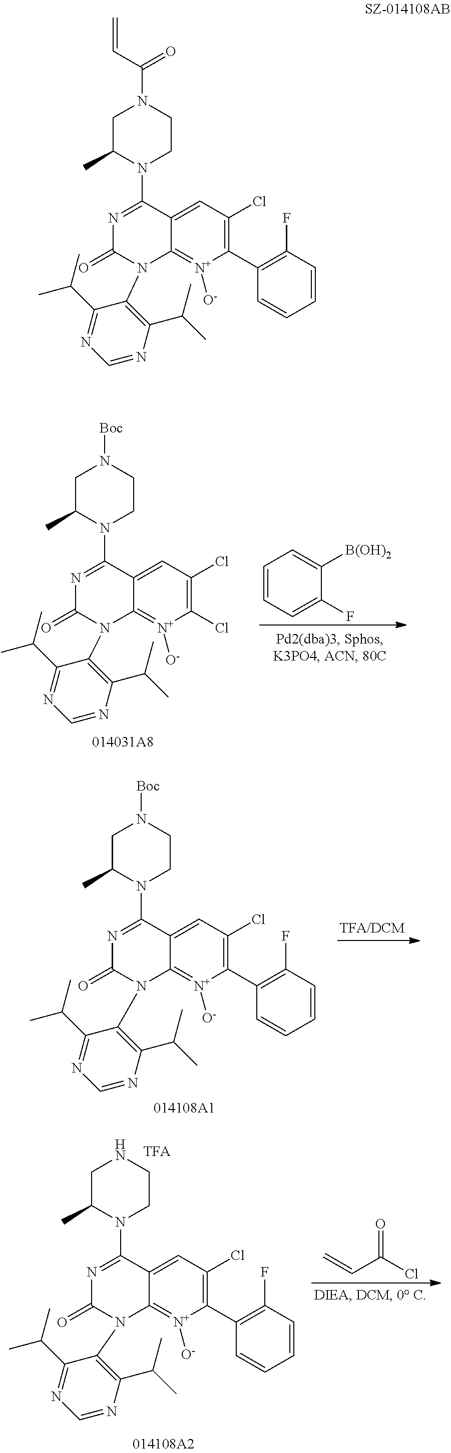

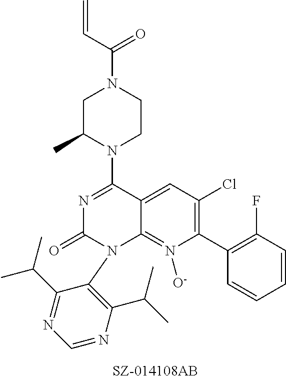

### Step 1: Synthesis of 014108A1

Compound 014031A8 (300 mg, 0.51 mmol), 2-fluorophenylboronic acid (213 mg, 1.52 mmol), potassium phosphate (322 mg, 1.52 mmol) and 2-dicyclohexylphosphine-2′,6′-dimethoxybiphenyl (63 mg, 0.153 mmol) were dissolved in anhydrous acetonitrile (25 mL). The system was purged with nitrogen three times, and then tris(dibenzylideneacetone)dipalladium (47 mg, 0.051 mmol) was added. The system was purged with nitrogen three times, and then the reaction mixture was stirred at 80° C. under nitrogen for 5 h. The reaction solution was cooled to room temperature and filtered. The filtrate was concentrated. Water (20 mL) was added to the residue, followed by extraction with ethyl acetate (3×15 mL). The organic phases were combined, washed with saturated brine, dried over anhydrous sodium sulfate, and filtered. The filtrate was concentrated under reduced pressure, and the resulting residue was purified by preparative high performance liquid chromatography to give compound 014108A1 as a yellow solid (20 mg, 6% yield). LCMS (M+H)⁺ m/z calculated 652.3. found 652.3. ¹H NMR (DMSO-d₆, 400 MHz): δ 8.87 (s, 1H), 8.01 (d, J=7.6 Hz, 1H), 7.58-7.53 (m, 1H), 7.40-7.18 (m, 3H), 4.92-4.83 (m, 1H), 4.21-4.18 (m, 1H), 3.99-3.96 (m, 1H), 3.86-3.71 (m, 2H), 3.12-3.04 (m, 1H), 2.89-2.76 (m, 2H), 2.02-1.97 (m, 1H), 1.45 (s, 9H), 1.35 (d, J=5.6 Hz, 3H), 1.08-0.99 (m, 12H).

### Step 2: Synthesis of 014108A2

Compound 014108A1 (20 mg, 0.031 mmol) was dissolved in dichloromethane (6 mL). Trifluoroacetic acid (1 mL) was added under an ice bath. The ice bath was removed, and the reaction mixture was stirred at room temperature for 0.5 h. The reaction mixture was concentrated under reduced pressure to give compound 014108A2 as a yellow solid (crude product, 42 mg, 100%), which was directly used in the next step without purification. LCMS (M+H)⁺ m/z calculated 552.2. found 552.2.

### Step 3: Synthesis of SZ-014108AB

Compound 014108A2 (crude product, 42 mg, 0.031 mmol) was dissolved in dichloromethane (3 mL). Acryloyl chloride (2.8 mg, 0.031 mmol) was slowly added under an ice bath, followed by diisopropylethylamine (20 mg, 0.155 mmol). After dropwise addition, the reaction phase was stirred at 0° C. for 15 min. Saturated ammonium chloride solution (15 mL) was added, followed by extraction with dichloromethane (10 mL×3). The organic phases were combined, washed with water (10 mL), and concentrated under reduced pressure, and the residue was purified by preparative high performance liquid chromatography (ammonium bicarbonate) to give compound SZ-014108AB as a yellow solid (6 mg, 32% yield).

Liquid chromatography-mass spectrometry [mobile phase: elution was carried out with a gradient from 70% water (containing 0.02% ammonium acetate) and 30% acetonitrile to 40% water (containing 0.02% ammonium acetate) and 60% acetonitrile at a flow rate of 1.5 mL per minute at a column temperature of 40° C. for 6 min. Column: waters XBridge C18 3.5 μm, 50×4.6 mm. 89.2% purity, Rt=3.438 min. LCMS (M+H)⁺ m/z calculated 606.2. found 606.3. ¹H NMR (DMSO-d₆, 400 MHz): δ 8.87 (s, 1H), 8.04 (d, J=3.6 Hz, 1H), 7.58-7.53 (m, 1H), 7.39-7.29 (m, 3H), 6.90-6.81 (m, 1H), 6.20 (d, J=23.2 Hz, 1H), 5.76 (dd, J₁=10.4 Hz, J₂=2.4 Hz, 1H), 4.92-4.90 (m, 1H), 4.42-4.00 (m, 3H), 3.83-3.60 (m, 2H), 3.28-3.02 (m, 1H), 2.89-2.76 (m, 2H), 2.02-1.95 (m, 1H), 1.33 (d, J=6.8 Hz, 3H), 1.08-0.99 (m, 12H).

### Example 21. SZ-014111AB

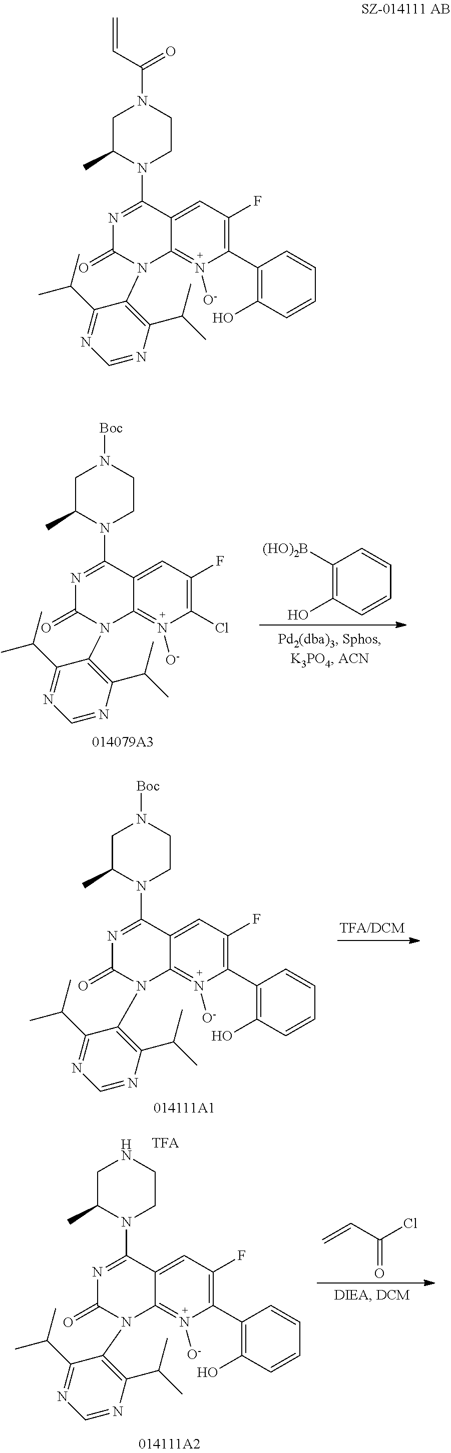

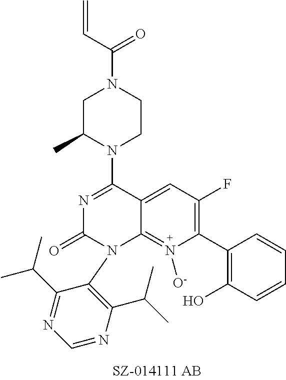

### Step 1: Synthesis of 014111A1

Compound 014079A3 (300 mg, 0.52 mmol), 2-hydroxyphenylboronic acid (574 mg, 4.16 mmol), potassium phosphate (331 mg, 1.56 mmol) and 2-dicyclohexylphosphine-2′,6′-dimethoxybiphenyl (64 mg, 0.156 mmol) were dissolved in anhydrous acetonitrile (20 mL). The system was purged with nitrogen three times, and then tris(dibenzylideneacetone)dipalladium (48 mg, 0.052 mmol) was added. The system was purged with nitrogen three times, and then the reaction mixture was stirred at 80° C. under nitrogen for 5 h. The reaction solution was cooled to room temperature and filtered. The filtrate was concentrated. Water (25 mL) was added to the residue, followed by extraction with ethyl acetate (20×3 mL). The organic phases were combined, washed with saturated brine, dried over anhydrous sodium sulfate, and filtered. The filtrate was concentrated under reduced pressure, and the resulting residue was purified by flash silica gel column chromatography to give compound 014111A1 as a red solid (218 mg, 66% yield). LCMS (M+H)⁺ m/z calculated 634.3. found 634.3. ¹H NMR (DMSO-d₆, 400 MHz): δ 9.80 (s, 1H), 8.88 (s, 1H), 7.88 (d, J=8.0 Hz, 1H), 7.34-7.30 (m, 1H), 7.18-7.12 (m, 1H), 6.94-6.85 (m, 2H), 4.92-4.83 (m, 1H), 4.23-4.12 (m, 1H), 3.92-3.82 (m, 1H), 3.75-3.63 (m, 1H), 3.41-3.31 (m, 2H), 3.16-3.04 (m, 1H), 2.83-2.77 (m, 2H), 1.45 (s, 9H), 1.34 (d, J=6.0 Hz, 3H), 1.08-1.03 (m, 12H).

### Step 2: Synthesis of 014111A2

Compound 014111A1 (210 mg, 0.33 mmol) was dissolved in dichloromethane (10 mL). Trifluoroacetic acid (2 mL) was added under an ice bath. The ice bath was removed, and the reaction mixture was stirred at room temperature for 0.5 h. The reaction mixture was concentrated under reduced pressure to give compound 014111A2 as a yellow solid (crude product, 380 mg, 100%), which was directly used in the next step without purification. LCMS (M+H)⁺ m/z calculated 534.3. found 534.2.

### Step 3: Synthesis of SZ-014111AB

Compound 014111A2 (crude product, 380 mg, 0.33 mmol) was dissolved in dichloromethane (10 mL). Acryloyl chloride (29.7 mg, 0.33 mmol) was slowly added under an ice bath, followed by diisopropylethylamine (213 mg, 1.65 mmol). After dropwise addition, the reaction phase was stirred at 0° C. for 15 min. Saturated ammonium chloride solution (10 mL) was added, followed by extraction with dichloromethane (10 mL×3). The organic phases were combined, washed with water (15 mL), dried over anhydrous sodium sulfate, and filtered. The filtrate was concentrated under reduced pressure, and the resulting residue was purified by preparative high performance liquid chromatography (ammonium bicarbonate) to give compound SZ-014111AB as a yellow solid (90 mg, 46% yield).

Liquid chromatography-mass spectrometry [mobile phase: elution was carried out with a gradient from 70% water (containing 0.02% ammonium acetate) and 30% acetonitrile to 40% water (containing 0.02% ammonium acetate) and 60% acetonitrile at a flow rate of 1.5 mL per minute at a column temperature of 40° C. for 6 min. Column: waters XBridge C18 3.5 μm, 50×4.6 mm. The purity was 95.9%, Rt=2.801 min. LCMS (M+H)⁺ m/z calculated 588.3. found 588.4. ¹H NMR (DMSO-d₆, 400 MHz): δ 9.79 (s, 1H), 8.88 (s, 1H), 7.91 (d, J=7.6 Hz, 1H), 7.34-7.30 (m, 1H), 7.14 (d, J=7.6 Hz, 1H), 6.93-6.81 (m, 3H), 6.20 (d, J=16.8 Hz, 1H), 5.76 (dd, J₁=10.4 Hz, J₂=2.0 Hz, 1H), 4.92-4.90 (m, 1H), 4.43-4.01 (m, 3H), 3.74-3.43 (m, 2H), 3.26-3.06 (m, 1H), 2.89-2.72 (m, 2H), 1.32 (d, J=6.8 Hz, 3H), 1.08-1.03 (m, 12H).

### Example 22. SZ-014089P1AB & SZ-014089P2AB

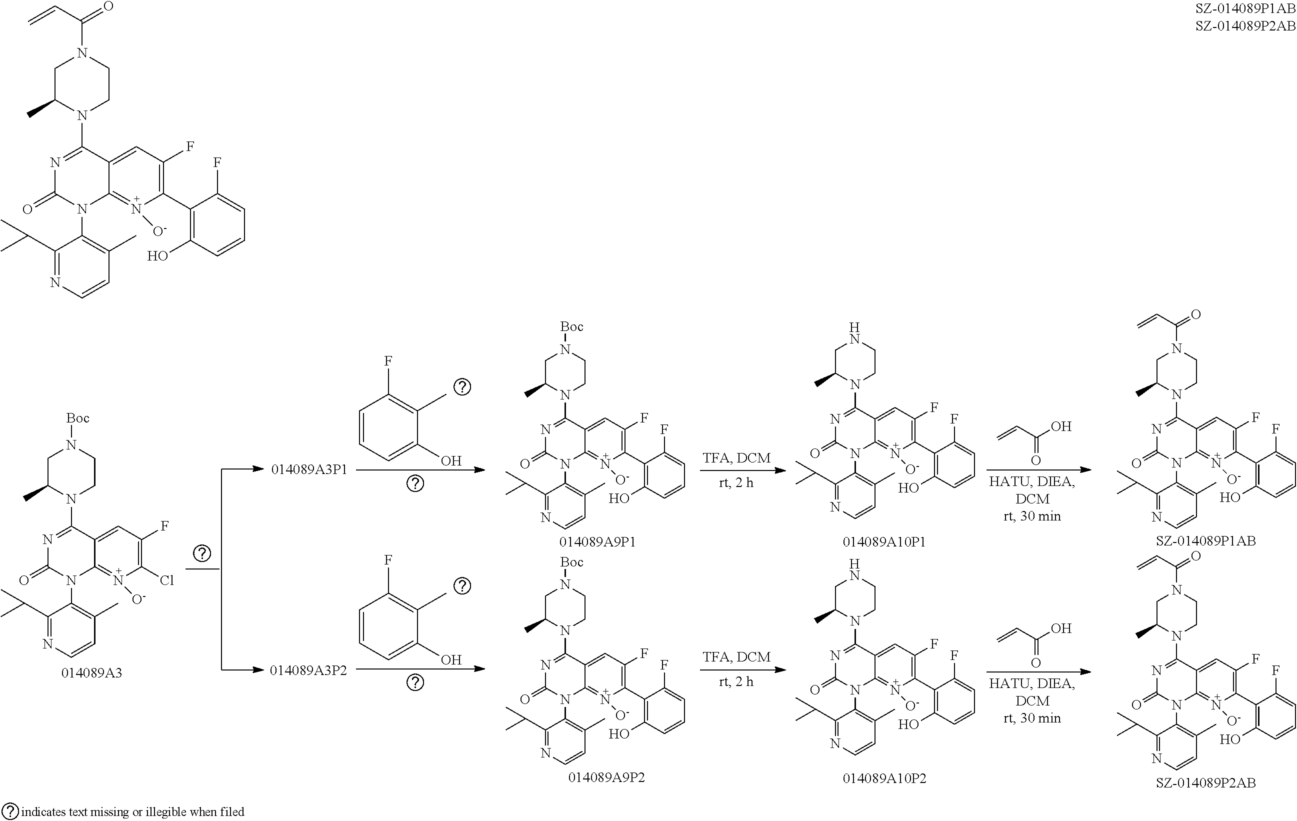

### Step 1: Synthesis of 014089A9PG-3C

014089A8 (1.1 g) was subjected to chiral resolution: Colum: Chiralpak 0B 250 mm×4.6 mm 5 (m, Mobile phase: Hex:EtOH=80:20, F: 1 mL/min, T=30, elution for 16 min. 014089A8P1 was obtained:

300 mg, RT=7.565.

¹HNMR (DMSO-d₆, 400 MHz): δ 8.34 (d, J=6.4 Hz, 1H), 7.95 (d, J=10.4 Hz, 1H), 7.11 (d, J=5.2 Hz, 1H), 4.80-4.76 (m, 1H), 4.12-3.80 (m, 2H), 3.70-3.62 (m, 3H), 3.20-3.03 (m, 1H), 2.81-2.74 (m, 1H), 1.99 (s, 3H), 1.45 (s, 9H), 1.32 (d, J=8.8 Hz, 3H), 1.11-0.99 (m, 6H).

014089A8P2:

290 mg, RT=9.411.

LCMS (M+H)⁺ m/z calculated 547.2. found 547.4.

¹HNMR (DMSO-d₆, 400 MHz): δ 8.35 (d, J=6.4 Hz, 1H), 7.96 (d, J=10.8 Hz, 1H), 7.31-7.26 (m, 1H), 4.81-4.79 (m, 1H), 4.13-3.83 (m, 2H), 3.70-3.62 (m, 3H), 3.24-3.04 (m, 1H), 2.75-2.71 (m, 1H), 2.05 (s, 3H), 1.47 (s, 9H), 1.32 (d, J=8.8 Hz, 3H), 1.10-0.99 (in, 6H).

### Step 2: Synthesis of 014089A9

Compound 014089A8P1 (350 mg, 0.64 mmol), 2-fluoro-6-hydroxyphenylboronic acid (142 mg, 1.28 mmol), potassium phosphate (270 mg, 1.28 mmol) and 2-dicyclohexylphosphine-2′,6′-dimethoxybiphenyl (78 mg, 0.20 mmol) were dissolved in 1,4-dioxane (30 mL). The system was purged with nitrogen several times, and then tris(dibenzylidene-BASE acetone)dipalladium(0) (117 mg, 0.128 mmol) was added. The system was purged with nitrogen several times, and then the reaction mixture was stirred at 80° C. for 3 h, cooled to room temperature, and filtered. The filtrate was diluted with ethyl acetate (100 mL) and washed with saturated brine. The organic phases were combined and dried over anhydrous sodium sulfate. The filtrate was concentrated under reduced pressure, and the resulting residue was purified by silica gel column chromatography (petroleum ether/ethyl acetate 1:2) to give compound 014089A9P1 as a pale yellow solid (250 mg, 62.5% yield). LCMS (M+H)⁺ m/z calculated 623.3. found 623.3. ¹HNMR (DMSO-d₆, 400 MHz): δ 10.39 (br s, 1H), 8.24 (d, J=4.8 Hz, 1H), 7.81 (d, J=7.6 Hz, 1H), 7.34 (q, J=3.6 Hz, 1H), 7.02 (t, J=5.6 Hz, 1H), 6.74 (q, J=8.4 Hz, 2H), 4.85-4.73 (m, 1H), 4.22-3.98 (m, 2H), 3.92-3.58 (m, 3H), 3.26-3.01 (m, 1H), 2.78-2.69 (m, 1H), 1.97 (s, 3H), 1.45 (s, 9H), 1.35 (d, J=6.4 Hz, 3H), 1.06-0.99 (m, 6H).

Compound 014089A8P2 (250 mg, 0.46 mmol), 2-fluoro-6-hydroxyphenylboronic acid (199 mg, 0.92 mmol), potassium phosphate (195 mg, 0.92 mmol) and 2-dicyclohexylphosphine-2′,6′-dimethoxybiphenyl (57 mg, 0.14 mmol) were dissolved in 1,4-dioxane (30 mL). The system was purged with nitrogen several times, and then tris(dibenzylidene-BASE acetone)dipalladium(0) (84 mg, 0.092 mmol) was added. The system was purged with nitrogen several times, and then the reaction mixture was stirred at 80° C. for 3 h, cooled to room temperature, and filtered. The filtrate was diluted with ethyl acetate (100 mL) and washed with saturated brine. The organic phases were combined and dried over anhydrous sodium sulfate. The filtrate was concentrated under reduced pressure, and the resulting residue was purified by silica gel column chromatography (petroleum ether/ethyl acetate 1:2), followed again by preparative high performance liquid chromatography (ammonium bicarbonate) to give compound 014089A9P2 as a pale yellow solid (10 mg, 3.5% yield). LCMS (M+H)⁺ m/z calculated 623.3. found 623.3.

### Step 3: Synthesis of 014089A10

Compound 014089A9P1 (250 mg, 0.40 mmol) was dissolved in dichloromethane (10 mL). Trifluoroacetic acid (1.82 g, 16.0 mmol) was added under an ice bath. The ice bath was removed, and the reaction mixture was stirred at room temperature (20° C.) for 2 h. The reaction phase was directly concentrated under reduced pressure to give 014089A10P1 as an orange oil (250 mg, crude product), which was directly used in the next step without purification. LCMS (M+H)⁺ m/z calculated 523.2. found 523.2.

Compound 014089A9P2 (10 mg, 0.016 mmol) was dissolved in dichloromethane (2 mL). Trifluoroacetic acid (73 mg, 0.64 mmol) was added under an ice bath. The ice bath was removed, and the reaction mixture was stirred at room temperature (20° C.) for 2 h. The reaction phase was directly concentrated under reduced pressure to give 014089A10P2 as an orange oil (9 mg, crude product), which was directly used in the next step without purification. LCMS (M+H)⁺ m/z calculated 523.2. found 523.2.

### Step 4: Synthesis of SZ-014089P1AB & SZ-014089P2AB

The crude compound 014089A10P1 (250 mg, trifluoroacetate salt, 0.40 mmol) was dissolved in dichloromethane (5.0 mL). Acryloyl chloride (32.4 mg, 0.36 mmol) was added, followed by slow dropwise addition of N,N-diisopropylethylamine (154.8 mg, 1.2 mmol). The reaction mixture was stirred at 0° C. for 5 min. The reaction mixture was diluted with dichloromethane (50 mL), quenched with saturated ammonium chloride solution (20 mL), washed with water (20 mL×2), and concentrated under reduced pressure. The residue was purified by preparative high performance liquid chromatography (ammonium bicarbonate) to give SZ-014089P1AB as a yellow solid (42.0 mg, 24.0% yield).

Liquid chromatography-mass spectrometry [mobile phase: elution was carried out with a gradient from 80% water (containing 0.02% ammonium acetate) and 20% acetonitrile to 50% water (containing 0.02% ammonium acetate) and 50% acetonitrile at a flow rate of 1.5 mL per minute at a column temperature of 40° C. for 6 min. Column: waters XBridge C18 3.5 μm, 4.6×50 mm]. The purity was 91.60% (isomer A, 42.56%, Rt=3.559 min; isomer B, 49.05%, Rt=3.654 min). LCMS (M+H)⁺ m/z calculated 577.2. found 577.2. ¹H NMR (DMSO-d₆, 400 MHz): δ 10.36 (d, J=9.2 Hz, 1H), 8.25 (d, J=4.8 Hz, 1H), 7.83 (t, J=8.0 Hz, 1H), 7.37 (q, J=8.0 Hz, 1H), 7.02 (t, J=4.8 Hz, 1H), 6.87-6.71 (m, 3H), 6.20 (d, J=16.0 Hz, 1H), 5.76 (dd, J₁=10.4 Hz, J₂=2.4 Hz, 1H), 4.85-4.78 (br s, 1H), 4.42-4.25 (m, 2H), 4.16-4.00 (m, 1H), 3.70-3.45 (m, 2H), 3.25-3.09 (m, 1H), 2.77-2.67 (m, 1H), 1.99 (s, 3H), 1.34 (d, J=6.4 Hz, 3H), 1.10-0.96 (m, 6H).

The crude compound 014089A10P2 (9 mg, trifluoroacetate salt, 0.016 mmol) was dissolved in dichloromethane (1.0 mL). Acryloyl chloride (1.35 mg, 0.015 mmol) was added, followed by slow dropwise addition of N,N-diisopropylethylamine (154.8 mg, 1.2 mmol). The reaction mixture was stirred at 0° C. for 5 min. The reaction mixture was diluted with 50 mL of dichloromethane, quenched with saturated ammonium chloride solution (10 mL), washed with water (10 mL×2), and concentrated under reduced pressure. The residue was purified by preparative high performance liquid chromatography (ammonium bicarbonate) to give SZ-014089P2AB as a yellow solid (1.6 mg, 17.4% yield).

Liquid chromatography-mass spectrometry [mobile phase: elution was carried out with a gradient from 70% water (containing 0.02% ammonium acetate) and 30% acetonitrile to 50% water (containing 0.02% ammonium acetate) and 50% acetonitrile at a flow rate of 1.0 mL per minute at a column temperature of 40° C. for 15 min. Column: waters XBridge C18 3.5 μm, 4.6×50 mm]. The purity was 87.90% (isomer A, 10.72%, Rt=9.451 min; isomer B, 77.18%, Rt=9.787 min). LCMS (M+H)⁺ m/z calculated 577.2. found 577.3.

### Example 23. SZ-014062AB

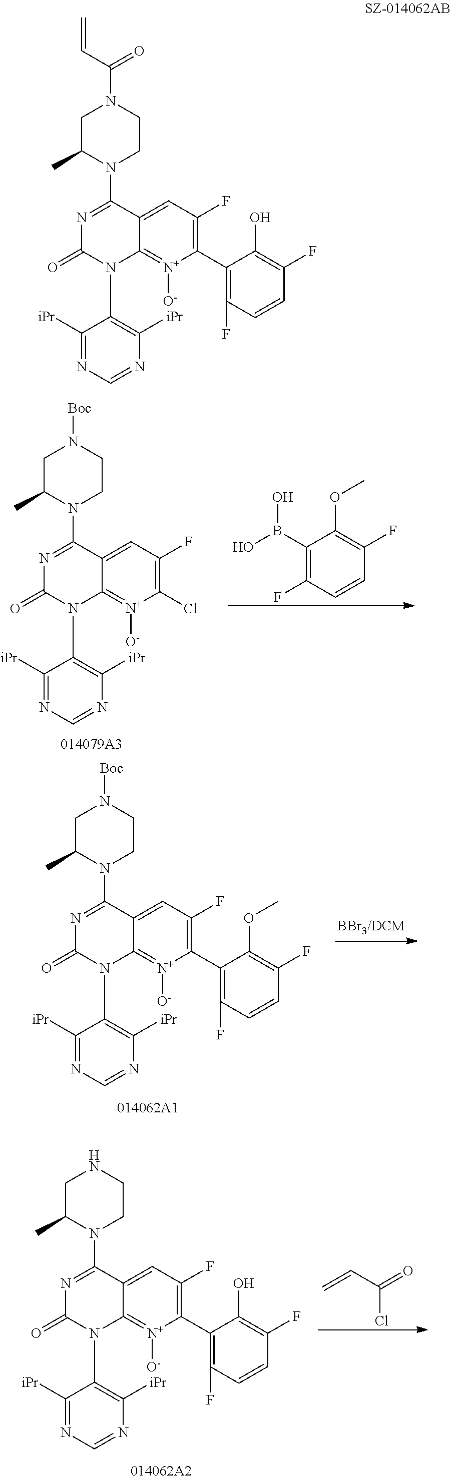

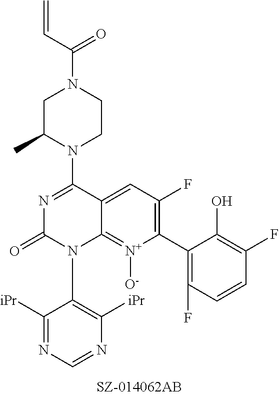 [SMILES: C=CC(=O)N1CCN(c2nc(=O)n(-c3c(C(C)C)ncnc3C(C)C)c3c2cc(F)c(-c2c(F)ccc(F)c2O)[n+]3[O-])[C@@H](C)C1]

### Step 1: Synthesis of 014062A1

Compound 014079A3 (576 mg, 1 mmol), 3,6-difluoro-2-methoxyphenylboronic acid (376 mg, 2 mmol), potassium phosphate (636 mg, 3 mmol) and 2-dicyclohexylphosphine-2′,6′-dimethoxybiphenyl (164 mg, 0.4 mmol) were dissolved in 1,4-dioxane (20 mL). The system was purged with nitrogen several times, and then tris(dibenzylideneacetone)dipalladium (183 mg, 0.2 mmol) was added. The system was purged with nitrogen several times, and then the reaction mixture was stirred at 90° C. overnight, cooled to room temperature, and filtered. The filtrate was concentrated under reduced pressure, and the resulting residue was purified by silica gel column chromatography (dichloromethane methanol=50:1), followed by preparative high performance liquid chromatography to give compound 014062A1 as a pale yellow solid (140 mg, 20% yield). LCMS (M+H)⁺ m/z calculated 684.3. found 684.3.

### Step 2: Synthesis of 014062A2

Compound 014062A1 (140 mg, 0.20 mmol) was dissolved in dichloromethane (14 mL). A solution of boron tribromide in dichloromethane (1.2 mL, 1.2 mmol) was added at −40° C. The reaction mixture was allowed to react for 2 h, with the temperature maintained at −40° C. The reaction mixture was quenched with methanol (15 mL), diluted with water (20 mL), and extracted with dichloromethane (20 mL×2). The organic phases were combined, dried over anhydrous sodium sulfate, and filtered. The filtrate was concentrated under reduced pressure, and the resulting residue was purified by preparative high performance liquid chromatography to give compound 014062A2 as a pale yellow solid (65 mg, 57% yield).

LCMS (M+H)⁺ m/z calculated 570.2. found 570.2.

### Step 3: Synthesis of SZ-014062AB

The crude compound 014062A2 (65 mg, 0.114 mmol) was dissolved in dichloromethane (3 mL). Acryloyl chloride (10 mg, 0.114 mmol) was slowly added under an ice bath, followed by diisopropylethylamine (44 mg, 0.342 mmol). After dropwise addition, the reaction phase was stirred at 0° C. for 10 min. The reaction mixture was diluted with dichloromethane (30 mL). Saturated aqueous ammonium chloride solution (20 mL) and saturated aqueous sodium bicarbonate solution (3 mL) were added. The organic phase was separated, washed twice with water (2×20 mL), dried over anhydrous magnesium sulfate, and filtered. The filtrate was concentrated under reduced pressure, and the residue was purified by preparative high performance liquid chromatography (ammonium bicarbonate) to give compound SZ-014062AB as a pale yellow solid (21 mg, 29% yield). Liquid chromatography-mass spectrometry [mobile phase: elution was carried out with a gradient from 80% water (containing 0.02% ammonium acetate) and 20% acetonitrile to 70% water (containing 0.02% ammonium acetate) and 30% acetonitrile at a flow rate of 1.5 mL per minute at a column temperature of 40° C. for 6 min. Column: waters XBridge C18 3.5 μm, 50×4.6 mm]. The purity was 95.06%, Rt=3.167 min. LCMS (M+H)⁺ m/z calculated 624.3. found 624.3.

¹H NMR (DMSO-d₆, 400 MHz): δ 8.89 (s, 1H), 7.99-7.93 (m, 1H), 7.35-7.34 (m, 1H), 6.88-6.81 (m, 1H), 6.74-6.71 (m, 1H), 6.21 (d, J=17.2 Hz, 1H), 5.77 (dd, J₁=10.4 Hz, J₂=2.4 Hz, 1H), 4.90 (br s, 1H), 4.43-4.01 (m, 3H), 3.64-3.31 (m, 2H), 3.27-3.07 (m, 1H), 2.86-2.75 (m, 2H), 1.33-1.27 (m, 3H), 1.08-0.97 (m, 12H).

### Example 24. SZ-014114

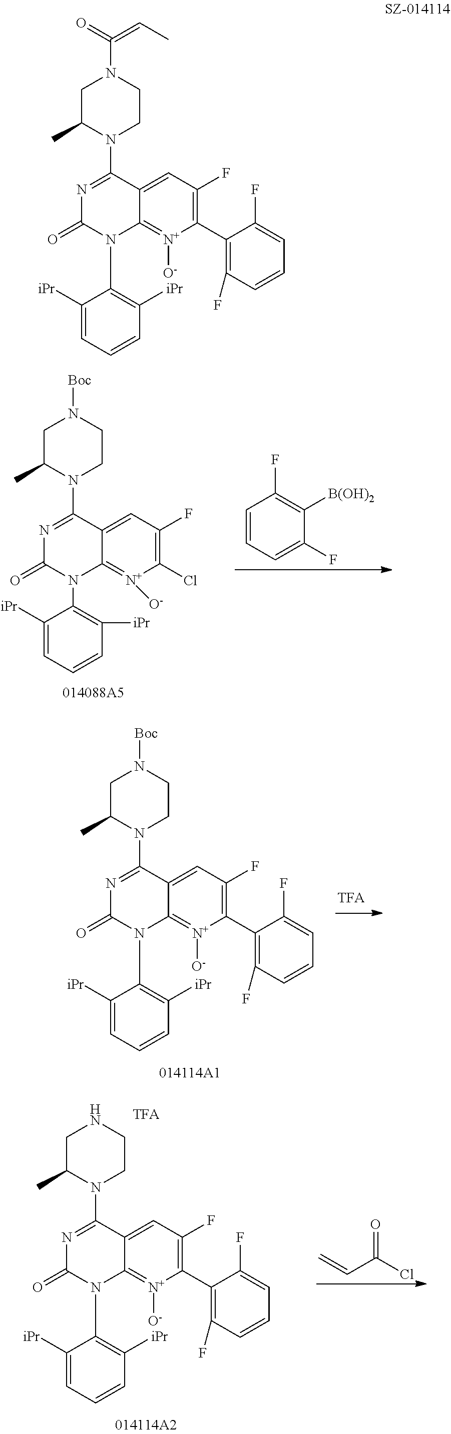

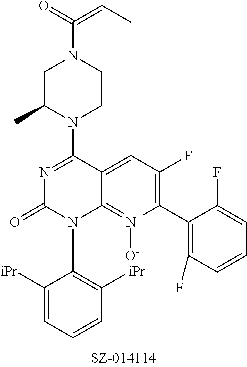

### Step 1: Synthesis of 014114A1

Compound 014088A5 (4.2 g, 7.32 mmol), 2,6-difluorophenylboronic acid (4.8 g, 21.96 mmol), potassium phosphate (3.1 g, 14.64 mmol) and Sphos (1.5 g, 3.66 mmol) were dissolved in 1,4-dioxane (100 mL). The system was purged with nitrogen several times, and then tris(dibenzylideneacetone)dipalladium (1.7 g, 1.83 mmol) was added. The system was purged with nitrogen several times, and then the reaction mixture was stirred at 120° C. overnight, cooled to room temperature, and filtered. The filtrate was concentrated under reduced pressure, and the resulting residue was purified by silica gel column chromatography (petroleum ether:ethyl acetate=3:1), followed by preparative high performance liquid chromatography to give compound 014114A1 as a pale yellow solid (581 mg, 11.4% yield).

LCMS (M+H)⁺ calculated 652.3. found 652.3. ¹H NMR (DMSO-d₆, 400 MHz): δ 7.87 (d, J=8.0 Hz, 1H), 7.69-7.65 (m, 1H), 7.28 (t, J=8.4 Hz, 2H), 7.19 (t, J=8.0 Hz, 1H), 7.10-7.03 (m, 2H), 4.79 (m, 2H), 4.15-4.12 (m, 1H), 4.01-3.85 (m, 1H), 3.84-3.78 (m, 1H), 3.65-3.58 (m, 1H), 3.25-3.17 (m, 1H), 2.62-2.54 (m, 2H), 1.44 (s, 9H), 1.33-1.31 (m, 3H), 1.13-1.08 (m, 6H), 1.04-0.91 (m, 6H).

### Step 2: Synthesis of 014114A2

Compound 014114A1 (146 mg, 0.223 mmol) was dissolved in dichloromethane (6 mL). Trifluoroacetic acid (2 mL) was added under an ice bath. The ice bath was removed, and the reaction mixture was stirred at room temperature (20° C.) for 2 h, and concentrated under reduced pressure and a water bath at 30° C. to give compound 014114A2 as a yellow oil (148 mg, crude product), which was directly used in the next step without purification.

LCMS (M+H)⁺ m/z calculated 552.3. found 552.3.

### Step 3: Synthesis of SZ-014114

The crude compound 014114A2 (148 mg, 0.223 mmol) was dissolved in dichloromethane (3 mL). Acryloyl chloride (20.1 mg, 0.223 mmol) was slowly added under an ice bath, followed by diisopropylethylamine (86 mg, 0.669 mmol). After dropwise addition, the reaction phase was stirred at 0° C. for 10 min. Then, the reaction mixture was diluted with dichloromethane (30 mL). Saturated aqueous ammonium chloride solution (20 mL) and saturated aqueous sodium bicarbonate solution (3 mL) were added. The organic phase was separated, washed twice with water (2×20 mL), dried over anhydrous magnesium sulfate, and filtered. The filtrate was concentrated under reduced pressure, and the residue was purified by preparative high performance liquid chromatography (ammonium bicarbonate) to give compound SZ-014114 as a pale yellow solid (41.1 mg, 30% yield). Liquid chromatography-mass spectrometry [mobile phase: elution was carried out with a gradient from 95% water (containing 0.02% ammonium acetate) and 5% acetonitrile to 5% water (containing 0.02% ammonium acetate) and 95% acetonitrile at a flow rate of 1.5 mL per minute at a column temperature of 40° C. for 6 min. Column: waters XBridge C18 3.5 μm, 50×4.6 mm]. The purity was 96.55%, Rt=4.262 min. LCMS (M+H)⁺ m/z calculated 606.3. found 606.3.

¹H NMR (DMSO-d₆, 400 MHz): δ 7.99-7.98 (m, 1H), 7.78-7.68 (m, 1H), 7.34 (t, J=11.6 Hz, 2H), 7.26 (t, J=10.0 Hz, 1H), 7.11 (d, J=10.8 Hz, 2H), 6.98-6.86 (m, 1H), 6.26 (d, J=21.2 Hz, 1H), 5.84-5.80 (m, 1H), 4.90 (br s, 1H), 4.51-4.06 (m, 3H), 3.73-3.50 (m, 2H), 3.39-3.10 (m, 1H), 2.69-2.60 (m, 2H), 1.36 (d, J=8.4 Hz, 3H), 1.06 (t, J=8.8 Hz, 6H), 1.04-0.91 (m, 6H).

### Example 25. SZ-014119

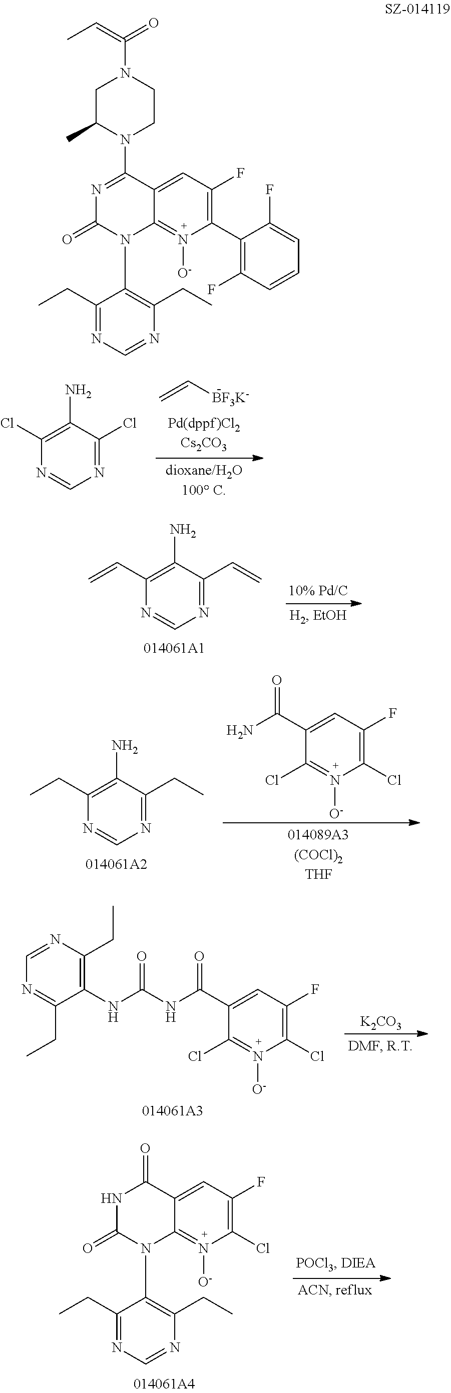

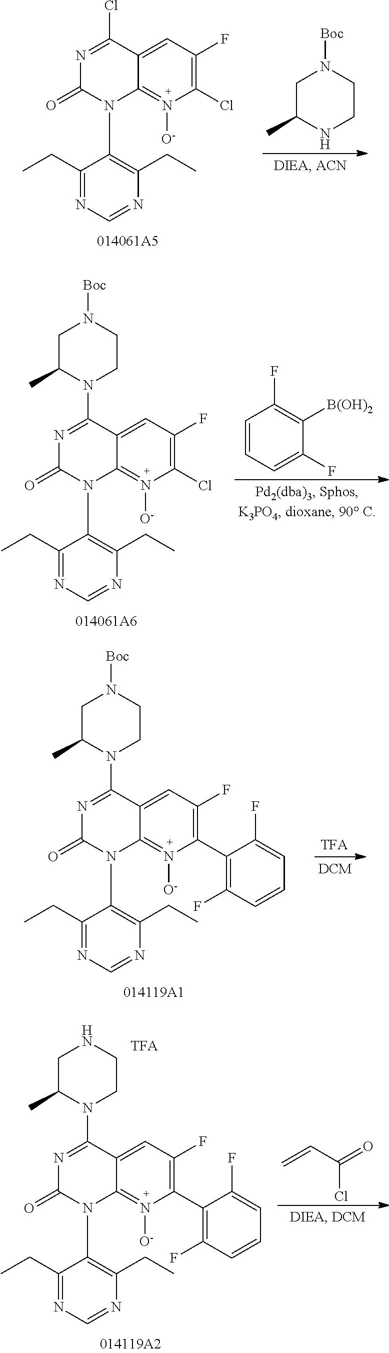

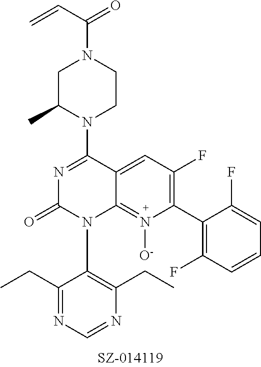

### Step 1: Synthesis of 014061A1

The compound 4,6-dichloro-5-aminopyrimidine (10.0 g, 60.9 mmol) was dissolved in 200 mL of 1,4-dioxane and 20 mL of water. Potassium trifluoro(vinyl)borate (32 g, 244 mmol) and cesium carbonate (49.7 g, 152 mmol) were added. The system was purged with nitrogen several times, and then [1,1′-bis(diphenylphosphino)ferrocene]palladium dichloride (2.2 g, 2.30 mmol) was added. The system was purged with nitrogen several times, and then the reaction mixture was heated to 100° C. and allowed to react overnight. The reaction mixture was cooled to room temperature and concentrated under reduced pressure. The residue was purified by silica gel column chromatography (dichloromethane:methanol=10:1) to give compound 014061A1 as a yellow solid (7.1 g, 79% yield). LCMS (M+H)⁺ m/z calculated 148.1. found 148.1.

### Step 2: Synthesis of 014061A2

Compound 014061A1 (7.1 g, 47.9 mmol) was dissolved in ethanol (50 mL). The system was purged with nitrogen several times, and then palladium on carbon (10% wt, 1.5 g) was added. The reaction mixture was allowed to react at room temperature under hydrogen (50 psi) overnight. The reaction mixture was filtered, and the filtrate was concentrated to give compound 014061A2 as a yellow solid (7.1 g, 79% yield). LCMS (M+H)⁺ m/z calculated 152.1. found 152.1. ¹HNMR (DMSO-d₆, 400 MHz): δ 8.26 (s, 1H), 5.02 (s, 2H), 2.62 (q, J=7.2 Hz, 4H), 1.18-1.14 (t, J=7.6 Hz, 6H).

### Step 3: Synthesis of 014061A3

Compound 014089A3 (3.3 g, 14.5 mmol) was dissolved in 40 mL of tetrahydrofuran. Oxalyl chloride (2.75 g, 21.7 mmol) was added at room temperature. The reaction mixture was heated at reflux for 1 h. The reaction mixture was cooled to room temperature, and compound 014061A2 (2.2 g, 14.5 mmol) was added. The mixture was stirred at room temperature for 1 h. The pH of the reaction mixture was adjusted to neutrality with saturated aqueous sodium bicarbonate solution, followed by extraction with ethyl acetate (50 mL×2). The organic phases were combined, dried over anhydrous sodium sulfate, and filtered. The filtrate was concentrated under reduced pressure. The residue was purified by silica gel column chromatography (petroleum ether:ethyl acetate=5:1) to give compound 014061A3 as a yellow solid (4.7 g, 81% yield). LCMS (M+H)⁺ m/z calculated 402.0. found 402.1.

### Step 4: Synthesis of 014061A4

Compound 014061A3 (3.8 g, 9.45 mmol) was dissolved in 15 mL of N,N-dimethylformamide. Anhydrous potassium carbonate (2.6 g, 18.9 mmol) was added. The reaction mixture was stirred at room temperature overnight. The reaction mixture was poured into water, and the pH of the resulting mixture was adjusted to neutrality with 2 N HCl, followed by extraction with ethyl acetate (30 mL×2). The organic phases were combined, dried over anhydrous sodium sulfate, and filtered. The filtrate was concentrated under reduced pressure. The residue was purified by silica gel column chromatography (petroleum ether:ethyl acetate=1:1) to give compound 014061A4 as a white solid (2.0 g, 59% yield). LCMS (M+H)⁺ m/z calculated 366.1. found 366.1.

### Step 5: Synthesis of 014061A5

Compound 014061A4 (1.26 g, 3.44 mmol) was dissolved in 5 mL of anhydrous acetonitrile. Phosphorus oxychloride (1.62 g, 10.3 mmol) and N,N′-diisopropylethylamine (1.64 g, 10.3 mmol) were added under an ice bath. The reaction mixture was refluxed for 1 h. The reaction mixture was cooled to room temperature and then concentrated to dryness by rotary evaporation to give 014061A5 as a red oil (865 mg, 100% yield). The crude product was directly used in the next step. LCMS (M+H)⁺ m/z calculated 384.0. found 384.1.

### Step 6: Synthesis of 014061A6

Compound 014061A5 (1.8 g, 4.93 mmol) was dissolved in 15 mL of acetonitrile. (S)-4-N-tert-butoxycarbonyl-2-methylpiperazine (1.5 g, 7.39 mmol) and DIEA (1.94 g, 15.0 mmol) were added. The reaction mixture was stirred at 0° C. for 1 h. The reaction mixture was concentrated to dryness by rotary evaporation, and the residue was purified by silica gel column chromatography (petroleum ether:ethyl acetate=1:1) to give compound 014061A6 as a red oil (1.6 g, 59% yield).

LCMS (M+H)⁺ m/z calculated 548.2. found 548.3. ¹HNMR (CDCl₃, 400 MHz): δ 9.06 (s, 1H), 7.35 (d, J=6.8 Hz, 1H), 4.05-4.04 (m, 1H), 4.22-3.95 (m, 3H), 3.64-3.62 (m, 1H), 3.24-3.13 (m, 2H), 2.57-2.46 (m, 4H), 1.53-1.45 (m, 12H), 1.35-1.22 (m, 6H).

### Step 7: Synthesis of 014119A1

Compound 014061A6 (630 mg, 1.15 mmol) and 2,6-difluorophenylboronic acid (1.1 g, 6.91 mmol) were dissolved in 25 mL of dioxane. Potassium phosphate (732 mg, 3.45 mmol), tris(dibenzylideneacetone)dipalladium (106 mg, 0.115 mmol) and 2-dicyclohexylphosphine-2′,6′-dimethoxybiphenyl (142 mg, 0.345 mmol) were added. The reaction mixture was allowed to react at 90° C. under nitrogen overnight. The reaction mixture was cooled to room temperature, and water (20 mL) was added, followed by extraction with ethyl acetate (3×20 mL). The organic phases were combined, washed with saturated brine, dried over anhydrous sodium sulfate, and filtered. The filtrate was concentrated under reduced pressure, and the resulting residue was purified by silica gel column chromatography (petroleum ether:ethyl acetate=78%-80%) to give compound 014119A1 as a brown solid (408 mg, 56% yield). LCMS (M+H)⁺ m/z calculated 626.3. found 626.3.

¹H NMR (DMSO-d₆, 400 MHz): δ 8.85 (s, 1H), 7.97 (d, J=8.4 Hz, 1H), 7.73-7.65 (m, 1H), 7.36-7.28 (m, 2H), 4.86-4.85 (m, 1H), 4.20 (d, J=13.2 Hz, 1H), 3.98-3.82 (m, 2H), 3.74-3.68 (m, 1H), 3.19-3.08 (m, 2H), 2.47-2.42 (m, 4H), 1.45 (s, 9H), 1.34 (d, J=6.8 Hz, 3H), 1.09-1.04 (m, 6H).

### Step 7: Synthesis of 014119A2

Compound 014119A1 (255 mg, 0.41 mmol) was dissolved in 20 mL of dichloromethane. 3 mL of trifluoroacetic acid was added. The reaction mixture was stirred at room temperature for 1.5 h. The reaction mixture was concentrated to dryness to give compound 014119A2 as a yellow oil (523 mg, crude product), which was directly used in the next step. LCMS (M+H)⁺ m/z calculated 526.2. found 526.2.

### Step 8: Synthesis of SZ-014119

Compound 014119A2 (crude product, 523 mg, 0.41 mmol) was dissolved in 20 mL of anhydrous dichloromethane. Acryloyl chloride (37 mg, 0.41 mmol) and N,N-diisopropylethylamine (265 mg, 2.05 mmol) were added. The reaction mixture was stirred at room temperature for 15 min. Saturated aqueous ammonium chloride solution (20 mL) was added, followed by extraction with dichloromethane (3×20 mL). The organic phases were combined, dried over anhydrous sodium sulfate, and filtered. The filtrate was concentrated under reduced pressure, and the residue was purified by preparative high performance liquid chromatography to give compound SZ-014119 as a yellow solid (146 mg, 61% yield). Liquid chromatography-mass spectrometry [mobile phase: elution was carried out with a gradient from 95% water (containing 0.02% ammonium acetate) and 5% acetonitrile to 5% water (containing 0.02% ammonium acetate) and 95% acetonitrile at a flow rate of 1.5 mL per minute at a column temperature of 40° C. for 6 min. Column: waters XBridge C18 3.5 μm, 50×4.6 mm. The purity was 97.40%, Rt=3.429 min. LCMS (M+H)⁺ m/z calculated 580.2. found 580.3.

¹H NMR (CD₃OD, 400 MHz): δ 8.85 (s, 1H), 7.96-7.92 (m, 1H), 7.65-7.58 (m, 1H), 7.18-7.11 (m, 2H), 6.88-6.78 (m, 1H), 6.31 (d, J=16.8 Hz, 1H), 5.85-5.82 (m, 1H), 5.06-5.03 (m, 1H), 4.57-4.41 (m, 2H), 4.22-4.07 (m, 1H), 3.88-3.83 (m, 1H), 3.72-3.54 (m, 1H), 3.34-3.18 (m, 1H), 2.62-2.55 (m, 4H), 1.46 (d, J=6.4 Hz, 3H), 1.18 (t, J=7.2 Hz, 6H).

### Example 26. SZ-014094

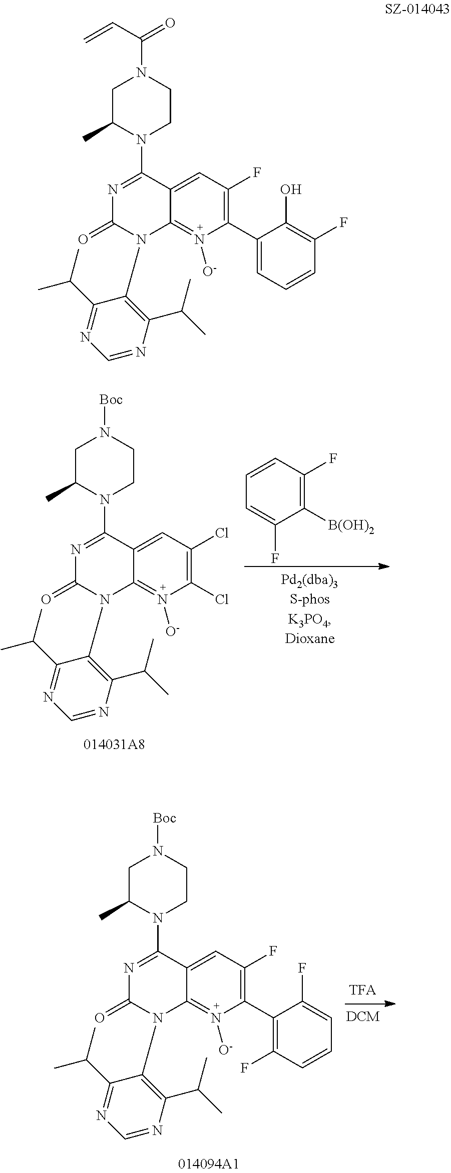

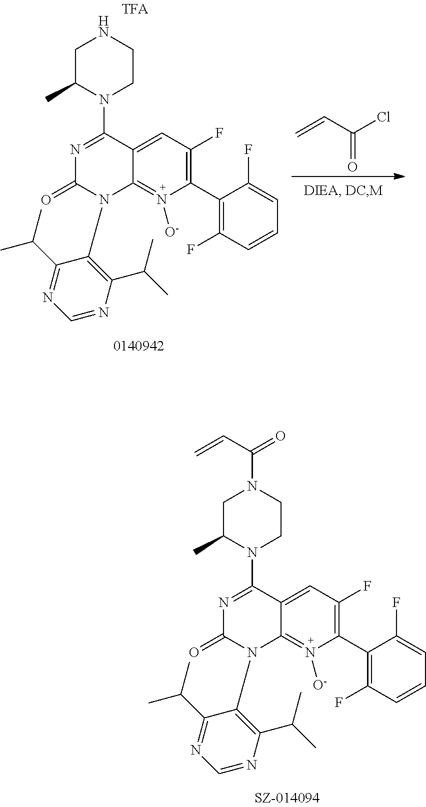

### Step 1: Synthesis of 014094A1

Compound 014031A8 (600 mg, 1.05 mmol), 2,6-difluorophenylboronic acid (800 mg, 5.07 mmol), potassium phosphate (660 mg, 3.03 mmol) and 2-dicyclohexylphosphine-2′,6′-dimethoxybiphenyl (130 mg, 0.303 mmol) were dissolved in 1,4-dioxane (15 mL). Tris(dibenzylideneacetone)dipalladium (100 mg, 0.105 mmol) was added. The system was purged with nitrogen several times, and then the reaction mixture was stirred at 95° C. overnight. The reaction mixture was cooled to room temperature and concentrated to remove the solvent. Water (15 mL) and ethyl acetate (15 mL) were added, followed by extraction with ethyl acetate (10 mL×3). The organic phases were combined, dried over anhydrous sodium sulfate, and filtered. The filtrate was concentrated under reduced pressure, and the resulting residue was purified by silica gel column chromatography (ethyl acetate/petroleum ether=50%) to give compound 014094A1 as a yellow solid (178 mg, 26% yield). LCMS (M+H)⁺ m/z calculated 670.3. found 670.3.

### Step 2: Synthesis of 014094A2

Compound 014094A1 (1.1 g, 1.66 mmol) was dissolved in dichloromethane (25 mL). Trifluoroacetic acid (5.0 mL) was added under an ice bath. The ice bath was removed, and the reaction mixture was stirred at room temperature (20° C.) for 1.5 h. The reaction mixture was concentrated under reduced pressure to give compound 014094A2 as a yellow oil (crude product, 1.8 g, 100% yield), which was directly used in the next step without purification.

LCMS (M+H)⁺ m/z calculated 570.2. found 570.2.

### Step 3: Synthesis of SZ-014094

The crude compound 014094A2 (crude product, 1.8 g, 1.66 mmol) was dissolved in dichloromethane (40 mL). Acryloyl chloride (180 mg, 1.99 mmol) was slowly added under an ice bath, followed by diisopropylethylamine (1.1 g, 8.30 mmol). After dropwise addition, the reaction phase was stirred at 0° C. for 30 min. Saturated ammonium chloride solution (8 mL) was added to the reaction mixture, followed by extraction with dichloromethane (5 mL×3). The organic phases were combined, washed twice with water (10 mL×2), dried over anhydrous sodium sulfate, and filtered. The filtrate was concentrated under reduced pressure, and the residue was purified by preparative high performance liquid chromatography (ammonium bicarbonate) to give compound SZ-014094 as a pale yellow solid (400 mg, 33% yield).

Liquid chromatography-mass spectrometry [mobile phase: elution was carried out with a gradient from 70% water (containing 0.02% ammonium acetate) and 30% acetonitrile to 30% water (containing 0.02% ammonium acetate) and 70% acetonitrile at a flow rate of 1.5 mL per minute at a column temperature of 40° C. for 6 min. Column: waters XBridge C18 5 μm, 50×4.6 mm. The purity was 97.92%, Rt=3.336 min.

LCMS (M+H)⁺ m/z calculated 624.2. found 624.2. ¹H NMR (CD₃OD, 400 MHz): δ 8.77 (s, 1H), 7.99 (d, J=6.8 Hz, 1H), 7.53-7.46 (m, 1H), 7.01 (t, J=8.0 Hz, 2H), 6.79-6.69 (m, 1H), 6.22 (d, J=16.4 Hz, 1H), 5.74 (dd, J₁=10.8 Hz, J₂=1.6 Hz, 1H), 5.02-4.90 (m, 1H), 4.47-4.32 (m, 2H), 4.14-3.98 (m, 1H), 3.80-3.48 (m, 2H), 3.26-3.22 (m, 1H), 2.85-2.79 (m, 2H), 1.38 (d, J=6.8 Hz, 3H), 1.08 (d, J=6.8 Hz, 6H), 1.03 (d, J=6.8 Hz, 6H).

### Example 27. SZ-014116AB

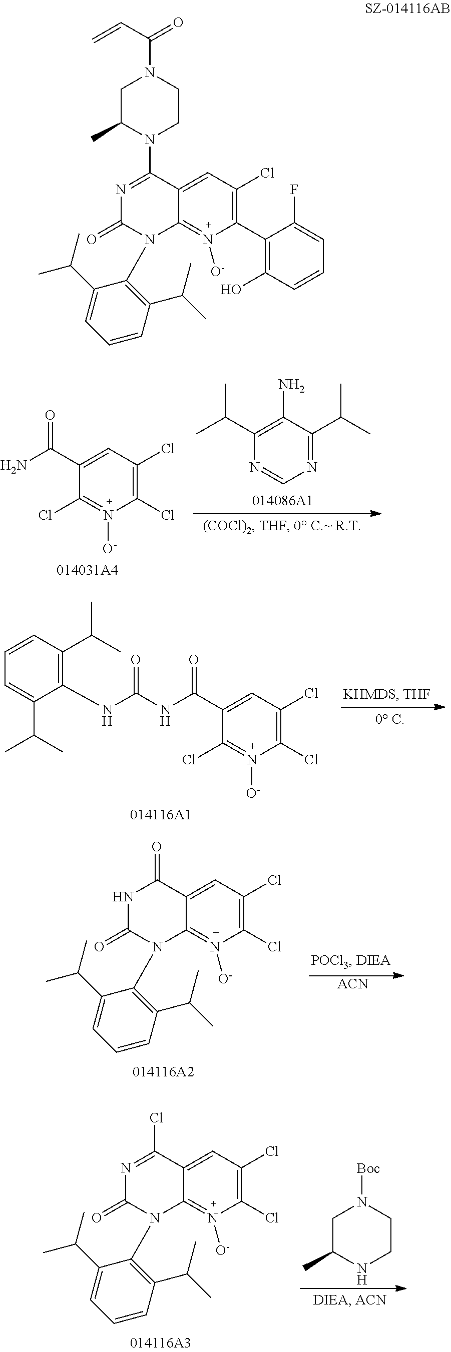

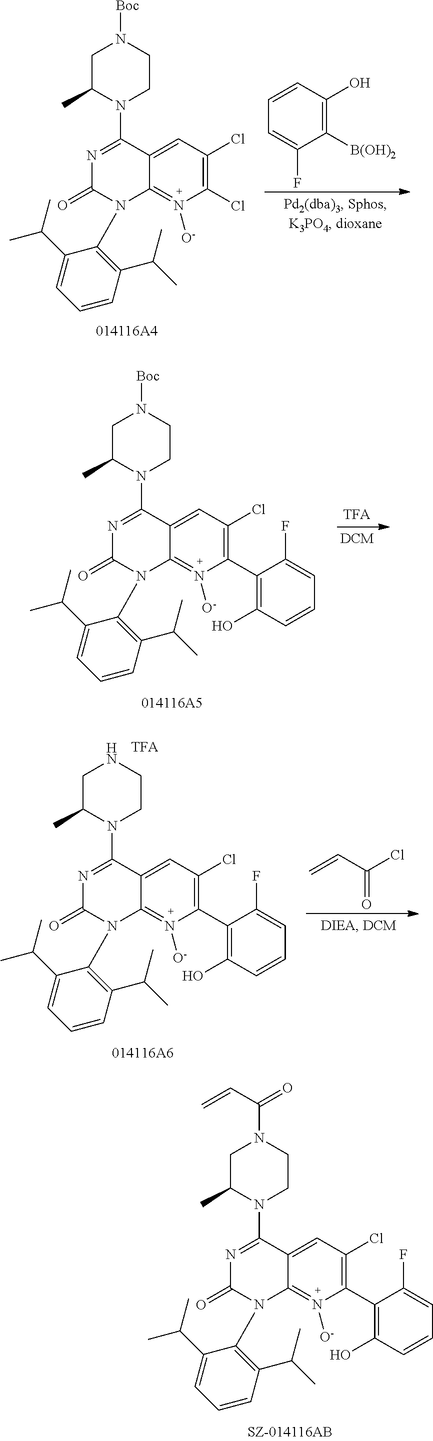

### Step 1: Synthesis of 014116A1

Compound 014031A4 (1.3 g, 5.39 mmol) was dissolved in tetrahydrofuran (40 mL). Oxalyl chloride (3.7 g, 29.65 mmol) was added at room temperature. The reaction mixture was heated at reflux for 1 h until it became clear and transparent. The reaction mixture was cooled to room temperature and concentrated. The residue was dissolved in tetrahydrofuran (30 mL). Compound 014086A1 (1.05 g, 5.93 mmol) was added at 0° C. The reaction mixture was stirred at room temperature for 1 h, and the reaction was completed. The reaction mixture was filtered, and the filter cake was rinsed with n-hexane, collected and dried to give compound 014116A1 as a yellow solid (1.8 g, 75% yield). LCMS (M+H)⁺ m/z calculated 444.0. found 444.0.

### Step 2: Synthesis of 014116A2

Compound 014116A1 (1.6 g, 3.6 mmol) was dissolved in tetrahydrofuran (30 mL). KHMDS (1.0 M in THF, 7.5 mL, 7.5 mmol) was added at 0° C. under nitrogen. The reaction was stirred at 0° C. for another 2 h. Saturated ammonium chloride solution (50 mL) was added to the reaction mixture, followed by extraction with ethyl acetate (40 mL×3). The organic phases were combined, washed with saturated brine, dried over anhydrous sodium sulfate, and filtered. The filtrate was concentrated under reduced pressure, and the residue was purified by silica gel column chromatography (ethyl acetate/petroleum ether=20%-35%) to give compound 014116A2 as a yellow solid (631 mg, 43% yield). LCMS (M+H)⁺ m/z calculated 408.0. found 408.0.

### Step 3: Synthesis of 014116A3

Compound 014116A2 (2.8 g, 6.86 mmol) was dissolved in anhydrous acetonitrile (150 mL). Phosphorus oxychloride (3.2 g, 20.58 mmol) and N,N′-diisopropylethylamine (2.7 g, 20.58 mmol) were added under an ice bath. The reaction mixture was heated to 70° C. and allowed to react for 2 h. The reaction mixture was cooled to room temperature and concentrated to give compound 014116A3 as a brown oil (4.5 g). The crude product was directly used in the next step. LCMS (M+H)⁺ m/z calculated 426.0. found 426.0.

### Step 4: Synthesis of 014116A4

The crude compound 014116A3 (4.5 g, 6.86 mmol) was dissolved in acetonitrile (60 mL). (S)-4-N-tert-butoxycarbonyl-2-methylpiperazine (2.1 g, 10.29 mmol) and diisopropylethylamine (2.7 g, 20.58 mmol) were added. The reaction mixture was stirred at 0° C. for 1 h. The reaction mixture was concentrated, and the residue was purified by silica gel column chromatography (ethyl acetate/petroleum ether=35%-60%) to give 014116A4 (3.4 g, 83% yield). LCMS (M+H)⁺ m/z calculated 590.2. found 590.2. ¹H NMR (DMSO-d₆, 400 MHz): δ 7.93 (s, 1H), 7.27 (t, J=7.6 Hz, 1H), 7.12-7.09 (m, 2H), 4.75 (br s, 1H), 4.09-3.97 (m, 2H), 3.84-3.80 (m, 1H), 3.66-3.60 (m, 1H), 3.28-3.04 (m, 2H), 2.58-2.53 (m, 2H), 1.44 (s, 9H), 1.29 (s, J=6.8 Hz, 3H), 1.04-0.85 (m, 12H).

### Step 5: Synthesis of 014116A5

Compound 014116A4 (300 mg, 0.51 mmol), 2-fluoro-6-hydroxyphenylboronic acid (476 mg, 3.06 mmol), potassium phosphate (325 mg, 1.53 mmol) and 2-dicyclohexylphosphine-2′,6′-dimethoxybiphenyl (63 mg, 0.153 mmol) were dissolved in 1,4-dioxane (15 mL). Tris(dibenzylideneacetone)dipalladium (47 mg, 0.051 mmol) was added. The system was purged with nitrogen several times, and then the reaction mixture was stirred at 95° C. overnight. The reaction mixture was cooled to room temperature and concentrated to remove the solvent. Water (15 mL) and ethyl acetate (15 mL) were added, followed by extraction with ethyl acetate (10 mL×3). The organic phases were combined, dried over anhydrous sodium sulfate, and filtered. The filtrate was concentrated under reduced pressure, and the resulting residue was purified by silica gel column chromatography (ethyl acetate/petroleum ether=40%) to give compound 014116A5 as a yellow solid (103 mg, 30% yield). LCMS (M+H)⁺ m/z calculated 666.2. found 666.2.

### Step 6: Synthesis of 014116A6

Compound 014116A5 (145 mg, 0.22 mmol) was dissolved in dichloromethane (10 mL). Trifluoroacetic acid (2.0 mL) was added under an ice bath. The ice bath was removed, and the reaction mixture was stirred at room temperature (20° C.) for 1.5 h. The reaction mixture was concentrated under reduced pressure to give compound 014116A6 as a yellow oil (crude product, 317 mg, 100% yield), which was directly used in the next step without purification.

LCMS (M+H)⁺ m/z calculated 566.2. found 566.2.

### Step 7: Synthesis of SZ-014116AB

The crude compound 014116A6 (317 mg, 0.22 mmol) was dissolved in dichloromethane (15 mL). Acryloyl chloride (20 mg, 0.22 mmol) was slowly added under an ice bath, followed by diisopropylethylamine (142 mg, 1.10 mmol). After dropwise addition, the reaction phase was stirred at 0° C. for 30 min. Saturated ammonium chloride solution (8 mL) was added to the reaction mixture, followed by extraction with dichloromethane (5 mL×3). The organic phases were combined, washed twice with water (10 mL×2), dried over anhydrous sodium sulfate, and filtered. The filtrate was concentrated under reduced pressure, and the residue was purified by preparative high performance liquid chromatography (ammonium bicarbonate) to give compound SZ-014116AB as a pale yellow solid (90 mg, 66% yield).

Liquid chromatography-mass spectrometry [mobile phase: elution was carried out with a gradient from 60% water (containing 0.02% ammonium acetate) and 40% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm. The purity was 93.41%, Rt=3.609 min.

LCMS (M+H)⁺ m/z calculated 620.3. found 620.4. ¹H NMR (CD₃OD, 400 MHz): δ 7.97 (s, 1H), 7.31-7.21 (m, 2H), 7.09 (d, J=8.0 Hz, 2H), 6.89-6.78 (m, 1H), 6.68 (d, J=8.4 Hz, 1H), 6.63 (t, J=8.4 Hz, 1H), 6.31 (m, 1H), 5.83 (dd, J₁=10.8 Hz, J₂=2.0 Hz, 1H), 5.04-4.96 (m, 1H), 4.59-4.33 (m, 2H), 4.22-4.06 (m, 1H), 3.83-3.54 (m, 2H), 3.19-3.13 (m, 1H), 2.70-2.64 (m, 2H), 1.47-1.44 (m, 3H), 1.16-1.03 (m, 12H).

### Example 28. SZ-014129

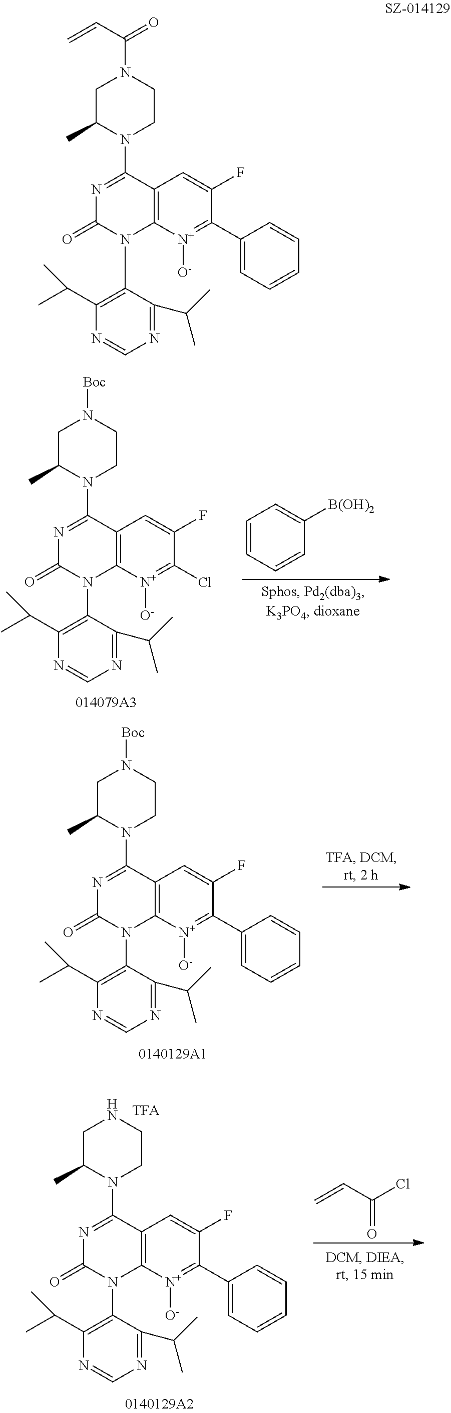

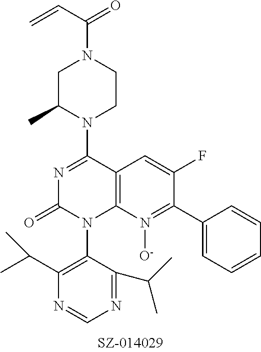

### Step 1: Synthesis of 014129A1

Compound 014079A3 (600 mg, 1.1 mmol), phenylboronic acid (330 mg, 2.7 mmol), potassium phosphate (465 mg, 2.2 mmol) and 2-dicyclohexylphosphine-2′,6′-dimethoxybiphenyl (180 mg, 0.44 mmol) were dissolved in 1,4-dioxane (20 mL). Tris(dibenzylideneacetone)dipalladium (201 mg, 0.22 mmol) was added. The system was purged with nitrogen several times. The reaction mixture was stirred at 105° C. for 5 h, cooled to room temperature, and filtered. The filtrate was diluted with ethyl acetate (30 mL) and washed with saturated brine. The organic phase was dried over anhydrous sodium sulfate and filtered. The filtrate was concentrated under reduced pressure, and the resulting residue was purified by preparative high performance liquid chromatography (ammonium bicarbonate) to give compound 014129A1 as a yellow solid (200 mg, 30.1% yield). LCMS (M+H)⁺ m/z calculated 618.3. found 618.3. ¹H NMR (CD₃OD, 400 MHz): δ 8.77 (s, 1H), 7.77 (d, J=4.2 Hz, 1H), 7.39 (m, 5H), 4.88-4.87 (m, 1H), 4.25-4.22 (m, 1H), 4.07-4.03 (m, 1H), 3.92-3.89 (m, 1H), 3.72-3.67 (m, 1H), 3.22-3.20 (m, 2H), 2.87-2.77 (m, 2H), 1.41 (s, 9H), 1.39-1.37 (m, 3H), 1.10-1.08 (m, 6H), 1.07-1.05 (m, 6H).

### Step 2: Synthesis of 014129A2

Compound 0140129A1 (200 mg, 0.32 mmol) was dissolved in dichloromethane (3.5 mL). Trifluoroacetic acid (1.5 mL) was added under an ice bath. The ice bath was removed, and the reaction mixture was stirred at room temperature for 2 h, and concentrated to give 014129A2 as a yellow oil (430 mg, crude product), which was directly used in the next step without purification. LCMS (M+H)⁺ m/z calculated 518.3. found 518.3.

### Step 3: Synthesis of SZ-014129

The crude compound 014129A2 (430 mg, 0.83 mmol) was dissolved in dichloromethane (5.0 mL). Acryloyl chloride (36 mg, 0.4 mmol) and N,N-diisopropylethylamine (108 mg, 0.83 mmol) were added. The reaction mixture was stirred at room temperature for 15 min. The reaction mixture was diluted with 20 mL of dichloromethane, quenched with saturated ammonium chloride solution (10 mL), washed with water (10 mL×2), and concentrated under reduced pressure. The residue was purified by preparative high performance liquid chromatography (ammonium bicarbonate) to give compound SZ-014129 as a yellow solid (80.0 mg, 43.2% yield over two steps).

Liquid chromatography-mass spectrometry [mobile phase: elution was carried out with a gradient from 30% water (containing 0.02% ammonium acetate) and 70% acetonitrile to 45% water (containing 0.02% ammonium acetate) and 55% acetonitrile at a flow rate of 1.5 mL per minute at a column temperature of 40° C. for 6 min. Column: XBridge C18 5 μm, 4.6×50 mm. The purity was 98.81%, Rt=3.533 min.

LCMS (M+H)⁺ m/z calculated 572.3. found 572.3. ¹H NMR (CD₃OD, 400 MHz): δ 8.77 (s, 1H), 7.82-7.77 (m, 1H), 7.39 (m, 5H), 6.76-6.72 (m, 1H), 6.22 (m, 1H), 5.74 (dd, J₁=10.4 Hz, J₂=2.4 Hz, 1H), 4.94 (br s, 1H), 4.49-4.30 (m, 2H), 4.14-4.00 (m, 1H), 3.79-3.60 (m, 2H), 3.47-3.08 (m, 1H), 2.83-2.81 (m, 2H), 1.36 (d, J₁=2.8 Hz, 3H), 1.09 (dd, J₁=6.4 Hz, J₂=1.6 Hz, 6H), 1.06 (d, J₁=6.8 Hz, 6H).

### Example 29. SZ-014130

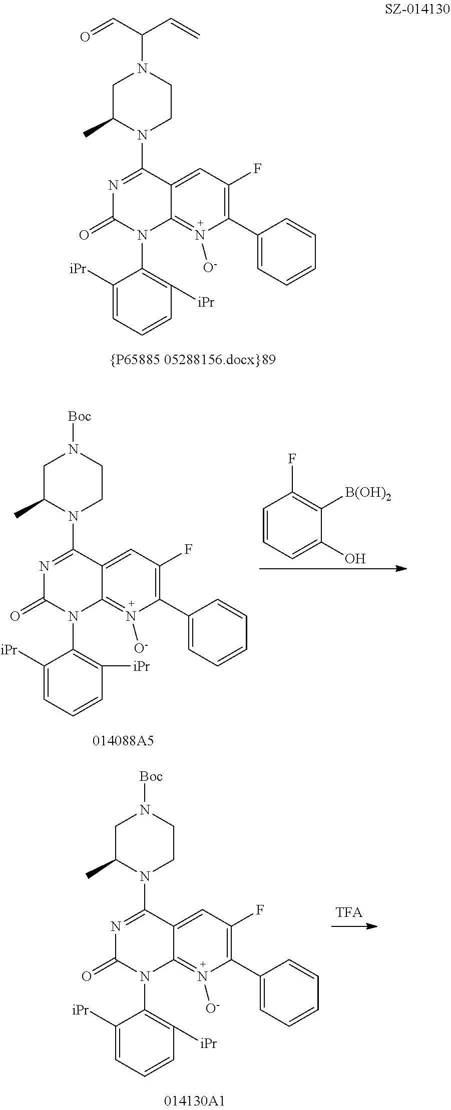

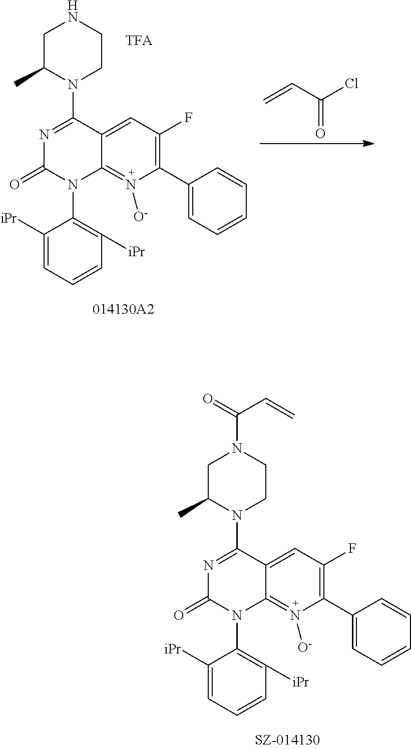

### Step 1: Synthesis of 014130A1

Compound 014088A5 (150 mg, 0.26 mmol), phenylboronic acid (48 mg, 0.39 mmol), potassium phosphate (110 mg, 0.52 mmol) and Sphos (21 mg, 0.0.52 mmol) were dissolved in 1,4-dioxane (10 mL). Pd₂(dba)₃ (47 mg, 0.052 mmol) was added. The system was purged with nitrogen several times, and then the reaction mixture was stirred at 90° C. overnight, cooled to room temperature, and filtered. The filtrate was diluted with ethyl acetate (100 mL) and washed with saturated brine. The organic phase was dried over anhydrous sodium sulfate and filtered. The filtrate was concentrated under reduced pressure, and the resulting residue was purified by silica gel column chromatography (petroleum ether/ethyl acetate=4:1 to methanol/dichloromethane=1:30) to give compound 014130A1 as a yellow solid (130 mg, 71% yield).

LCMS (M+H)⁺ m/z calculated 616.3. found 616.2.

¹H NMR (DMSO-d₆, 400 MHz): δ 7.87-7.82 (m, 1H), 7.58-7.35 (m, 5H), 7.33-7.20 (m, 2H), 7.10-7.08 (m, 1H), 4.90-4.80 (m, 1H), 4.29-3.96 (m, 2H), 3.89-3.84 (m, 1H), 3.68-3.59 (m, 2H), 3.37-3.07 (m, 1H), 2.78-2.45 (m, 2H), 1.50 (s, 9H), 1.48-1.33 (m, 3H), 1.26-0.87 (m, 12H).

### Step 2: Synthesis of 014130A2

Compound 014130A1 (130 mg, 0.21 mmol) was dissolved in dichloromethane (10 mL). Trifluoroacetic acid (720 mg, 6.00 mmol) was added under an ice bath. The ice bath was removed, and the reaction mixture was stirred at room temperature (20° C.) for 2 h, and concentrated under reduced pressure and a water bath at 30° C. to give compound 014130A2 as a yellow oil (120 mg, TFA salt crude product), which was directly used in the next step without purification. LCMS (M+H)⁺ m/z calculated 516.3. found 516.2.

### Step 3: Synthesis of SZ-014130

The crude compound 014130A2 (120 mg, 0.232 mmol) was dissolved in dichloromethane (4 mL). Acryloyl chloride (21 mg, 0.232 mmol) was slowly added under an ice bath, followed by diisopropylethylamine (90 mg, 0.696 mmol). After dropwise addition, the reaction phase was stirred at 0° C. for 10 min. The reaction mixture was diluted with dichloromethane (30 mL). Saturated ammonium chloride solution (20 mL) and saturated sodium bicarbonate solution (3 mL) were added. The organic phase was separated, washed twice with water (20 mL×2), dried over anhydrous magnesium sulfate, and filtered. The filtrate was concentrated under reduced pressure, and the residue was purified by preparative high performance liquid chromatography (ammonium bicarbonate) to give compound SZ-014130 as a pale yellow solid (36 mg, 28% yield).

Liquid chromatography-mass spectrometry [mobile phase: elution was carried out with a gradient from 50% water (containing 0.02% ammonium acetate) and 50% acetonitrile to 50% water and 50% acetonitrile at a flow rate of 1.5 mL per minute at a column temperature of 40° C. for 6 min. Column: waters XBridge C18 3.5 μm, 50×4.6 mm]. The purity was 96.97%, Rt=3.512 min.

LCMS (M+H)⁺ m/z calculated 570.3. found 570.4.

¹H NMR (CD₃OD, 400 MHz): δ 7.72 (t, J=7.2 Hz, 1H), 7.37 (s, 5H), 7.15 (t, J=7.6 Hz, 1H), 7.01 (d, J=7.2 Hz, 2H), 6.77-6.68 (m, 1H), 6.21 (m, 1H), 5.73 (dd, J₁=10.8 Hz, J₂=2.0 Hz, 1H), 4.92-4.83 (m, 1H), 4.50-4.35 (m, 1H), 4.26-3.96 (m, 2H), 3.70-3.46 (m, 2H), 3.22-3.08 (m, 1H), 2.58-2.53 (m, 2H), 1.34 (d, J=6.8 Hz, 3H), 1.07-0.96 (m, 12H).

### Example 30. SZ-014131

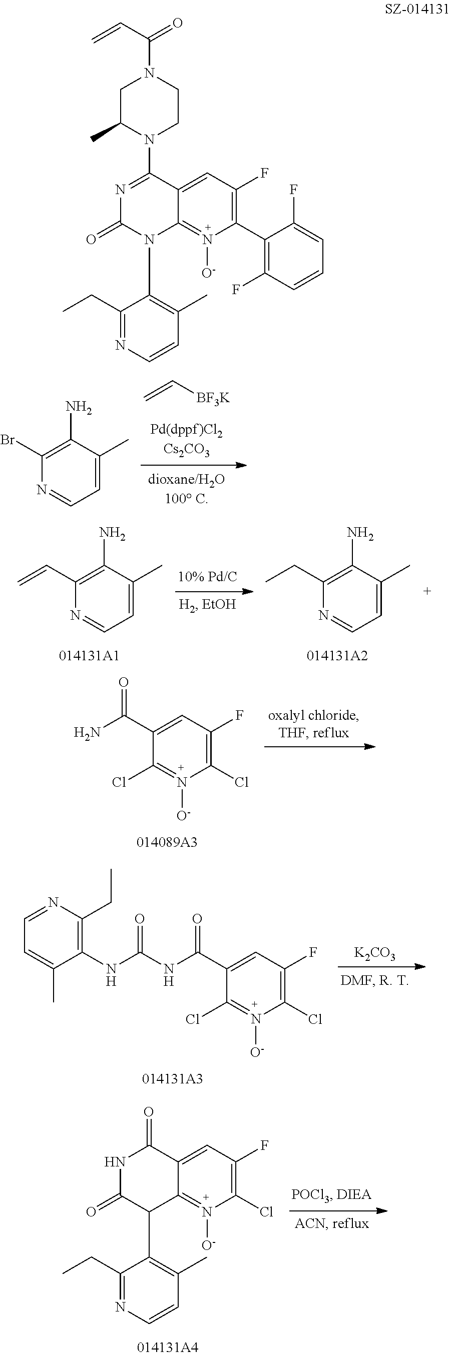

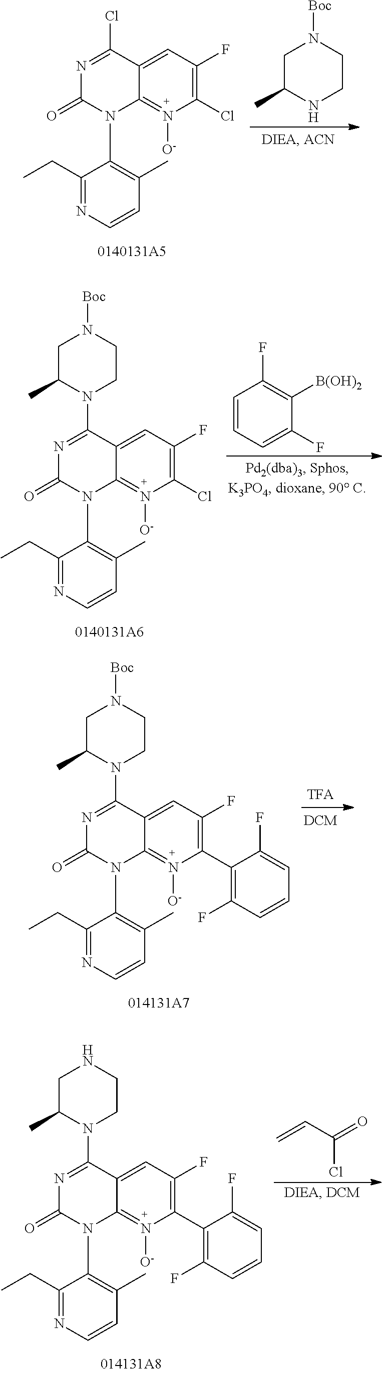

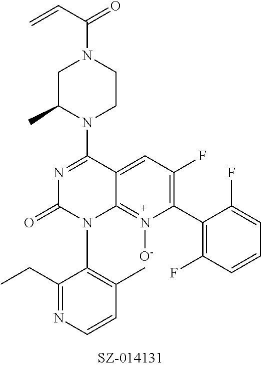

### Step 1: Synthesis of 014131A1

The compound 2-bromo-3-amino-4-methylpyridine (5.0 g, 26.7 mmol) was dissolved in 100 mL of 1,4-dioxane and 20 mL of water. Potassium trifluoro(vinyl)borate (5.37 g, 40.1 mmol), cesium carbonate (17.3 g, 53.4 mmol) and [1,1′-bis(diphenylphosphino)ferrocene]palladium dichloride (975 mg, 1.33 mmol) were added. The reaction mixture was heated to 100° C. under nitrogen and allowed to react overnight. The reaction mixture was cooled to room temperature and concentrated under reduced pressure. The residue was purified by silica gel column chromatography (dichloromethane:methanol=20:1) to give 014131A1 as a yellow solid (3.0 g, 84% yield). LCMS (M+H)⁺ m/z calculated 135.1. found 135.1.

### Step 2: Synthesis of 014131A2

Compound 014131A1 (2.8 g, 20.7 mmol) was dissolved in ethanol (20 mL). Palladium on carbon (5%, 700 mg) was added. The reaction mixture was allowed to react at room temperature under hydrogen (50 psi) overnight. The reaction mixture was filtered, and the filtrate was concentrated to give 014131A2 as a yellow solid (2.6 g, 93% yield). LCMS (M+H)⁺ m/z calculated 137.1. found 137.1.

### Step 3: Synthesis of 014131A3

Compound 014089A3 (5.5 g, 24.5 mmol) was dissolved in 40 mL of tetrahydrofuran. Oxalyl chloride (5.18 g, 40.8 mmol) was added at room temperature. The reaction mixture was heated at reflux for 1 h. The reaction mixture was cooled to 0° C., and compound 014131A2 (2.8 g, 20.4 mmol) was added. The mixture was stirred at room temperature for 1 h. The pH of the reaction mixture was adjusted to neutrality with saturated sodium bicarbonate solution, followed by extraction with ethyl acetate. The extract was dried over anhydrous sodium sulfate and filtered. The filtrate was concentrated under reduced pressure. The residue was purified by silica gel column chromatography (petroleum ether:ethyl acetate=1:1) to give 014131A3 as a yellow solid (4.5 g, 57.6% yield).

LCMS (M+H)⁺ m/z calculated 387.0. found 387.1.

### Step 4: Synthesis of 014131A4

Compound 014131A3 (3.2 g, 8.27 mmol) was dissolved in 30 mL tetrahydrofuran. Potassium hexamethyldisilazide (16.5 mL, 16.5 mmol) was added. The reaction mixture was stirred at 0° C. for 3 h. The reaction mixture was poured into aqueous ammonium chloride solution, and the pH of the resulting mixture was adjusted to neutrality with 2 N HCl, followed by extraction with ethyl acetate. The extract was dried over anhydrous sodium sulfate and filtered. The filtrate was concentrated under reduced pressure. The residue was purified by silica gel column chromatography (petroleum ether:ethyl acetate=1:4) to give 014131A4 as a yellow solid (1.0 g, 34% yield). LCMS (M+H)⁺ m/z calculated 351.1. found 351.1.

### Step 5: Synthesis of 014131A5

Compound 014131A4 (1.0 g, 2.85 mmol) was dissolved in 5 mL of anhydrous acetonitrile. Phosphorus oxychloride (1.34 g, 8.55 mmol) and N,N′-diisopropylethylamine (1.1 g, 8.55 mmol) were added under an ice bath. The reaction mixture was heated to 65° C. and allowed to react for 1 h. The reaction mixture was cooled and then concentrated to dryness by rotary evaporation to give 014131A5 as a red oil (1.1 g, 100% yield). The crude product was directly used in the next step. LCMS (M+H)⁺ m/z calculated 369.0. found 369.1.

### Step 6: Synthesis of 014131A6

Compound 014131A5 (1.2 g, 2.85 mmol) was dissolved in 15 mL of acetonitrile. (S)-4-N-tert-butoxycarbonyl-2-methylpiperazine (1.14 g, 5.7 mmol) and DIPEA (3 mL) were added. The reaction mixture was stirred at 0° C. for 1 h. The reaction mixture was concentrated to dryness by rotary evaporation, and the residue was purified by silica gel column chromatography (petroleum ether:ethyl acetate=1:1) to give 014131A6 as a yellow solid (925 mg, 61% yield). LCMS (M+H)⁺ m/z calculated 533.2. found 533.3.

### Step 7: Synthesis of 014131A7

Compound 014131A6 (600 mg, 1.15 mmol) and 2,6-difluorophenylboronic acid (1.1 g, 6.91 mmol) were dissolved in 25 mL of dioxane. Potassium phosphate (732 mg, 3.45 mmol), tris(dibenzylideneacetone)dipalladium (106 mg, 0.115 mmol) and 2-dicyclohexylphosphine-2′,6′-dimethoxybiphenyl (142 mg, 0.345 mmol) were added. The reaction mixture was allowed to react at 90° C. under nitrogen overnight. The reaction mixture was cooled, and water (20 mL) was added, followed by extraction with ethyl acetate (20 mL×3). The organic phases were combined, washed with saturated brine, and concentrated. The residue was purified by silica gel column chromatography (petroleum ether:ethyl acetate=78%-80%) to give 014131A7 as a brown solid (350 mg, 51% yield). LCMS (M+H)⁺ m/z calculated 611.3. found 611.3. ¹H NMR (DMSO-d₆, 400 MHz): δ 8.22 (d, J=4.8 Hz, 1H), 7.92 (dd, J=17.2, 8.4 Hz, 1H), 7.72-7.64 (m, 1H), 7.32-7.27 (m, 2H), 7.06 (d, J=4.4 Hz, 1H), 4.86-4.81 (m, 1H), 4.21-4.14 (m, 1H), 4.01-3.96 (m, 1H), 3.85-3.80 (m, 1H), 3.70-3.65 (m, 1H), 3.19-3.04 (m, 1H), 2.43-2.32 (m, 3H), 2.01 (d, J=9.2 Hz, 3H), 1.45 (s, 9H), 1.40-1.23 (m, 3H), 1.05-1.02 (m, 3H).

### Step 8: Synthesis of 014131A8

Compound 014131A7 (150 mg, 0.245 mmol) was dissolved in 10 mL of dichloromethane. 3 mL of trifluoroacetic acid was added. The reaction mixture was stirred at room temperature for 1.5 h. The reaction mixture was concentrated to dryness to give 105 mg of trifluoroacetate salt 014131A8 as a yellow oil, which was directly used in the next step. LCMS (M+H)⁺ m/z calculated 511.2. found 511.2.

### Step 9: Synthesis of SZ-014131

The crude compound 014131A8 (105 mg, 0.215 mmol) was dissolved in 10 mL of anhydrous dichloromethane. Acryloyl chloride (33 mg, 0.368 mmol) was added dropwise, followed by addition of N,N-diisopropylethylamine (158 mg, 1.23 mmol). The reaction mixture was stirred at room temperature for 15 min. The reaction mixture was quenched by addition of ammonium chloride and extracted with dichloromethane. The organic phase was concentrated to dryness by rotary evaporation, and the residue was purified by preparative high performance liquid chromatography to give SZ-014131 as a yellow solid (75 mg, 54% yield).

Liquid chromatography-mass spectrometry [mobile phase: elution was carried out with a gradient from 80% water (containing 0.02% ammonium acetate) and 20% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm. The purity was 92.06%, Rt=3.347 min. LCMS (M+H)⁺ m/z calculated 565.2. found 565.3.

¹H NMR (CD₃OD, 400 MHz): δ 8.26 (d, J=5.2 Hz, 1H), 7.97-7.89 (m, 1H), 7.67-7.59 (m, 1H), 7.17-7.12 (m, 3H), 6.87-6.80 (m, 1H), 6.33 (m, 1H), 5.85 (dd, J=10.4, 1.6 Hz, 1H), 5.08-5.01 (m, 1H), 4.58-4.38 (m, 2H), 4.25-4.09 (m, 1H), 3.89-3.59 (m, 2H), 3.26-3.15 (m, 1H), 2.59-2.52 (m, 2H), 2.18 (d, J=3.6 Hz, 3H), 1.51-1.45 (m, 3H), 1.16-1.12 (m, 3H).

### Example 31. SZ-014136

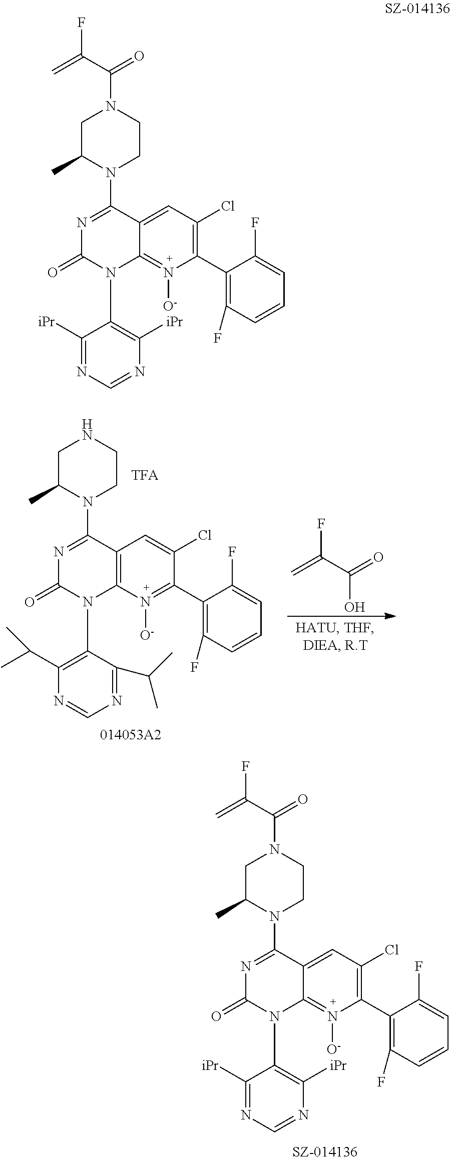

Synthesis of SZ-014136

Compound 014053A2 (2.4 g, 4.25 mmol, trifluoroacetate salt crude product) was dissolved in 100 mL of anhydrous tetrahydrofuran. 2-Fluoroacrylic acid (855 mg, 9.50 mmol) and 2-(7-azobenzotriazole)-N,N,N′,N′-tetramethyluronium hexafluorophosphate (3.6 g, 9.5 mmol) were added. N,N-diisopropylethylamine (1.6 g, 12.75 mmol) was slowly added to the reaction mixture with stirring at room temperature until pH=9-10. The reaction mixture was stirred overnight and poured into 300 mL of cold water, followed by extraction with dichloromethane. The extracts were combined and concentrated to dryness by rotary evaporation, and the residue was purified by preparative high performance liquid chromatography to give SZ-014136 as a pale yellow solid (600 mg, 22.5% yield). LCMS (M+H)⁺ m/z calculated 626.2. found 626.2.

¹H NMR (DMSO-d₆, 400 MHz): δ 8.93 (s, 1H), 8.04 (d, J=10.8 Hz, 1H), 7.77-7.67 (m, 1H), 7.33 (t, J=11.6 Hz, 2H), 5.43-5.25 (m, 2H), 4.97 (br s, 1H), 4.34-4.15 (m, 2H), 4.07-3.79 (m, 2H), 3.73-3.46 (m, 1H), 3.27-3.22 (m, 1H), 2.93-2.81 (m, 2H), 1.38 (d, J=8.8 Hz, 3H), 1.11-1.03 (m, 12H).

### Comparative Example 1. SZ-014137

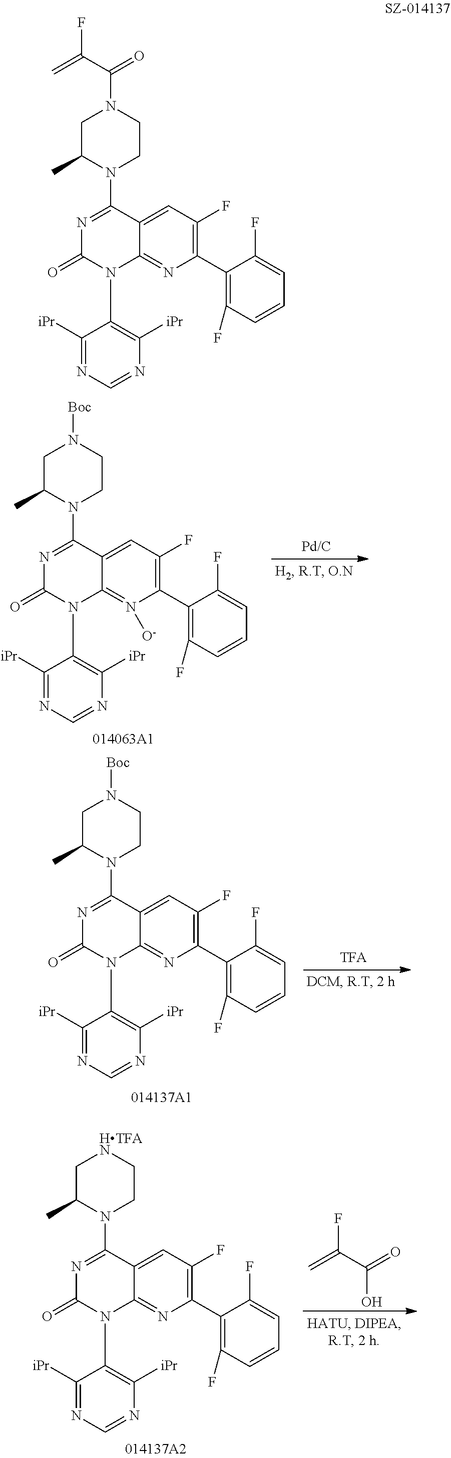

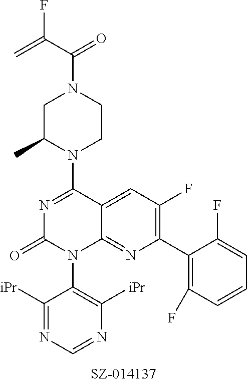 [SMILES: C=C(F)C(=O)N1CCN(c2nc(=O)n(-c3c(C(C)C)ncnc3C(C)C)c3nc(-c4c(F)cccc4F)c(F)cc23)[C@@H](C)C1]

### Step 1: Synthesis of 014137A1

Compound 014053A1 (300 mg, 0.5 mmol) was dissolved in a solvent mixture of tetrahydrofuran and methanol (20 mL, in a ratio of 1:1). Palladium on carbon (60 mg) was added. The reaction mixture was stirred under hydrogen at normal atmospheric pressure at room temperature overnight. The reaction mixture was filtered and concentrated, and the residue was purified by silica gel column chromatography (petroleum ether:ethyl acetate=1:1) to give compound 014137A1 as a pale yellow oil (crude product, 250 mg, 78.6% yield), which was directly used in the next step.

LCMS (M+H)⁺ m/z calculated 638.2. found 638.2.

¹H NMR (DMSO-d₆, 400 MHz): δ 9.06 (s, 1H), 8.43-8.39 (m, 1H), 7.64-7.57 (m, 1H), 7.30-7.22 (m, 2H), 4.94-4.88 (m, 1H), 4.33-4.20 (m, 1H), 4.04-3.98 (m, 1H), 3.94-3.84 (m, 1H), 3.75-3.67 (m, 1H), 3.23-3.15 (m, 2H), 2.75-2.64 (m, 2H), 1.49 (s, 9H), 1.40-1.29 (m, 3H), 1.09-1.03 (m, 6H), 0.90-0.84 (m, 6H).

### Step 2: Synthesis of SZ-014137A2

Compound 014137A1 (250 mg, 0.39 mmol) was dissolved in dichloromethane (6 mL). Trifluoroacetic acid (3 mL) was added. The reaction mixture was stirred at room temperature for 2 h. The reaction mixture was concentrated to give a 014137A2 crude product (260 mg, brown oil, trifluoroacetate salt), which was directly used in the next step. LCMS (M+H)⁺ m/z calculated 538.2. found 538.2.

### Step 3: Synthesis of SZ-014137

Compound 014137A2 (260 mg, 0.39 mmol) was dissolved in anhydrous tetrahydrofuran (10 mL). 2-Fluoroacrylic acid (70 mg, 0.78 mmol) and 2-(7-azobenzotriazole)-N,N,N′,N′-tetramethyluronium hexafluorophosphate (296 mg, 0.78 mmol) were added. N,N-diisopropylethylamine (151 mg, 1.17 mmol) was slowly added to the reaction mixture with stirring at room temperature until pH=9-10. The reaction mixture was stirred at room temperature for 2 h and poured into 30 mL of cold water, followed by extraction with dichloromethane. The extract was concentrated, and the concentrate was purified by preparative high performance liquid chromatography to give compound SZ-014137 as a pale yellow solid (8.0 mg, 4.0% yield).

Liquid chromatography-mass spectrometry [mobile phase: elution was carried out with a gradient from 70% water (containing 0.02% ammonium acetate) and 30% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm. 214 nm. The purity was 97.01%, Rt=3.946 min. LCMS (M+H)⁺ m/z calculated 610.2. found 610.2.

¹H NMR (DMSO-d₆, 400 MHz): δ 9.08 (s, 1H), 8.44 (d, J=8.8 Hz, 1H), 7.65-7.57 (m, 1H), 7.25 (t, J=8.0 Hz, 2H), 5.41-5.26 (m, 2H), 5.01 (br s, 1H), 4.41-4.38 (m, 1H), 4.26-4.00 (m, 2H), 3.87-3.60 (m, 2H), 3.32-3.24 (m, 1H), 2.76-2.69 (m, 2H), 1.38 (d, J=6.4 Hz, 3H), 1.08 (dd, J₁=6.4 Hz, J₂=0.8 Hz, 6H), 0.91 (d, J=6.8 Hz, 6H).

### Comparative Example 2. SZ-014138

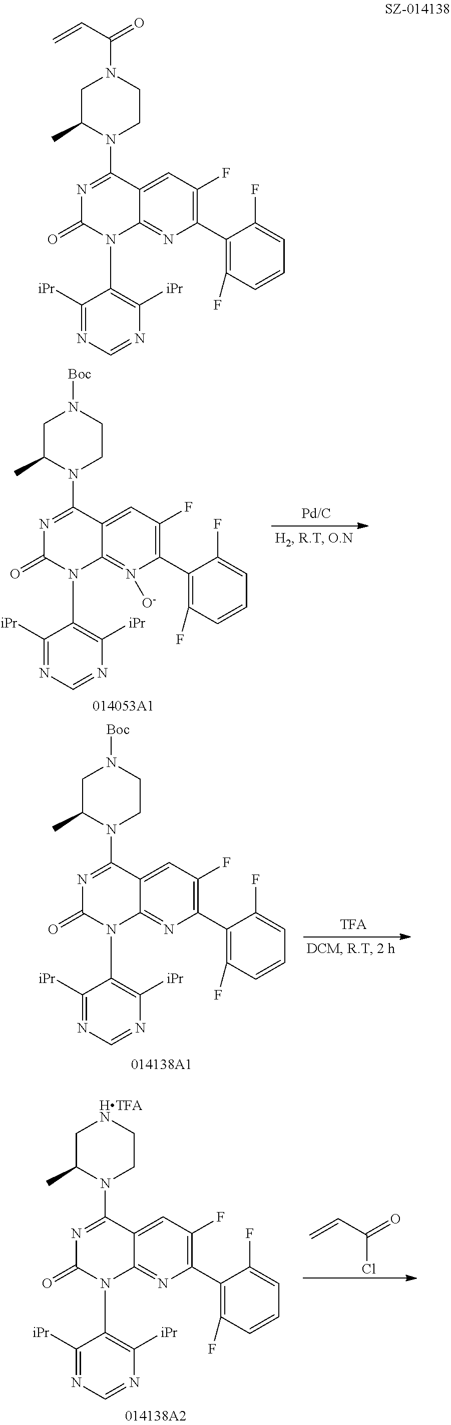

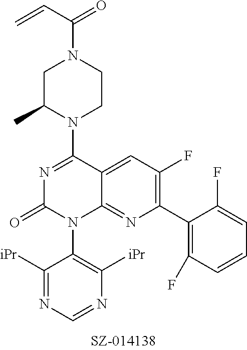 [SMILES: C=CC(=O)N1CCN(c2nc(=O)n(-c3c(C(C)C)ncnc3C(C)C)c3nc(-c4c(F)cccc4F)c(F)cc23)[C@@H](C)C1]

### Step 1: Synthesis of 014138A1

Compound 014053A1 (1.3 g, 2.03 mmol) was dissolved in a solvent mixture of tetrahydrofuran and methanol (200 mL, in a ratio of 1:1). Palladium on carbon (360 mg) was added. The reaction mixture was stirred under hydrogen at normal atmospheric pressure at room temperature overnight. The reaction was filtered, and the filtrate was concentrated to give compound 014138A1 as a pale yellow solid (crude product, 1.2 g, 92% yield), which was directly used in the next step. LCMS (M+H)⁺ m/z calculated 638.2. found 638.2.

### Step 2: Synthesis of SZ-014138A2

Compound 014038A1 (500 mg, 0.785 mmol) was dissolved in dichloromethane (10 mL). Trifluoroacetic acid (3 mL) was added. The reaction mixture was stirred at room temperature for 2 h. The reaction mixture was concentrated to give compound 014138A2 as a brown oil (500 mg, trifluoroacetate salt crude product), which was directly used in the next step. LCMS (M+H)⁺ m/z calculated 538.2. found 538.2.

### Step 3: Synthesis of SZ-014138

Compound 014138A2 (500 mg, 0.33 mmol) was dissolved in dichloromethane (10 mL). Acryloyl chloride (56 mg, 0.628 mmol) was slowly added under an ice bath, followed by diisopropylethylamine (303 mg, 2.355 mmol). After dropwise addition, the reaction phase was stirred at 0° C. for 30 min. Water (20 mL) was added, followed by extraction with dichloromethane (10 mL×3). The organic phases were combined, washed with water (15 mL), dried over anhydrous sodium sulfate, and filtered. The filtrate was concentrated under reduced pressure, and the resulting residue was purified by preparative high performance liquid chromatography (ammonium bicarbonate) to give compound SZ-014138 as a yellow solid (100 mg, 22% yield).

Liquid chromatography-mass spectrometry [mobile phase: elution was carried out with a gradient from 70% water (containing 0.02% ammonium acetate) and 30% acetonitrile to 30% water (containing 0.02% ammonium acetate) and 70% acetonitrile at a flow rate of 1.5 mL per minute at a column temperature of 40° C. for 6 min. Column: waters XBridge C18 3.5 μm, 50×4.6 mm. 214 nm. The purity was 99.4%, Rt=3.823 min. LCMS (M+H)⁺ m/z calculated 592.4. found 592.4.

¹H NMR (DMSO-d₆, 400 MHz): δ 9.07 (s, 1H), 8.47-8.41 (m, 1H), 7.65-7.57 (m, 1H), 7.25 (t, J=8.4 Hz, 2H), 6.92-6.83 (m, 1H), 6.24-6.17 (m, 1H), 5.78 (dd, J₁=10.8 Hz, J₂=2.4 Hz, 1H), 5.00 (br s, 1H), 4.42-4.28 (m, 2H), 4.17-4.03 (m, 1H), 3.82-3.77 (m, 1H), 3.67-3.51 (m, 1H), 3.33-3.15 (m, 1H), 2.73-2.70 (m, 2H), 1.35 (d, J=6.8 Hz, 3H), 1.08 (d, J=6.4 Hz, 6H), 0.91 (d, J=6.4 Hz, 6H).

### Comparative Example 3. SZ-014141

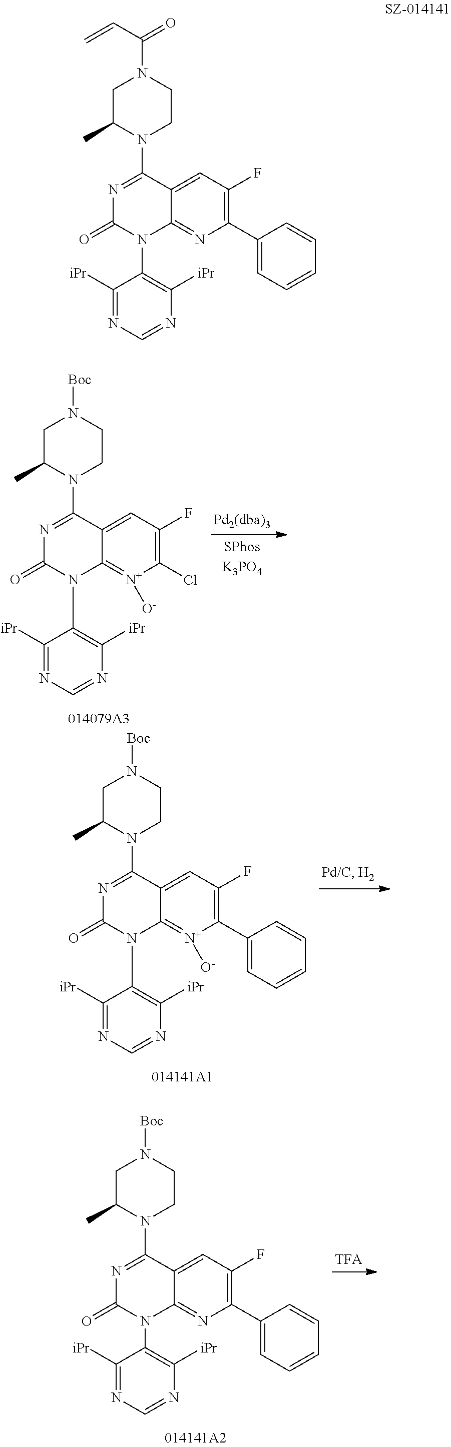 [SMILES: C=CC(=O)N1CCN(c2nc(=O)n(-c3c(C(C)C)ncnc3C(C)C)c3nc(-c4ccccc4)c(F)cc23)[C@@H](C)C1 ; CC(C)c1ncnc(C(C)C)c1-n1c(=O)nc(N2CCN(C(=O)OC(C)(C)C)C[C@@H]2C)c2cc(F)c(Cl)[n+]([O-])c21 ; CC(C)c1ncnc(C(C)C)c1-n1c(=O)nc(N2CCN(C(=O)OC(C)(C)C)C[C@@H]2C)c2cc(F)c(-c3ccccc3)[n+]([O-])c21 ; CC(C)c1ncnc(C(C)C)c1-n1c(=O)nc(N2CCN(C(=O)OC(C)(C)C)C[C@@H]2C)c2cc(F)c(-c3ccccc3)nc21]

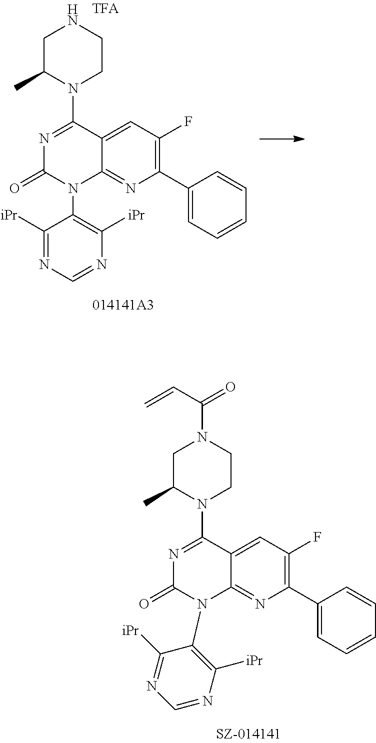 [SMILES: CC(C)c1ncnc(C(C)C)c1-n1c(=O)nc(N2CCNC[C@@H]2C)c2cc(F)c(-c3ccccc3)nc21 ; C=CC(=O)N1CCN(c2nc(=O)n(-c3c(C(C)C)ncnc3C(C)C)c3nc(-c4ccccc4)c(F)cc23)[C@@H](C)C1]

### Step 1: Synthesis of 014141A1

Compound 014079A3 (800 mg, 1.39 mmol), phenylboronic acid (339 mg, 2.78 mmol), potassium phosphate (589 mg, 2.78 mmol) and 2-dicyclohexylphosphine-2′,6′-dimethoxybiphenyl (228 mg, 0.556 mmol) were dissolved in 1,4-dioxane (15 mL). Tris(dibenzylideneacetone)dipalladium (509 mg, 0.556 mmol) was added. The system was purged with nitrogen several times, and then the reaction mixture was stirred at 90° C. overnight. The reaction mixture was cooled to room temperature and concentrated to remove the solvent. Water (30 mL) and ethyl acetate (50 mL) were added, followed by extraction with ethyl acetate (50 mL×3). The organic phases were combined, dried over anhydrous sodium sulfate, and filtered. The filtrate was concentrated under reduced pressure, and the resulting residue was purified by silica gel column chromatography (methanol/dichloromethane=1:30) to give compound 014141A1 as a yellow solid (750 mg, 87.6% yield). LCMS (M+H)⁺ m/z calculated 618.3. found 618.3.

### Step 2: Synthesis of 014141A2

Compound 014141A1 (800 mg, 1.29 mmol) was dissolved in methanol (10 mL). Palladium on carbon (300 mg) was added. The reaction mixture was stirred under hydrogen at normal atmospheric pressure at room temperature overnight. The reaction mixture was filtered, and the resulting filtrate was concentrated to dryness to give compound 014141A2 as a pale yellow solid (crude product, 780 mg, 100% yield). The solid product was directly used in the next step without purification. LCMS (M+H)⁺ m/z calculated 602.3. found 602.3.

### Step 3: Synthesis of 014141A3

Compound 014141A2 (780 mg, 1.29 mmol) was dissolved in dichloromethane (10 mL). Trifluoroacetic acid (5 mL) was added. The reaction mixture was stirred at room temperature for 2 h. The reaction mixture was concentrated to give compound 014141A3 as a brown oil (trifluoroacetate salt crude product, 750 mg, 100% yield), which was directly used in the next step.

LCMS (M+H)⁺ m/z calculated 502.3. found 502.3.

### Step 3: Synthesis of SZ-014141

Compound 014141A3 (750 mg, 1.50 mmol) was dissolved in dichloromethane (10 mL). Acryloyl chloride (135 mg, 1.50 mmol) was slowly added under an ice bath, followed by diisopropylethylamine (580 mg, 4.5 mmol). After dropwise addition, the reaction phase was stirred at 0° C. for 30 min. Water (20 mL) was added, followed by extraction with dichloromethane (10 mL×3). The organic phases were combined, washed with water (15 mL), dried over anhydrous sodium sulfate, and filtered. The filtrate was concentrated under reduced pressure, and the resulting residue was purified by preparative high performance liquid chromatography (ammonium bicarbonate) to give compound SZ-014141 as a yellow solid (260 mg, 31.3% yield).

Liquid chromatography-mass spectrometry [mobile phase: elution was carried out with a gradient from 70% water (containing 0.1% trifluoroacetic acid) and 30% acetonitrile to 30% water (containing 0.1% trifluoroacetic acid) and 70% acetonitrile at a flow rate of 1.5 mL per minute at a column temperature of 40° C. for 6 min. Column: waters XBridge C18 3.5 μm, 50×4.6 mm. 214 nm. The purity was 99.12%, Rt=3.832 min. LCMS (M+H)⁺ m/z calculated 556.3. found 556.3.

¹H NMR (DMSO-d₆, 400 MHz): δ 9.21 (s, 1H), 8.42-8.35 (m, 1H), 7.72-7.70 (m, 2H), 7.51-7.45 (m, 3H), 6.97-6.85 (m, 1H), 6.25 (d, J=21.6 Hz, 1H), 5.80 (dd, J₁=14.0 Hz, J₂=3.2 Hz, 1H), 5.00 (br s, 1H), 4.45-4.31 (m, 2H), 4.19-4.05 (m, 1H), 3.78-3.48 (m, 2H), 3.33-3.12 (m, 1H), 2.76-2.53 (m, 2H), 1.37 (d, J=9.2 Hz, 3H), 1.19-1.08 (m, 6H), 0.95-0.92 (m, 6H).

### Comparative Example 4. SZ-014142

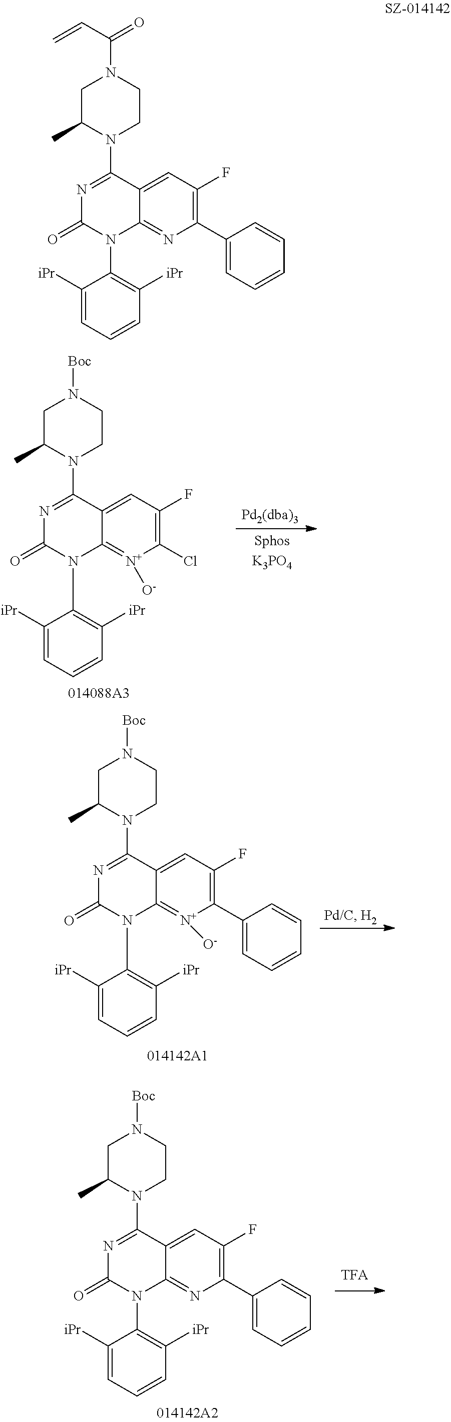

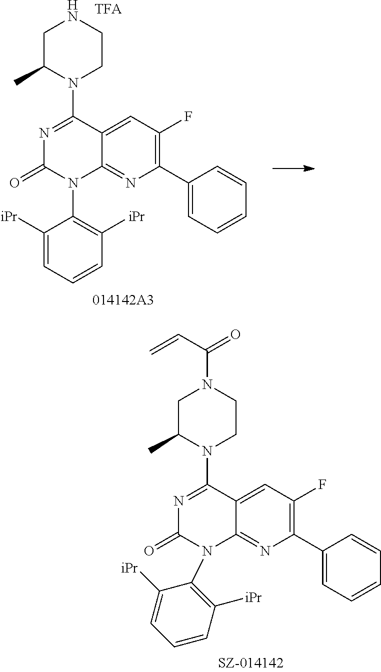 [SMILES: CC(C)c1cccc(C(C)C)c1-n1c(=O)nc(N2CCNC[C@@H]2C)c2cc(F)c(-c3ccccc3)nc21 ; C=CC(=O)N1CCN(c2nc(=O)n(-c3c(C(C)C)cccc3C(C)C)c3nc(-c4ccccc4)c(F)cc23)[C@@H](C)C1]

### Step 1: Synthesis of 014142A1

Compound 014088A3 (500 mg, 0.87 mmol), phenylboronic acid (212 mg, 1.74 mmol), potassium phosphate (368 mg, 1.74 mmol) and 2-dicyclohexylphosphine-2′,6′-dimethoxybiphenyl (142 mg, 0.35 mmol) were dissolved in 1,4-dioxane (10 mL). Tris(dibenzylideneacetone)dipalladium (320 mg, 0.35 mmol) was added. The system was purged with nitrogen several times, and then the reaction mixture was stirred at 90° C. overnight. The reaction mixture was cooled to room temperature and concentrated to remove the solvent. Water (30 mL) and ethyl acetate (40 mL) were added, followed by extraction with ethyl acetate (40 mL×3). The organic phases were combined, dried over anhydrous sodium sulfate, and filtered. The filtrate was concentrated under reduced pressure, and the resulting residue was purified by silica gel column chromatography (methanol/dichloromethane=1:30) to give compound 014142A1 as a yellow solid (450 mg, 84% yield). LCMS (M+H)⁺ m/z calculated 616.3. found 616.3.

### Step 2: Synthesis of -014142A2

Compound 014142A1 (450 mg, 0.73 mmol) was dissolved in methanol (10 mL). Palladium on carbon (200 mg) was added. The reaction mixture was stirred under hydrogen at normal atmospheric pressure at room temperature overnight. The reaction mixture was filtered, and the resulting filtrate was concentrated to dryness to give 014142A2 as a pale yellow solid (crude product, 440 mg, 100% yield), which was directly used in the next step without purification. LCMS (M+H)⁺ m/z calculated 600.3. found 600.3.

### Step 3: Synthesis of 014142A3

Compound 014142A2 (440 mg, 0.73 mmol) was dissolved in dichloromethane (10 mL). Trifluoroacetic acid (4 mL) was added. The reaction mixture was stirred at room temperature for 2 h. The reaction mixture was concentrated to dryness to give 014142A3 as a brown oil (trifluoroacetate salt crude product, 470 mg, 100% yield), which was directly used in the next step. LCMS (M+H)⁺ m/z calculated 500.3. found 500.3.

### Step 3: Synthesis of SZ-014142

The crude compound 014142A3 (470 mg, 0.73 mmol) was dissolved in dichloromethane (6 mL). Acryloyl chloride (66 mg, 0.73 mmol) was slowly added under an ice bath, followed by diisopropylethylamine (282 mg, 2.19 mmol). After dropwise addition, the reaction phase was stirred at 0° C. for 30 min. The reaction mixture was extracted with dichloromethane (10 mL×3). The organic phases were combined, washed with water (15 mL), dried over anhydrous sodium sulfate, and filtered. The filtrate was concentrated under reduced pressure, and the resulting residue was purified by preparative high performance liquid chromatography (ammonium bicarbonate) to give compound SZ-014142 as a yellow solid (170 mg, 42.1% yield).

Liquid chromatography-mass spectrometry [mobile phase: elution was carried out with a gradient from 60% water (containing 0.1% trifluoroacetic acid) and 40% acetonitrile to 5% water (containing 0.1% trifluoroacetic acid) and 95% acetonitrile at a flow rate of 1.5 mL per minute at a column temperature of 40° C. for 6 min. Column: waters Sunfire C18 3.5 μm, 50×4.6 mm. 214 nm. The purity was 99.83%, Rt=3.650 min. LCMS (M+H)⁺ m/z calculated 554.3. found 554.4.

¹H NMR (DMSO-d₆, 400 MHz): δ 8.36-8.29 (m, 1H), 7.72 (d, J=10.4 Hz, 2H), 7.50-7.41 (m, 4H), 7.34 (d, J=10.0 Hz, 2H), 6.97-6.85 (m, 1H), 6.24 (d, J=22.4 Hz, 1H), 5.80 (dd, J₁=13.6 Hz, J₂=2.8 Hz, 1H), 4.95 (br s, 1H), 4.46-4.31 (m, 2H), 4.21-4.04 (m, 1H), 3.76-3.52 (m, 2H), 3.33-3.12 (m, 1H), 2.52-2.47 (m, 2H), 1.35 (d, J=8.8 Hz, 3H), 1.10 (dd, J₁=9.2 Hz, J₂=2.4 Hz, 6H), 0.90-0.86 (m, 6H).

### Comparative Example 5. SZ-014139

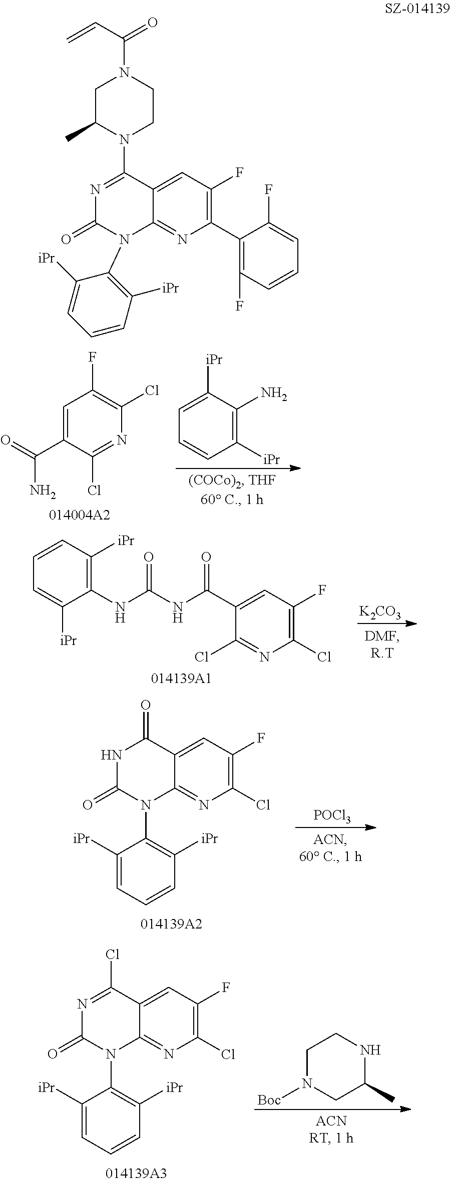

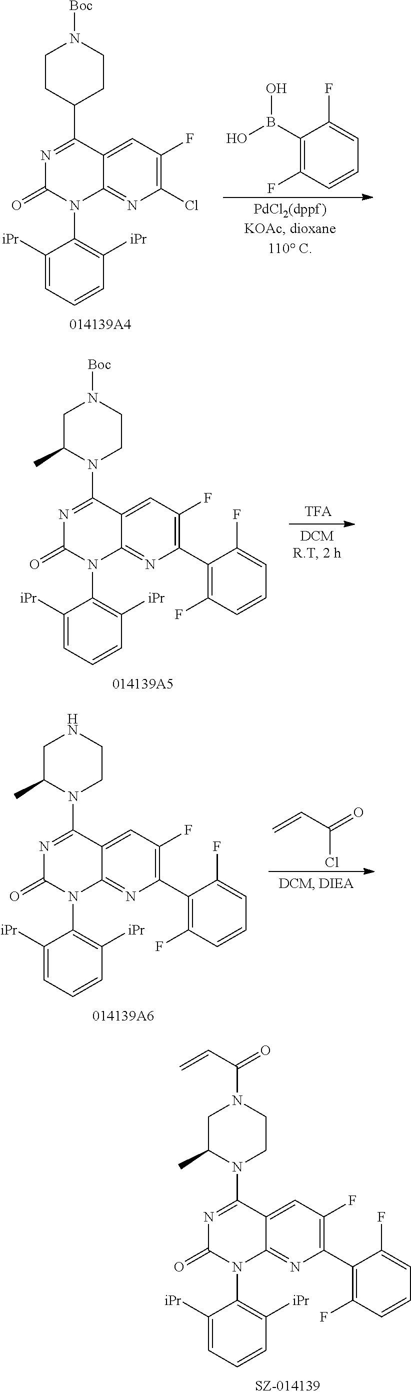

### Step 1: Synthesis of 014139A1

Compound 014004A2 (8.5 g, 49.42 mmol) was dissolved in tetrahydrofuran (10 mL). Oxalyl chloride (7.5 g, 59.30 mmol) was added at 0° C. The reaction mixture was stirred at 60° C. for 1 h, and removed from the heat to cool to room temperature. A solution of compound 2,6-diisopropylaniline (8.7 g, 49.42 mmol) in tetrahydrofuran (30 mL) was added dropwise at 0° C. The reaction mixture was stirred at 0° C. for 1 h. The reaction mixture was quenched with saturated aqueous sodium bicarbonate solution (50 mL) and then extracted with ethyl acetate (100 mL×2). The organic phases were combined, dried over anhydrous magnesium sulfate, and filtered. The filtrate was concentrated to give crude compound 014139A1 as a yellow solid (21.7 g, crude product).

LCMS (M+H⁺) m/z calculated 412.1. found 412.1.

### Step 2: Synthesis of 014139A2

The crude compound 014139A1 (21.7 g, 49.42 mmol) was dissolved in N,N-dimethylformamide (200 mL). Potassium carbonate (20.4 g, 148.26 mmol) was added at room temperature. The reaction mixture was stirred at room temperature overnight. Water (600 mL) was added. The resulting mixture was adjusted to pH 6-7 with 1 N HCl solution and extracted with ethyl acetate (300 mL×2). The organic phases were combined, washed with saturated brine (200 mL×2), dried over anhydrous magnesium sulfate, and filtered under vacuum. The filtrate was concentrated, and the residue was purified by column chromatography (ethyl acetate:petroleum ether=10:1 to 5:1) to give compound 014139A2 as a pale yellow solid (5.3 g, 28.6% yield). LCMS (M+H⁺) m/z calculated 376.1. found 376.1.

¹H NMR (DMSO-d₆, 400 MHz): δ 12.20 (s, 1H), 8.47 (d, J=7.2 Hz, 1H), 7.46 (d, J=8.0 Hz, 1H), 7.32 (d, J=7.6 Hz, 2H), 2.66-2.63 (m, 2H), 1.09 (d, J=6.8 Hz, 6H), 0.95 (d, J=6.4 Hz, 6H).

### Step 3: Synthesis of 014139A3

Compound 014139A2 (4.2 g, 11.17 mmol) was dissolved in anhydrous acetonitrile (50 mL). Phosphorus oxychloride (5.2 g, 33.51 mmol) and N,N-diisopropylethylamine (4.3 g, 33.51 mmol) were added under an ice bath. After dropwise addition, the reaction mixture was stirred at 60° C. for 1 h, cooled to room temperature and concentrated to give compound 014139A3 as a brown oil (5.2 g, crude product), which was used in the next step without purification. LCMS (M+H⁺) m/z calculated 394.1. found 394.1.

### Step 4: Synthesis of 014139A4

The brown oily crude compound 014139A3 (5.2 g, 11.17 mmol) was dissolved in anhydrous acetonitrile (50 mL). N,N-diisopropylethylamine (4.3 g, 33.51 mmol) and (S)-4-N-tert-butoxycarbonyl-2-methylpiperazine (4.68 g, 23.4 mmol) were added. The reaction mixture was stirred at room temperature for 1 h. Ethyl acetate (150 mL) was then added, followed by washing with saturated brine (100 mL). The aqueous phase was extracted with ethyl acetate (100 mL×2). The organic phases were combined, dried over anhydrous sodium sulfate, and filtered. The residue was purified by silica gel column chromatography (petroleum ether/ethyl acetate=4:1) to give compound 014139A4 as a yellow solid (5.0 g, 77% yield).

LCMS (M+H⁺) m/z calculated 558.3. found 558.3.

### Step 5: Synthesis of 014139A5

Compound 014139A4 (1.0 g, 1.8 mmol) and 2,6-difluorophenylboronic acid (1.42 g, 9.0 mmol) were dissolved in 1,4-dioxane (50 mL). Potassium acetate (882 mg, 9.0 mmol) and PdCl₂(dppf) (197 mg, 0.27 mmol) were added. The system was purged with nitrogen three times, and then the reaction mixture was stirred at 110° C. overnight, cooled to room temperature, and filtered. The filtrate was diluted with ethyl acetate (100 mL) and washed with saturated brine (100 mL). The organic phase was dried over anhydrous sodium sulfate and filtered. The filtrate was concentrated under reduced pressure, and the resulting residue was purified by silica gel column chromatography (petroleum ether/ethyl acetate=4:1) to give compound 014139A5 as a yellow solid (500 mg, 45% yield).

LCMS (M+H⁺) m/z calculated 636.3. found 636.3.

### Step 6: Synthesis of 014139A6

Compound 014139A5 (500 mg, 0.79 mmol) was dissolved in dichloromethane (10 mL). Trifluoroacetic acid (3 mL) was added under an ice bath. The ice bath was removed, and the reaction mixture was stirred at room temperature (20° C.) for 2 h, and concentrated to give compound 014139A6 as a yellow solid, which was directly used in the next step without purification. (500 mg, crude product). LCMS (M+H)⁺ m/z calculated 536.3. found m/z 536.3.

### Step 7: Synthesis of SZ-014139

The crude compound 014139A6 (500 mg, 0.0.79 mmol) was dissolved in dichloromethane (20 mL). Acryloyl chloride (71 mg, 0.79 mmol) and N,N-diisopropylethylamine (305 mg, 2.37 mmol) were added under an ice bath. The reaction mixture was stirred under the ice bath for 30 min. The reaction mixture was washed with saturated sodium bicarbonate (20 mL). The organic phase was separated and concentrated to dryness by rotary evaporation, and the residue was purified by preparative high performance liquid chromatography (ammonium bicarbonate) to give compound SZ-014139 as a yellow solid (100 mg, 21% yield).

Liquid chromatography-mass spectrometry [mobile phase: elution was carried out with a gradient from 70% water (containing 0.02% ammonium acetate) and 30% acetonitrile to 5% water (containing 0.02% ammonium acetate) and 95% acetonitrile at a flow rate of 1.5 mL per minute at a column temperature of 40° C. for 6 min. Column: waters Sunfire C18 3.5 μm, 50×4.6 mm]. The purity was 99.10%, Rt=3.726 min.

LCMS (M+H)⁺ m/z calculated 590.3. found 590.3.

¹H NMR (DMSO-d₆, 400 MHz): δ 8.45-8.39 (m, 1H), 7.67-7.57 (m, 1H), 7.38-7.33 (m, 1H), 7.28-7.23 (m, 4H), 6.94-6.85 (m, 1H), 6.27-6.21 (m, 1H), 5.82-5.78 (m, 1H), 6.97 (br s, 1H), 4.46-4.04 (m, 3H), 3.74-3.53 (m, 2H), 3.29-3.16 (m, 1H), 2.54-2.48 (m, 2H), 1.36 (d, J=9.2 Hz, 3H), 1.08 (d, J=8.8 Hz, 6H), 0.91 (d, J=8.4 Hz, 6H).

### Experiment on Biological Activities

Assay for Binding Rate to KRAS G12C Protein by LC-MS Method

A 10 mM stock solution of a test compound in DMSO was prepared. The KRAS G12C protein was diluted with a buffer (20 mM Hepes, pH 7.5, 50 mM NaCl, 0.5 mM MgCl₂) to 103 μM. An equal volume of GDP buffer (20 mM Hepes, pH 7.5, 50 mM NaCl, 0.5 mM MgCl₂, 10 mM EDTA, 2 mM DTT, GDP) was added to prepare KRASG12C protein loaded with GDP.

The KRASG12C protein loaded with GDP was diluted to 20 μM by adding a dilution solution (12.5 mM Hepes, pH 7.5, 75 mM NaCl, 10 mM MgCl₂). A reaction system was prepared from the following components: GDP-KRAS-4B-G12C (20 μM, 5 μL), a test compound (10% DMSO, 5 μL), a buffer (125 mM Hepes, pH 7.5, 750 mM NaCl, 10 mM MgCl2; 5 μL), and purified water (35 μL). After incubation at room temperature for 5 min and 30 min, the reaction was terminated by addition of 5 μL of 5% formic acid. After being centrifuged at 15,000 rpm for 10 min, the mixture was subjected to LC-MS analysis and data analysis. The parameters for LC and MS are shown in Tables 2 and 3, respectively.

TABLE 1
UPLC conditions

| | Parameter | |
|---|---|---|
| Device information | Waters Acquity I Class | |
| Chromatography column | Sepax Bio-C4, 2.1 × 50 mm, 3 μm | |
| Flowrate | 0.6 | ml/min |
| Sample load | 10 | μL |

| | |
|---|---|
| Mobile phase | A: 0.1% formic Acid in water |
| | B: 0.1% Formic acid in CAN |
| Column temperature | 65° C. |
| Detector | TOF MS |

TABLE 2
LC time gradient settings

| Time (min) | A (%) | B (%) |
|---|---|---|
| 0 | 95 | 5 |
| 0.75 | 95 | 5 |
| 1 | 75 | 25 |
| 6 | 50 | 50 |
| 6.25 | 0 | 100 |
| 7.5 | 0 | 100 |
| 7.75 | 95 | 5 |
| 9 | 95 | 5 |

TABLE 3
TOFMS parameters are as follows:

| | Parameter |
|---|---|
| Device information | Xevo G2-XS Qtof |
| Capilary (kV) | 4 |
| Sampling Cone(V) | 60 |
| Source temperature (° C.) | 120 |
| Cone Gas (L/h) | 50 |
| Desolvation Gas (L/h) | 1000 |
| Interface Type | ES, Postitive |
| Analyser Mode | Sensitivity |
| Scan Range | 500-2000 m/z |

Scan Range 500-2000 m/z

The percent binding of a test compound to KRASG12C protein was calculated as: Percent binding to KRAS G12C (%)=peak height of conjugate of test compound and KRAS G12C protein/[peak height of conjugate of test compound and KRAS G12C protein+peak height of free KRAS G12C protein] x 100. Specific bioanalytical data are shown in Table 4.

In-Cell Western Blot Assay for ERK Phosphorylation in H358 Cells

H358 cells were thawed and pre-cultured for 3 days to be in a good state (RPMI 1640+10% FBS+1% P/S). The cells were plated on to a 384-well plate, and a test compound, positive control compounds (AMG510 and isomers thereof) and a negative control were added. The compound was at concentrations from 10000 nM to 0.051 nM. 3-fold dilution was performed. The mixture was well mixed and incubated at 37° C. and 5% CO₂. The cells were washed with PBS and suspended in methanol. After another washing with PBS, a blocking buffer was added. After 1 h of blocking at room temperature, a primary antibody mixture (rabbit anti pERK, mouse anti GAPDH) was added, followed by overnight incubation at 4° C. The cells were washed 3 times with PBST, and a secondary antibody mixture (goat anti rabbit 800CW and goat anti mouse 680RD) was added, followed by incubation at room temperature in the dark. The 384-well plate was reversed and centrifuged at 1000 rpm for 1 min, and read on an Odyssey CLx fluorescence imaging system for fluorescence signal values, which was corrected using DMSO and ARS1620. The specific calculation is as follows:

Relative Signal=Signal Value(total channel 800)/Signal Value(total channel 700)

H=Ave (DMSO)

L=Ave (ARS1620)

SD (H)=STDEV (DMSO)

SD (L)=STDEV (ARS1620)

CV % (DMSO)=100×(SD_H/Ave_H)

CV % (ARS1620)=100×SD_L/Ave_L

Z′=1-3×(SD_H+SD_L)/(Ave_H−Ave_L)

Relative pERK=(Sample−Ave_L)/(Ave_H−Ave_L).

The IC₅₀ values of the compounds were calculated using a four-parameter fitting algorithm, specifically as follows:

Y=Bottom+(Top−Bottom)/(1+10\hat{ }((Log IC50−X)×HillSlope))

X: Log of cpd concentration

Y: Ave (relative pERK)

Top and Bottom: Plateaus in same units as Y

log IC50: same log units as X

HillSlope: Slope factor or Hill slope.

Specific bioanalytical data are shown in Table 4.

TABLE 4
Bioanalytical data

| | | Binding rate to | | |
|---|---|---|---|---|
| | | KRAS G12C protein | | H358 |
| | | (LC-MS POC, %) | | p-ERK |
| | | 3 uM/ | 3 uM/ | IC₅₀, |
| Examples | Compound No. | 5 min | 30 min | nM |
| | SZ-014004A | 95 | 96 | 12 |
| | (AMG510) | | | |
| | SZ-014004B | 20 | 75 | 146 |
| | (isomer of | | | |
| | AMG510) | | | |
| 1 | SZ-014088A | 94.8 | 98.1 | 14 |
| 1 | SZ-014088B | 11.2 | 12.4 | 3510 |
| 2 | SZ-014089A/B | — | — | 143 |
| 3 | SZ-014010A/B | 47.7 | 84.9 | 441.50 |
| 4 | SZ-014011A/B | 37.8 | 75.5 | 404.58 |
| 5 | SZ-014079A | 100 | 100 | 18 |
| 5 | SZ-014079B | 4.6 | 14.5 | 7095 |
| 6 | SZ-014016A | — | — | 296.21 |
| 8 | SZ-014041A | — | — | 576.60 |
| 9 | SZ-014043 | 58.5 | 90.0 | 493.80 |
| 10 | SZ-014044 | 84.1 | 97.2 | 145.31 |
| 11 | SZ-014013A/B | — | — | 36 |
| 12 | SZ-014031A | — | — | 11.4 |
| 13 | SZ-014051A/B | — | — | 44 |
| 14 | SZ-014053 | 85.6 | 100.0 | 41 |
| 16 | SZ-014032A | — | — | 2738 |
| 16 | SZ-014032B | — | — | 388 |
| 17 | SZ-014066A | — | — | 7.8 |
| 17 | SZ-014066B | — | — | 2075 |
| 18 | SZ-014077A/B | — | — | 191 |
| 19 | SZ-014082A/B | — | — | 60 |
| 20 | SZ-014108A/B | — | — | 9.4 |
| 21 | SZ-014111A/B | — | — | 31 |
| 22 | SZ-014089P1A/B | — | — | 71 |
| 23 | SZ-014062AB | — | — | 207.1 |
| 24 | SZ-014114 | 78.5 | 98.7 | 32 |
| 25 | SZ-014119 | 20.8 | 79.2 | 691 |
| 26 | SZ-014094 | — | — | 8.3 |
| 27 | SZ-014116AB | — | — | 2.8 |
| 28 | SZ-014129 | — | — | 90 |
| 29 | SZ-014130 | — | — | 139 |
| 30 | SZ-014131 | — | — | 2087 |
| 31 | SZ-014136 | — | — | 4401 |
| Comparative | SZ-014137 | | | 3535 |
| Example 1 | | | | |
| Comparative | SZ-014138 | | | 34 |
| Example 2 | | | | |
| Comparative | SZ-014141 | | | 170 |
| Example 3 | | | | |
| Comparative | SZ-014142 | | | 296 |
| Example 4 | | | | |
| Comparative | SZ-014139 | | | 81 |
| Example 5 | | | | |

Example 41 of WO2018217651A1 discloses the structure of AMG510 as shown below:

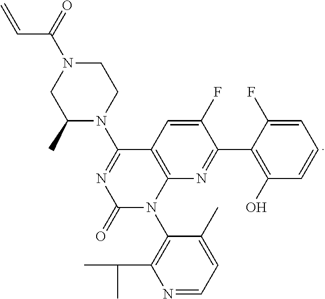 [SMILES: C=CC(=O)N1CCN(c2nc(=O)n(-c3c(C)ccnc3C(C)C)c3nc(-c4c(O)cccc4F)c(F)cc23)[C@@H](C)C1]

Human (H), Rat (R) and Mouse (M) Plasma Protein Binding (PPB)

A test compound and a positive control were each mixed with blank plasma to a final concentration of 1 μM. The mixture was added to a RED plate plasma sample chamber, followed by addition of a dialysis buffer in the buffer chamber. The sample was prepared in triplicate for each compound. The chambers were sealed, and the plate was incubated at 37° C. with shaking at 60 rpm for 5 h. After incubation, the samples were transferred out of the plasma chamber and the buffer chamber and treated as shown in Table 5. After centrifugation at 5594 g for 15 min, the supernatant was collected and analyzed by LC/MS/MS.

TABLE 5
Treatment conditions for PPB samples

| | | From | | Volume of addition (μL) | | |
|---|---|---|---|---|---|---|
| | | plasma | From | | | Internal |
| | | sample | buffer | | | standard |
| | Type of | chamber | chamber | Blank | | working |
| Test group | sample | (μL) | (μL) | plasma | PBS | solution |
| Compound | Plasma | 25 | / | / | 25 | 200 |
| | sample | | | | | |
| | Buffer | / | 25 | 25 | / | 200 |
| | sample | | | | | |
| Positive | Plasma | 25 | / | / | 25 | 200 |
| control | sample | | | | | |
| | Buffer | / | 25 | 25 | / | 200 |
| | sample | | | | | |

The compound concentration in each sample was expressed in terms of the ratio between peak areas (ratio of peak area of compound to peak area of internal standard) by mass spectrometry analysis, and the plasma protein binding was calculated according to the following formulas.

% free state=(buffer chamber peak area ratio/plasma chamber peak area ratio)×100%

% bound state=100%−% free state

The PPB assay data are shown in Table 6.

TABLE 6
PPB assay data

| | | PPB (% free state) |
|---|---|---|
| Examples | Compound No. | H/R/M |
| | SZ-014004A | 6.8/14.5/11.2 |
| | (AMG510) | |
| 5 | SZ-014079A | 75.5/52.6/35.7 |
| 14 | SZ-014053 | 58.7/61.6/37.7 |
| 24 | SZ-014114 | 12.5/16.9/4.4 |
| 26 | SZ-014094 | 53.2/57.1/19.8 |
| 28 | SZ-014129 | 45.5/51.7/33.6 |
| 29 | SZ-014130 | 7.4/18.8/3.9 |
| 31 | SZ-014136 | 51.2/52.8/16.1 |
| Comparative | SZ-014137 | 7.6/11.4/0.5 |
| Example 1 | | |
| Comparative | SZ-014138 | 12.9/20.3/1.1 |
| Example 2 | | |
| Comparative | SZ-014141 | 4.0/12.7/0.8 |
| Example 3 | | |
| Comparative | SZ-014142 | 0.2/0.8/4.7 |
| Example 4 | | |
| Comparative | SZ-014139 | 1.5/2.2/0.7 |
| Example 5 | | |

In human plasma, the molar concentration of the compound SZ-014053 of the present invention not bound to plasma proteins is no less than 4 times higher than that of the reference compound SZ-014138 not bound to plasma proteins, and the in vitro drug effects of the two compounds are substantially the same, indicating that the therapeutically effective concentration of compound SZ-014053 in vivo is higher and thus the compound is superior in treating diseases. Similarly, where the in vitro drug effects are substantially the same, in human plasma, the molar concentration of the compound SZ-014129 of the present invention not bound to plasma proteins is no less than 10 times higher than that of the reference compound SZ-014141 not bound to plasma proteins; in human plasma, the molar concentration of the compound SZ-014130 of the present invention not bound to plasma proteins is no less than 30 times higher than that of the reference compound SZ-014142 not bound to plasma proteins; in human plasma, the molar concentration of the compound SZ-014136 of the present invention not bound to plasma proteins is no less than 6 times higher than that of the reference compound SZ-014137 not bound to plasma proteins; in human plasma, the molar concentration of the compound SZ-014114 of the present invention not bound to plasma proteins is no less than 8 times higher than that of the reference compound SZ-014139 not bound to plasma proteins.

1. A compound having a structure of formula I or a pharmaceutically acceptable salt, ester, isomer, solvate, prodrug or isotopically labeled compound thereof: 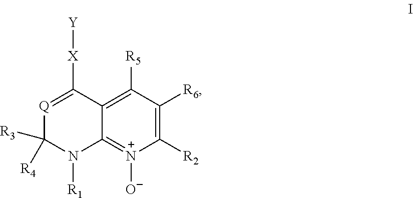
wherein,
R₁ is selected from C₆₋₁₀ aryl and 5- to 10-membered heteroaryl unsubstituted or substituted with R₇;
R₂ is selected from C₆₋₁₀ aryl and 5- to 10-membered heteroaryl unsubstituted or substituted with R₈;
R₃ and R₄ are each independently selected from hydrogen, deuterium, and C₁₋₆ alkyl, or R₃ and R₄ are joined to form 3- to 7-membered cycloalkyl or 3- to 7-membered heterocycloalkyl unsubstituted or optionally substituted with 1-3 substituents selected from deuterium, halogen, hydroxy and C₁₋₆ alkyl, or R₃ and R₄ form ═O, S ═N—CN or ═CH₂;
R₅ and R₆ are each independently selected from hydrogen, deuterium and halogen;
each R₇ and R₈ are independently selected from hydrogen, deuterium, cyano, halogen, hydroxy, amino, C₁₋₆ alkyl, —NHC₁₋₆ alkyl, —N(C₁₋₆ alkyl)₂, C₃₋₄ cycloalkyl, C₁₋₄ alkoxy, C₂₋₆ alkenyl, C₂₋₆ alkynyl and —COOC₁₋₆ alkyl, wherein the amino, alkyl, cycloalkyl, alkenyl, and alkynyl are unsubstituted or substituted with 1-3 substituents selected from halogen, hydroxy, amino, acetyl and deuterium atom;
X is a 4- to 9-membered heterocyclyl unsubstituted or substituted with R₉, wherein each R₉ is independently selected from hydrogen, deuterium, cyano, halogen, hydroxy, amino, C₁₋₆ alkyl and C₁₋₆ alkoxy, wherein the amino and alkyl are unsubstituted or substituted with 1-3 substituents selected from halogen, cyano, hydroxy, amino and deuterium atom;
Y is 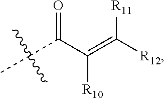
wherein R₁₀, R₁₁ and R₁₂ are each independently selected from hydrogen, deuterium, halogen, cyano, C₁₋₆ alkyl, C₃₋₆ cycloalkyl, 3- to 7-membered heterocyclyl, C₂₋₆ alkenyl, C₂₋₆ alkynyl, acetyl, propionyl, butyryl, and —COOC₁₋₆ alkyl, wherein the alkyl, cycloalkyl, alkenyl, alkynyl, acetyl, propionyl and butyryl are unsubstituted or substituted with 1-3 substituents selected from deuterium, halogen, cyano, hydroxy, amino, C₁₋₆ alkyl, —NHC₁₋₆ alkyl, —N(C₁₋₆ alkyl)₂ and 3- to 7-membered heterocyclyl; or R₁₀ and R₁₂ are joined to each other to form a triple bond;
Q is N or C-Q′, wherein Q′ is selected from hydrogen, deuterium, cyano, halogen and C₁₋₆ alkyl.

2. The compound or the pharmaceutically acceptable salt, ester, isomer, solvate, prodrug or isotopically labeled compound thereof according to claim 1, wherein Q is N.

3. The compound or the pharmaceutically acceptable salt, ester, isomer, solvate, prodrug or isotopically labeled compound thereof according to claim 1, wherein R₁ is selected from C₆₋₁₀ aryl and 5- to 10-membered heteroaryl unsubstituted or substituted with R₇, wherein the substitution with R₇ occurs at an ortho position of the atom in the C₆₋₁₀ aryl and 5- to 10-membered heteroaryl connected to the N atom.

4. The compound or the pharmaceutically acceptable salt, ester, isomer, solvate, prodrug or isotopically labeled compound thereof according to claim 1, wherein R₁ is selected from C₆₋₁₀ aryl and 5- to 6-membered heteroaryl unsubstituted or substituted with R₇, wherein the 5- to 6-membered heteroaryl contains 1-3 heteroatoms or heteroatom groups selected from N, O, S(O)ₘ, wherein m is an integer from 0 to 2.

5. The compound or the pharmaceutically acceptable salt, ester, isomer, solvate, prodrug or isotopically labeled compound thereof according to claim 1, wherein R₁ is selected from C₆₋₁₀ aryl and 5- to 10-membered heteroaryl unsubstituted or substituted with R₇, wherein the C₆₋₁₀ aryl is selected from phenyl, naphthyl, tetrahydronaphthyl and 2,3-dihydroindenyl; the 5- to 10-membered heteroaryl is selected from thienyl, pyridinyl, pyridine N-oxide group, pyrimidinyl, pyrazinyl, pyridazinyl, pyridonyl, pyrazinonyl, pyrimidinonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuranyl, quinolinyl, isoquinolinyl and quinazolinyl; preferably, the C₆₋₁₀ aryl is phenyl; the 5- to 10-membered heteroaryl is selected from pyridinyl and pyrimidinyl.

6. The compound or the pharmaceutically acceptable salt, ester, isomer, solvate, prodrug or isotopically labeled compound thereof according to claim 1, wherein each R₇ is independently selected from hydrogen, deuterium, cyano, halogen, hydroxy, amino, C₁₋₆ alkyl, C₃₋₆ cycloalkyl and C₁₋₆ haloalkyl.

7. The compound or the pharmaceutically acceptable salt, ester, isomer, solvate, prodrug or isotopically labeled compound thereof according to claim 6, wherein each R₇ is independently selected from hydrogen, deuterium, methyl, CH₂F, CHF₂, CF₃, ethyl, propyl, isopropyl, butyl, sec-butyl, isobutyl, tert-butyl, cyclopropyl, cyclobutyl, cyclopentyl and cyclohexyl.

8. The compound or the pharmaceutically acceptable salt, ester, isomer, solvate, prodrug or isotopically labeled compound thereof according to claim 7, wherein R₂ is selected from C₆₋₁₀ aryl and 5- to 6-membered heteroaryl unsubstituted or substituted with R₈, wherein the 5- to 6-membered heteroaryl contains 1-3 heteroatoms or heteroatom groups selected from N, O, S(O)ᵣ, wherein r is an integer from 0 to 2.

9. The compound or the pharmaceutically acceptable salt, ester, isomer, solvate, prodrug or isotopically labeled compound thereof according to claim 1, wherein R₂ is selected from C₆₋₁₀ aryl and 5- to 10-membered heteroaryl unsubstituted or substituted with R₈, wherein the C₆₋₁₀ aryl is selected from phenyl, naphthyl, tetrahydronaphthyl and 2,3-dihydroindenyl; the 5- to 10-membered heteroaryl is selected from thienyl, pyridinyl, pyridine N-oxide group, pyrimidinyl, pyrazinyl, pyridazinyl, pyridonyl, pyrazinonyl, pyrimidinonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, benzothienyl, indolyl, benzimidazolyl, benzothiazolyl, benzofuranyl, quinolinyl, isoquinolinyl and quinazolinyl.

10. The compound or the pharmaceutically acceptable salt, ester, isomer, solvate, prodrug or isotopically labeled compound thereof according to claim 9, wherein R₂ is selected from phenyl, imidazolyl, pyrrolyl, pyridine N-oxide group, pyridinyl, pyridonyl, naphthyl, quinolinyl, isoquinolinyl and quinazolinyl unsubstituted or substituted with R₈.

11. The compound or the pharmaceutically acceptable salt, ester, isomer, solvate, prodrug or isotopically labeled compound thereof according to claim 1, wherein each R₈ is independently selected from hydrogen, deuterium, cyano, halogen, hydroxy, amino, C₁₋₆ alkyl, —NHC₁₋₆ alkyl, —N(C₁₋₆ alkyl)₂ and C₁₋₆ alkoxy, wherein the amino and alkyl are unsubstituted or substituted with 1-3 substituents selected from halogen, hydroxy, amino, acetyl and deuterium atom.

12. The compound or the pharmaceutically acceptable salt, ester, isomer, solvate, prodrug or isotopically labeled compound thereof according to claim 1, wherein each R₈ is independently selected from hydrogen, deuterium, fluorine, chlorine, hydroxy and amino.

13. The compound or the pharmaceutically acceptable salt, ester, isomer, solvate, prodrug or isotopically labeled compound thereof according to claim 1, wherein R₃ and R₄ are independently selected from hydrogen, deuterium and C₁₋₆ alkyl, or R₃ and R₄ are joined to form cyclopropyl, or R₃ and R₄ form ═O, ═S or ═N—CN.

14. The compound or the pharmaceutically acceptable salt, ester, isomer, solvate, prodrug or isotopically labeled compound thereof according to claim 13, wherein R₃ and R₄ form ═O.

15. The compound or the pharmaceutically acceptable salt, ester, isomer, solvate, prodrug or isotopically labeled compound thereof according to claim 1, wherein R₅ and R₆ are each independently selected from hydrogen, deuterium, fluorine and chlorine.

16. The compound or the pharmaceutically acceptable salt, ester, isomer, solvate, prodrug or isotopically labeled compound thereof according to claim 1, wherein X is a 4- to 9-membered heterocyclyl unsubstituted or substituted with R₉, wherein the atom in the 4- to 9-membered heterocyclyl connected to Y is N.

17. The compound or the pharmaceutically acceptable salt, ester, isomer, solvate, prodrug or isotopically labeled compound thereof according to claim 1, wherein X is a 4- to 9-membered heterocyclyl unsubstituted or substituted with R₉, wherein the 4- to 9-membered heterocyclyl includes a monocyclic ring, a fused ring, a bridged ring and a spiro ring.

18. The compound or the pharmaceutically acceptable salt, ester, isomer, solvate, prodrug or isotopically labeled compound thereof according to claim 1, wherein X is a 6- to 7-membered heterocyclyl unsubstituted or substituted with R₉, wherein the 6- to 7-membered heterocyclyl does not contain a double bond or contains 1 or 2 double bonds.

19. The compound or the pharmaceutically acceptable salt, ester, isomer, solvate, prodrug or isotopically labeled compound thereof according to claim 16, wherein X is a 6- to 7-membered heterocyclyl unsubstituted or substituted with R₉, wherein the 6- to 7-membered heterocyclyl is selected from

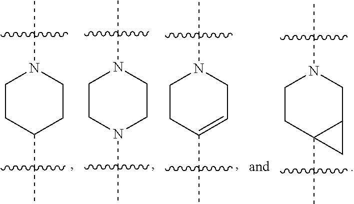

20. The compound or the pharmaceutically acceptable salt, ester, isomer, solvate, prodrug or isotopically labeled compound thereof according to claim 1, wherein each R₉ is independently selected from hydrogen, deuterium, methyl, ethyl, —CH₂OH, —CH₂CN and —CH₂F.

21. The compound or the pharmaceutically acceptable salt, ester, isomer, solvate, prodrug or isotopically labeled compound thereof according to claim 1, wherein X is the following groups:

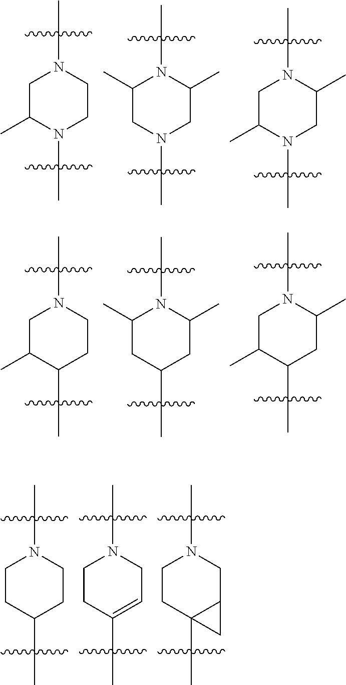

22. The compound or the pharmaceutically acceptable salt, ester, isomer, solvate, prodrug or isotopically labeled compound thereof according to claim 1, wherein Y is 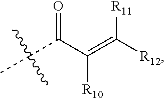
wherein R₁₀ is selected from hydrogen, deuterium and fluorine, Ru is selected from hydrogen and deuterium, and R₁₂ is selected from hydrogen, deuterium, acetyl, dimethylaminomethyl, piperidinyl and aminocyclopropyl; preferably, Y is selected from

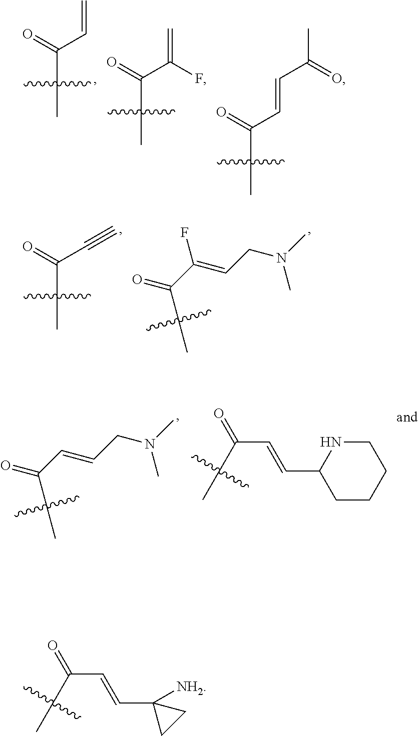

23. The compound or the pharmaceutically acceptable salt, ester, isomer, solvate, prodrug or isotopically labeled compound thereof according to claim 1, wherein
R₁ is selected from C₆₋₁₀ aryl and 5- to 10-membered heteroaryl unsubstituted or substituted with 1-3 R₇;
R₂ is selected from C₆₋₁₀ aryl and 5- to 10-membered heteroaryl unsubstituted or substituted with 1-3 R₈;
R₃ and R₄ are each independently selected from hydrogen, deuterium and C₁₋₆ alkyl, or R₃ and R₄ are joined to form cyclopropyl, or R₃ and R₄ form ═O;
R₅ and R₆ are each independently selected from hydrogen, deuterium and halogen;
each R₇ is independently selected from hydrogen, deuterium, cyano, halogen, hydroxy, amino, C₁₋₆ alkyl and C₃₋₆ cycloalkyl;
each Rₛ is independently selected from hydrogen, deuterium, cyano, halogen, hydroxy, amino, C₁₋₆ alkyl, —NHC₁₋₆ alkyl, —N(C₁₋₆ alkyl)₂ and C₁₋₆ alkoxy, wherein the amino and alkyl are unsubstituted or substituted with 1-3 substituents selected from halogen, hydroxy, amino, acetyl and deuterium atom;
X is a 6- to 7-membered heterocyclic ring unsubstituted or substituted with 1-3 R₉, wherein the atom in the 6- to 7-membered heterocyclic ring connected to Y is N, and each R₉ is independently selected from hydrogen, deuterium, methyl, ethyl, —CH₂OH, —CH₂CN and —CH₂F;
Y is 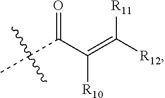
wherein R₁₀ is selected from hydrogen, deuterium and fluorine, R₁₁ is selected from hydrogen and deuterium, and R₁₂ is selected from acetyl, dimethylaminomethyl, piperidinyl and aminocyclopropyl;
Q is N or C-Q′, wherein Q′ is selected from hydrogen, deuterium and cyano.

24. The compound or the pharmaceutically acceptable salt, ester, isomer, solvate, prodrug or isotopically labeled compound thereof according to claim 23, wherein
R₁ is selected from C₆₋₁₀ aryl and 5- to 10-membered heteroaryl unsubstituted or substituted with 1-3 R₇, wherein the C₆₋₁₀ aryl is selected from phenyl, naphthyl, tetrahydronaphthyl and 2,3-dihydroindenyl; the 5- to 10-membered heteroaryl is selected from thienyl, pyridinyl, pyridine N-oxide group, pyrimidinyl, pyrazinyl, pyridazinyl, pyridonyl, pyrazinonyl, pyrimidinonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuranyl, quinolinyl, isoquinolinyl and quinazolinyl;
R₂ is selected from C₆₋₁₀ aryl and 5- to 10-membered heteroaryl unsubstituted or substituted with 1-3 R₈, wherein the C₆₋₁₀ aryl is selected from phenyl, naphthyl, tetrahydronaphthyl and 2,3-dihydroindenyl; the 5- to 10-membered heteroaryl is selected from thienyl, pyridinyl, pyridine N-oxide group, pyrimidinyl, pyrazinyl, pyridazinyl, pyridonyl, pyrazinonyl, pyrimidinonyl, pyridazinonyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuranyl, quinolinyl, isoquinolinyl and quinazolinyl;
R₃ and R₄ form ═O;
R₅ and R₆ are each independently selected from hydrogen, deuterium, chlorine and fluorine;
each R₇ is independently selected from hydrogen, deuterium, methyl, ethyl, propyl, isopropyl, butyl, sec-butyl, isobutyl, tert-butyl, cyclopropyl, cyclobutyl, cyclopentyl and cyclohexyl;
each R₈ is independently selected from hydrogen, deuterium, fluorine, chlorine, hydroxy and amino;
X is a 6- to 7-membered heterocyclic ring unsubstituted or substituted with 1-2 R₉, wherein the 6- to 7-membered heterocyclic ring is selected from 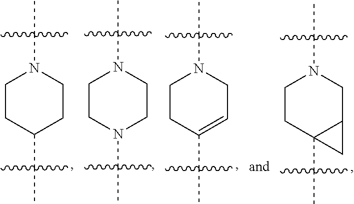
and each R₉ is independently selected from hydrogen, deuterium, methyl, ethyl, —CH₂CN, —CH₂OH and —CH₂F;
Y is 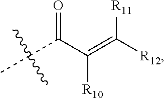
wherein R₁₀ is selected from hydrogen, deuterium and fluorine, R₁₁ is selected from hydrogen and deuterium, and R₁₂ is selected from hydrogen, deuterium, acetyl, dimethylaminomethyl, piperidinyl and aminocyclopropyl;
Q is N or C-Q′, wherein Q′ is selected from hydrogen, deuterium and cyano.

25. The compound or the pharmaceutically acceptable salt, ester, isomer, solvate, prodrug or isotopically labeled compound thereof according to claim 1, wherein the compound of formula I has a structure of formula I-A, I-B, I-C, I-D, I-E or I-F: 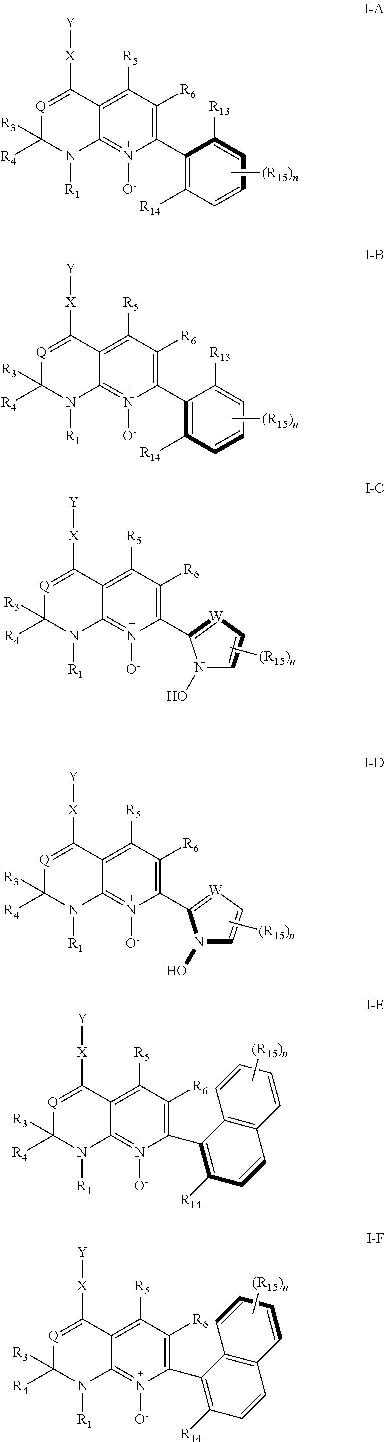
wherein R₁₃ and each R₁₅ are independently selected from hydrogen, deuterium, cyano, halogen, hydroxy, amino, C₁₋₆ alkyl, —NHC₁₋₆ alkyl, —N(C₁₋₆ alkyl)₂ and C₁₋₄ alkoxy, wherein the amino and alkyl are unsubstituted or substituted with 1-3 substituents selected from halogen, hydroxy, amino, acetyl and deuterium atom; n is an integer from 0 to 3; R₁₄ is selected from hydrogen, deuterium, fluorine, hydroxy and amino; W is selected from N, CH, CCH₃, CC₂H₅ and CCH(CH₃)₂.

26. The compound or the pharmaceutically acceptable salt, ester, isomer, solvate, prodrug or isotopically labeled compound thereof according to claim 25, wherein the compound of formula I has a structure of formula I-A or I-B, wherein n is 0; one of R₁₃ and R₁₄ is hydrogen, and the other is hydroxy or F, or R₁₃ and R₁₄ are both hydroxy or F, or one of R₁₃ and R₁₄ is hydroxy and the other is F; preferably, R₁₃ and R₁₄ are both hydroxy or F, or one of R₁₃ and R₁₄ is hydroxy, and the other is F; more preferably, one of R₁₃ and R₁₄ is hydroxy, and the other is F.

27. A compound or a pharmaceutically acceptable salt, ester, hydrate, solvate, stereoisomer, tautomer, cis-trans isomer, isotopically labeled compound or prodrug thereof, wherein the compound is any one of:

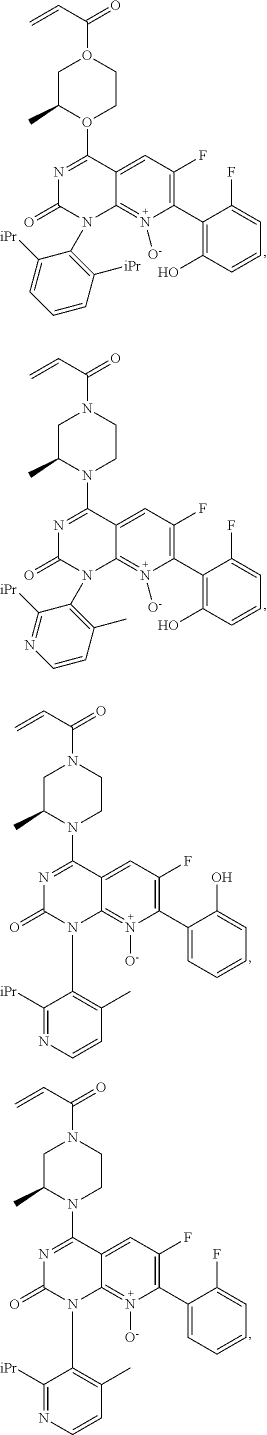

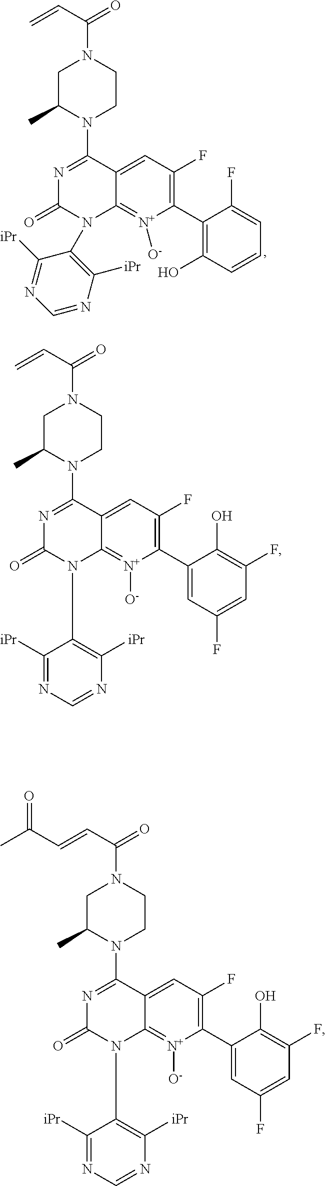

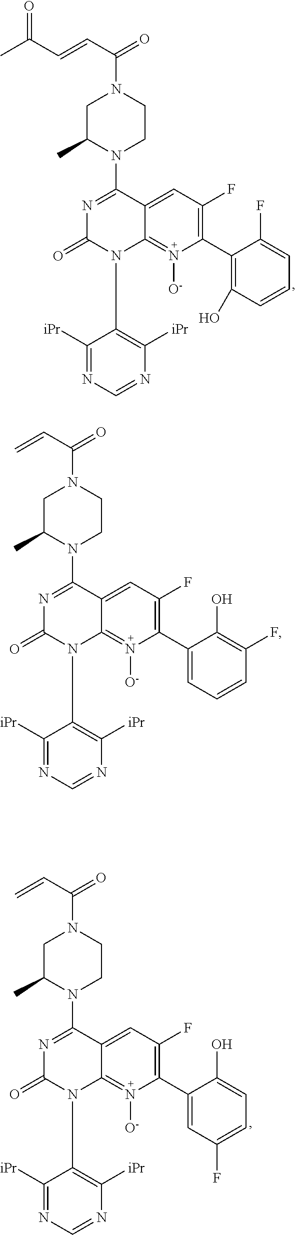

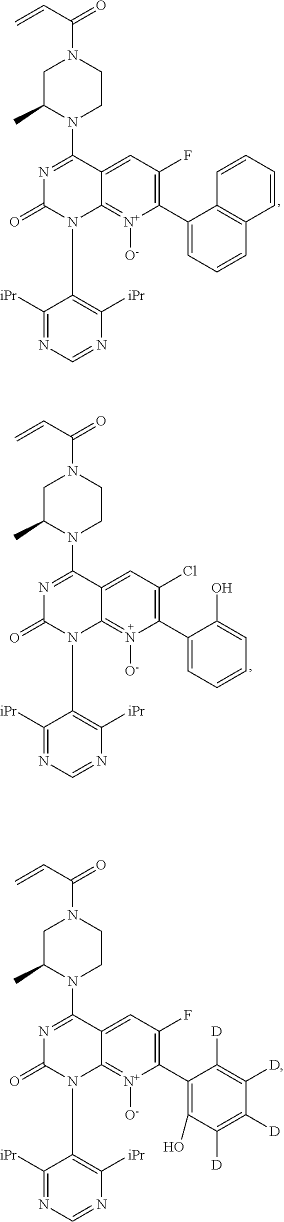

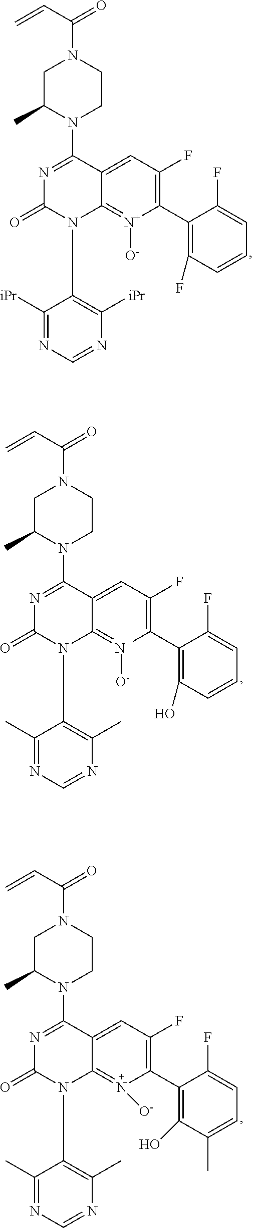

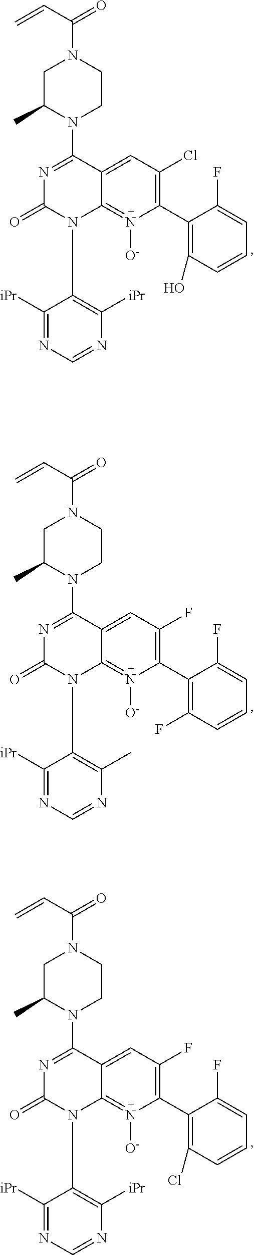

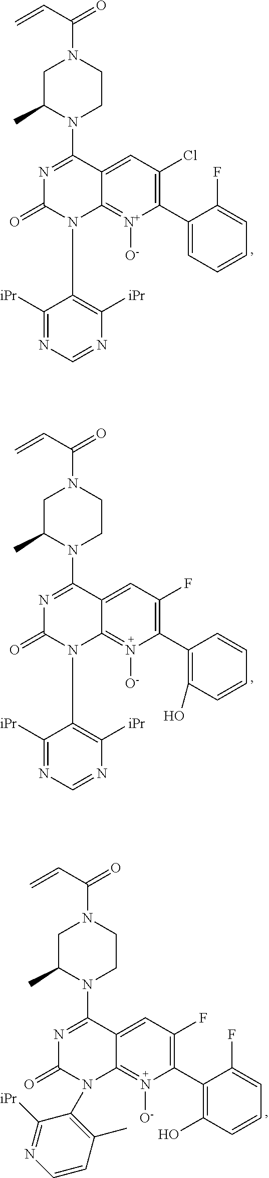

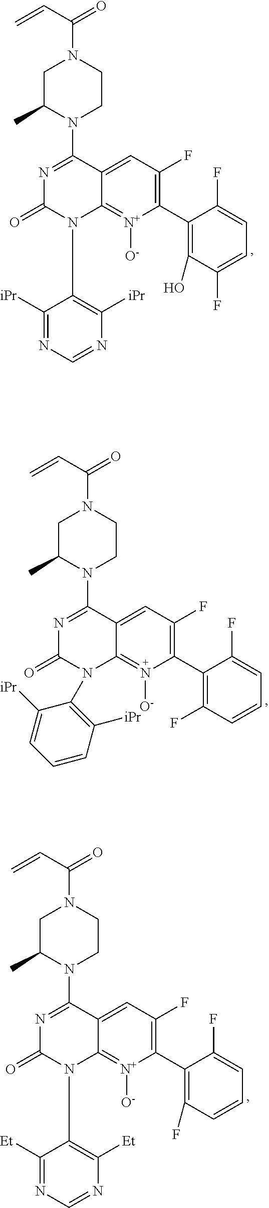

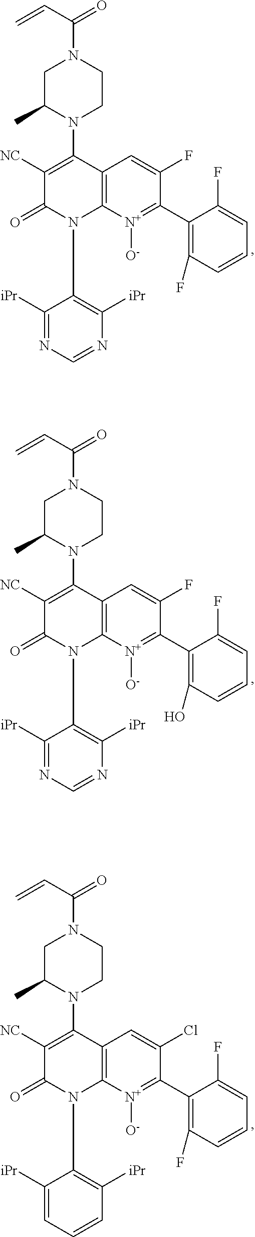 [SMILES: C=CC(=O)N1CCN(c2c(C#N)c(=O)n(-c3c(C(C)C)ncnc3C(C)C)c3c2cc(F)c(-c2c(F)cccc2F)[n+]3[O-])[C@@H](C)C1 ; C=CC(=O)N1CCN(c2c(C#N)c(=O)n(-c3c(C(C)C)ncnc3C(C)C)c3c2cc(F)c(-c2c(O)cccc2F)[n+]3[O-])[C@@H](C)C1 ; C=CC(=O)N1CCN(c2c(C#N)c(=O)n(-c3c(C(C)C)cccc3C(C)C)c3c2cc(Cl)c(-c2c(F)cccc2F)[n+]3[O-])[C@@H](C)C1]

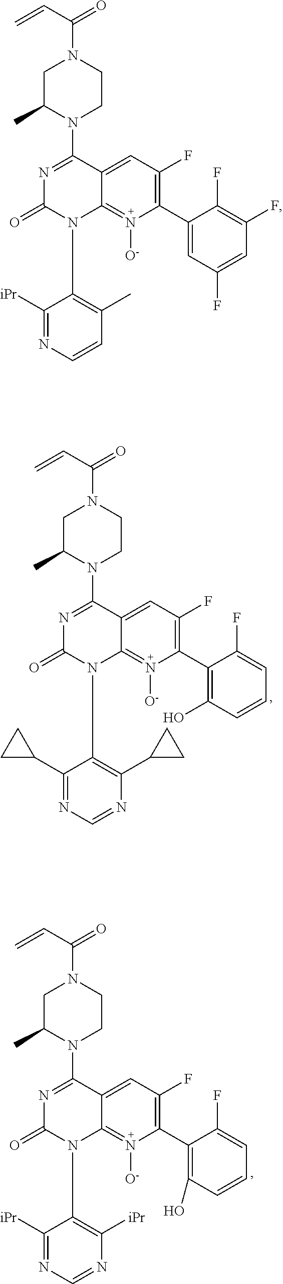

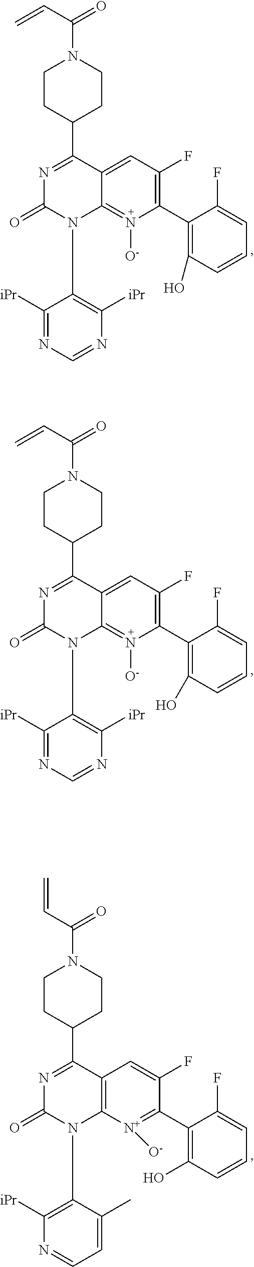

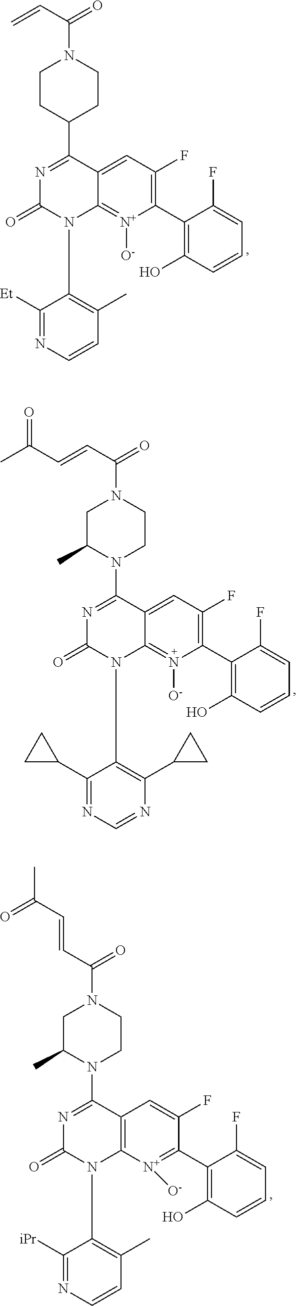

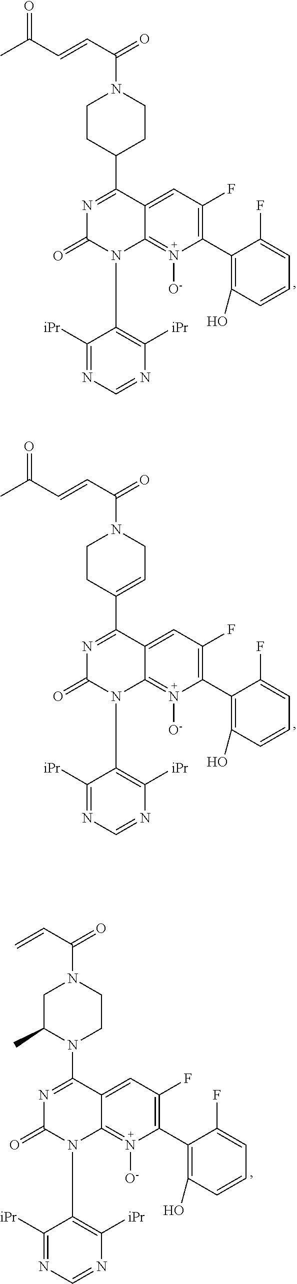

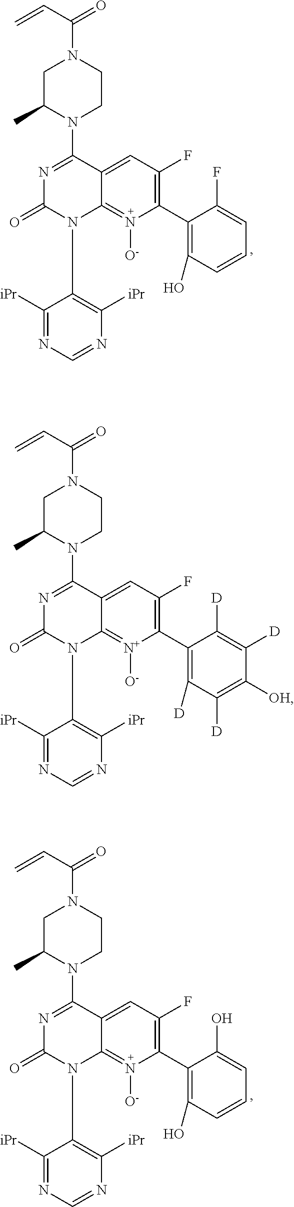

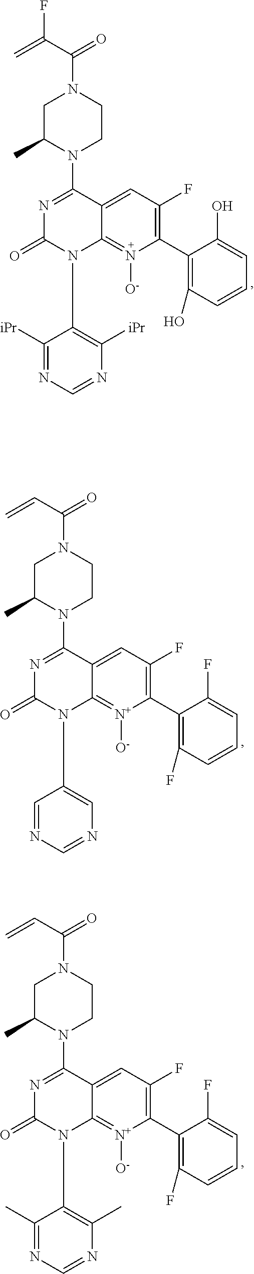

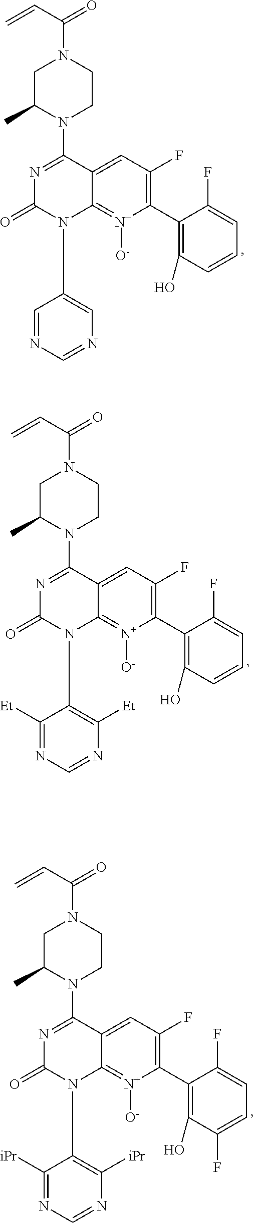 [SMILES: C=CC(=O)N1CCN(c2nc(=O)n(-c3cncnc3)c3c2cc(F)c(-c2c(O)cccc2F)[n+]3[O-])[C@@H](C)C1 ; C=CC(=O)N1CCN(c2nc(=O)n(-c3c(CC)ncnc3CC)c3c2cc(F)c(-c2c(O)cccc2F)[n+]3[O-])[C@@H](C)C1 ; C=CC(=O)N1CCN(c2nc(=O)n(-c3c(C(C)C)ncnc3C(C)C)c3c2cc(F)c(-c2c(F)ccc(F)c2O)[n+]3[O-])[C@@H](C)C1]

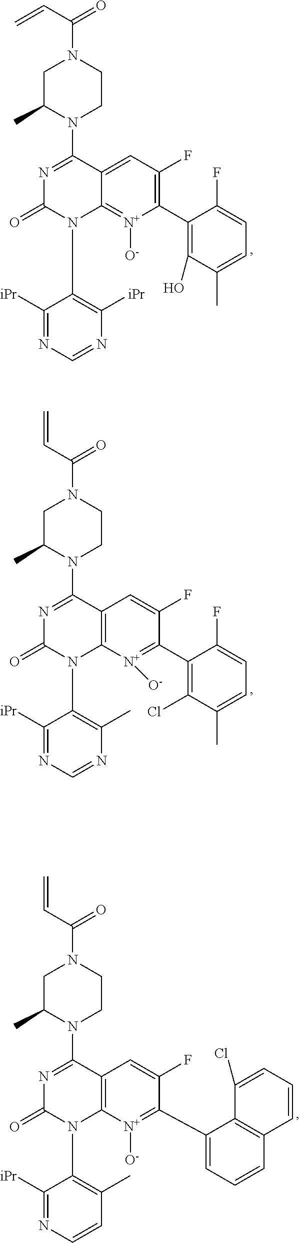 [SMILES: C=CC(=O)N1CCN(c2nc(=O)n(-c3c(C(C)C)ncnc3C(C)C)c3c2cc(F)c(-c2c(F)ccc(C)c2O)[n+]3[O-])[C@@H](C)C1 ; C=CC(=O)N1CCN(c2nc(=O)n(-c3c(C)ncnc3C(C)C)c3c2cc(F)c(-c2c(F)ccc(C)c2Cl)[n+]3[O-])[C@@H](C)C1 ; C=CC(=O)N1CCN(c2nc(=O)n(-c3c(C)ccnc3C(C)C)c3c2cc(F)c(-c2cccc4cccc(Cl)c24)[n+]3[O-])[C@@H](C)C1]

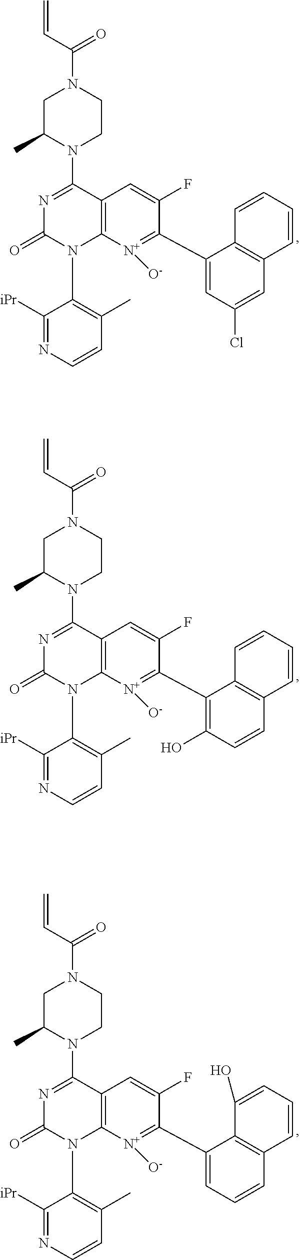 [SMILES: C=CC(=O)N1CCN(c2nc(=O)n(-c3c(C)ccnc3C(C)C)c3c2cc(F)c(-c2cc(Cl)cc4ccccc24)[n+]3[O-])[C@@H](C)C1 ; C=CC(=O)N1CCN(c2nc(=O)n(-c3c(C)ccnc3C(C)C)c3c2cc(F)c(-c2c(O)ccc4ccccc24)[n+]3[O-])[C@@H](C)C1 ; C=CC(=O)N1CCN(c2nc(=O)n(-c3c(C)ccnc3C(C)C)c3c2cc(F)c(-c2cccc4cccc(O)c24)[n+]3[O-])[C@@H](C)C1]

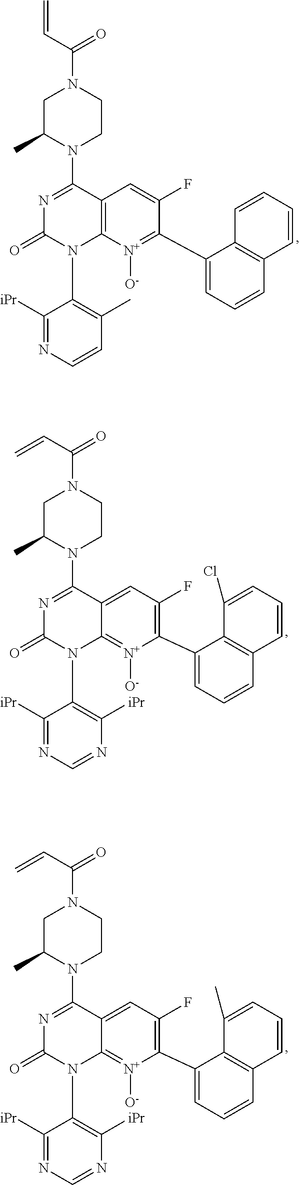 [SMILES: C=CC(=O)N1CCN(c2nc(=O)n(-c3c(C)ccnc3C(C)C)c3c2cc(F)c(-c2cccc4ccccc24)[n+]3[O-])[C@@H](C)C1 ; C=CC(=O)N1CCN(c2nc(=O)n(-c3c(C(C)C)ncnc3C(C)C)c3c2cc(F)c(-c2cccc4cccc(Cl)c24)[n+]3[O-])[C@@H](C)C1 ; C=CC(=O)N1CCN(c2nc(=O)n(-c3c(C(C)C)ncnc3C(C)C)c3c2cc(F)c(-c2cccc4cccc(C)c24)[n+]3[O-])[C@@H](C)C1]

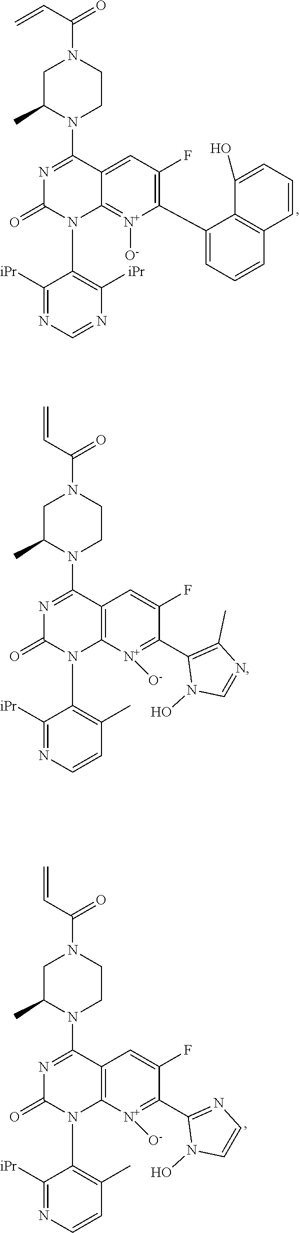 [SMILES: C=CC(=O)N1CCN(c2nc(=O)n(-c3c(C(C)C)ncnc3C(C)C)c3c2cc(F)c(-c2cccc4cccc(O)c24)[n+]3[O-])[C@@H](C)C1 ; C=CC(=O)N1CCN(c2nc(=O)n(-c3c(C)ccnc3C(C)C)c3c2cc(F)c(-c2c(C)ncn2O)[n+]3[O-])[C@@H](C)C1 ; C=CC(=O)N1CCN(c2nc(=O)n(-c3c(C)ccnc3C(C)C)c3c2cc(F)c(-c2nccn2O)[n+]3[O-])[C@@H](C)C1]

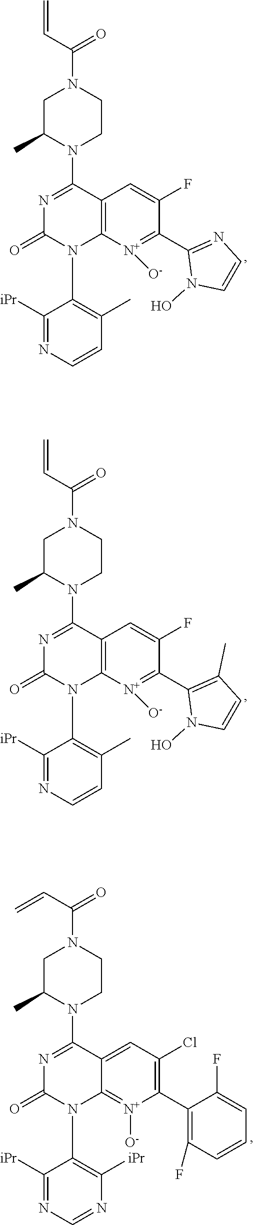 [SMILES: C=CC(=O)N1CCN(c2nc(=O)n(-c3c(C)ccnc3C(C)C)c3c2cc(F)c(-c2nccn2O)[n+]3[O-])[C@@H](C)C1 ; C=CC(=O)N1CCN(c2nc(=O)n(-c3c(C)ccnc3C(C)C)c3c2cc(F)c(-c2c(C)ccn2O)[n+]3[O-])[C@@H](C)C1 ; C=CC(=O)N1CCN(c2nc(=O)n(-c3c(C(C)C)ncnc3C(C)C)c3c2cc(Cl)c(-c2c(F)cccc2F)[n+]3[O-])[C@@H](C)C1]

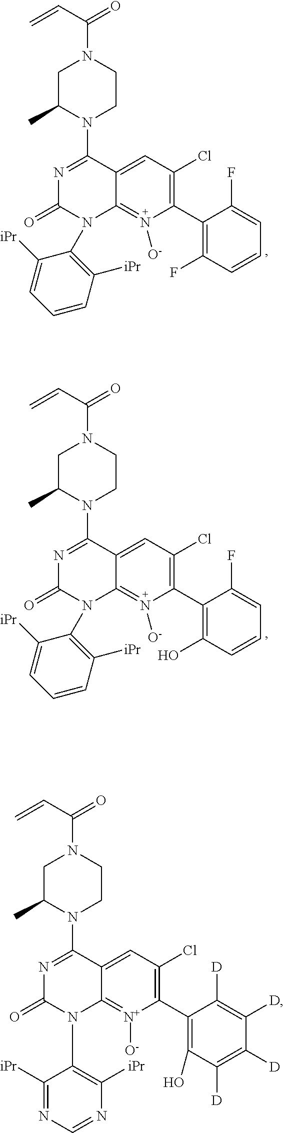

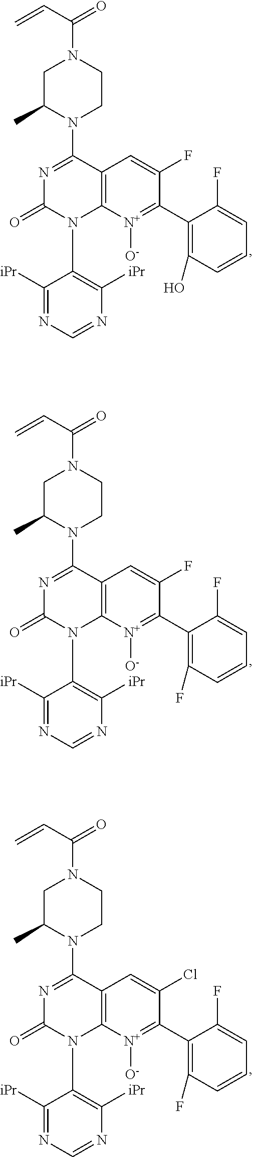 [SMILES: C=CC(=O)N1CCN(c2nc(=O)n(-c3c(C(C)C)ncnc3C(C)C)c3c2cc(F)c(-c2c(O)cccc2F)[n+]3[O-])[C@@H](C)C1 ; C=CC(=O)N1CCN(c2nc(=O)n(-c3c(C(C)C)ncnc3C(C)C)c3c2cc(F)c(-c2c(F)cccc2F)[n+]3[O-])[C@@H](C)C1 ; C=CC(=O)N1CCN(c2nc(=O)n(-c3c(C(C)C)ncnc3C(C)C)c3c2cc(Cl)c(-c2c(F)cccc2F)[n+]3[O-])[C@@H](C)C1]

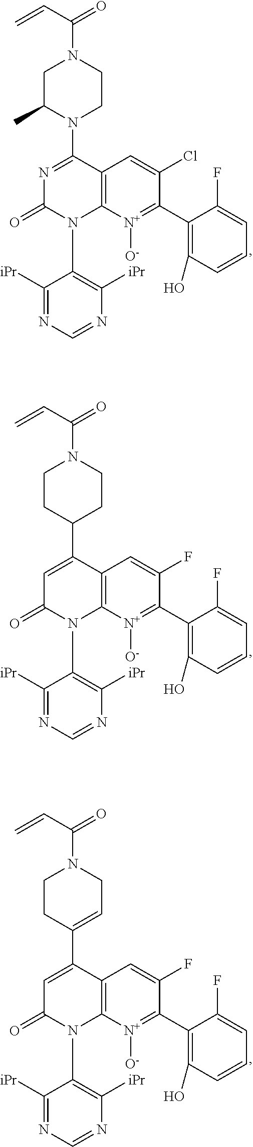 [SMILES: C=CC(=O)N1CCN(c2nc(=O)n(-c3c(C(C)C)ncnc3C(C)C)c3c2cc(Cl)c(-c2c(O)cccc2F)[n+]3[O-])[C@@H](C)C1 ; C=CC(=O)N1CCC(c2cc(=O)n(-c3c(C(C)C)ncnc3C(C)C)c3c2cc(F)c(-c2c(O)cccc2F)[n+]3[O-])CC1 ; C=CC(=O)N1CC=C(c2cc(=O)n(-c3c(C(C)C)ncnc3C(C)C)c3c2cc(F)c(-c2c(O)cccc2F)[n+]3[O-])CC1]

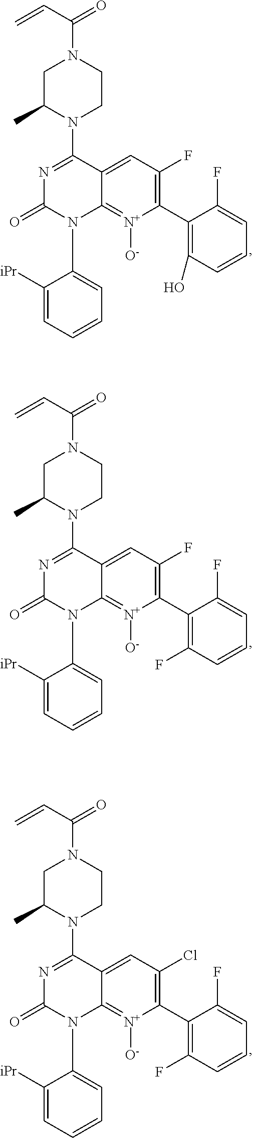 [SMILES: C=CC(=O)N1CCN(c2nc(=O)n(-c3ccccc3C(C)C)c3c2cc(F)c(-c2c(O)cccc2F)[n+]3[O-])[C@@H](C)C1 ; C=CC(=O)N1CCN(c2nc(=O)n(-c3ccccc3C(C)C)c3c2cc(F)c(-c2c(F)cccc2F)[n+]3[O-])[C@@H](C)C1 ; C=CC(=O)N1CCN(c2nc(=O)n(-c3ccccc3C(C)C)c3c2cc(Cl)c(-c2c(F)cccc2F)[n+]3[O-])[C@@H](C)C1]

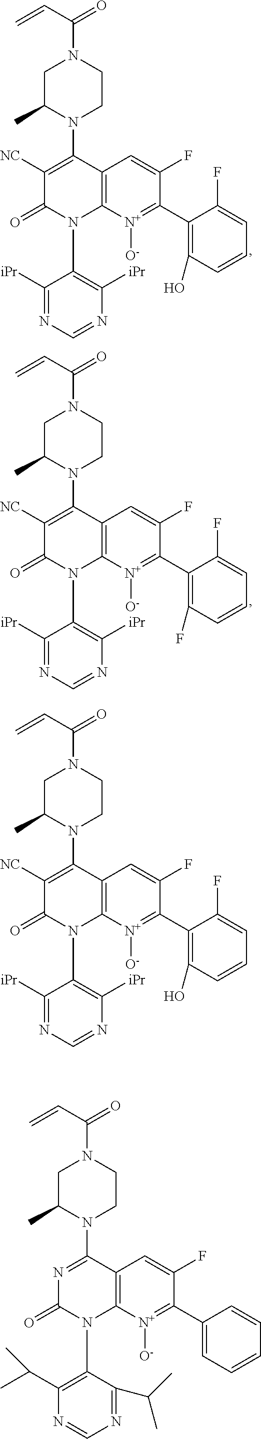

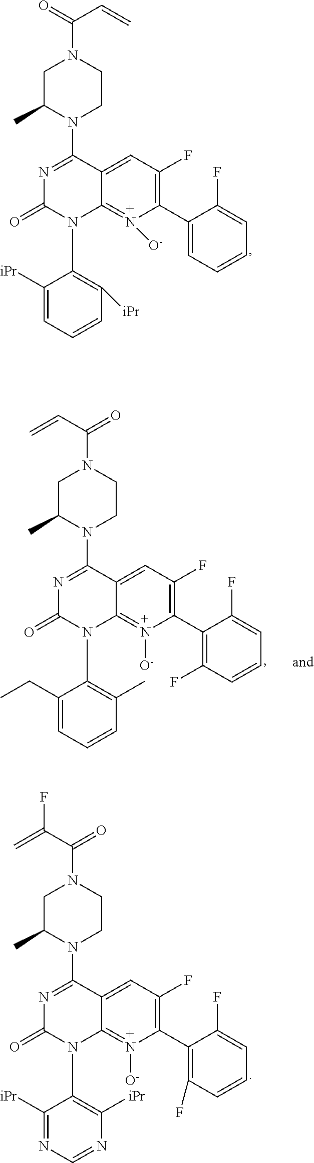

28. A pharmaceutical composition comprising the compound or the pharmaceutically acceptable salt, ester, isomer, solvate, hydrate, prodrug or isotopiccally labeled compound thereof according to claim 1.

29. Use of the compound or the pharmaceutically acceptable salt, ester, hydrate, solvate, stereoisomer, tautomer, cis-trans isomer, isotopically labeled compound or prodrug thereof according to claim 1 or the pharmaceutical composition according to claim 28 in preparing a medicament for preventing and/or treating a KRAS G12C-mediated disease.

30. The use according to claim 29, wherein the disease includes lung cancer, pancreatic cancer, pancreatic ductal carcinoma, colon cancer, rectal cancer, appendiceal cancer, esophageal squamous carcinoma, head and neck squamous carcinoma and breast cancer.
